(19) United States
(12) Patent Application Publication
(10) Pub. No.: US 2021/0296591 A1
(43) Pub. Date: Sep. 23, 2021

(54) ORGANIC ELECTROLUMINESCENCE DEVICE AND ELECTRONIC APPLIANCE USING THE SAME

(71) Applicant: IDEMITSU KOSAN CO.,LTD., Tokyo (JP)

(72) Inventors: Yuki NAKANO, Sodegaura-shi (JP); Satomi TASAKI, Sodegaura-shi (JP); Kazuki NISHIMURA, Sodegaura-shi (JP); Tomoki KATO, Sodegaura-shi (JP)

(73) Assignee: IDEMITSU KOSAN CO.,LTD., Tokyo (JP)

(21) Appl. No.: 17/251,339

(22) PCT Filed: Jun. 14, 2019
(86) PCT No.: PCT/JP2019/023611

§ 371 Date: Dec. 11, 2020

(30) Foreign Application Priority Data
Jun. 15, 2018 (JP) 2018-114580
Sep. 26, 2018 (JP) 2018-180465
Dec. 5, 2018 (JP) 2018-228047
Jan. 30, 2019 (JP) 2019-014218

(51) Int. Cl. H01L 51/00; C07D 487/22; C09K 11/06; C07D 491/22; C07D 209/86; C07C 211/61; C07D 307/91; C07F 7/08; C07D 405/12

(52) U.S. Cl. CPC H01L 51/0061; C07D 487/22; C09K 11/06; C07D 491/22; H01L 51/0071; C07D 209/86; H01L 51/5012; H01L 51/006; H01L 51/0052; C07D 307/91; C07F 7/081; H01L 51/0094; C07D 405/12; C07C 211/61

(57) ABSTRACT

An organic electroluminescence device comprising: cathode; anode; and organic layer disposed between cathode and anode, wherein organic layer comprises a emitting layer and a first layer, the first layer being disposed between anode and emitting layer, and immediately adjoining emitting layer, emitting layer comprising a compound represented by formula (A1) and the first layer comprising a compound represented by formula (B1) or formula (C1) below.

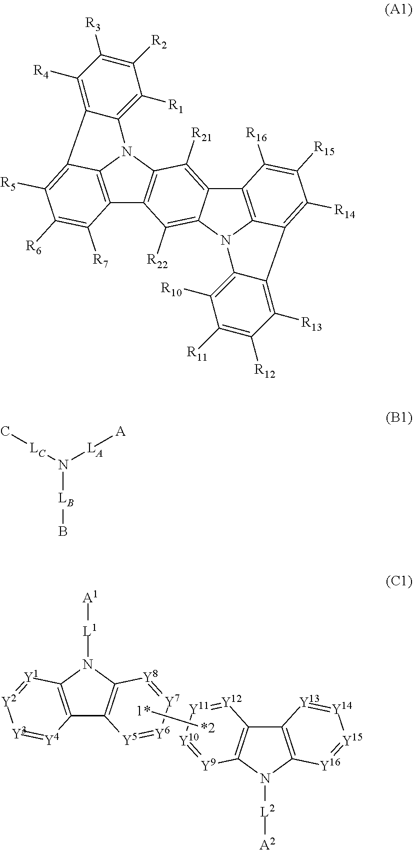

### TECHNICAL FIELD

The invention relates to an organic electroluminescence device and an electronic appliance using the same.

### BACKGROUND ART

When voltage is applied to an organic electroluminescence device (hereinafter, referred to as an organic EL device in several cases), holes and electrons are injected into an emitting layer from an anode and a cathode, respectively. Then, thus injected holes and electrons are recombined in the emitting layer, and excitons are formed therein.

Patent Document 1 discloses the use of a compound having a specific fused-ring structure as a material for an emitting layer of an organic EL device.

### RELATED ART DOCUMENTS

### Patent Documents

[Patent Document 1] WO2018/151065A1

### SUMMARY OF THE INVENTION

It is an object of the invention to provide an organic EL device excellent in luminous efficiency and an electronic appliance using the organic EL device.

According to the invention, the following organic EL device and electronic appliance are provided.

1. An organic electroluminescence device comprising:

a cathode;

an anode; and

an organic layer disposed between the cathode and the anode, wherein

the organic layer comprises an emitting layer and a first layer;

the first layer is disposed between the anode and the emitting layer and is directly adjacent to the emitting layer;

the emitting layer comprises a compound represented by the following formula (A1); and

the first layer comprises a compound represented by the following formula (B1) or the following formula (C1):

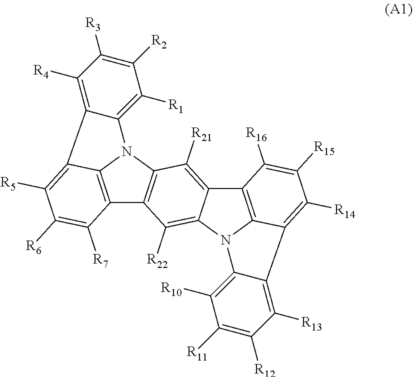

wherein in the formula (A1),

one or more sets of adjacent two or more among R₁ to R₇ and R₁₀ to R₁₆ form a substituted or unsubstituted, saturated or unsaturated ring by bonding with each other, or do not form a substituted or unsubstituted, saturated or unsaturated ring;

R₁ to R₇ and R₁₀ to R₁₆ which do not form the substituted or unsubstituted, saturated or unsaturated ring, and R₂₁ and R₂₂ are independently a hydrogen atom or substituent;

the substituent is

a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 carbon atoms that form a ring (hereinafter referred to as “ring carbon atoms”),
—Si(R₉₀₁)(R₉₀₂)(R₉₀₃),

### —O—(R₉₀₄),

### —S—(R₉₀₅),

—N(R₉₀₆)(R₉₀₇),
a halogen atom, a cyano group, a nitro group,
a substituted or unsubstituted aryl group including 6 to 50 ring carbon atoms, or
a substituted or unsubstituted monovalent heterocyclic group including 5 to 50 atoms that form a ring (hereinafter referred to as “ring atoms”);

R₉₀₁ to R₉₀₇ are independently

a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms;

when two or more of each of R₉₀₁ to R₉₀₇ are present, the two or more of each of R₉₀₁ to R₉₀₇ may be the same or different; and

provided that the formula (A1) satisfies one or both of the following requirements (i) and (ii):

(i) one or more sets of adjacent two or more among R₁ to R₇ and R₁₀ to R₁₆ form a substituted or unsubstituted, saturated or unsaturated ring by bonding with each other,

(ii) one or more among R₁ to R₇, R₁₀ to R₁₆, R₂₁ and R₂₂ is the substituent:

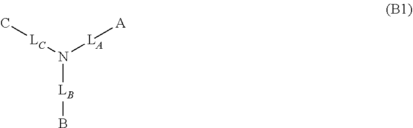

wherein in the formula (B1),

L_{A}, L_{B} and L_{C} are independently a single bond, a substituted or unsubstituted arylene group including 6 to 18 ring carbon atoms, or a substituted or unsubstituted divalent heterocyclic group including 5 to 13 ring atoms;

A, B and C are independently

a substituted or unsubstituted aryl group including 6 to 30 ring carbon atoms,
a substituted or unsubstituted monovalent heterocyclic group including 5 to 30 ring atoms, or
—Si(R′₉₀₁)(R′902)(R′₉₀₃),

R′₉₀₁ to R′₉₀₃ are independently a substituted or unsubstituted aryl group including 6 to 30 ring carbon atoms; and

when two or more of each of one or more among R′₉₀₁ to R′₉₀₃ are present, each of the two or more R′₉₀₁ to R′₉₀₃ may be the same or different;

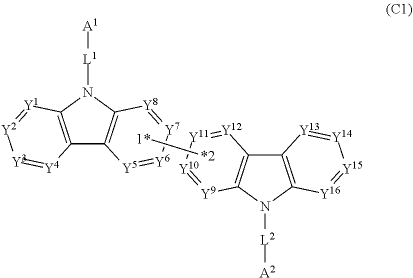

wherein in the formula (C1),

A¹ and A² are independently a substituted or unsubstituted aryl group including 6 to 30 ring carbon atoms, or a substituted or unsubstituted monovalent heterocyclic group including 5 to 30 ring atoms;

one of Y⁵ to Y⁸ is a carbon atom which is bonded to *1;

one of Y⁹ to Y¹² is a carbon atom which is bonded to *2;

Y¹ to Y⁴, Y¹³ to Y¹⁶, Y⁵ to Y⁸ which are not bonded to *1, and Y⁹ to Y¹² which are bonded to *2 are independently CR;

when a plurality of R's is present, one or more sets of adjacent two or more among the plurality of R's form a substituted or unsubstituted, saturated or unsaturated ring by bonding with each other, or do not form a substituted or unsubstituted, saturated or unsaturated ring;

R which does not form the substituted or unsubstituted, saturated or unsaturated ring is

a hydrogen atom,
a cyano group,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

a halogen atom, a nitro group,
a substituted or unsubstituted aryl group including 6 to 50 ring carbon atoms, or
a substituted or unsubstituted monovalent heterocyclic group including 5 to 50 ring atoms;

R₉₀₁ to R₉₀₄ are as defined in the formula (A1);

when a plurality of R's is present, the plurality of R's may be the same with or different from each other; and

L¹ and L² are independently a single bond, a substituted or unsubstituted arylene group including 6 to 30 ring carbon atoms, or a substituted or unsubstituted divalent heterocyclic group including 5 to 30 ring atoms.

2. An electronic appliance, wherein the organic electroluminescence device according to 1 is provided.

According to the invention, an organic EL device excellent in luminous efficiency and an electronic appliance using the organic EL device can be provided.

### BRIEF DESCRIPTION OF THE DRAWINGS

FIG. 1 is a diagram showing a schematic configuration of an aspect of an organic EL device according to an aspect of the invention.

### MODE FOR CARRYING OUT THE INVENTION

### Definition

In this specification, a hydrogen atom means an atom including isotopes different in the number of neutrons, namely, a protium, a deuterium and a tritium.

In this specification, to a bondable position in which a symbol such as “R”, or “D” representing a deuterium atom is not specified in a chemical formula, a hydrogen atom, that is, a light hydrogen atom, a deuterium atom, or a tritium atom is bonded thereto.

In this specification, a term “ring carbon atoms” represents the number of carbon atoms among atoms forming a subject ring itself of a compound having a structure in which atoms are bonded in a ring form (for example, a monocyclic compound, a fused ring compound, a cross-linked compound, a carbocyclic compound or a heterocyclic compound). When the subject ring is substituted by a substituent, the carbon contained in the substituent is not included in the number of ring carbon atoms. The same shall apply to the “ring carbon atoms” described below, unless otherwise noted. For example, a benzene ring has 6 ring carbon atoms, a naphthalene ring has 10 ring carbon atoms, a pyridine ring has 5 ring carbon atoms, and a furan ring has 4 ring carbon atoms. Further, for example, a 9,9-diphenylfluorenyl group has 13 ring carbon atoms, and a 9,9′-spirobifluorenyl group has 25 ring carbon atoms.

Further, when the benzene ring or the naphthalene ring is substituted by an alkyl group as a substituent, for example, the number of carbon atoms of the alkyl group is not included in the ring carbon atoms.

In this specification, a term “ring atoms” represents the number of atoms forming a subject ring itself of a compound having a structure in which atoms are bonded in a ring form (for example, a monocycle, a fused ring and a ring assembly) (for example, a monocyclic compound, a fused ring compound, a cross-linked compound, a carbocyclic compound or a heterocyclic compound). The term “ring atoms” does not include atoms which do not form the ring (for example, a hydrogen atom which terminates a bond of the atoms forming the ring) or atoms contained in a substituent when the ring is substituted by the substituent. The same shall apply to the “ring atoms” described below, unless otherwise noted. For example, a pyridine ring has 6 ring atoms, a quinazoline ring has 10 ring atoms, and a furan ring has 5 ring atoms. A hydrogen atom bonded with a carbon atom of the pyridine ring or the quinazoline ring or an atom forming the substituent is not included in the number of the ring atoms.

In this specification, a term “XX to YY carbon atoms” in an expression of “substituted or unsubstituted ZZ group including XX to YY carbon atoms” represents the number of carbon atoms when the ZZ group is unsubstituted. The number of carbon atoms of a substituent when the ZZ group is substituted is not included. Here, “YY” is larger than “XX”, and “XX” and “YY” each mean an integer of 1 or more.

In this specification, a term “XX to YY atoms” in an expression of “substituted or unsubstituted ZZ group including XX to YY atoms” represents the number of atoms when the ZZ group is unsubstituted. The number of atoms of a substituent when the group is substituted is not included. Here, “YY” is larger than “XX”, and “XX” and “YY” each mean an integer of 1 or more.

A term “unsubstituted” in the case of “substituted or unsubstituted ZZ group” means that the ZZ group is not substituted by a substituent, and a hydrogen atom is bonded therewith. Alternatively, a term “substituted” in the case of “substituted or unsubstituted ZZ group” means that one or more hydrogen atoms in the ZZ group are substituted by a substituent. Similarly, a term “substituted” in the case of “BB group substituted by an AA group” means that one or more hydrogen atoms in the BB group are substituted by the AA group.

Hereinafter, the substituent described herein will be described.

The number of the ring carbon atoms of the “unsubstituted aryl group” described herein is 6 to 50, preferably 6 to 30, and more preferably 6 to 18, unless otherwise specified.

The number of the ring carbon atoms of the “unsubstituted heterocyclic group” described herein is 5 to 50, preferably 5 to 30, and more preferably 5 to 18, unless otherwise specified.

The number of the carbon atoms of the “unsubstituted alkyl group” described herein is 1 to 50, preferably 1 to 20, and more preferably 1 to 6, unless otherwise specified.

The number of the carbon atoms of the “unsubstituted alkenyl group” described herein is 2 to 50, preferably 2 to 20, and more preferably 2 to 6, unless otherwise specified.

The number of the carbon atoms of the “unsubstituted alkynyl group” described herein is 2 to 50, preferably 2 to 20, and more preferably 2 to 6, unless otherwise specified.

The number of the ring carbon atoms of the “unsubstituted cycloalkyl group” described herein is 3 to 50, preferably 3 to 20, and more preferably 3 to 6, unless otherwise specified.

The number of the ring carbon atoms of the “unsubstituted arylene group” described herein is 6 to 50, preferably 6 to 30, and more preferably 6 to 18, unless otherwise specified.

The number of the ring atoms of the “unsubstituted divalent heterocyclic group” described herein is 5 to 50, preferably 5 to 30, and more preferably 5 to 18, unless otherwise specified.

The number of the carbon atoms of the “unsubstituted alkylene group” described herein is 1 to 50, preferably 1 to 20, and more preferably 1 to 6, unless otherwise specified.

Specific examples (specific example group G1) of the “substituted or unsubstituted aryl group” described herein include an unsubstituted aryl group and a substituted aryl group described below. (Here, a term “unsubstituted aryl group” refers to a case where the “substituted or unsubstituted aryl group” is the “unsubstituted aryl group,” and a term “substituted aryl group” refers to a case where the “substituted or unsubstituted aryl group” is the “substituted aryl group”. Hereinafter, a case of merely “aryl group” includes both the “unsubstituted aryl group” and the “substituted aryl group”.

The “substituted aryl group” refers to a case where the “unsubstituted aryl group” has a substituent, and specific examples thereof include a group in which the “unsubstituted aryl group” has the substituent, and a substituted aryl group described below. It should be noted that examples of the “unsubstituted aryl group” and examples of the “substituted aryl group” listed herein are only one example, and the “substituted aryl group” described herein also includes a group in which a group in which “unsubstituted aryl group” has a substituent further has a substituent, and a group in which “substituted aryl group” further has a substituent, and the like.

An unsubstituted aryl group:

a phenyl group,
a p-biphenyl group,
a m-biphenyl group,
an o-biphenyl group,
a p-terphenyl-4-yl group,
a p-terphenyl-3-yl group,
a p-terphenyl-2-yl group,
a m-terphenyl-4-yl group,
a m-terphenyl-3-yl group,
a m-terphenyl-2-yl group,
an o-terphenyl-4-yl group,
an o-terphenyl-3-yl group,
an o-terphenyl-2-yl group,
a 1-naphthyl group,
a 2-naphthyl group,
an anthryl group,
a benzanthryl group,
a phenanthryl group,
a benzophenanthryl group,
a phenalenyl group,
a pyrenyl group,
a chrysenyl group,
a benzochrysenyl group,
a triphenylenyl group,
a benzotriphenylenyl group,
a tetracenyl group,
a pentacenyl group,
a fluorenyl group,
a 9,9′-spirobifluorenyl group,
a benzofluorenyl group,
a dibenzofluorenyl group,
a fluoranthenyl group,
a benzofluoranthenyl group, and
a perylenyl group.

A substituted aryl group:

an o-tolyl group,
a m-tolyl group,
a p-tolyl group,
a p-xylyl group,
a m-xylyl group,
an o-xylylgroup,
a p-isopropyl phenyl group,
a m-isopropyl phenyl group,
an o-isopropyl phenyl group,
a p-t-butylphenyl group,
a m-t-butylphenyl group,
an o-t-butylphenyl group,
a 3,4,5-trimethylphenyl group,
a 9,9-dimethylfluorenyl group,
a 9,9-diphenylfluorenyl group
a 9,9-di(4-methylphenyl)fluorenyl group,
a 9,9-di(4-isopropylphenyl)fluorenyl group,
a 9,9-di(4-t-butylphenyl)fluorenyl group,
a cyanophenyl group,
a triphenylsilylphenyl group,
a trimethylsilylphenyl group,
a phenylnaphthyl group, and
a naphthylphenyl group.

The “heterocyclic group” described herein is a ring group including at least one hetero atom in the ring atom. Specific examples of the hetero atom include a nitrogen atom, an oxygen atom, a sulfur atom, a silicon atom, a phosphorus atom and a boron atom.

The “heterocyclic group” described herein may be a monocyclic group, or a fused ring group.

The “heterocyclic group” described herein may be an aromatic heterocyclic group, or an aliphatic heterocyclic group.

Specific examples (specific example group G2) of the “substituted or unsubstituted heterocyclic group” include an unsubstituted heterocyclic group and a substituted heterocyclic group described below. (Here, the unsubstituted heterocyclic group refers to a case where the “substituted or unsubstituted heterocyclic group” is the “unsubstituted heterocyclic group,” and the substituted heterocyclic group refers to a case where the “substituted or unsubstituted heterocyclic group” is the “substituted heterocyclic group”. Hereinafter, the case of merely “heterocyclic group” includes both the “unsubstituted heterocyclic group” and the “substituted heterocyclic group”.

The “substituted heterocyclic group” refers to a case where the “unsubstituted heterocyclic group” has a substituent, and specific examples thereof include a group in which the “unsubstituted heterocyclic group” has a substituent, and a substituted heterocyclic group described below. It should be noted that examples of the “unsubstituted heterocyclic group” and examples of the “substituted heterocyclic group” listed herein are merely one example, and the “substituted heterocyclic group” described herein also includes a group in which “unsubstituted heterocyclic group” which has a substituent further has a substituent, and a group in which “substituted heterocyclic group” further has a substituent, and the like.

An unsubstituted heterocyclic group including a nitrogen atom:

a pyrrolyl group,
an imidazolyl group,
a pyrazolyl group,
a triazolyl group,
a tetrazolyl group,
an oxazolyl group,
an isoxazolyl group,
an oxadiazolyl group,
a thiazolyl group,
an isothiazolyl group,
a thiadiazolyl group,
a pyridyl group,
a pyridazinyl group,
a pyrimidinyl group,
a pyrazinyl group,
a triazinyl group,
an indolyl group,
an isoindolyl group,
an indolizinyl group,
a quinolizinyl group,
a quinolyl group,
an isoquinolyl group,
a cinnolyl group,
a phthalazinyl group,
a quinazolinyl group,
a quinoxalinyl group,
a benzimidazolyl group,
an indazolyl group,
a phenanthrolinyl group,
a phenanthridinyl group
an acridinyl group,
a phenazinyl group,
a carbazolyl group,
a benzocarbazolyl group,
a morpholino group,
a phenoxazinyl group,
a phenothiazinyl group,
an azacarbazolyl group, and
a diazacarbazolyl group.

An unsubstituted heterocyclic group including an oxygen atom:

a furyl group,
an oxazolyl group,
an isoxazolyl group,
an oxadiazolyl group,
a xanthenyl group,
a benzofuranyl group,
an isobenzofuranyl group,
a dibenzofuranyl group,
a naphthobenzofuranyl group,
a benzoxazolyl group,
a benzisoxazolyl group,
a phenoxazinyl group,
a morpholino group,
a dinaphthofuranyl group,
an azadibenzofuranyl group,
a diazadibenzofuranyl group,
an azanaphthobenzofuranyl group, and
a diazanaphthobenzofuranyl group.

An unsubstituted heterocyclic group including a sulfur atom:

a thienyl group,
a thiazolyl group,
an isothiazolyl group,
a thiadiazolyl group,
a benzothiophenyl group,
an isobenzothiophenyl group,
a dibenzothiophenyl group,
a naphthobenzothiophenyl group,
a benzothiazolyl group,
a benzisothiazolyl group,
a phenothiazinyl group,
a dinaphthothiophenyl group,
an azadibenzothiophenyl group,
a diazadibenzothiophenyl group,
an azanaphthobenzothiophenyl group, and
a diazanaphthobenzothiophenyl group.

A substituted heterocyclic group including a nitrogen atom:

a (9-phenyl)carbazolyl group,
a (9-biphenylyl)carbazolyl group,
a (9-phenyl)phenylcarbazolyl group,
a (9-naphthyl)carbazolyl group,
a diphenylcarbazol-9-yl group,
a phenylcarbazol-9-yl group,
a methylbenzimidazolyl group,
an ethylbenzimidazolyl group,
a phenyltriazinyl group,
a biphenylyltriazinyl group,
a diphenyltriazinyl group,
a phenylquinazolinyl group, and
a biphenylylquinazolinyl group.

A substituted heterocyclic group including an oxygen atom:

a phenyldibenzofuranyl group,
a methyldibenzofuranyl group,
a t-butyldibenzofuranyl group, and
a monovalent residue of spiro[9H-xanthene-9,9′-[9H]fluorene].

A substituted heterocyclic group including a sulfur atom:

a phenyldibenzothiophenyl group,
a methyldibenzothiophenyl group,
a t-butyldibenzothiophenyl group, and
a monovalent residue of spiro[9H-thioxantene-9,9′-[9H]fluorene].

A monovalent group derived from the following unsubstituted heterocyclic ring containing at least one of a nitrogen atom, an oxygen atom and a sulfur atom by removal of one hydrogen atom bonded to the ring atoms thereof, and a group in which a monovalent group derived from the following unsubstituted heterocyclic ring by removal of one hydrogen atom bonded to the ring atoms thereof has a substituent:

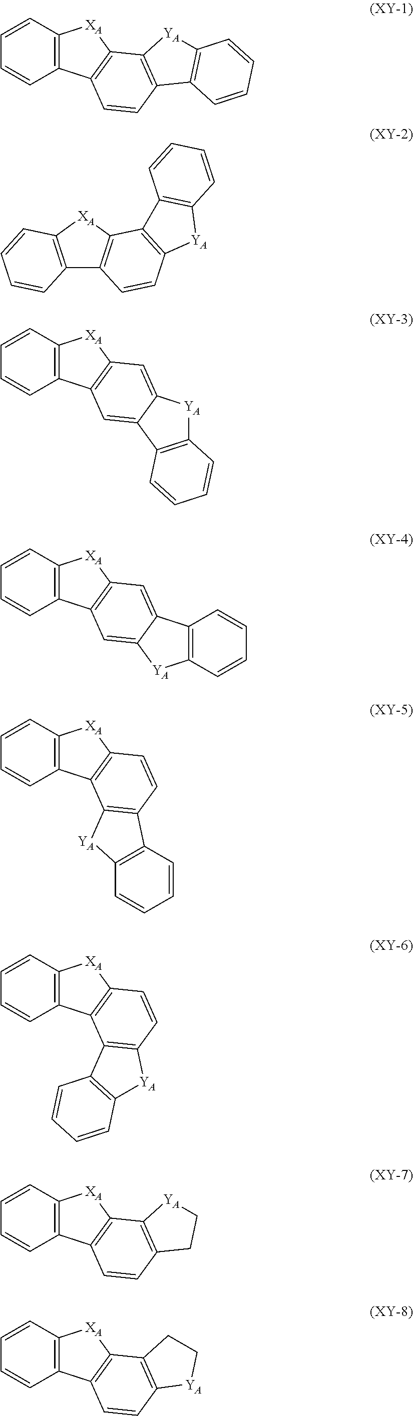

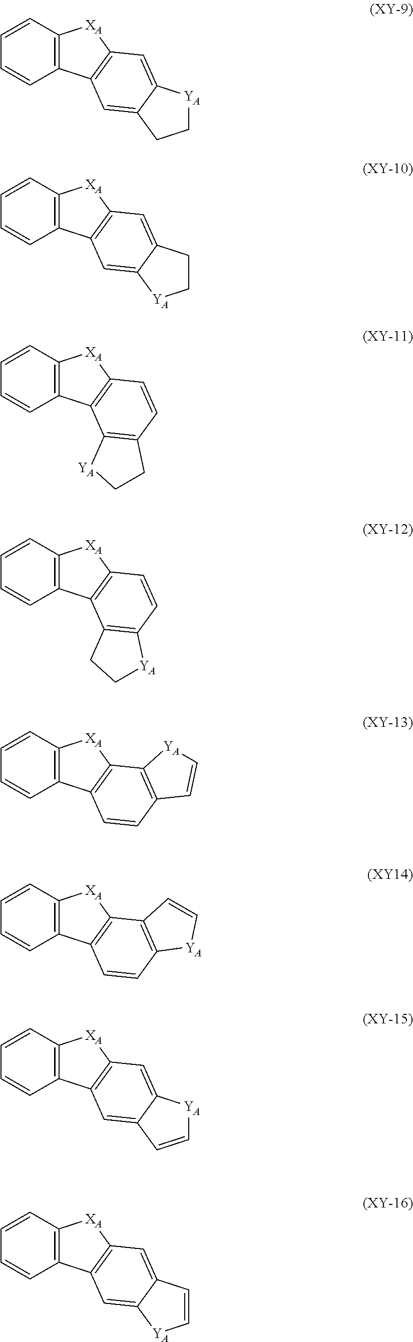

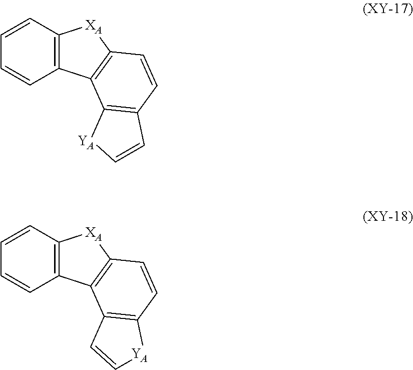

In the formulas (XY-1) to (XY-18), X_{A} and Y_{A} are independently an oxygen atom, a sulfur atom, NH or CH₂. However, at least one of X_{A} and Y_{A} is an oxygen atom, a sulfur atom or NH.

The heterocyclic ring represented by the formulas (XY-1) to (XY-18) becomes a monovalent heterocyclic group including a bond at an arbitrary position.

An expression “the monovalent group derived from the unsubstituted heterocyclic ring represented by the formulas (XY-1) to (XY-18) has a substituent” refers to a case where the hydrogen atom bonded with the carbon atom which constitutes a skeleton of the formulas is substituted by a substituent, or a state in which X_{A} or Y_{A} is NH or CH₂, and the hydrogen atom in the NH or CH₂ is replaced with a substituent.

Specific examples (specific example group G3) of the “substituted or unsubstituted alkyl group” include an unsubstituted alkyl group and a substituted alkyl group described below. (Here, the unsubstituted alkyl group refers to a case where the “substituted or unsubstituted alkyl group” is the “unsubstituted alkyl group,” and the substituted alkyl group refers to a case where the “substituted or unsubstituted alkyl group” is the “substituted alkyl group”). Hereinafter, the case of merely “alkyl group” includes both the “unsubstituted alkyl group” and the “substituted alkyl group”.

The “substituted alkyl group” refers to a case where the “unsubstituted alkyl group” has a substituent, and specific examples thereof include a group in which the “unsubstituted alkyl group” has a substituent, and a substituted alkyl group described below. It should be noted that examples of the “unsubstituted alkyl group” and examples of the “substituted alkyl group” listed herein are merely one example, and the “substituted alkyl group” described herein also includes a group in which “unsubstituted alkyl group” has a substituent further has a substituent, a group in which “substituted alkyl group” further has a substituent, and the like.

An unsubstituted alkyl group:

a methyl group,
an ethyl group,
a n-propyl group,
an isopropyl group,
a n-butyl group,
an isobutyl group,
a s-butyl group, and
a t-butyl group.

A substituted alkyl group:

a heptafluoropropyl group (including an isomer),
a pentafluoroethyl group,
a 2,2,2-trifluoroethyl group, and
a trifluoromethyl group.

Specific examples (specific example group G4) of the “substituted or unsubstituted alkenyl group” include an unsubstituted alkenyl group and a substituted alkenyl group described below. (Here, the unsubstituted alkenyl group refers to a case where the “substituted or unsubstituted alkenyl group” is the “unsubstituted alkenyl group,” and the substituted alkenyl group refers to a case where the “substituted or unsubstituted alkenyl group” is the “substituted alkenyl group”). Hereinafter, the case of merely “alkenyl group” includes both the “unsubstituted alkenyl group” and the “substituted alkenyl group”.

The “substituted alkenyl group” refers to a case where the “unsubstituted alkenyl group” has a substituent, and specific examples thereof include a group in which the “unsubstituted alkenyl group” has a substituent, and a substituted alkenyl group described below. It should be noted that examples of the “unsubstituted alkenyl group” and examples of the “substituted alkenyl group” listed herein are merely one example, and the “substituted alkenyl group” described herein also includes a group in which “unsubstituted alkenyl group” has a substituent further has a substituent, a group in which “substituted alkenyl group” further has a substituent, and the like.

An unsubstituted alkenyl group and a substituted alkenyl group:

a vinyl group,
an allyl group,
a 1-butenyl group,
a 2-butenyl group,
a 3-butenyl group,
a 1,3-butanedienyl group,
a 1-methylvinyl group,
a 1-methylallyl group,
a 1,1-dimethylallyl group,
a 2-methylallyl group, and
a 1,2-dimethylallyl group.

Specific examples (specific example group G5) of the “substituted or unsubstituted alkynyl group” include an unsubstituted alkynyl group described below. (Here, the unsubstituted alkynyl group refers to a case where the “substituted or unsubstituted alkynyl group” is the “unsubstituted alkynyl group”). Hereinafter, a case of merely “alkynyl group” includes both the “unsubstituted alkynyl group” and the “substituted alkynyl group”.

The “substituted alkynyl group” refers to a case where the “unsubstituted alkynyl group” has a substituent, and specific examples thereof include a group in which the “unsubstituted alkynyl group” described below has a substituent.

An unsubstituted alkynyl group:

an ethynyl group.

Specific examples (specific example group G6) of the “substituted or unsubstituted cycloalkyl group” described herein include an unsubstituted cycloalkyl group and a substituted cycloalkyl group described below. (Here, the unsubstituted cycloalkyl group refers to a case where the “substituted or unsubstituted cycloalkyl group” is the “unsubstituted cycloalkyl group,” and the substituted cycloalkyl group refers to a case where the “substituted or unsubstituted cycloalkyl group” is the “substituted cycloalkyl group”). Hereinafter, a case of merely “cycloalkyl group” includes both the “unsubstituted cycloalkyl group” and the “substituted cycloalkyl group”.

The “substituted cycloalkyl group” refers to a case where the “unsubstituted cycloalkyl group” a the substituent, and specific examples thereof include a group in which the “unsubstituted cycloalkyl group” has a substituent, and a substituted cycloalkyl group described below. It should be noted that examples of the “unsubstituted cycloalkyl group” and examples of the “substituted cycloalkyl group” listed herein are merely one example, and the “substituted cycloalkyl group” described herein also includes a group in which “unsubstituted cycloalkyl group” has a substituent further has a substituent, a group in which “substituted cycloalkyl group” further has a substituent, and the like.

An unsubstituted aliphatic ring group:

a cyclopropyl group,
a cyclobutyl group,
a cyclopentyl group,
a cyclohexyl group,
a 1-adamantyl group,
a 2-adamantyl group,
a 1-norbornyl group, and
a 2-norbornyl group.

A substituted cycloalkyl group:

a 4-methylcyclohexyl group.

Specific examples (specific example group G7) of the group represented by —Si(R₉₀₁)(R₉₀₂)(R₉₀₃) described herein include

### —Si(G1)(G1)(G1),

### —Si(G1)(G2)(G2),

### —Si(G1)(G1)(G2),

### —Si(G2)(G2)(G2),

### —Si(G3)(G3)(G3),

### —Si(G5)(G5)(G5) and

### —Si(G6)(G6)(G6).

In which,

G1 is the “aryl group” described in the specific example group G1.

G2 is the “heterocyclic group” described in the specific example group G2.

G3 is the “alkyl group” described in the specific example group G3.

G5 is the “alkynyl group” described in the specific example group G5.

G6 is the “cycloalkyl group” described in the specific example group G6.

Specific examples (specific example group G8) of the group represented by —O—(R₉₀₄) described herein include

### —O(G1),

### —O(G2),

### —O(G3) and

### —O(G6).

In which,

G1 is the “aryl group” described in the specific example group G1.

G2 is the “heterocyclic group” described in the specific example group G2.

G3 is the “alkyl group” described in the specific example group G3.

G6 is the “cycloalkyl group” described in the specific example group G6.

Specific examples (specific example group G9) of the group represented by —S—(R₉₀₅) described herein include

### —S(G1),

### —S(G2),

### —S(G3) and

### —S(G6).

In which,

G1 is the “aryl group” described in the specific example group G1.

G2 is the “heterocycle group” described in the specific example group G2.

G3 is the “alkyl group” described in the specific example group G3.

G6 is the “cycloalkyl group” described in the specific example group G6.

Specific examples (specific example group G10) of the group represented by —N(R₉₀₆)(R₉₀₇) described herein include

### —N(G1)(G1),

### —N(G2)(G2),

### —N(G1)(G2),

### —N(G3)(G3) and

### —N(G6) (G6).

In which,

G1 is the “aryl group” described in the specific example group G1.

G2 is the “heterocycle group” described in the specific example group G2.

G3 is the “alkyl group” described in the specific example group G3.

G6 is the “cycloalkyl group” described in the specific example group G6.

Specific examples (specific example group G11) of the “halogen atom” described herein include a fluorine atom, a chlorine atom, a bromine atom and an iodine atom.

Specific examples of the “alkoxy group” described herein include a group represented by —O(G3), where G3 is the “alkyl group” described in the specific example group G3. The number of carbon atoms of the “unsubstituted alkoxy group” are 1 to 50, preferably 1 to 30, and more preferably 1 to 18, unless otherwise specified.

Specific examples of the “alkylthio group” described herein include a group represented by —S(G3), where G3 is the “alkyl group” described in the specific example group G3. The number of carbon atoms of the “unsubstituted alkylthio group” are 1 to 50, preferably 1 to 30, and more preferably 1 to 18, unless otherwise specified.

Specific examples of the “aryloxy group” described herein include a group represented by —O(G1), where G1 is the “aryl group” described in the specific example group G1. The number of ring carbon atoms of the “unsubstituted aryloxy group” are 6 to 50, preferably 6 to 30, and more preferably 6 to 18, unless otherwise specified.

Specific examples of the “arylthio group” described herein include a group represented by —S(G1), where G1 is the “aryl group” described in the specific example group G1. The number of ring carbon atoms of the “unsubstituted arylthio group” are 6 to 50, preferably 6 to 30, and more preferably 6 to 18, unless otherwise specified.

Specific examples of the “aralkyl group” described herein include a group represented by -(G3)-(G1), where G3 is the “alkyl group” described in the specific example group G3, and G1 is the “aryl group” described in the specific example group G1. Accordingly, the “aralkyl group” is one embodiment of the “substituted alkyl group” substituted by the “aryl group”. The number of carbon atoms of the “unsubstituted aralkyl group,” which is the “unsubstituted alkyl group” substituted by the “unsubstituted aryl group,” are 7 to 50, preferably 7 to 30, and more preferably 7 to 18, unless otherwise specified.

Specific example of the “aralkyl group” include a benzyl group, a 1-phenylethyl group, a 2-phenylethyl group, a 1-phenylisopropyl group, a 2-phenylisopropyl group, a phenyl-t-butyl group, an a-naphthylmethyl group, a 1-a-naphthylethyl group, a 2-a-naphthylethyl group, a 1-a-naphthylisopropyl group, a 2-a-naphthylisopropyl group, a β-naphthylmethyl group, a 1-β-naphthylethyl group, a 2-β-naphthylethyl group, a 1-β-naphthylisopropyl group, and a 2-β-naphthylisopropyl group.

The substituted or unsubstituted aryl group described herein is, unless otherwise specified, preferably a phenyl group, a p-biphenyl group, a m-biphenyl group, an o-biphenyl group, a p-terphenyl-4-yl group, a p-terphenyl-3-yl group, a p-terphenyl-2-yl group, a m-terphenyl-4-yl group, a m-terphenyl-3-yl group, a m-terphenyl-2-yl group, an o-terphenyl-4-yl group, an o-terphenyl-3-yl group, an o-terphenyl-2-yl group, a 1-naphthyl group, a 2-naphthyl group, an anthryl group, a phenanthryl group, a pyrenyl group, a chrysenyl group, a triphenylenyl group, a fluorenyl group, a 9,9′-spirobifluorenyl group, a 9,9-diphenylfluorenyl group, or the like.

The substituted or unsubstituted heterocyclic group described herein is, unless otherwise specified, preferably a pyridyl group, a pyrimidinyl group, a triazinyl group, a quinolyl group, an isoquinolyl group, a quinazolinyl group, a benzimidazolyl group, a phenanthrolinyl group, a carbazolyl group (a 1-carbazolyl group, a 2-carbazolyl group, a 3-carbazolyl group, a 4-carbazolyl group, or a 9-carbazolyl group), a benzocarbazolyl group, an azacarbazolyl group, a diazacarbazolyl group, a dibenzofuranyl group, a naphthobenzofuranyl group, an azadibenzofuranyl group, a diazadibenzofuranyl group, a dibenzothiophenyl group, a naphthobenzothiophenyl group, an azadibenzothiophenyl group, a diazadibenzothiophenyl group, a (9-phenyl)carbazolyl group (a (9-phenyl)carbazol-1-yl group, a (9-phenyl)carbazol-2-yl group, a (9-phenyl)carbazol-3-yl group, or a (9-phenyl)carbazol-4-yl group), a (9-biphenylyl)carbazolyl group, a (9-phenyl)phenylcarbazolyl group, a diphenylcarbazole-9-yl group, a phenylcarbazol-9-yl group, a phenyltriazinyl group, a biphenylyltriazinyl group, diphenyltriazinyl group, a phenyldibenzofuranyl group, a phenyldibenzothiophenyl group, an indrocarbazolyl group, a pyrazinyl group, a pyridazinyl group, a quinazolinyl group, a cinnolinyl group, a phthalazinyl group, a quinoxalinyl group, a pyrrolyl group, an indolyl group, a pyrrolo[3,2,1-jk]carbazolyl group, a furanyl group, a benzofuranyl group, a thiophenyl group, a benzothiophenyl group, a pyrazolyl group, an imidazolyl group, a benzimidazolyl group, a triazolyl group, an oxazolyl group, a benzoxazolyl group, a thiazolyl group, a benzothiazolyl group, an isothiazolyl group, a benzisothiazolyl group, a thiadiazolyl group, an isoxazolyl group, a benzisoxazolyl group, a pyrrolidinyl group, a piperidinyl group, a piperazinyl group, an imidazolidinyl group, an indro[3,2,1-jk]carbazolyl group, a dibenzothiophenyl group, or the like.

The dibenzofuranyl group and the dibenzothiophenyl group as described above are specifically any group described below, unless otherwise specified.

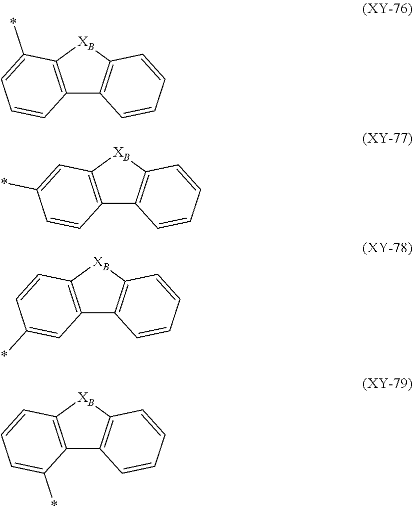

In the formulas (XY-76) to (XY-79), X_{B} is an oxygen atom or a sulfur atom.

The substituted or unsubstituted alkyl group described herein is, unless otherwise specified, preferably a methyl group, an ethyl group, a propyl group, an isopropyl group, a n-butyl group, an isobutyl group, a t-butyl group, or the like.

The “substituted or unsubstituted arylene group” descried herein refers to a group in which the above-described “aryl group” is converted into divalence, unless otherwise specified. Specific examples (specific example group G12) of the “substituted or unsubstituted arylene group” include a group in which the “aryl group” described in the specific example group G1 is converted into divalence. Namely, specific examples (specific example group G12) of the “substituted or unsubstituted arylene group” refer to a group derived from the “aryl group” described in specific example group G1 by removal of one hydrogen atom bonded to the ring carbon atoms thereof.

Specific examples (specific example group G13) of the “substituted or unsubstituted divalent heterocyclic group” include a group in which the “heterocyclic group” described in the specific example group G2 is converted into divalence. Namely, specific examples (specific example group G13) of the “substituted or unsubstituted divalent heterocyclic group” refer to a group derived from the “heterocyclic group” described in specific example group G2 by removal of one hydrogen atom bonded to the ring atoms thereof.

Specific examples (specific example group G14) of the “substituted or unsubstituted alkylene group” include a group in which the “alkyl group” described in the specific example group G3 is converted into divalence. Namely, specific examples (specific example group G14) of the “substituted or unsubstituted alkylene group” refer to a group derived from the “alkyl group” described in specific example group G3 by removal of one hydrogen atom bonded to the carbon atoms constituting the alkane structure thereof.

The substituted or unsubstituted arylene group described herein is any group described below, unless otherwise specified.

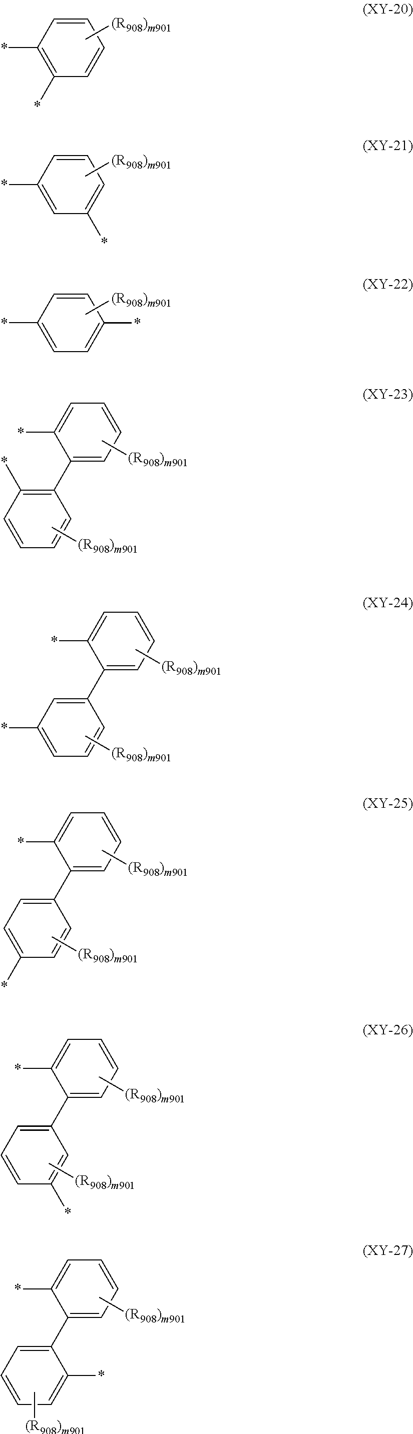

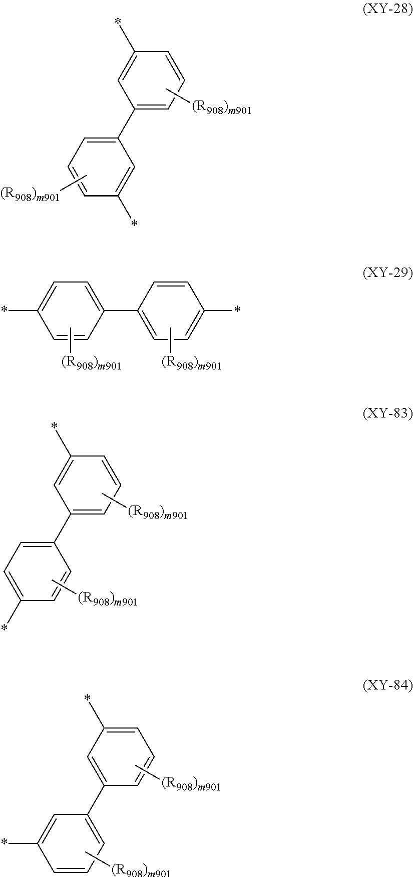

In the formulas (XY-20) to (XY-29), (XY-83) and (XY-84), R₉₀₈ is a substituent.

Then, m901 is an integer of 0 to 4, and when m901 is 2 or more, a plurality of R₉₀₈ may be the same with or different from each other.

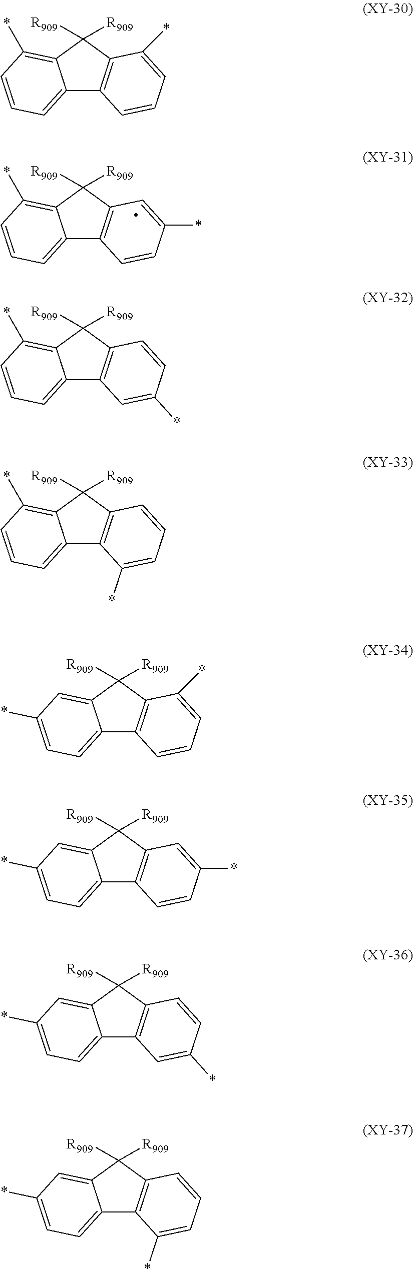

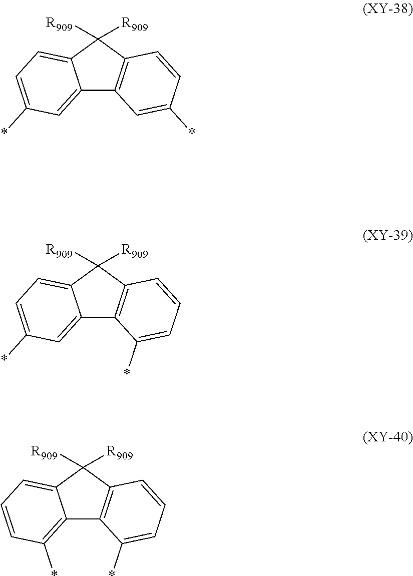

In the formulas (XY-30) to (XY-40), R₉₀₉ is independently a hydrogen atom or a substituent. Two of R₉₀₉ may form a ring by bonding with each other through a single bond.

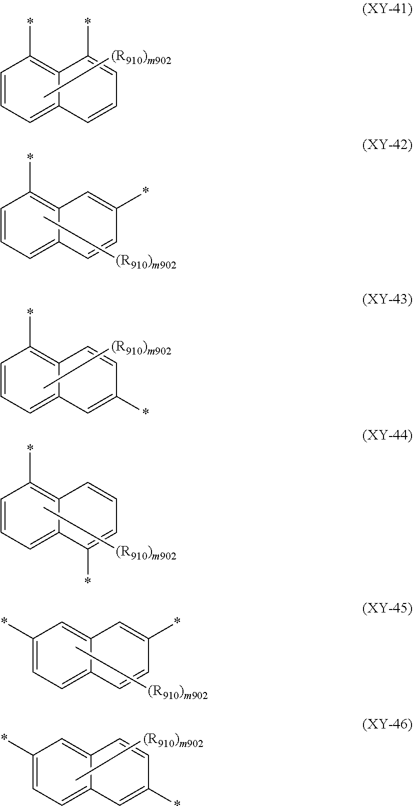

In the formulas (XY-41) to (XY-46), R₉₁₀ is a substituent.

Then, m902 is an integer of 0 to 6. When m902 is 2 or more, a plurality of R₉₁₀ may be the same with or different from each other.

The substituted or unsubstituted divalent heterocyclic group described herein is preferably any group described below, unless otherwise specified.

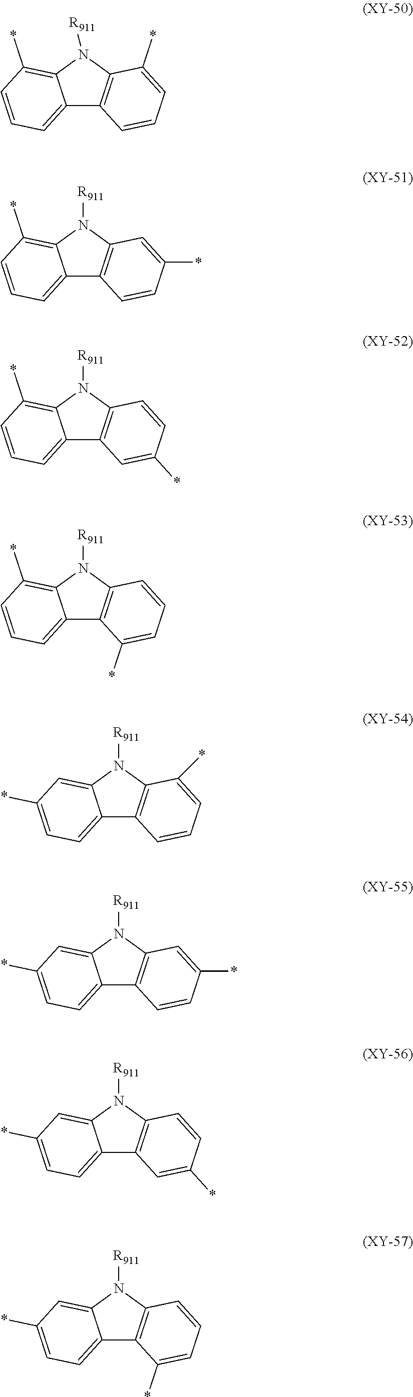

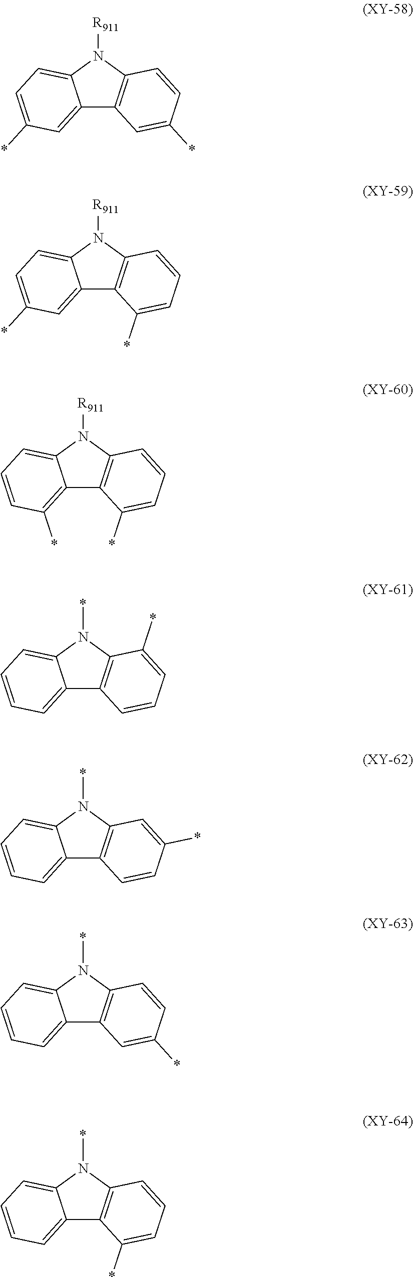

In the formulas (XY-50) to (XY-60), R₉₁₁ is a hydrogen atom or a substituent.

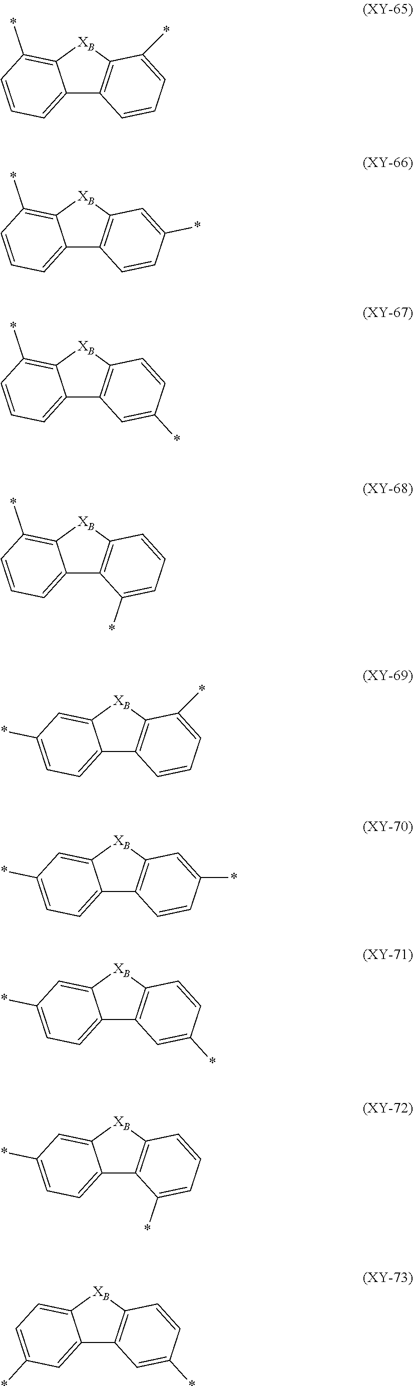

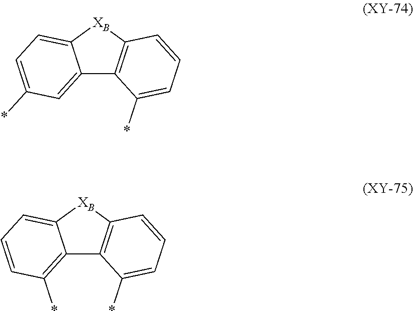

In the formulas (XY-65) to (XY-75), X_{B} is an oxygen atom or a sulfur atom.

Herein, a case where “one or more sets of two or more groups adjacent to each other form a substituted or unsubstituted and saturated or unsaturated ring by bonding with each other” will be described by taking, as an example, a case of an anthracene compound represented by the following formula (XY-80) in which a mother skeleton is an anthracene ring.

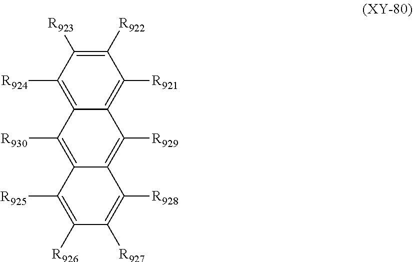

For example, two adjacent to each other into one set when “one or more sets of two or more groups adjacent to each other form the ring by bonding with each other” among R₉₂₁ to R₉₃₀ include R₉₂₁ and R₉₂₂, R₉₂₂ and R₉₂₃, R₉₂₃ and R₉₂₄, R₉₂₄ and R₉₃₀, R₉₃₀ and R₉₂₅, R₉₂₅ and R₉₂₆, R₉₂₆ and R₉₂₇, R₉₂₇ and R₉₂₈, R₉₂₈ and R₉₂₉, and R₉₂₉ and R₉₂₁.

The above-described “one or more sets” means that two or more sets of two groups adjacent to each other may simultaneously form the ring. For example, a case where R₉₂₁ and R₉₂₂ form a ring A by bonding with each other, and simultaneously R₉₂₅ and R₉₂₆ form a ring B by bonding with each other is represented by the following formula (XY-81).

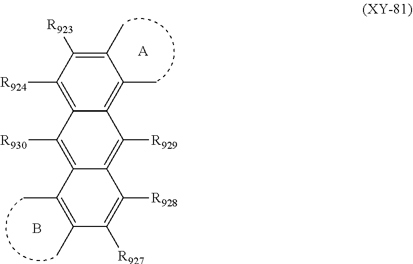

A case where “two or more groups adjacent to each other” form a ring means that, for example, R₉₂₁ and R₉₂₂ form a ring A by bonding with each other, and R₉₂₂ and R₉₂₃ form a ring C by bonding with each other. A case where the ring A and ring C sharing R₉₂₂ are formed, in which the ring A and the ring C are fused to the anthracene mother skeleton by three of R₉₂₁ to R₉₂₃ adjacent to each other, is represented by the following (XY-82).

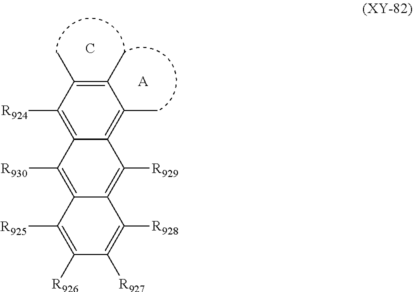

The rings A to C formed in the formulas (XY-81) and (XY-82) are a saturated or unsaturated ring.

A term “unsaturated ring” means an aromatic hydrocarbon ring or an aromatic heterocyclic ring. A term “saturated ring” means an aliphatic hydrocarbon ring or an aliphatic heterocyclic ring.

For example, the ring A formed by R₉₂₁ and R₉₂₂ being bonded with each other, represented by the formula (XY-81), means a ring formed by a carbon atom of the anthracene skeleton bonded with R₉₂₁, a carbon atom of the anthracene skeleton bonded with R₉₂₂, and one or more arbitrary elements. Specific examples include, when the ring A is formed by R₉₂₁ and R₉₂₂, a case where an unsaturated ring is formed of a carbon atom of an anthracene skeleton bonded with R₉₂₁, a carbon atom of the anthracene skeleton bonded with R₉₂₂, and four carbon atoms, in which a ring formed by R₉₂₁ and R₉₂₂ is formed into a benzene ring. Further, when a saturated ring is formed, the ring is formed into a cyclohexane ring.

Here, “arbitrary elements” are preferably a C element, a N element, an O element and a S element. In the arbitrary elements (for example, a case of the C element or the N element), the bond(s) that is(are) not involved in the formation of the ring may be terminated by a hydrogen atom, or may be substituted by an arbitrary substituent. When the ring contains the arbitrary elements other than the C element, the ring to be formed is a heterocyclic ring.

The number of “one or more arbitrary elements” forming the saturated or unsaturated ring is preferably 2 or more and 15 or less, more preferably 3 or more and 12 or less, and further preferably 3 or more and 5 or less.

As specific examples of the aromatic hydrocarbon ring, a structure in which the aryl group described in specific example group G1 is terminated with a hydrogen atom may be mentioned.

As specific examples of the aromatic heterocyclic ring, a structure in which the aromatic heterocyclic group described in specific example group G2 is terminated with a hydrogen atom may be mentioned.

As specific examples of the aliphatic hydrocarbon ring, a structure in which the cycloalkyl group described in specific example group G6 is terminated with a hydrogen atom may be mentioned.

When the above-described “saturated or unsaturated ring” has a substituent, the substituent is an “arbitrary substituent” as described below, for example. When the above-mentioned “saturated or unsaturated ring” has a substituent, specific examples of the substituent refer to the substituents described in above-mentioned “the substituent described herein”.

In one embodiment of this specification, the substituent (hereinafter, referred to as an “arbitrary substituent” in several cases) in the case of the “substituted or unsubstituted” is a group selected from the group consisting of

an unsubstituted alkyl group including 1 to 50 carbon atoms,
an unsubstituted alkenyl group including 2 to 50 carbon atoms,
an unsubstituted alkynyl group including 2 to 50 carbon atoms,
an unsubstituted cycloalkyl group including 3 to 50 ring carbon atoms,
—Si(R₉₀₁)(R₉₀₂)(R₉₀₃),

### —O—(R₉₀₄),

### —S—(R₉₀₅)

—N(R₉₀₆)(R₉₀₇)
wherein,
R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms; and when two or
more of R₉₀₁ to R₉₀₇ exist, two or more of R₉₀₄ to R₉₀₇ may be the same with or different from each other,
a halogen atom, a cyano group, a nitro group,
an unsubstituted aryl group including 6 to 50 ring carbon atoms, and
an unsubstituted monovalent heterocyclic group including 5 to 50 ring atoms.

In one embodiment, the substituent in the case of “substituted or unsubstituted” is a group selected from the group consisting of

an alkyl group including 1 to 50 carbon atoms,
an aryl group including 6 to 50 ring carbon atoms, and
a monovalent heterocyclic group including 5 to 50 ring atoms.

In one embodiment, the substituent in the case of “substituted or unsubstituted” is a group selected from the group consisting of

an alkyl group including 1 to 18 carbon atoms,
an aryl group including 6 to 18 ring carbon atoms, and
a monovalent heterocyclic group including 5 to 18 ring atoms.

Specific examples of each group of the arbitrary substituent described above are as described above.

Herein, unless otherwise specified, the saturated or unsaturated ring (preferably substituted or unsubstituted and saturated or unsaturated five-membered or six-membered ring, more preferably a benzene ring) may be formed by the arbitrary substituents adjacent to each other.

Herein, unless otherwise specified, the arbitrary substituent may further have the substituent. Specific examples of the substituent that the arbitrary substituent further has include to the ones same as the arbitrary substituent described above.

[Organic EL Device]

An organic electroluminescence device according to an aspect of the invention containing: a cathode; an anode; and an organic layer disposed between the cathode and the anode. The organic layer contains an emitting layer and a first layer, wherein the first layer is disposed between the anode and the emitting layer and is directly adjacent to the emitting layer; the emitting layer contains a compound represented by the formula (A1); and the first layer contains a compound represented by the formula (B1) or the formula (C1).

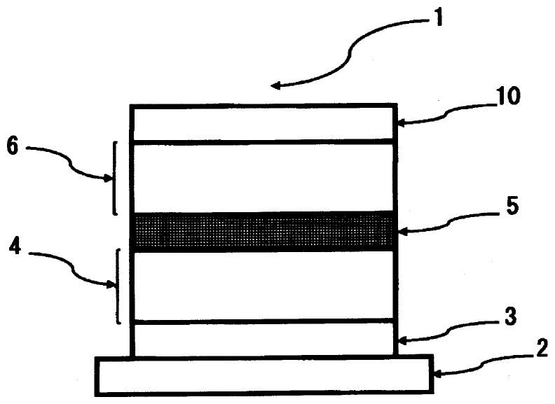

Schematic configuration of organic EL device according to one aspect of the invention will be explained referring to FIG. 1.

The organic EL device 1 according to an aspect of the invention comprises: a substrate 2, an anode 3, an emitting layer 5 as an organic layer, a cathode 10, an organic layer 4 between the anode 3 and the emitting layer 5, and an organic layer 6 between the emitting layer 5 and the cathode 10.

Each of the organic layer 4 and the organic layer 6 may be a single layer or may consist of a plurality of layers.

The first layer is arranged between the anode 3 and the emitting layer 5, i.e. in the organic layer 4, and is directly adjacent to the emitting layer 5. When the organic layer 4 is composed of a plurality of layers, the first layer is a layer directly adjacent to the emitting layer 5 among the plurality of layers. In addition to the first layer, the organic layer 4 may contain, for example, a hole-transporting layer. The first layer has, for example, the function of an electron barrier layer.

The compound represented by the formula (A1) is contained in the emitting layer 5 between the anode 3 and the cathode 10.

The compound represented by the formula (B1) or (C1) is contained in the first layer which is disposed between the anode 3 and the emitting layer 5 and is directly adjacent to the emitting layer 5.

In one embodiment, the organic EL device further contains a second layer. The second layer functions, for example, as a electron-transporting layer. The second layer is disposed between the cathode 10 and the emitting layer 5, i.e. in the organic layer 6.

In one embodiment, the compound represented by the formula (D1) is contained in the second layer disposed between the cathode 10 and the emitting layer 5. When the organic layer 6 contains a plurality of layers, a layer other than the second layer may also contain the compound represented by the formula (D1).

Hereinafter, the compounds represented by the formulas (A1), (B1), (C1) and (D1) will be described.

### (Compound Represented by the Formula (A1))

The compound represented by the following formula (A1) is contained in emitting layer.

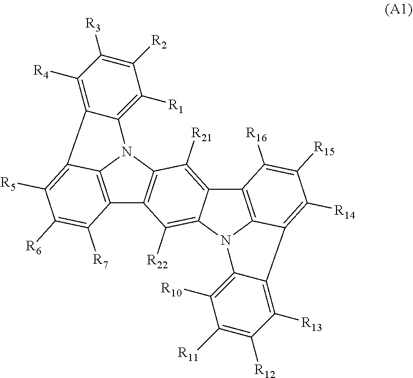

In the formula (A1),

one or more sets of adjacent two or more among R₁ to R₇ and R₁₀ to R₁₆ form a substituted or unsubstituted, saturated or unsaturated ring by bonding with each other, or do not form a substituted or unsubstituted, saturated or unsaturated ring;

R₁ to R₇ and R₁₀ to R₁₆ which do not form the substituted or unsubstituted, saturated or unsaturated ring, and R₂₁ and R₂₂ are independently a hydrogen atom or substituent.

The substituent is

a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

### —S—(R₉₀₅),

—N(R₉₀₆)(R₉₀₇),
a halogen atom, a cyano group, a nitro group,
a substituted or unsubstituted aryl group including 6 to 50 ring carbon atoms, or
a substituted or unsubstituted monovalent heterocyclic group including 5 to 50 ring atoms.

R₉₀₁ to R₉₀₇ are independently

a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms.

When two or more of each of R₉₀₁ to R₉₀₇ are present, the two or more of each of R₉₀₁ to R₉₀₇ may be the same or different.

Provided that the formula (A1) satisfies one or both of the following requirements (i) and (ii).

(i) One or more sets of adjacent two or more among R₁ to R₇ and R₁₀ to R₁₆ form a substituted or unsubstituted, saturated or unsaturated ring by bonding with each other.

(ii) One or more among R₁ to R₇, R₁₀ to R₁₆, R₂₁ and R₂₂ is the substituent.

Since an electron barrier layer using the compound represented by the formula (B1) or (C1) has good hole-injecting property into the emitting layer, it is considered that a device using the compound has high efficiency. However, when the intermolecular interaction of the dopant used in the emitting layer in the device is strong, such characteristics of the compound represented by the formula (B1) or (C1) are not sufficiently exhibited.

Conventionally, a device in which a compound having no substituent or a fused ring structure in the central skeleton of a compound represented by the formula (A1) is combined with a compound represented by the formula (B1) or (C1) has been known. However, a compound having no substituent or a fused ring structure in the central skeleton of a compound represented by the formula (A1) has strong intermolecular interaction, and the effect of a compound represented by the formula (B1) or (C1) was not sufficiently obtained.

In one embodiment of the invention, by using a compound having a substituent or a fused ring structure in the central skeleton represented by the formula (A1) in the emitting layer, an intermolecular interaction is suppressed, and as a result, an improvement in luminous efficiency by a compound represented by the formula (B1) or (C1) is exhibited.

In one embodiment, the compound represented by the formula (A1) satisfies only the requirement (i).

In one embodiment, the compound represented by the formula (A1) satisfies only the requirement (ii).

In one embodiment, the compound represented by the formula (A1) satisfies the requirements (i) and (ii).

In one embodiment, one or more among R₁ to R₇ and R₁₀ to R₁₆ of the formula (A1) is —N(R₉₀₆)(R₉₀₇).

In one embodiment, two or more of R₁ to R₇ and R₁₀ to R₁₆ of the formula (A1) are —N(R₉₀₆)(R₉₀₇).

In one embodiment, the compound represented by the formula (A1) is a compound represented by the following formula (A10).

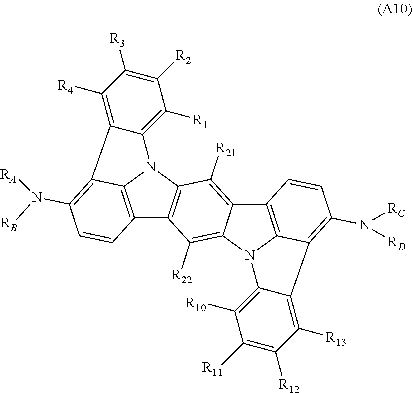

In the formula (A10),

R₁ to R₄, R₁₀ to R₁₃, R₂₁ and R₂₂ are as defined in the formula (A1).

R_{A}, R_{B}, R_{C} and R_{D} are independently a substituted or unsubstituted aryl group including 6 to 18 ring carbon atoms, or a substituted or unsubstituted monovalent heterocyclic group including 5 to 18 ring atoms.

In one embodiment, the compound represented by the formula (A10) is a compound represented by the following formula (A11).

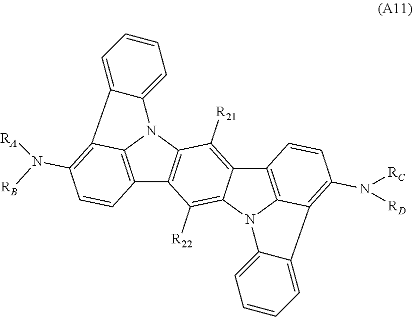

In the formula (A11), R₂₁, R₂₂, R_{A}, R_{B}, R_{C} and R_{D} are as defined in the formula (A10).

In one embodiment, R_{A}, R_{B}, R_{C} and R_{D} in the formula (A11) are independently a substituted or unsubstituted aryl group including 6 to 18 ring carbon atoms.

In one embodiment, R_{A}, R_{B}, R_{C} and R_{D} are independently a substituted or unsubstituted phenyl group.

In one embodiment, one or more sets selected from the group consisting of R₁ and R₂, R₂ and R₃, R₃ and R₄, R₁₀ and R₁₁, R₁₁ and R₁₂, and R₁₂ and R₁₃ of the formula (A1) form a ring represented by the following formula (X).

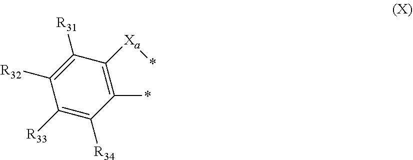

In the formula (X),

two “*”s are respectively bonded to R₁ and R₂, R₂ and R₃, R₃ and R₄, R₁₀ and R₁₁, R₁₁ and R₁₂ or R₁₂ and R₁₃ of the formula (A1).

Xₐ is selected from the group consisting of O, S and N(R₃₅), and when two or more Xₐ's are present, the plurality of Xₐ's may be the same with or different from each other.

R₃₅ forms a substituted or unsubstituted, saturated or unsaturated ring by bonding with R₃₁, or does not form the ring.

R₃₁ which does not form the ring with R₃₅, and R₃₂ to R₃₄ are independently a hydrogen atom, a substituted or unsubstituted alkyl group including 1 to 50 carbon atoms, or a substituted or unsubstituted aryl group including 6 to 50 ring carbon atoms.

R₃₅ which does not form the ring is

a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms.

In one embodiment, the compound represented by the formula (A1) is a compound represented by the following formula (A12).

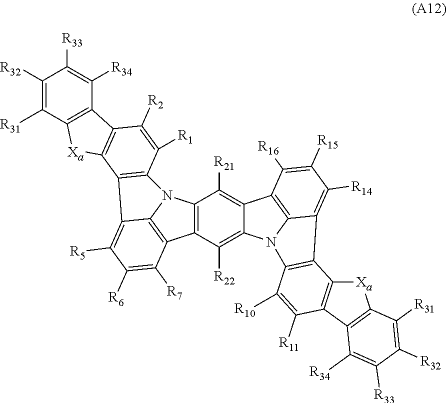

In the formula (A12), R₁, R₂, R₅ to R₇, R₁₀, R₁₁, R₁₄ to R₁₆, R₂₁, R₂₂, R₃₁ to R₃₄ and Xₐ are as defined in the formula (A1) and the formula (X).

In one embodiment, the compound represented by the formula (A1) is a compound represented by the following formula (A13).

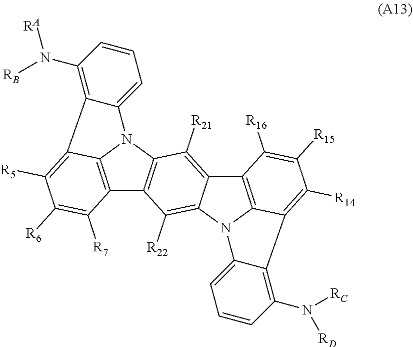

In the formula (A13), R₅ to R₇, R₁₄ to R₁₆, R₂₁, R₂₂, R_{A}, R_{B}, R_{C} and R_{D} are as defined in the formula (A1) and formula (A10).

In one embodiment, the compound represented by the formula (A13) is a compound represented by the following formula (A14).

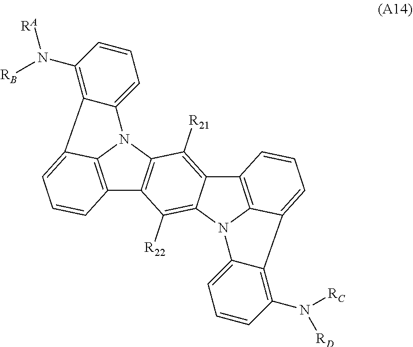

In the formula (A14), R₂₁, R₂₂, R_{A}, R_{B}, R_{C} and R_{D} are as defined in the formula (A1) and formula (A10).

In one embodiment, the compound represented by the formula (A1) is a compound represented by the following formula (A15).

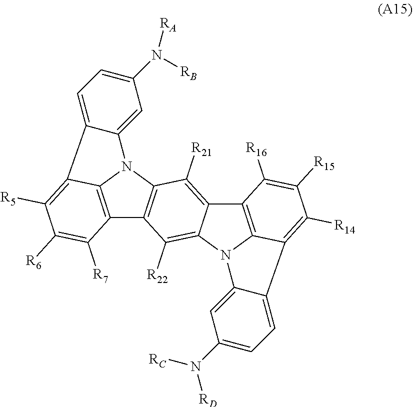

In the formula (A15), R₅ to R₇, R₁₄ to R₁₆, R₂₁, R₂₂, R_{A}, R_{B}, R_{C} and R_{D} are as defined in the formula (A1) and formula (A10).

In one embodiment, the compound represented by the formula (A15) is a compound represented by the following formula (A16).

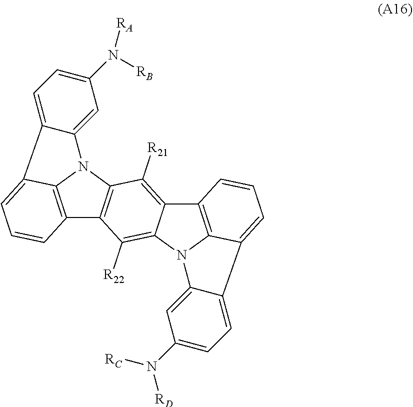

In the formula (A16), R₂₁, R₂₂, R_{A}, R_{B}, R_{C} and R_{D} are as defined in the formula (A1) and formula (A10).

In one embodiment, R₂₁ and R₂₂ of the formula (A1) are hydrogen atoms.

Specific examples of the compound represented by the formula (A1) include the following compounds. In the following specific examples, “Ph” represents a phenyl group, and “D” represents a deuterium atom.

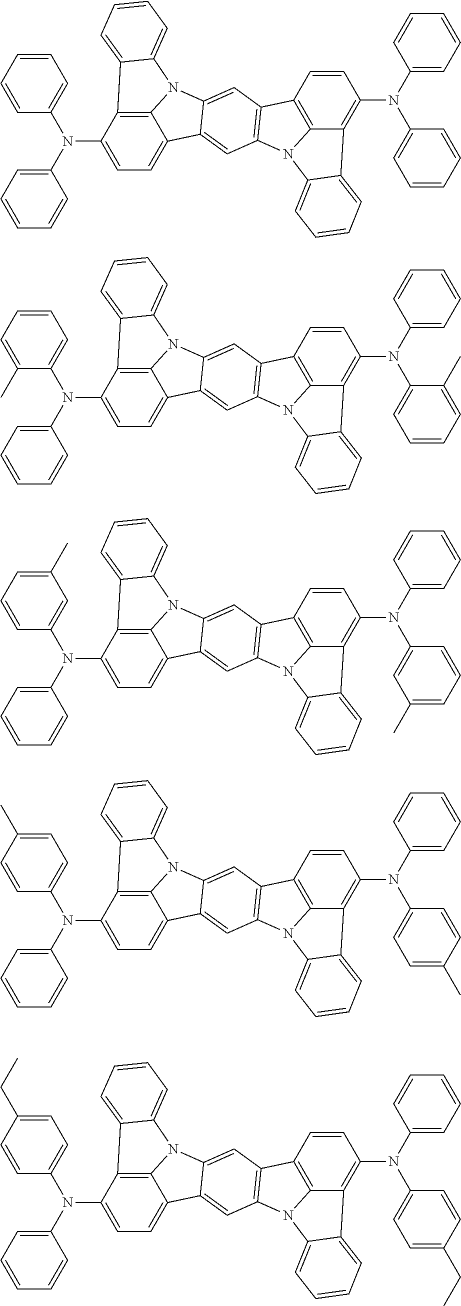 [SMILES: c1ccc(N(c2ccccc2)c2ccc3c4cc5c(cc4n4c6ccccc6c2c34)c2ccc(N(c3ccccc3)c3ccccc3)c3c4ccccc4n5c23)cc1 ; Cc1ccccc1N(c1ccccc1)c1ccc2c3cc4c(cc3n3c5ccccc5c1c23)c1ccc(N(c2ccccc2)c2ccccc2C)c2c3ccccc3n4c12 ; Cc1cccc(N(c2ccccc2)c2ccc3c4cc5c(cc4n4c6ccccc6c2c34)c2ccc(N(c3ccccc3)c3cccc(C)c3)c3c4ccccc4n5c23)c1 ; Cc1ccc(N(c2ccccc2)c2ccc3c4cc5c(cc4n4c6ccccc6c2c34)c2ccc(N(c3ccccc3)c3ccc(C)cc3)c3c4ccccc4n5c23)cc1 ; CCc1ccc(N(c2ccccc2)c2ccc3c4cc5c(cc4n4c6ccccc6c2c34)c2ccc(N(c3ccccc3)c3ccc(CC)cc3)c3c4ccccc4n5c23)cc1]

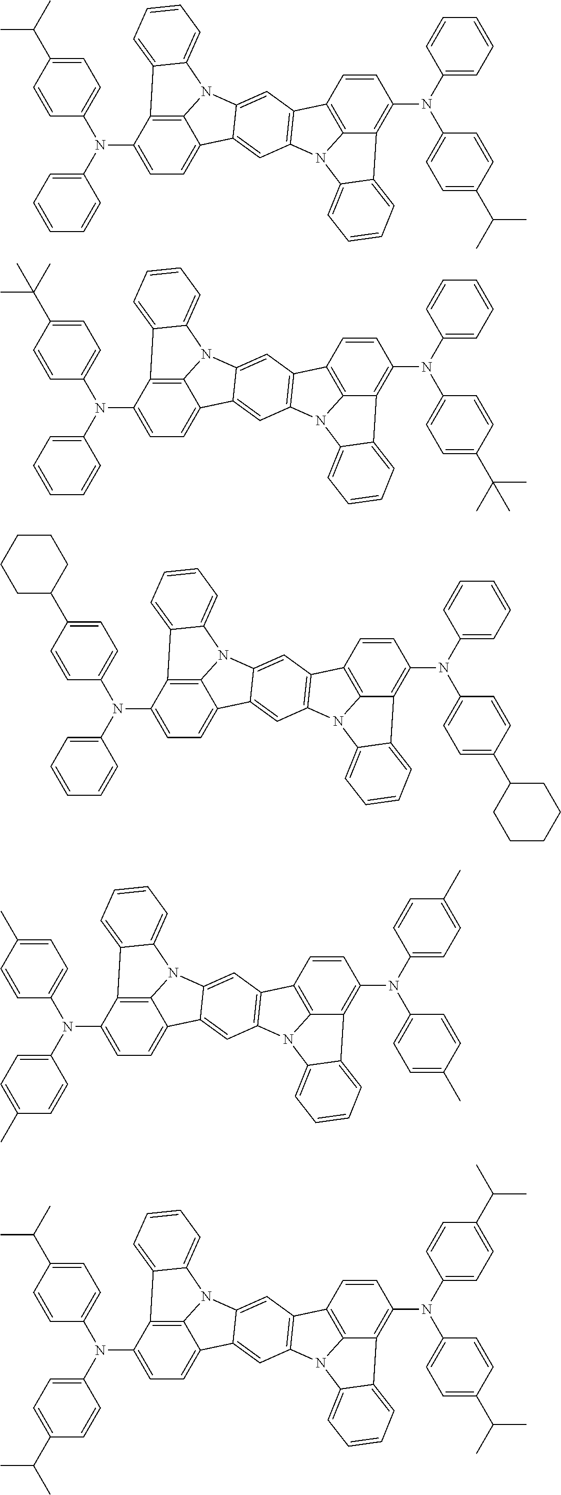

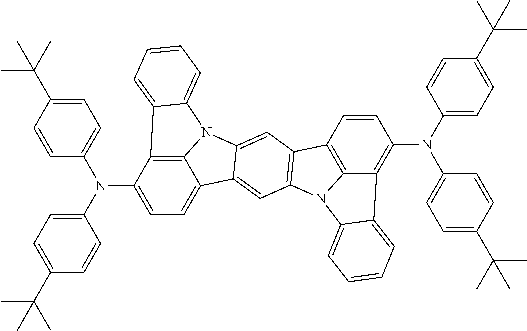 [SMILES: CC(C)(C)c1ccc(N(c2ccc(C(C)(C)C)cc2)c2ccc3c4cc5c(cc4n4c6ccccc6c2c34)c2ccc(N(c3ccc(C(C)(C)C)cc3)c3ccc(C(C)(C)C)cc3)c3c4ccccc4n5c23)cc1]

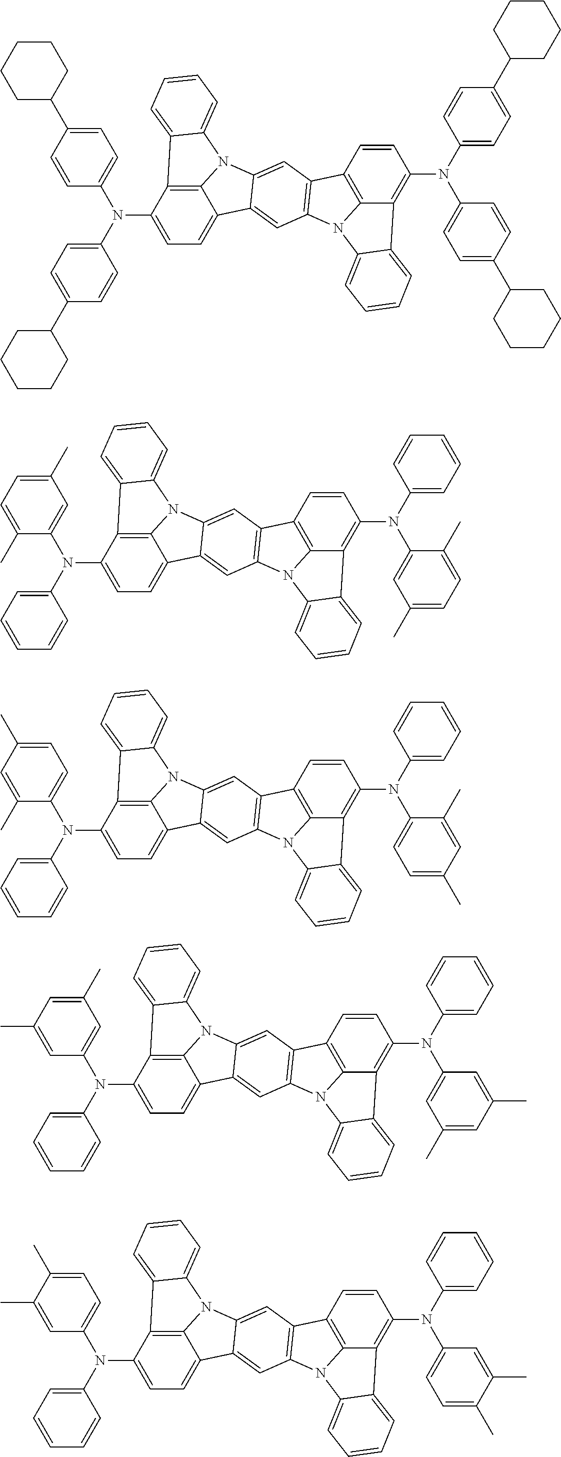

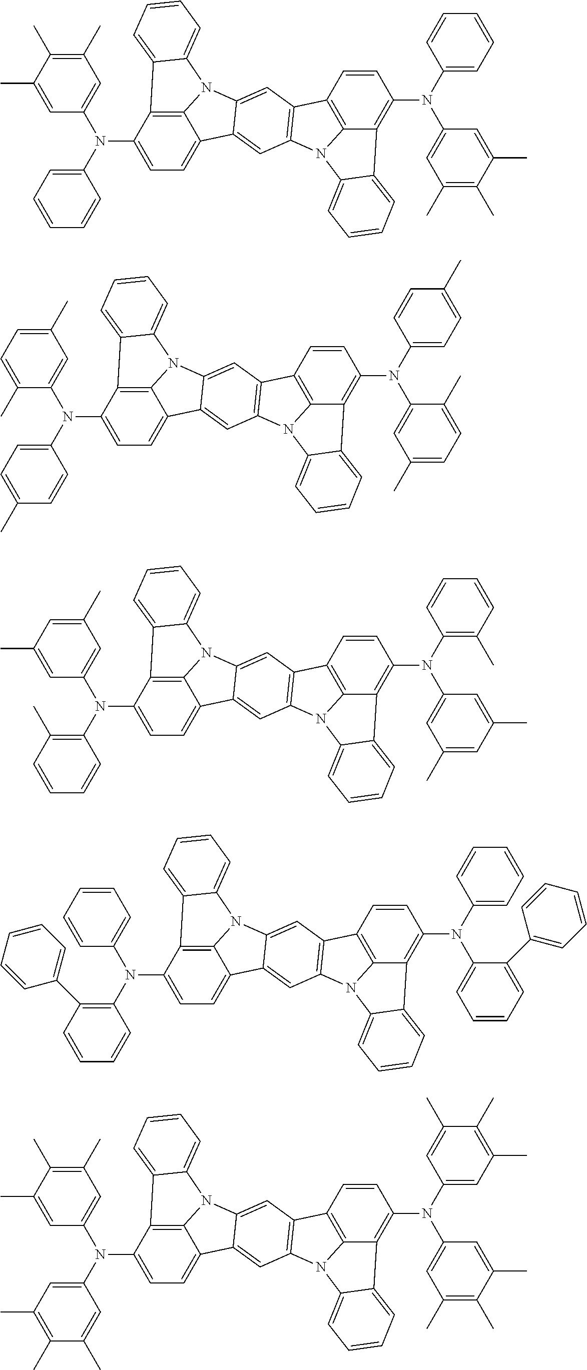

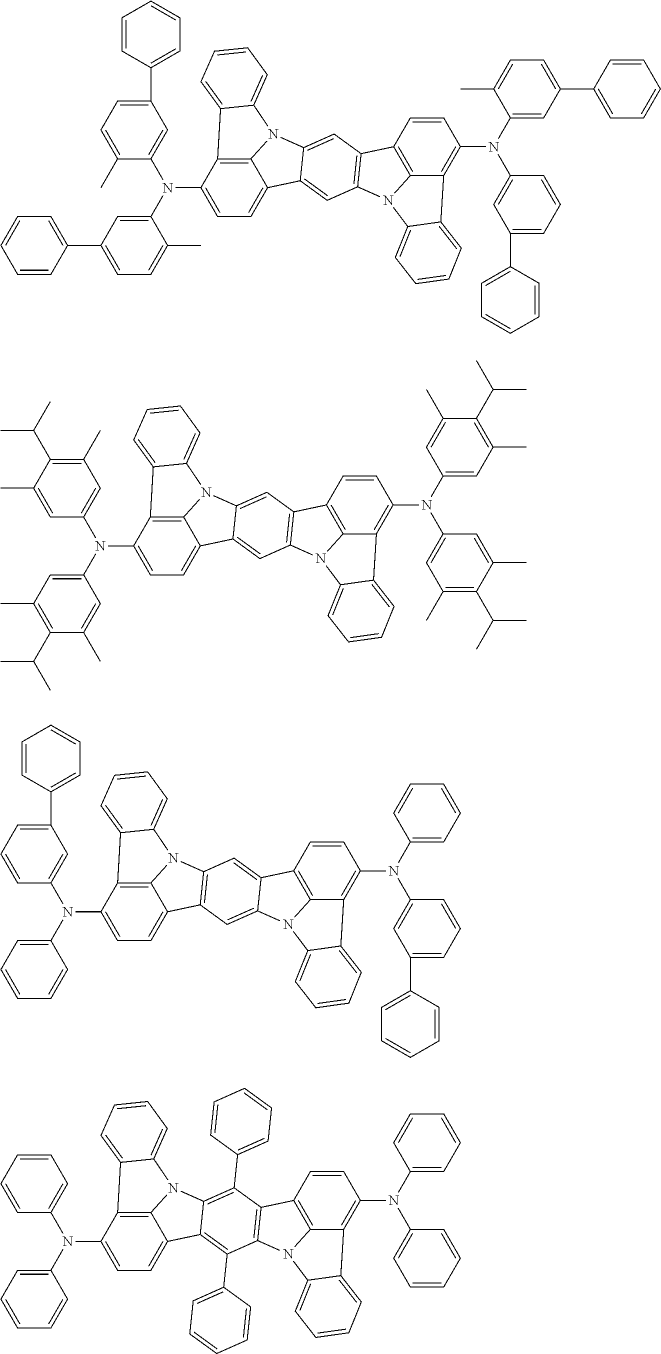

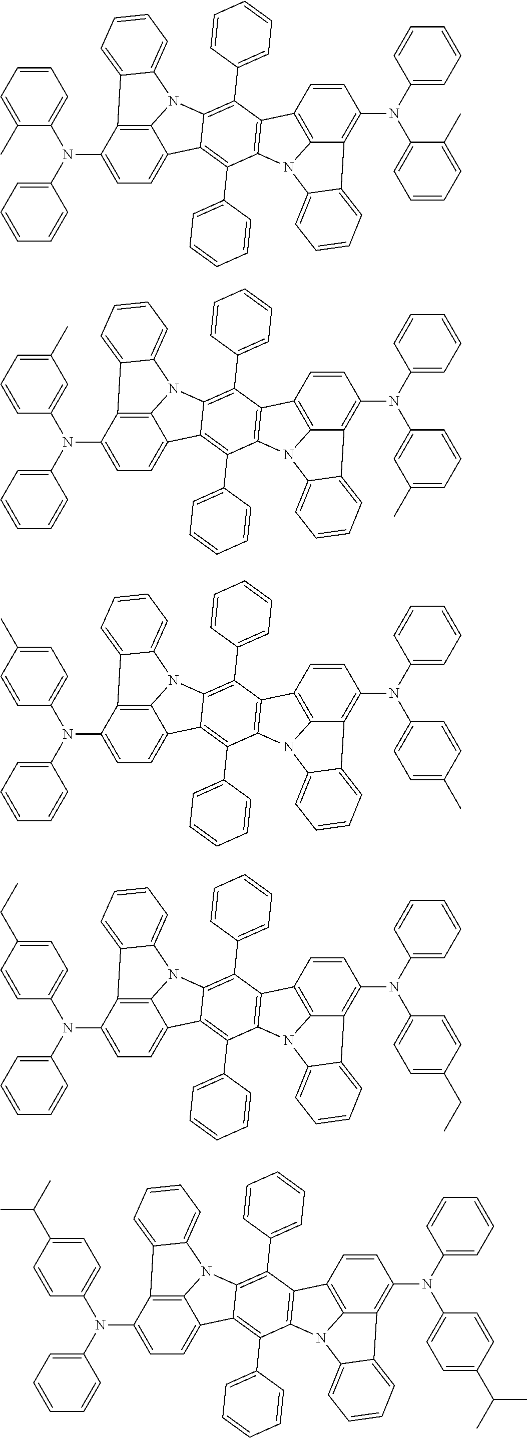

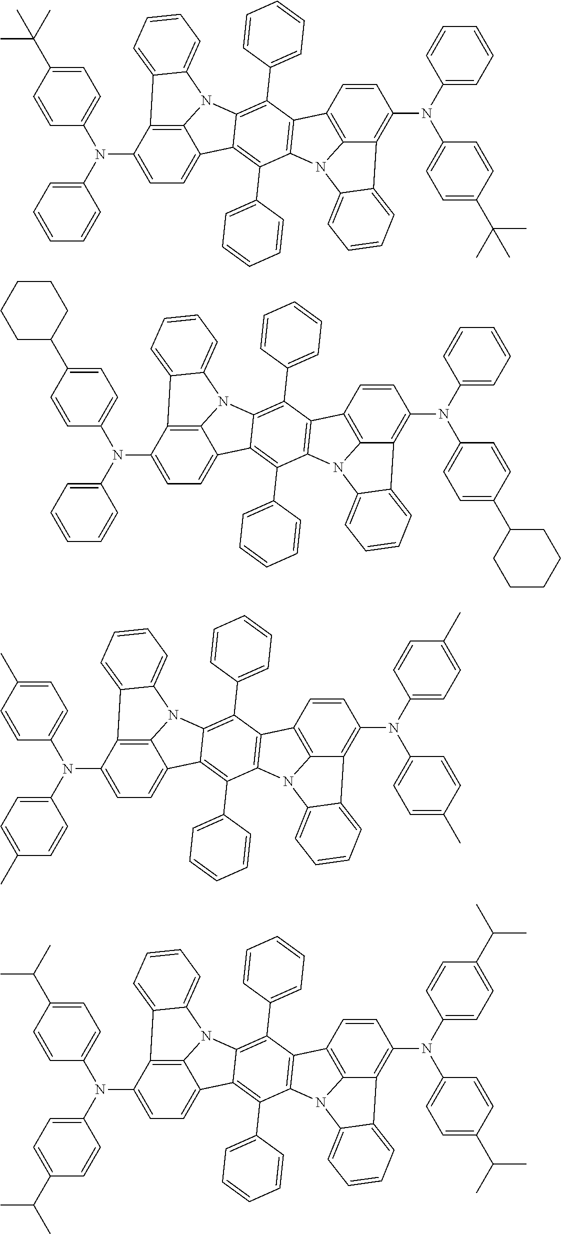

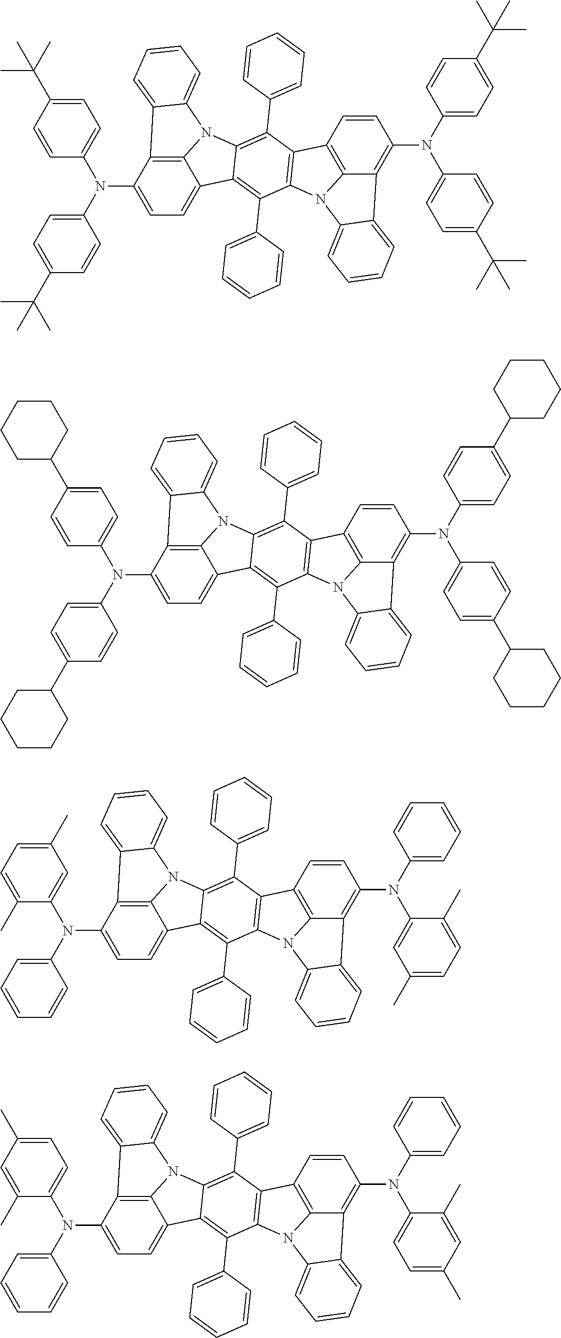

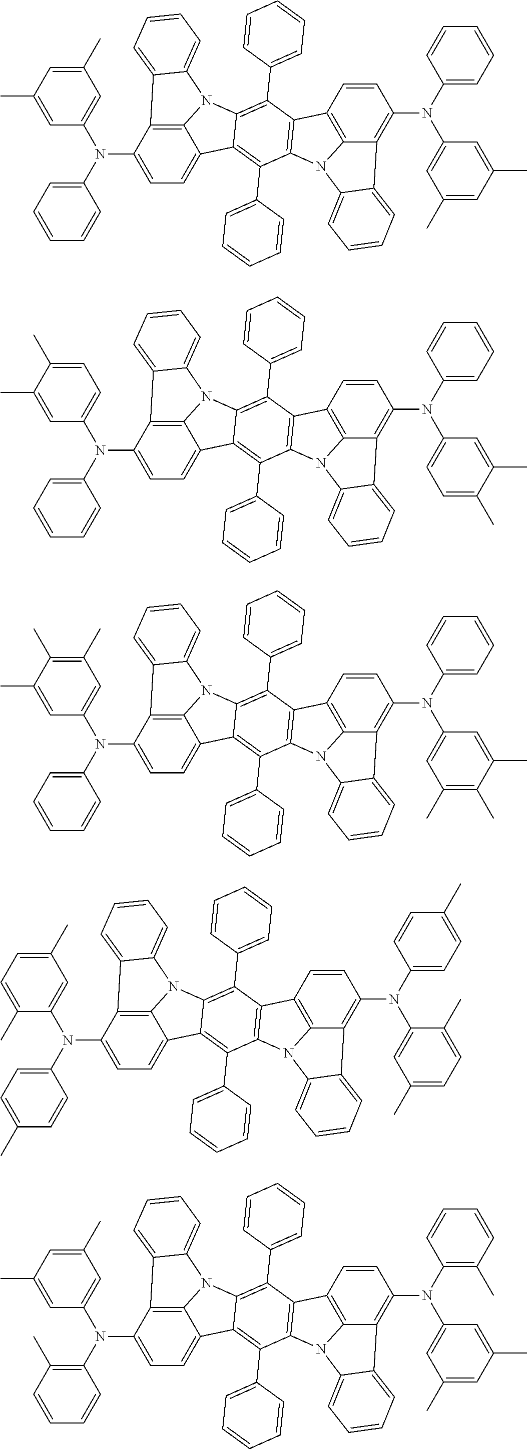

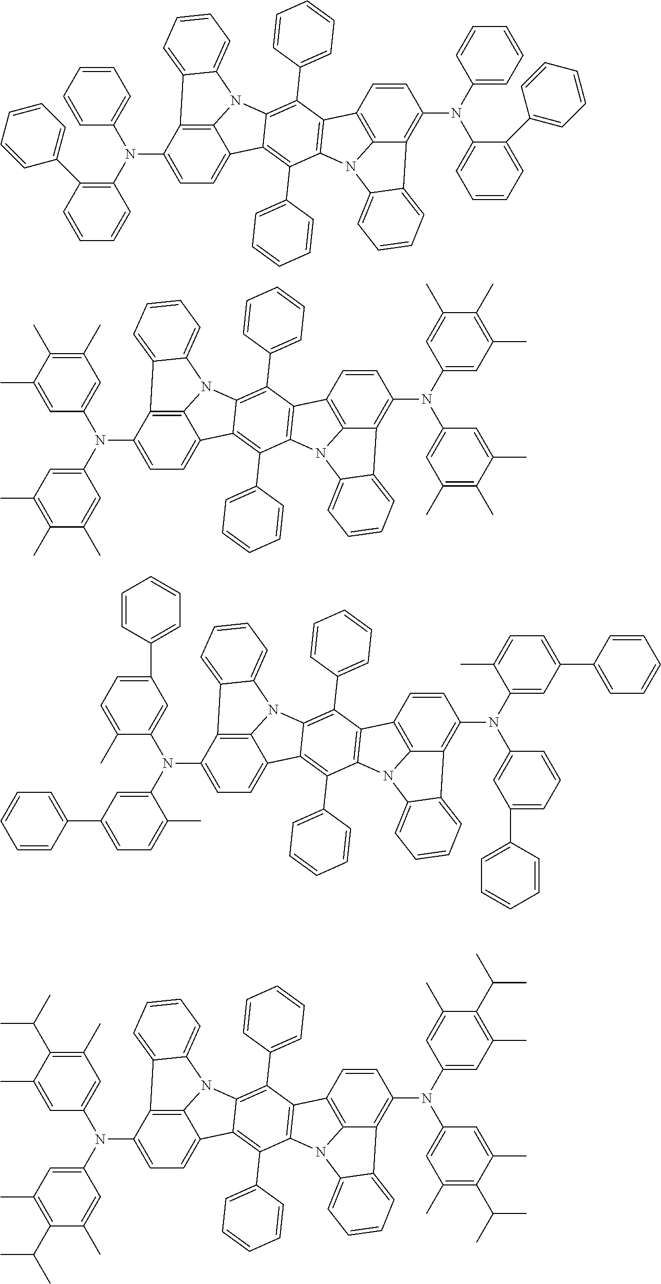

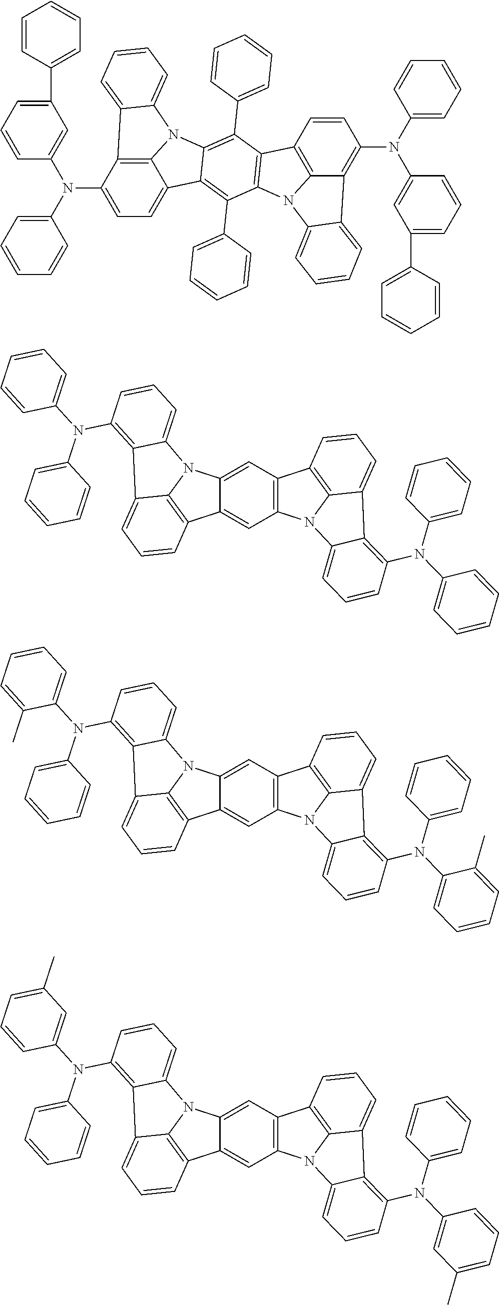 [SMILES: c1ccc(-c2cccc(N(c3ccccc3)c3ccc4c5c(-c6ccccc6)c6c(c(-c7ccccc7)c5n5c7ccccc7c3c45)c3ccc(N(c4ccccc4)c4cccc(-c5ccccc5)c4)c4c5ccccc5n6c34)c2)cc1 ; c1ccc(N(c2ccccc2)c2cccc3c2c2cccc4c5cc6c(cc5n3c42)c2cccc3c4c(N(c5ccccc5)c5ccccc5)cccc4n6c23)cc1 ; Cc1ccccc1N(c1ccccc1)c1cccc2c1c1cccc3c4cc5c(cc4n2c31)c1cccc2c3c(N(c4ccccc4)c4ccccc4C)cccc3n5c12 ; Cc1cccc(N(c2ccccc2)c2cccc3c2c2cccc4c5cc6c(cc5n3c42)c2cccc3c4c(N(c5ccccc5)c5cccc(C)c5)cccc4n6c23)c1]

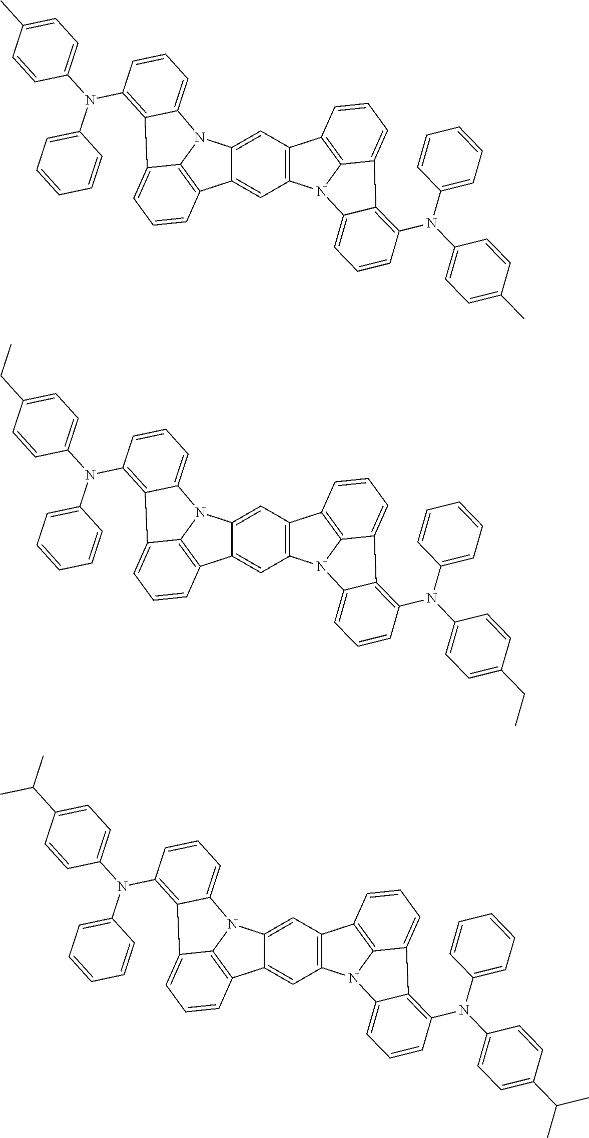

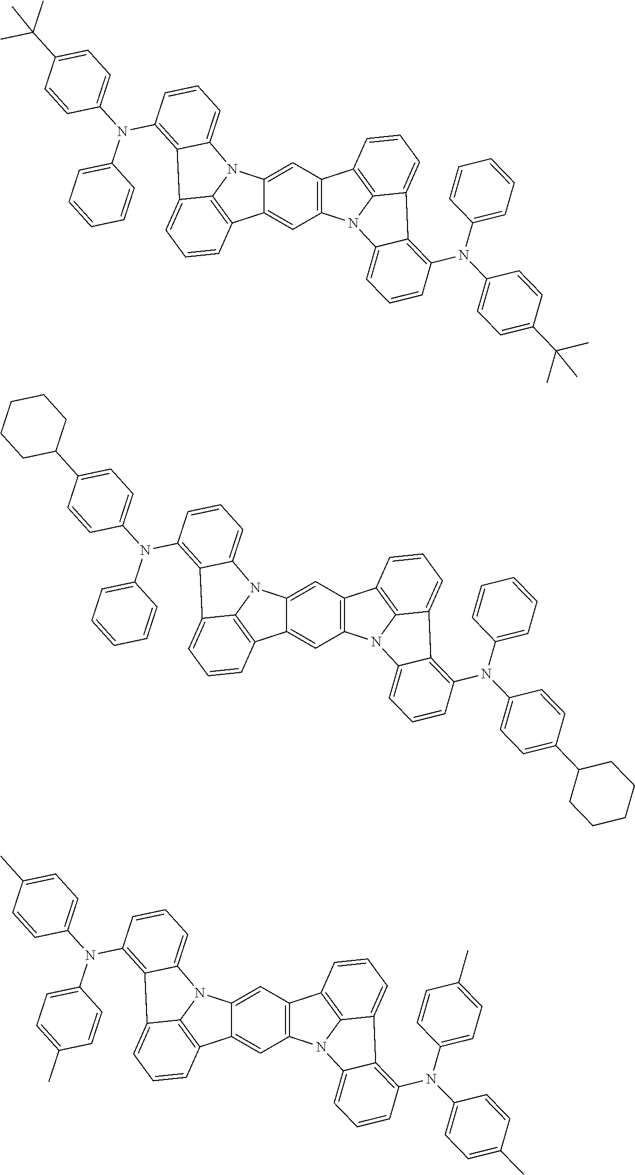

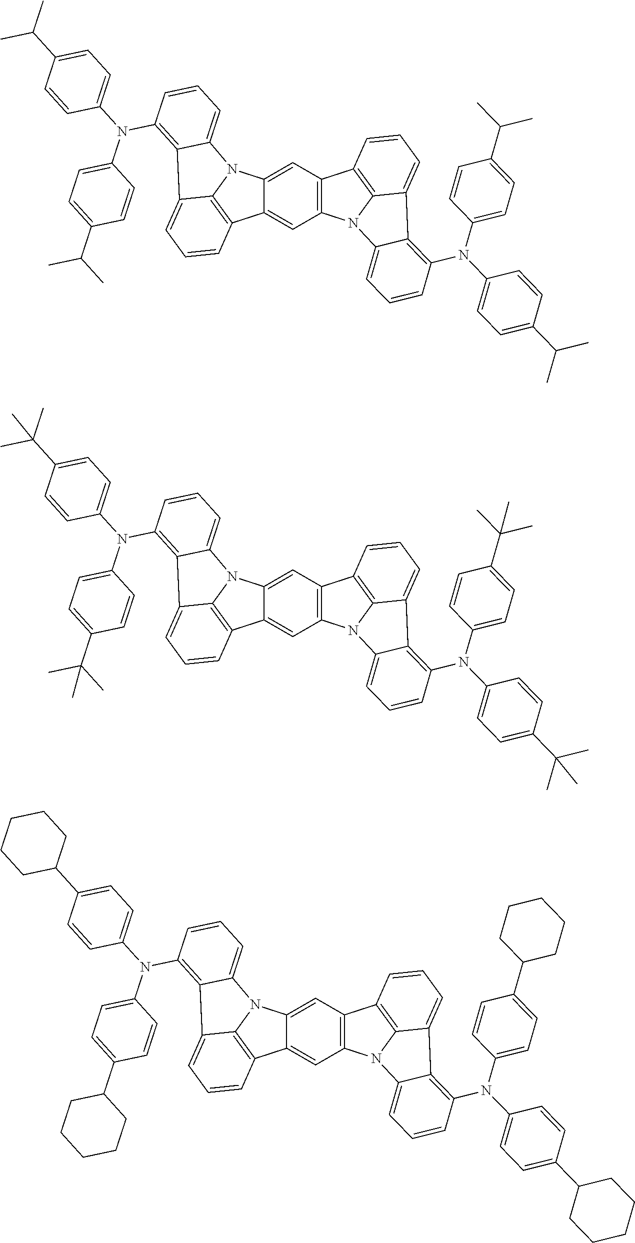

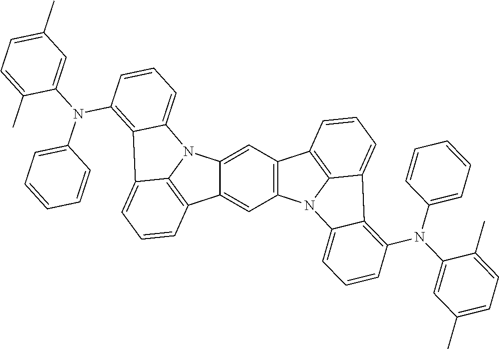 [SMILES: Cc1ccc(C)c(N(c2ccccc2)c2cccc3c2c2cccc4c5cc6c(cc5n3c42)c2cccc3c4c(N(c5ccccc5)c5cc(C)ccc5C)cccc4n6c23)c1]

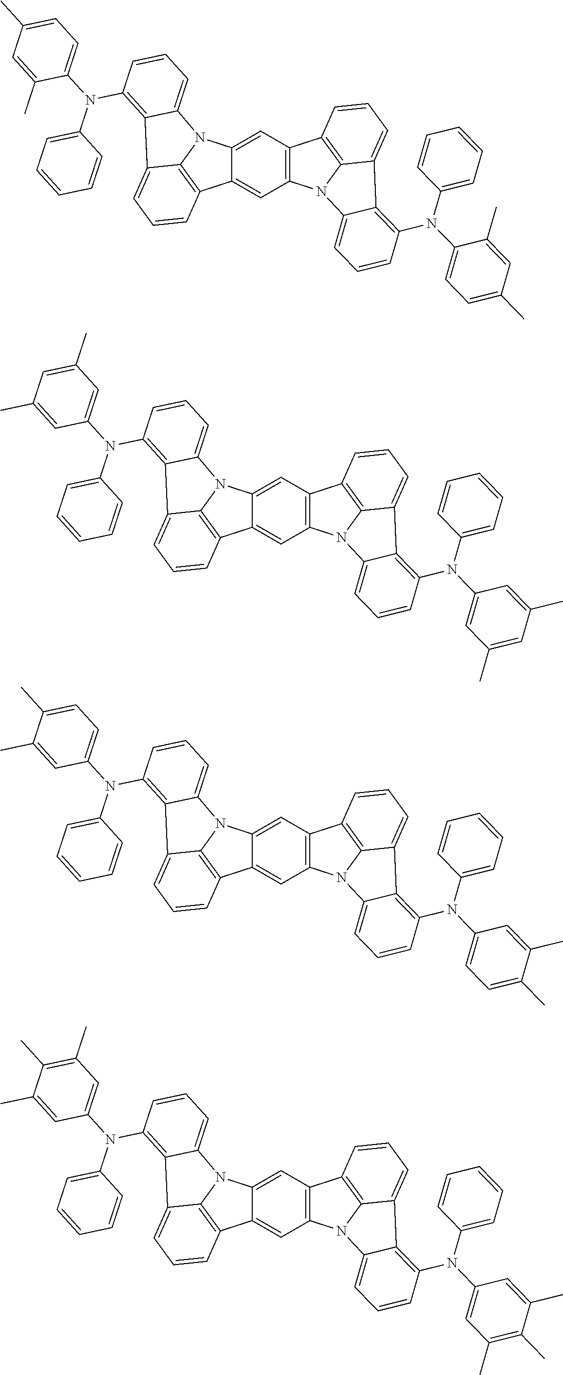

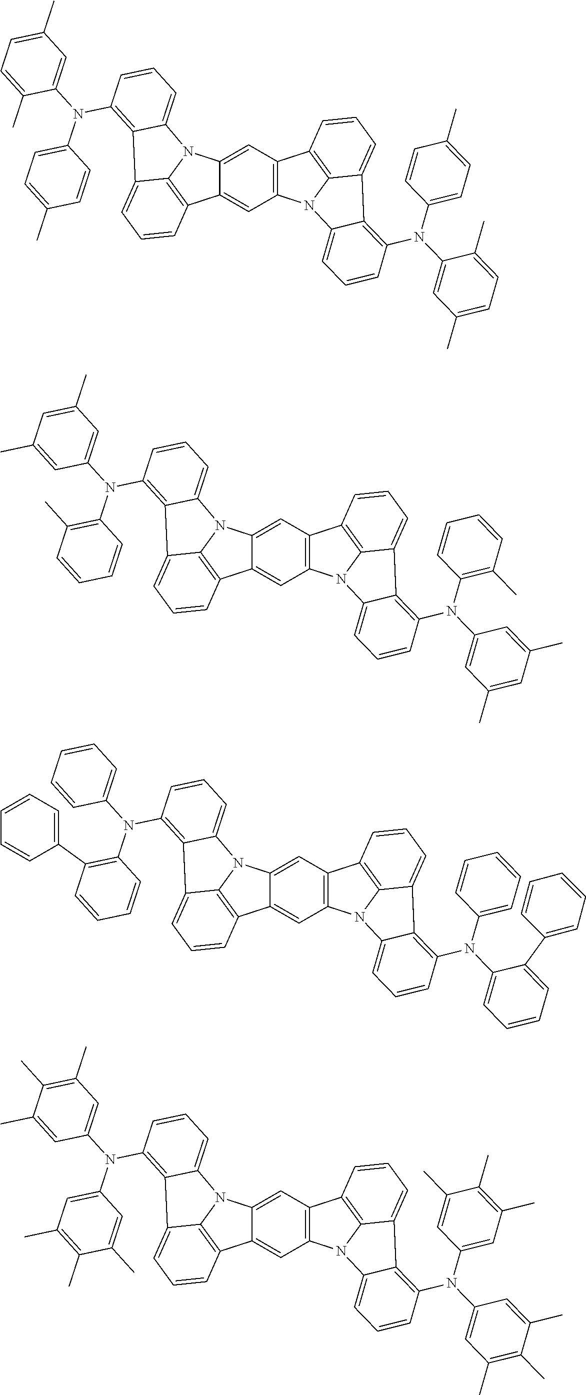

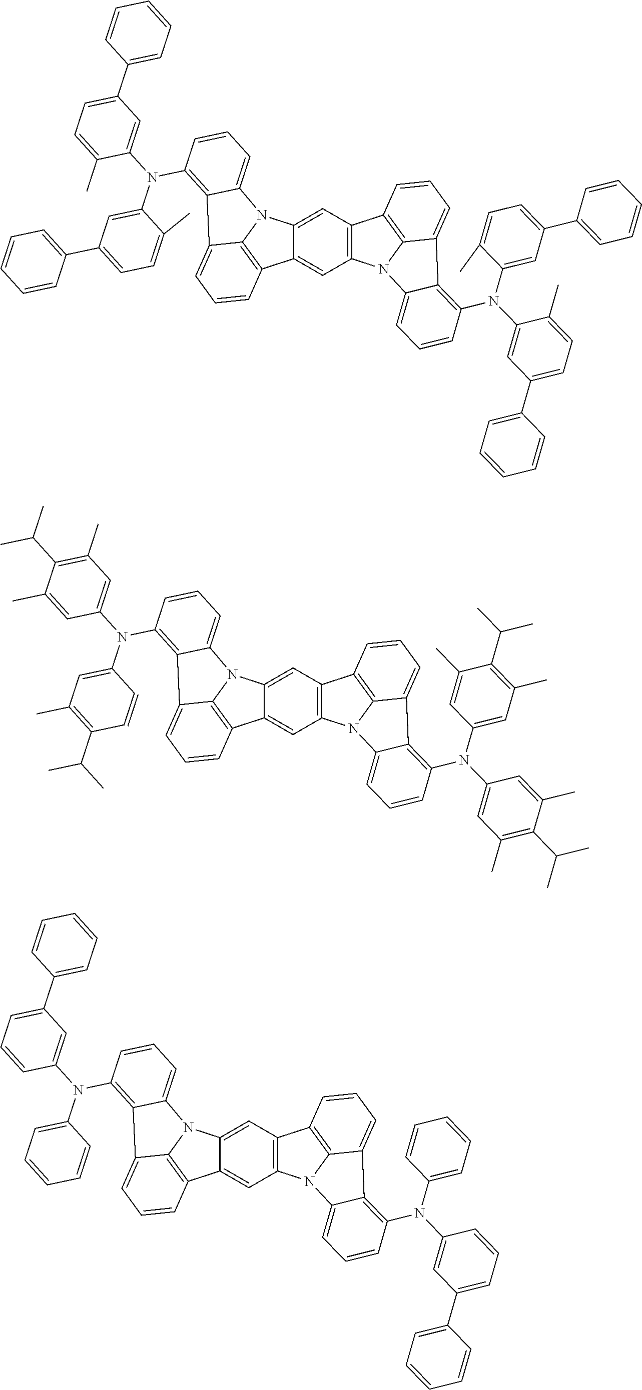 [SMILES: Cc1ccc(-c2ccccc2)cc1N(c1cc(-c2ccccc2)ccc1C)c1cccc2c1c1cccc3c4cc5c(cc4n2c31)c1cccc2c3c(N(c4cc(-c6ccccc6)ccc4C)c4cc(-c6ccccc6)ccc4C)cccc3n5c12 ; Cc1cc(N(c2cc(C)c(C(C)C)c(C)c2)c2cccc3c2c2cccc4c5cc6c(cc5n3c42)c2cccc3c4c(N(c5cc(C)c(C(C)C)c(C)c5)c5cc(C)c(C(C)C)c(C)c5)cccc4n6c23)ccc1C(C)C ; c1ccc(-c2cccc(N(c3ccccc3)c3cccc4c3c3cccc5c6cc7c(cc6n4c53)c3cccc4c5c(N(c6ccccc6)c6cccc(-c8ccccc8)c6)cccc5n7c34)c2)cc1]

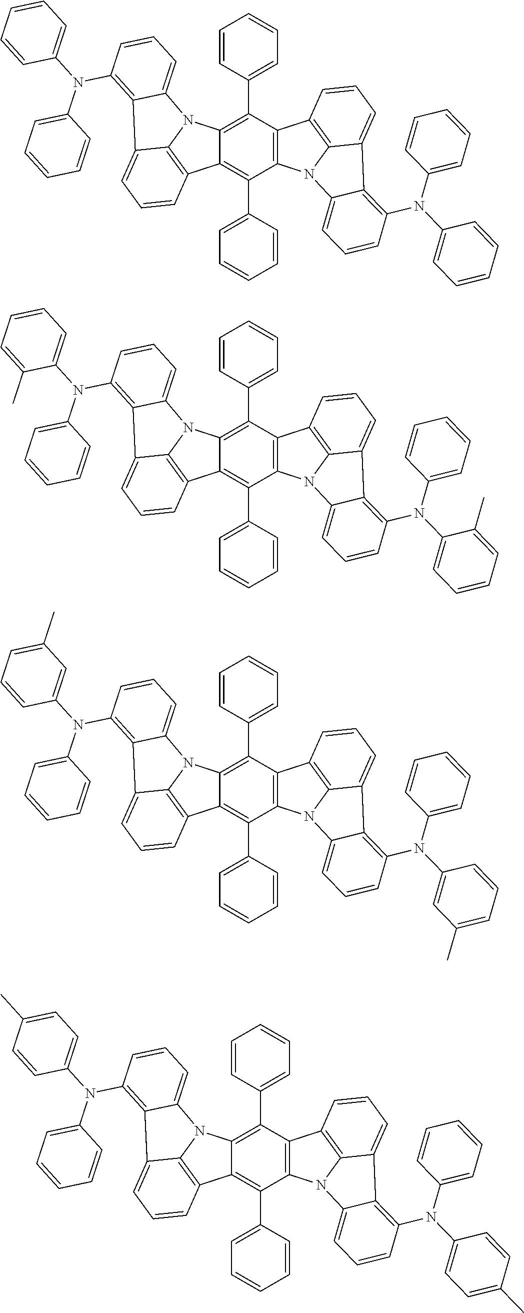

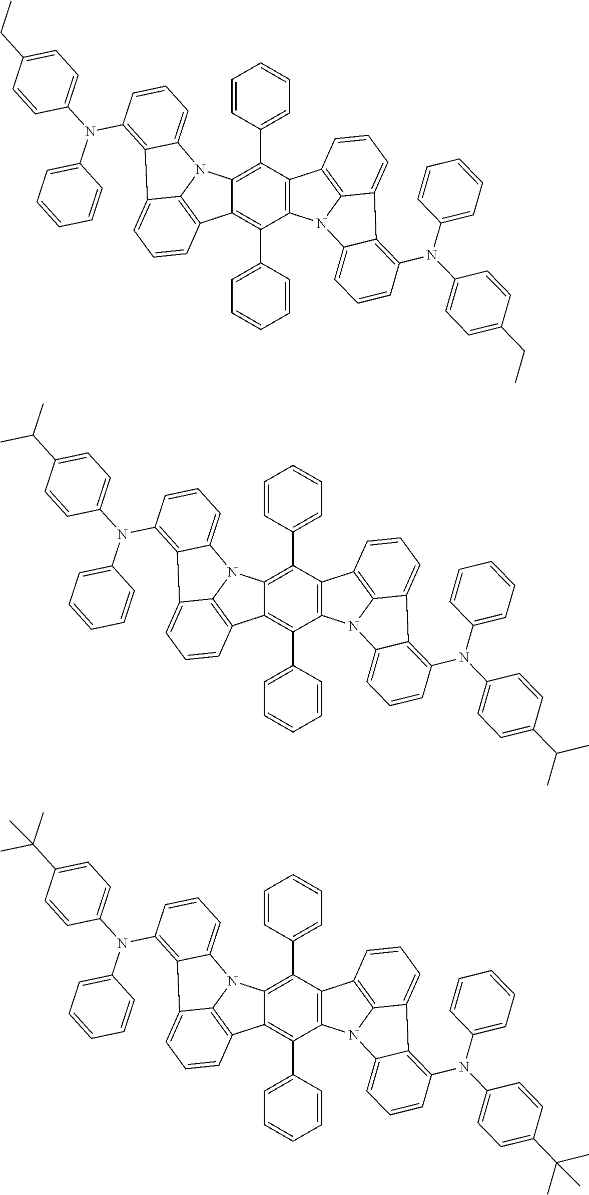

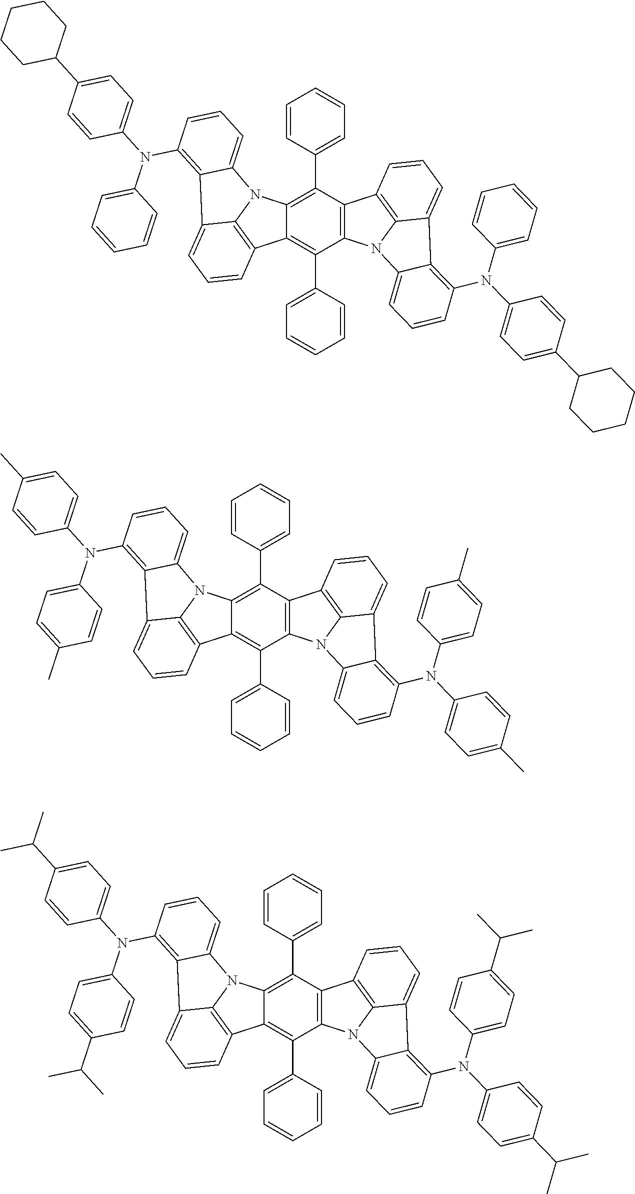

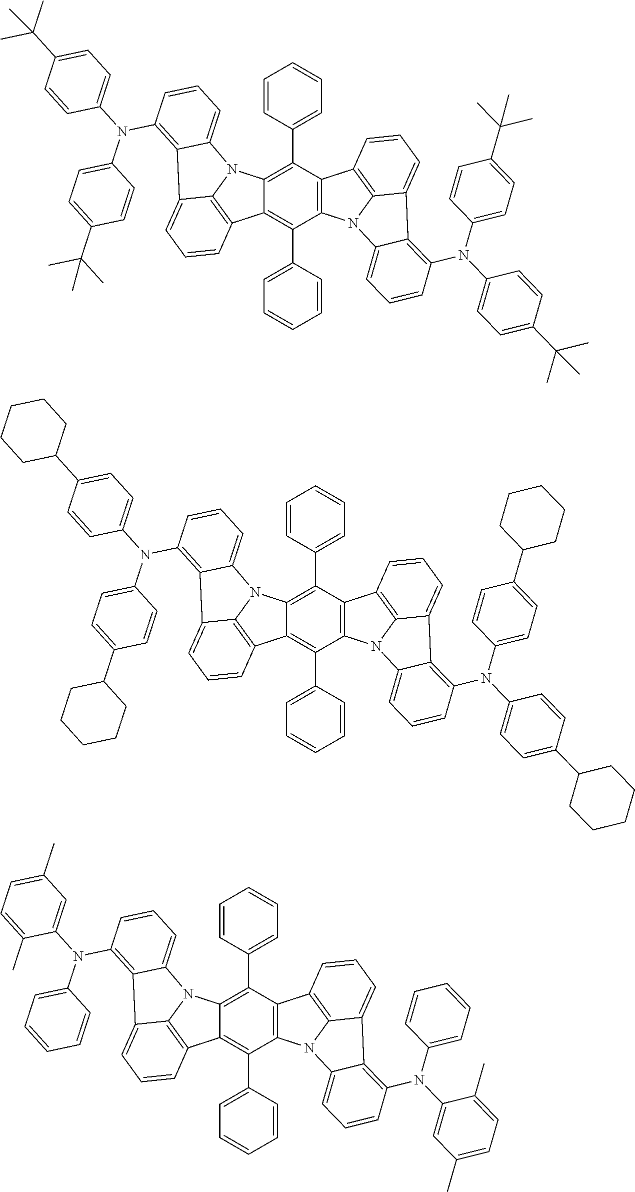 [SMILES: CC(C)(C)c1ccc(N(c2ccc(C(C)(C)C)cc2)c2cccc3c2c2cccc4c5c(-c6ccccc6)c6c(c(-c7ccccc7)c5n3c24)c2cccc3c4c(N(c5ccc(C(C)(C)C)cc5)c5ccc(C(C)(C)C)cc5)cccc4n6c32)cc1 ; c1ccc(-c2c3c4cccc5c6c(N(c7ccc(C8CCCCC8)cc7)c7ccc(C8CCCCC8)cc7)cccc6n(c3c(-c3ccccc3)c3c6cccc7c8c(N(c9ccc(C%10CCCCC%10)cc9)c9ccc(C%10CCCCC%10)cc9)cccc8n(c23)c76)c54)cc1 ; Cc1ccc(C)c(N(c2ccccc2)c2cccc3c2c2cccc4c5c(-c6ccccc6)c6c(c(-c7ccccc7)c5n3c24)c2cccc3c4c(N(c5ccccc5)c5cc(C)ccc5C)cccc4n6c32)c1]

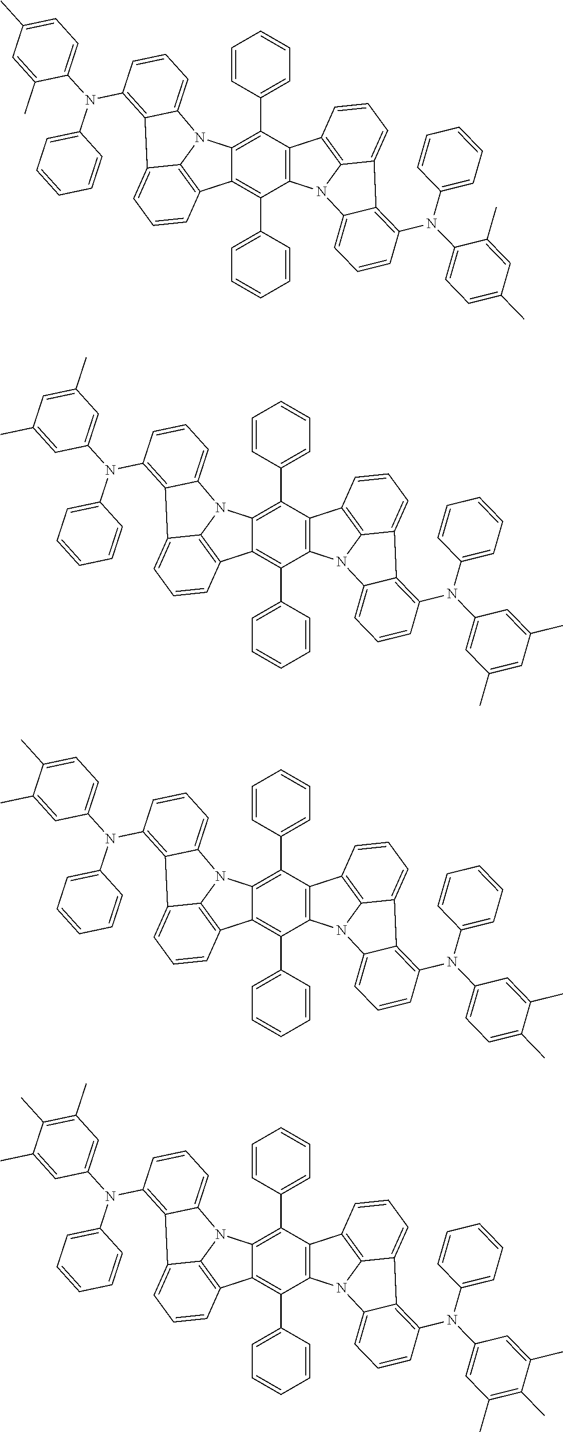

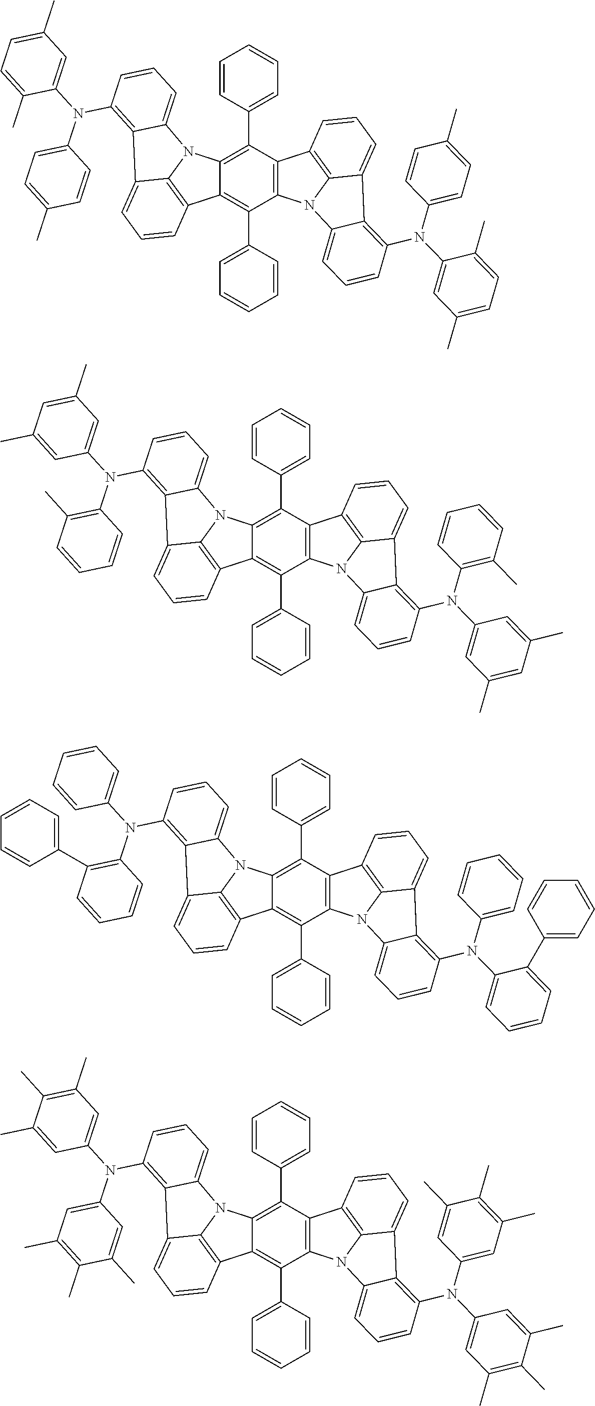

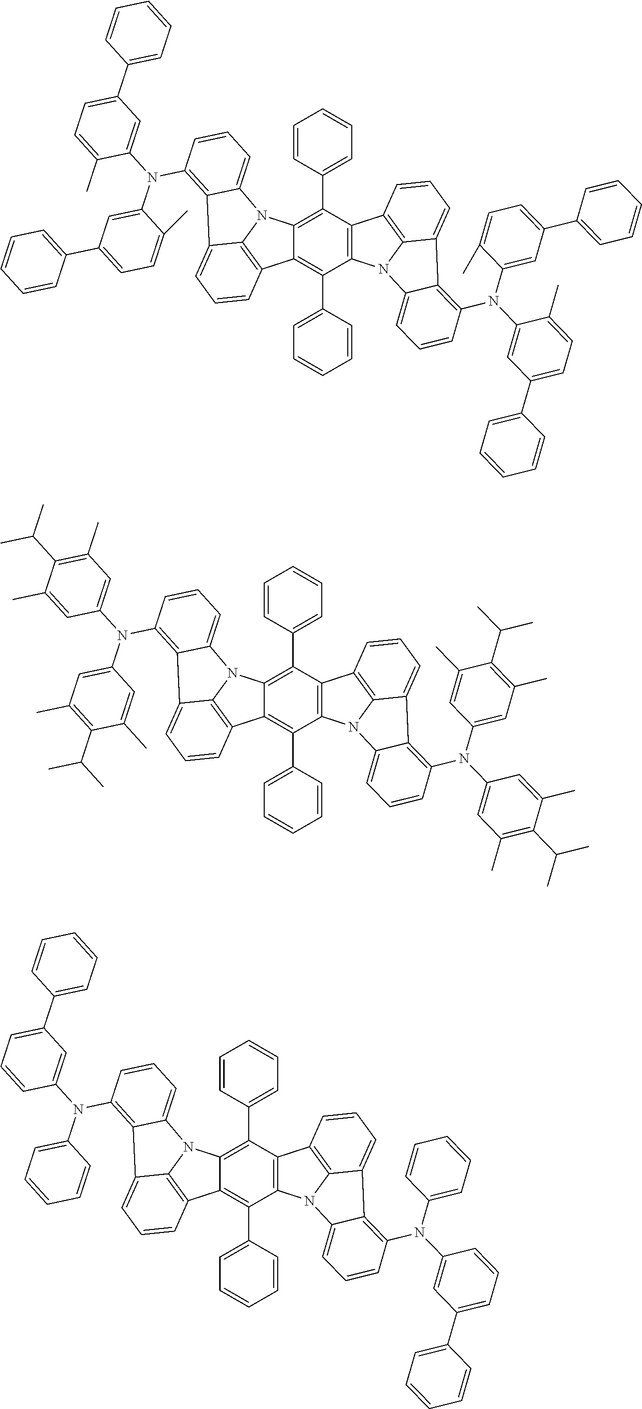 [SMILES: Cc1ccc(-c2ccccc2)cc1N(c1cc(-c2ccccc2)ccc1C)c1cccc2c1c1cccc3c4c(-c5ccccc5)c5c(c(-c6ccccc6)c4n2c13)c1cccc2c3c(N(c4cc(-c6ccccc6)ccc4C)c4cc(-c6ccccc6)ccc4C)cccc3n5c21 ; Cc1cc(N(c2cc(C)c(C(C)C)c(C)c2)c2cccc3c2c2cccc4c5c(-c6ccccc6)c6c(c(-c7ccccc7)c5n3c24)c2cccc3c4c(N(c5cc(C)c(C(C)C)c(C)c5)c5cc(C)c(C(C)C)c(C)c5)cccc4n6c32)cc(C)c1C(C)C ; c1ccc(-c2cccc(N(c3ccccc3)c3cccc4c3c3cccc5c6c(-c7ccccc7)c7c(c(-c8ccccc8)c6n4c35)c3cccc4c5c(N(c6ccccc6)c6cccc(-c8ccccc8)c6)cccc5n7c43)c2)cc1]

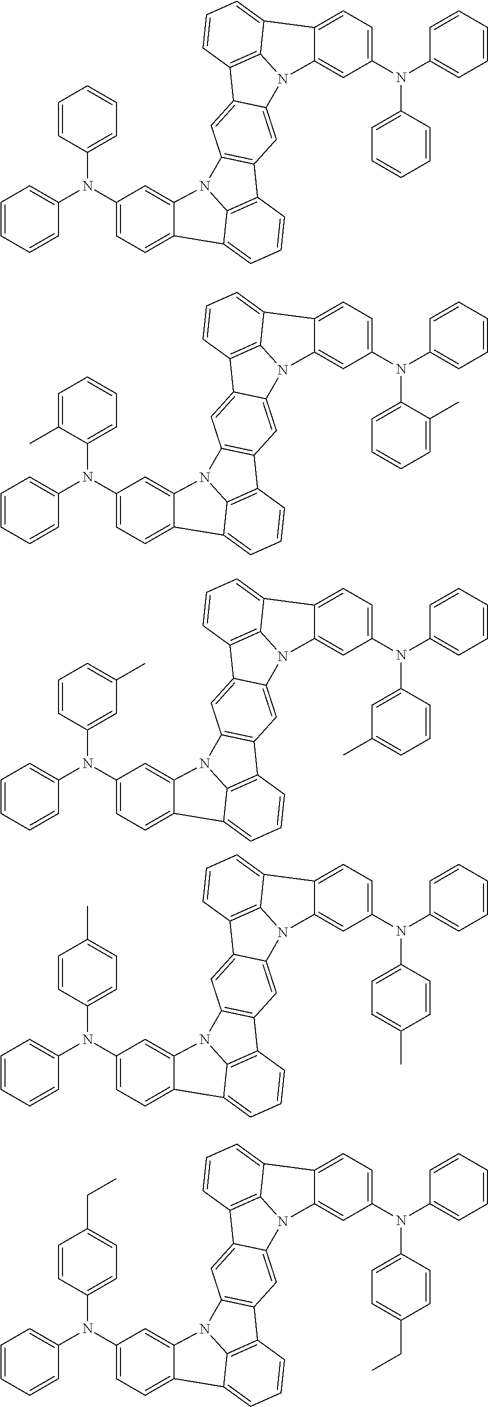

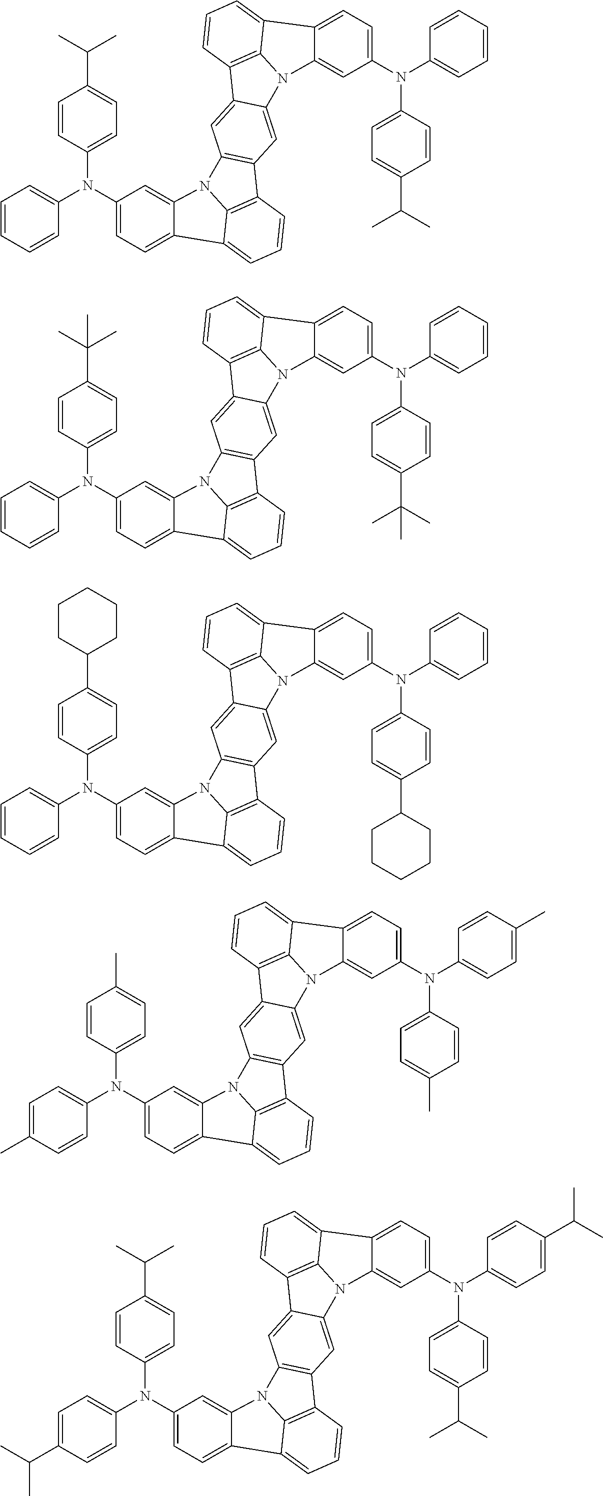

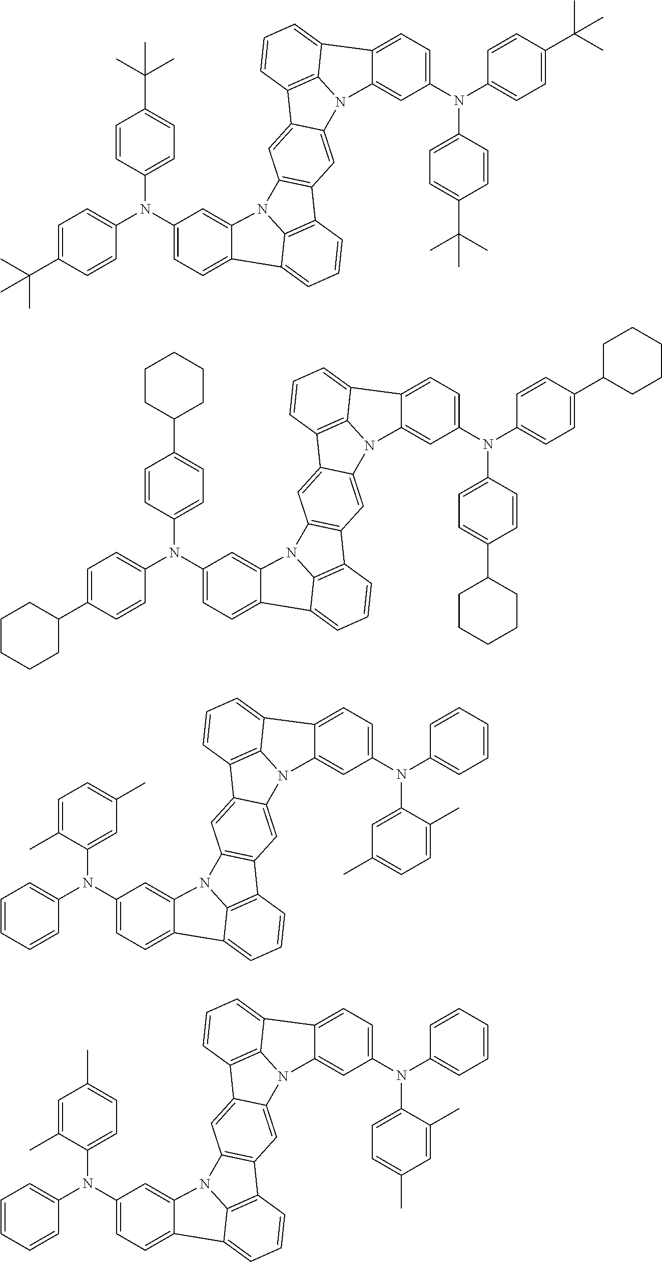

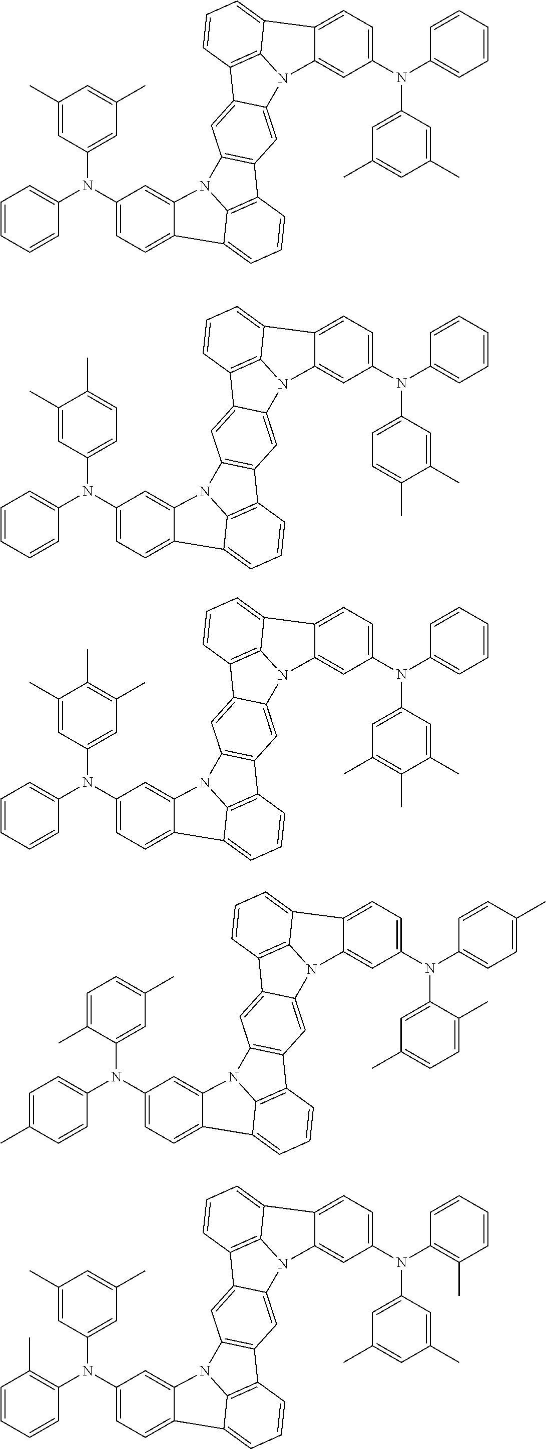

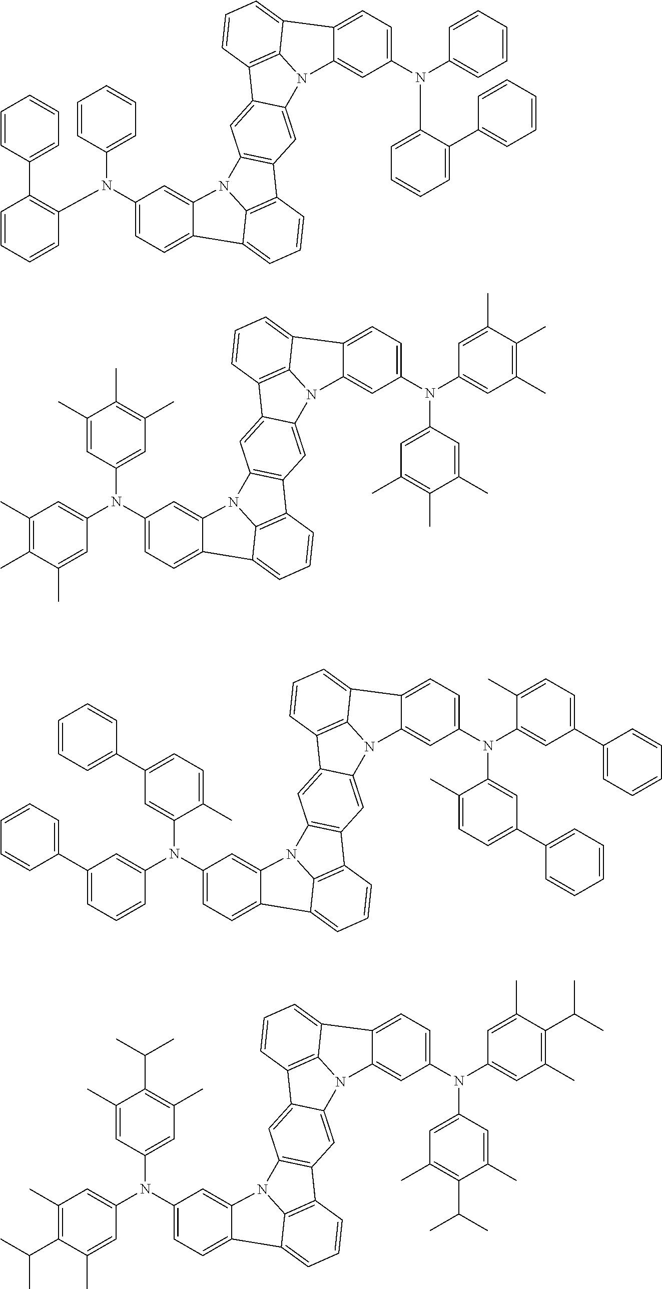

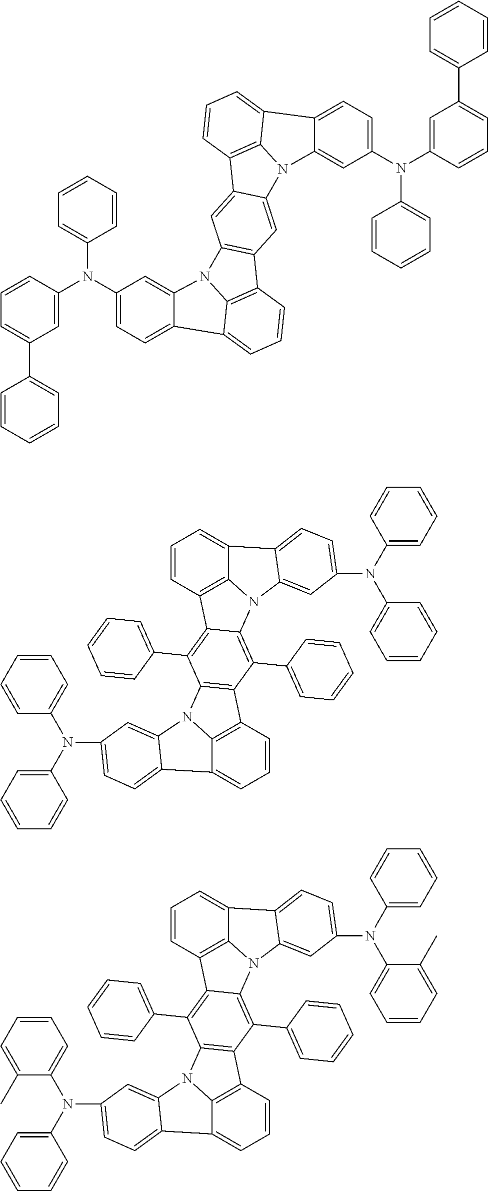 [SMILES: c1ccc(-c2cccc(N(c3ccccc3)c3ccc4c5cccc6c7cc8c(cc7n(c4c3)c56)c3cccc4c5ccc(N(c6ccccc6)c6cccc(-c7ccccc7)c6)cc5n8c43)c2)cc1 ; c1ccc(-c2c3c4cccc5c6ccc(N(c7ccccc7)c7ccccc7)cc6n(c3c(-c3ccccc3)c3c6cccc7c8ccc(N(c9ccccc9)c9ccccc9)cc8n(c23)c76)c54)cc1 ; Cc1ccccc1N(c1ccccc1)c1ccc2c3cccc4c5c(-c6ccccc6)c6c(c(-c7ccccc7)c5n(c2c1)c34)c1cccc2c3ccc(N(c4ccccc4)c4ccccc4C)cc3n6c21]

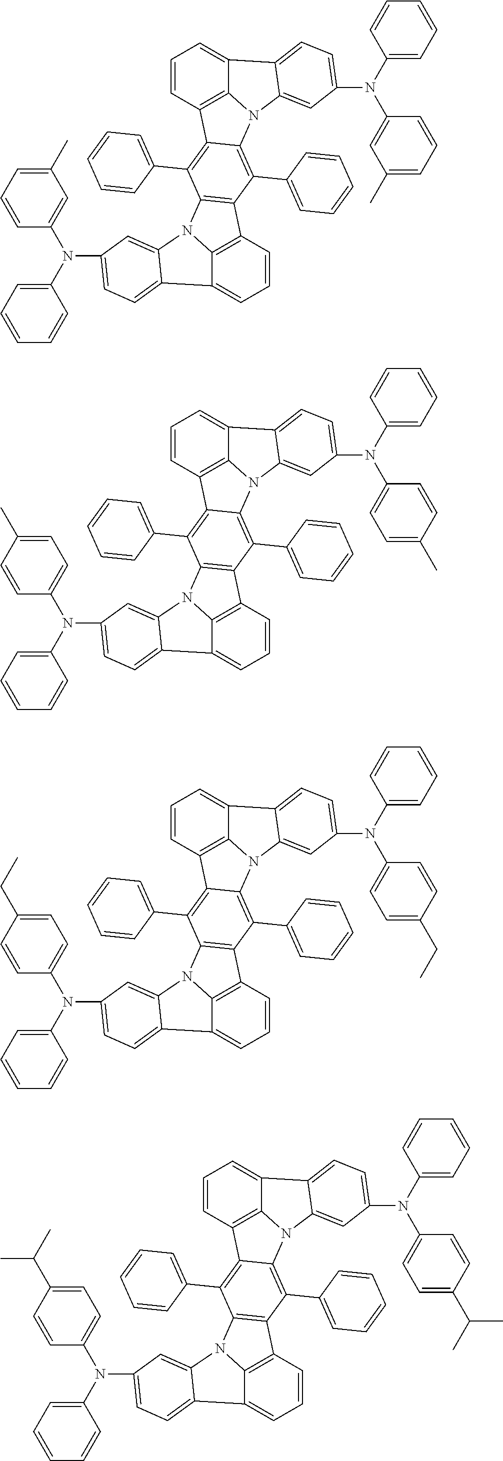

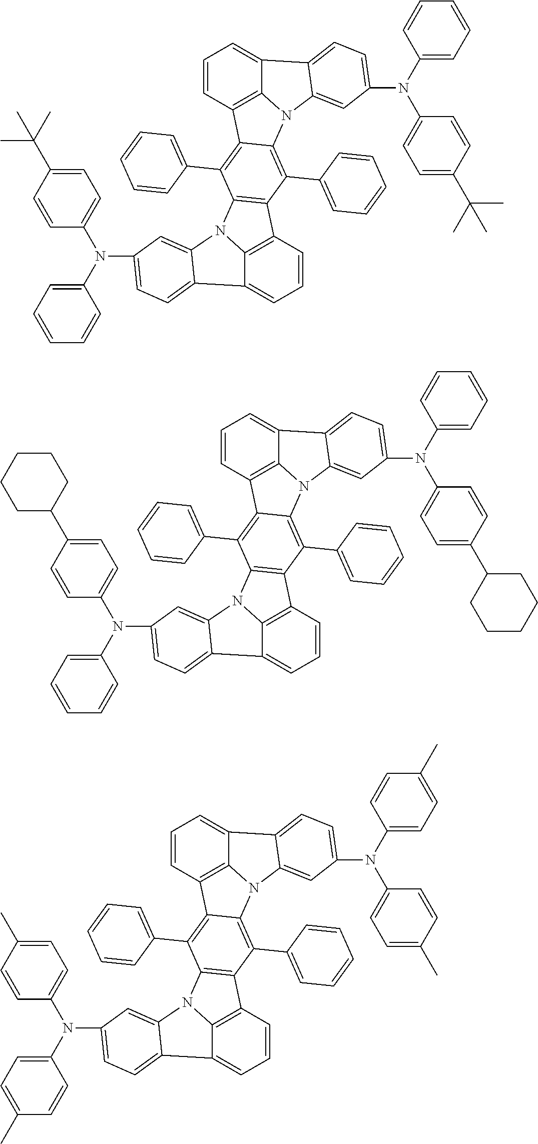 [SMILES: CC(C)(C)c1ccc(N(c2ccccc2)c2ccc3c4cccc5c6c(-c7ccccc7)c7c(c(-c8ccccc8)c6n(c3c2)c45)c2cccc3c4ccc(N(c5ccccc5)c5ccc(C(C)(C)C)cc5)cc4n7c32)cc1 ; c1ccc(-c2c3c4cccc5c6ccc(N(c7ccccc7)c7ccc(C8CCCCC8)cc7)cc6n(c3c(-c3ccccc3)c3c6cccc7c8ccc(N(c9ccccc9)c9ccc(C%10CCCCC%10)cc9)cc8n(c23)c76)c54)cc1 ; Cc1ccc(N(c2ccc(C)cc2)c2ccc3c4cccc5c6c(-c7ccccc7)c7c(c(-c8ccccc8)c6n(c3c2)c45)c2cccc3c4ccc(N(c5ccc(C)cc5)c5ccc(C)cc5)cc4n7c32)cc1]

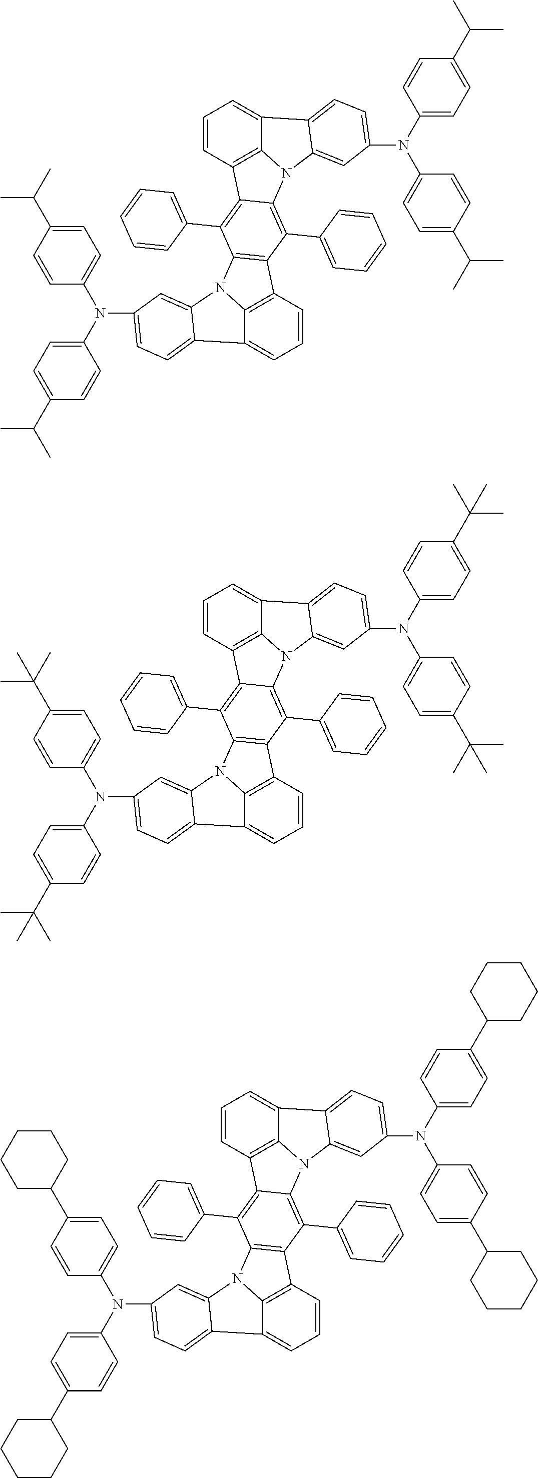

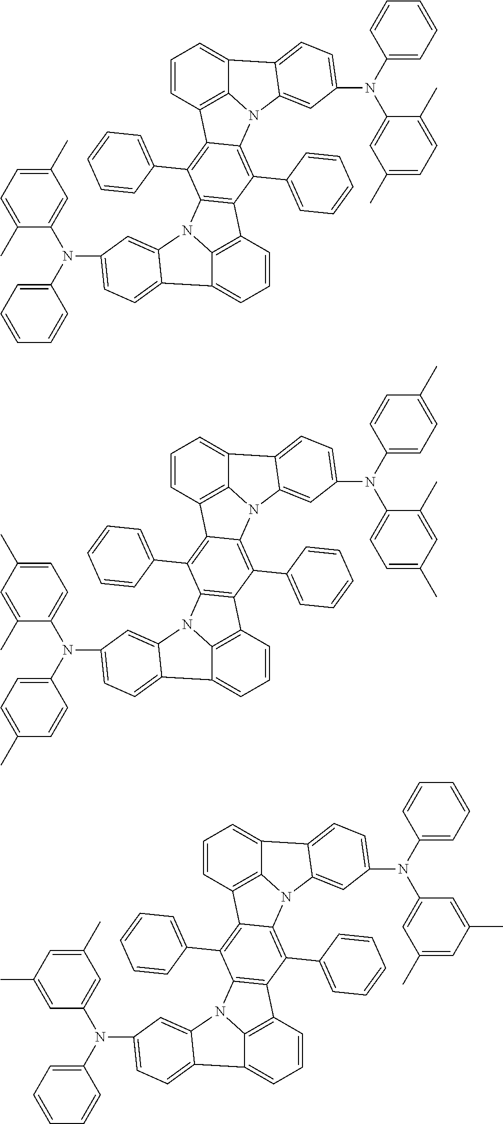

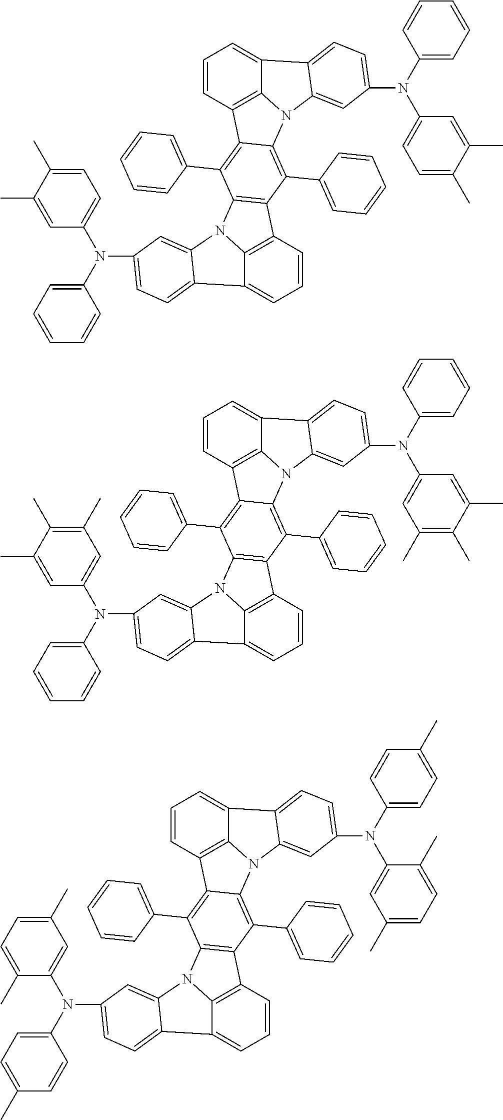

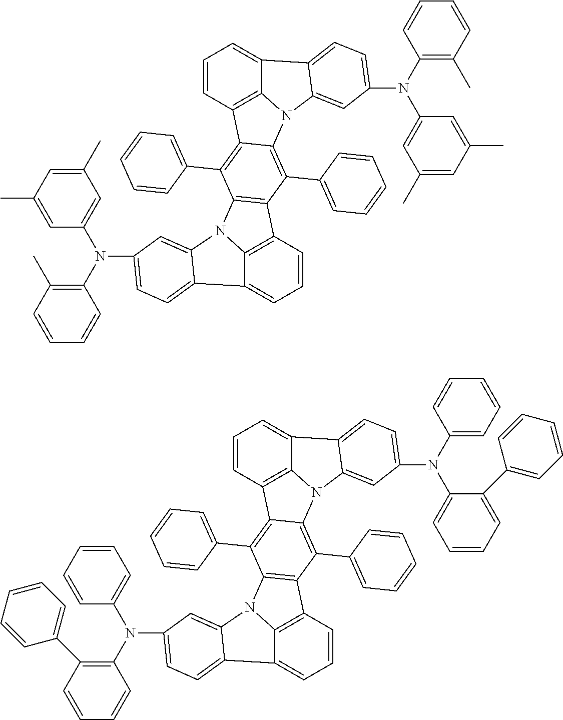

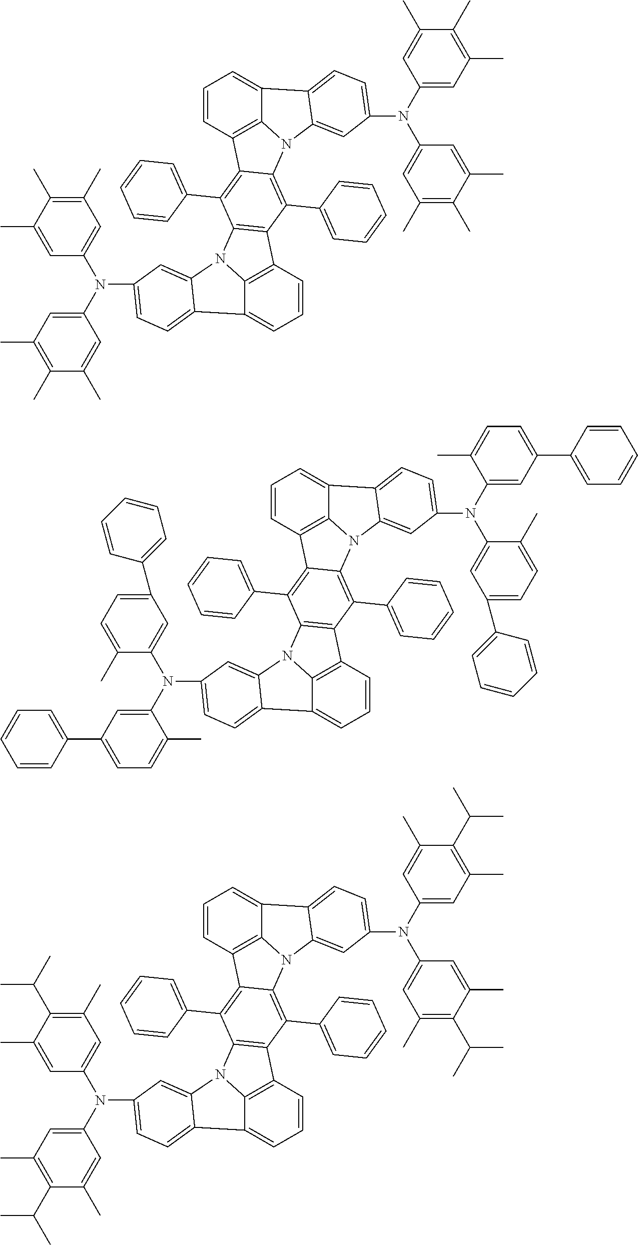

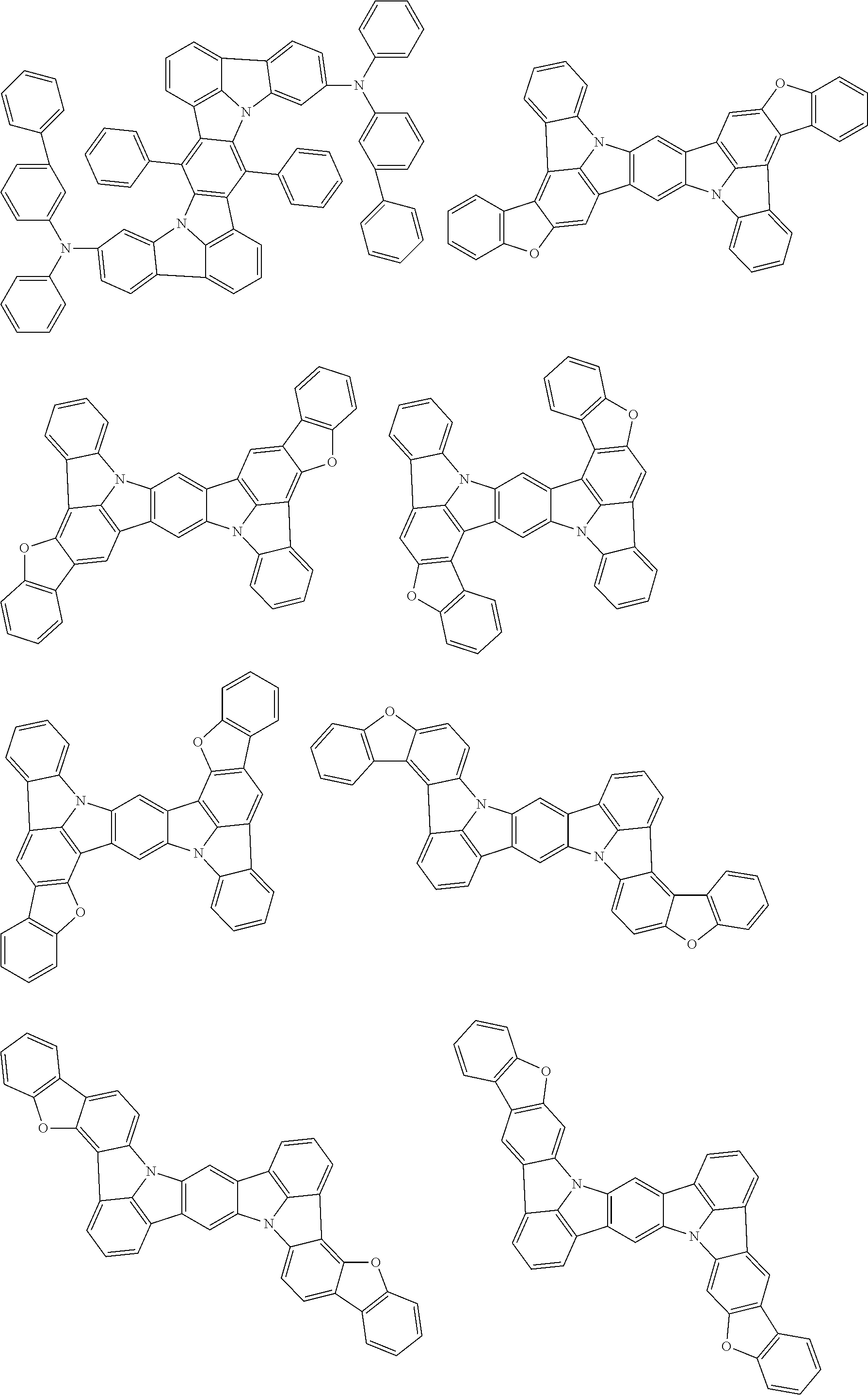

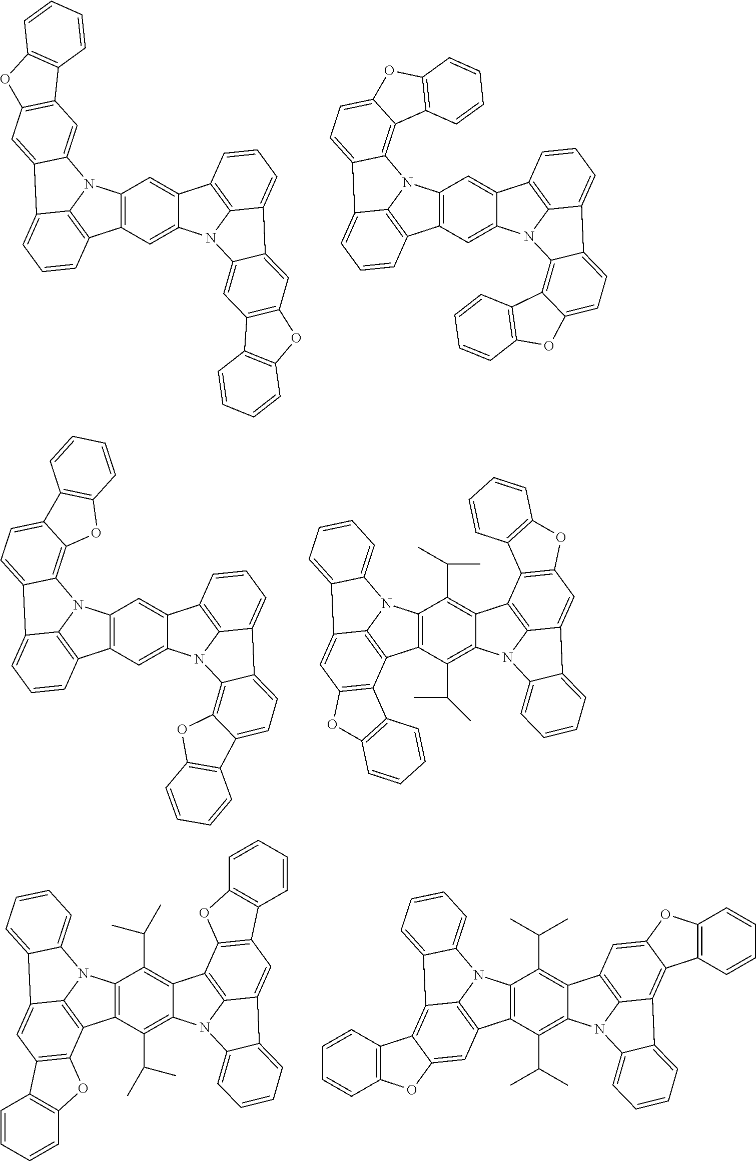

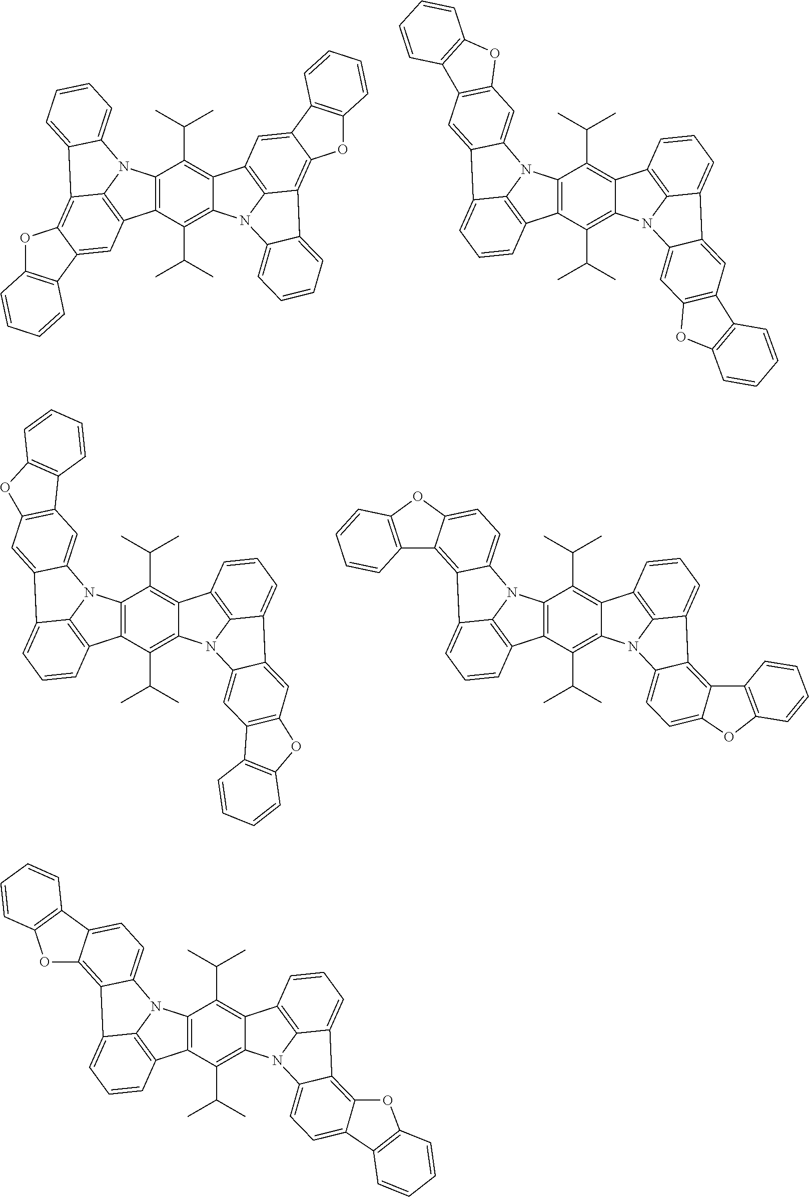 [SMILES: CC(C)c1c2c3cc4c5ccccc5oc4c4c5ccccc5n(c2c(C(C)C)c2c5cc6c7ccccc7oc6c6c7ccccc7n(c12)c56)c34 ; CC(C)c1c2c3cccc4c5cc6c(cc5n(c2c(C(C)C)c2c5cccc7c8cc9c(cc8n(c12)c75)oc1ccccc19)c43)oc1ccccc16 ; CC(C)c1c2c3cccc4c5cc6oc7ccccc7c6cc5n(c2c(C(C)C)c2c5cccc6c7cc8oc9ccccc9c8cc7n(c12)c65)c43 ; CC(C)c1c2c3cccc4c5c6c(ccc5n(c2c(C(C)C)c2c5cccc7c8c9c(ccc8n(c12)c57)oc1ccccc19)c34)oc1ccccc16 ; CC(C)c1c2c3cccc4c5c6oc7ccccc7c6ccc5n(c2c(C(C)C)c2c5cccc6c7c8oc9ccccc9c8ccc7n(c12)c56)c34]

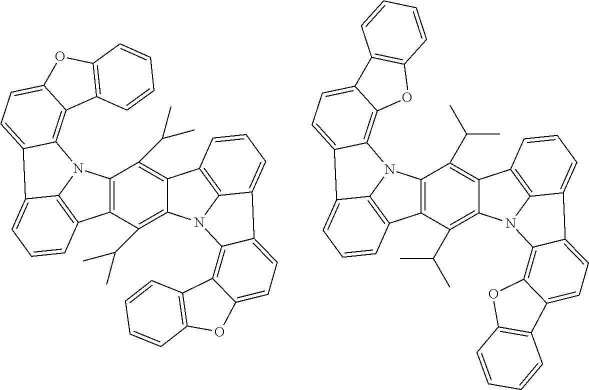 [SMILES: CC(C)c1c2c3cccc4c5ccc6oc7ccccc7c6c5n(c2c(C(C)C)c2c5cccc6c7ccc8oc9ccccc9c8c7n(c12)c65)c43 ; CC(C)c1c2c3cccc4c5ccc6c7ccccc7oc6c5n(c2c(C(C)C)c2c5cccc6c7ccc8c9ccccc9oc8c7n(c12)c65)c43]

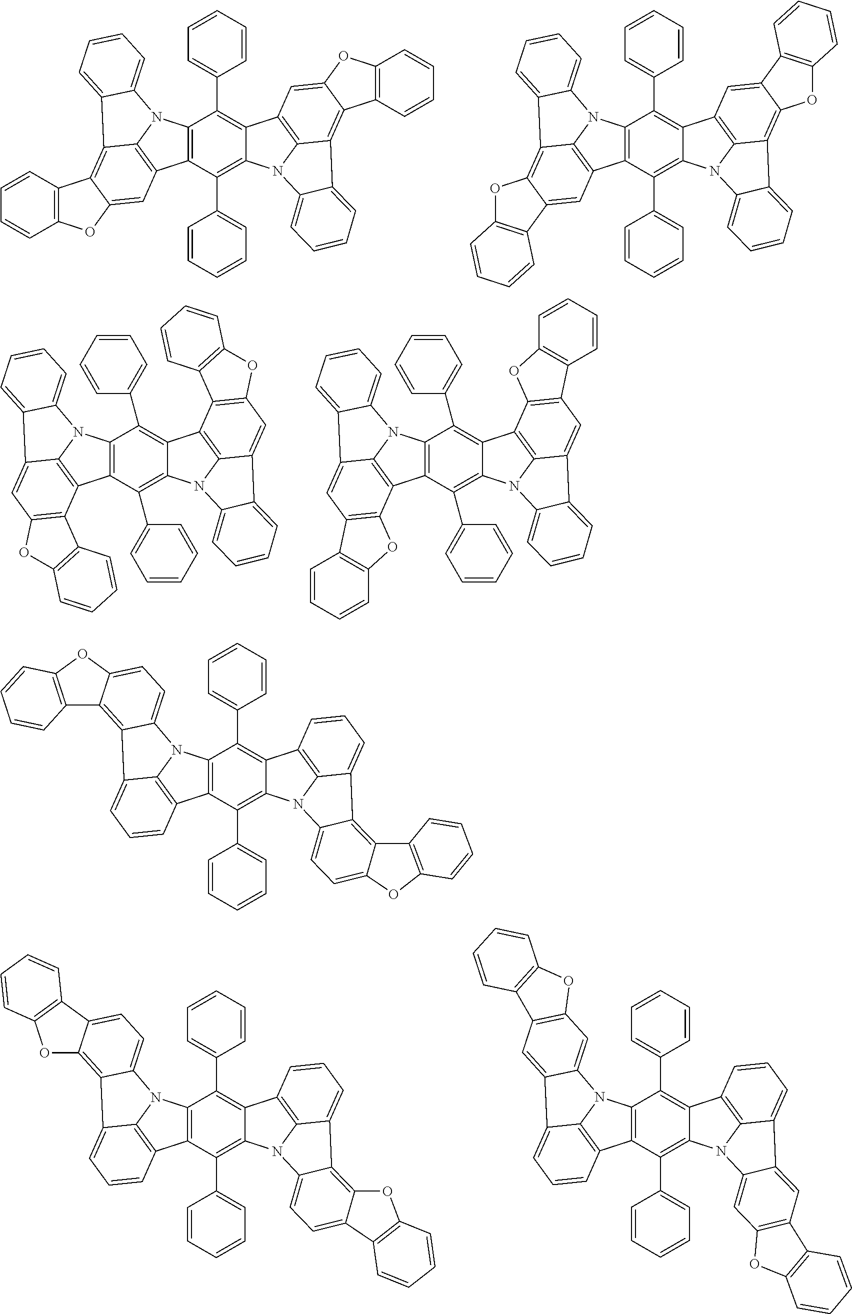

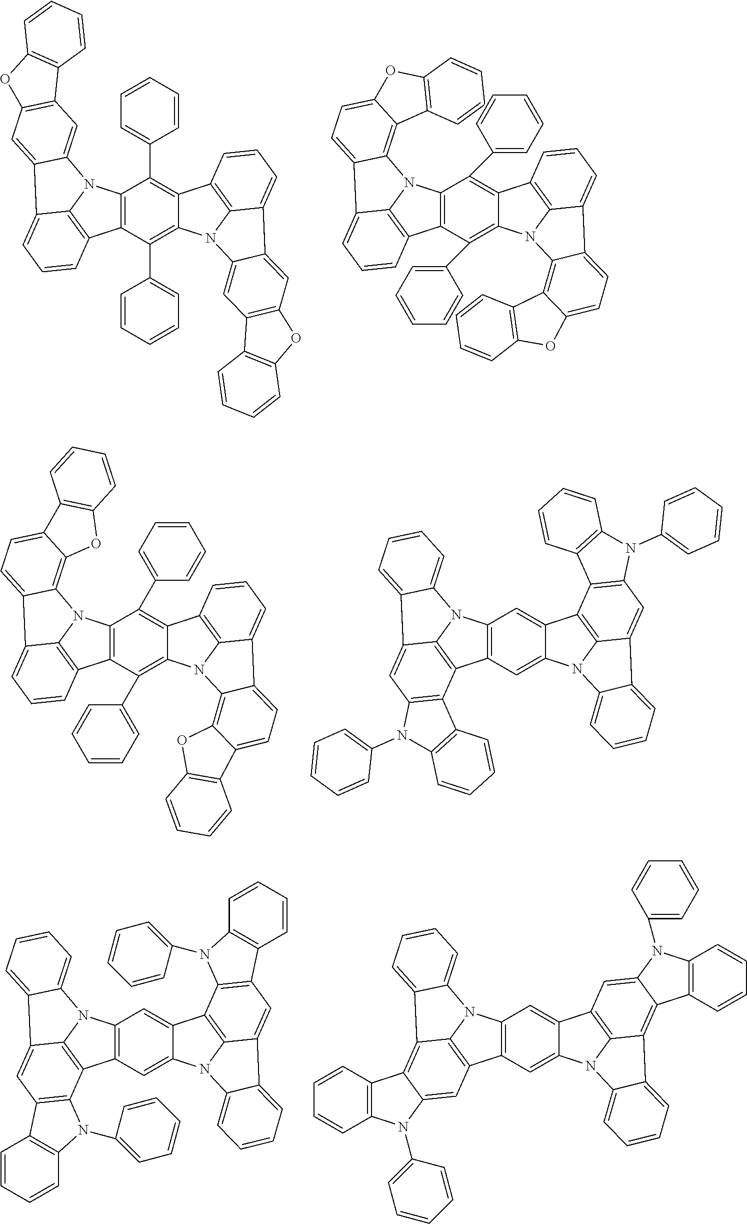

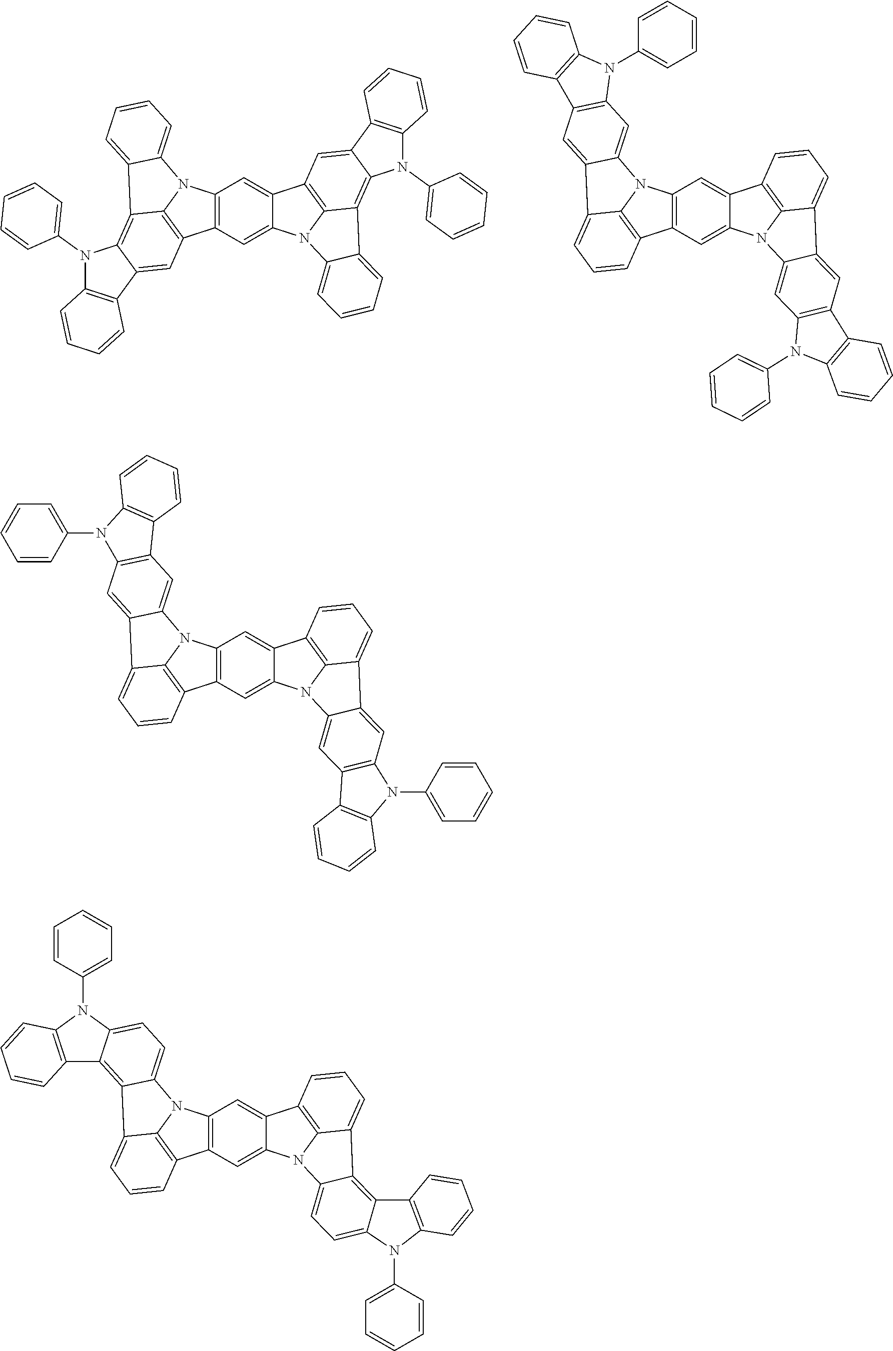 [SMILES: c1ccc(-n2c3ccccc3c3cc4c5cc6c(cc5n5c7ccccc7c(c32)c45)c2cc3c4ccccc4n(-c4ccccc4)c3c3c4ccccc4n6c23)cc1 ; c1ccc(-n2c3ccccc3c3cc4c5cccc6c7cc8c(cc7n(c4cc32)c65)c2cccc3c4cc5c6ccccc6n(-c6ccccc6)c5cc4n8c23)cc1 ; c1ccc(-n2c3ccccc3c3cc4c(cc32)c2cccc3c5cc6c(cc5n4c23)c2cccc3c4cc5c(cc4n6c32)c2ccccc2n5-c2ccccc2)cc1 ; c1ccc(-n2c3ccccc3c3c4c5cccc6c7cc8c(cc7n(c4ccc32)c65)c2cccc3c4c5c6ccccc6n(-c6ccccc6)c5ccc4n8c23)cc1]

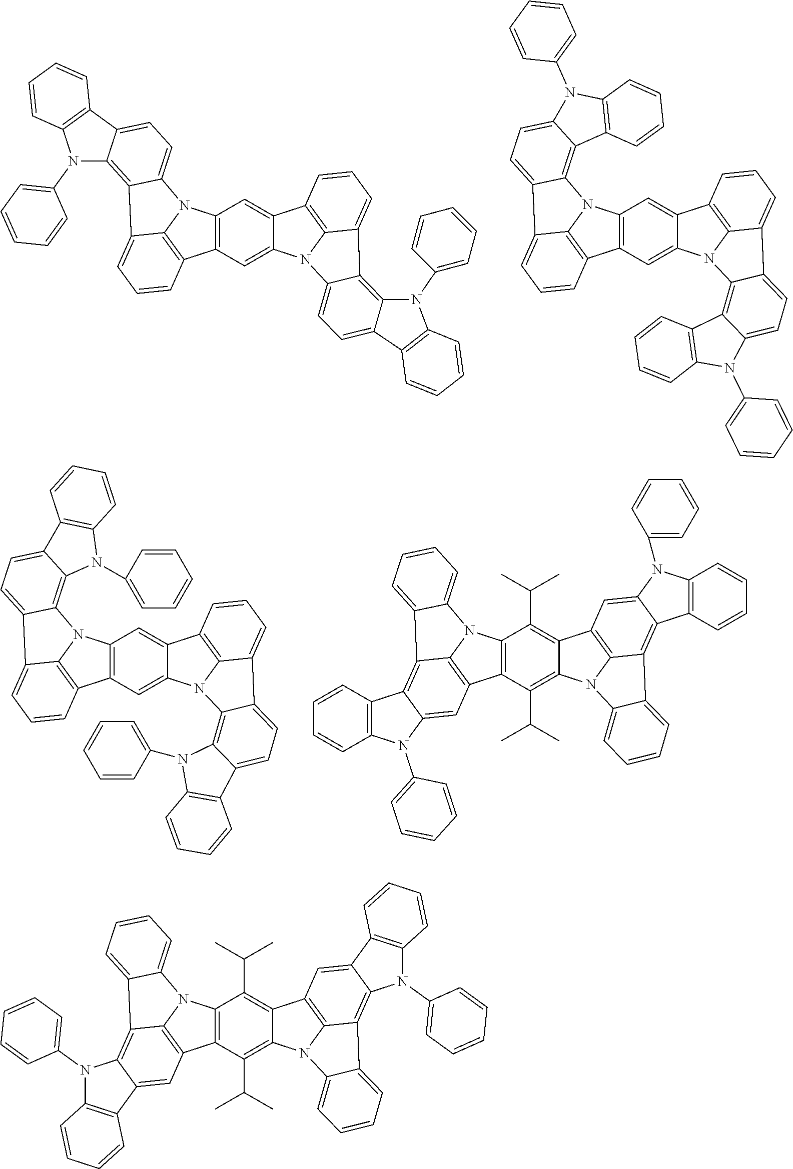

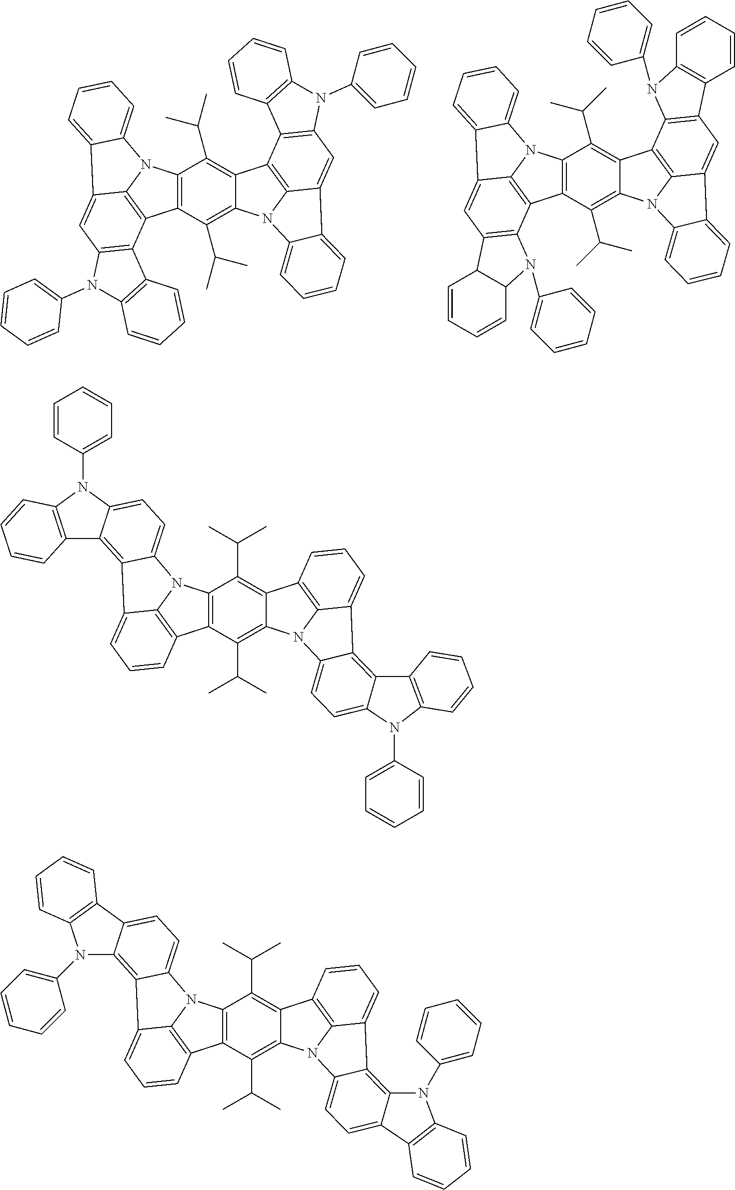

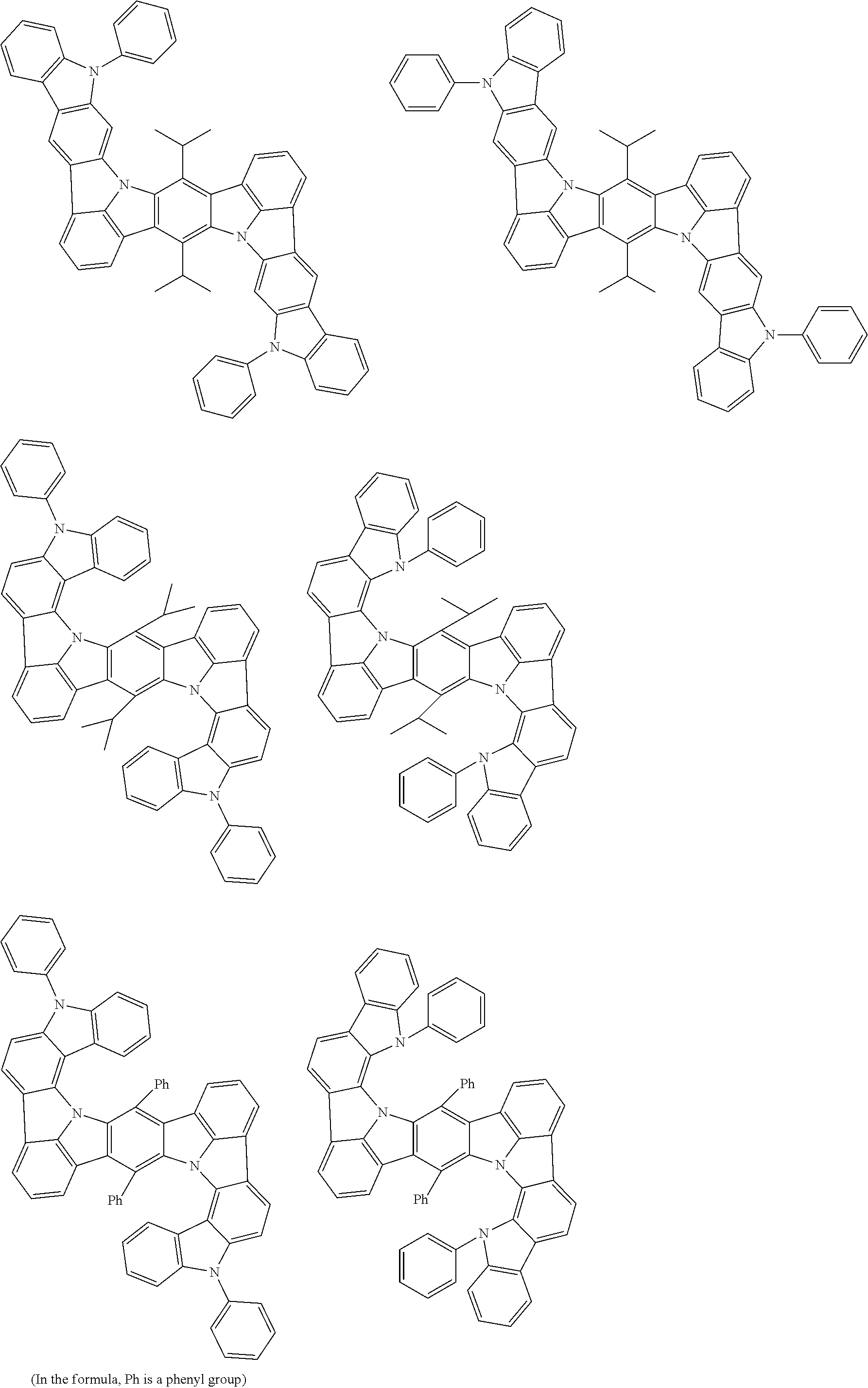

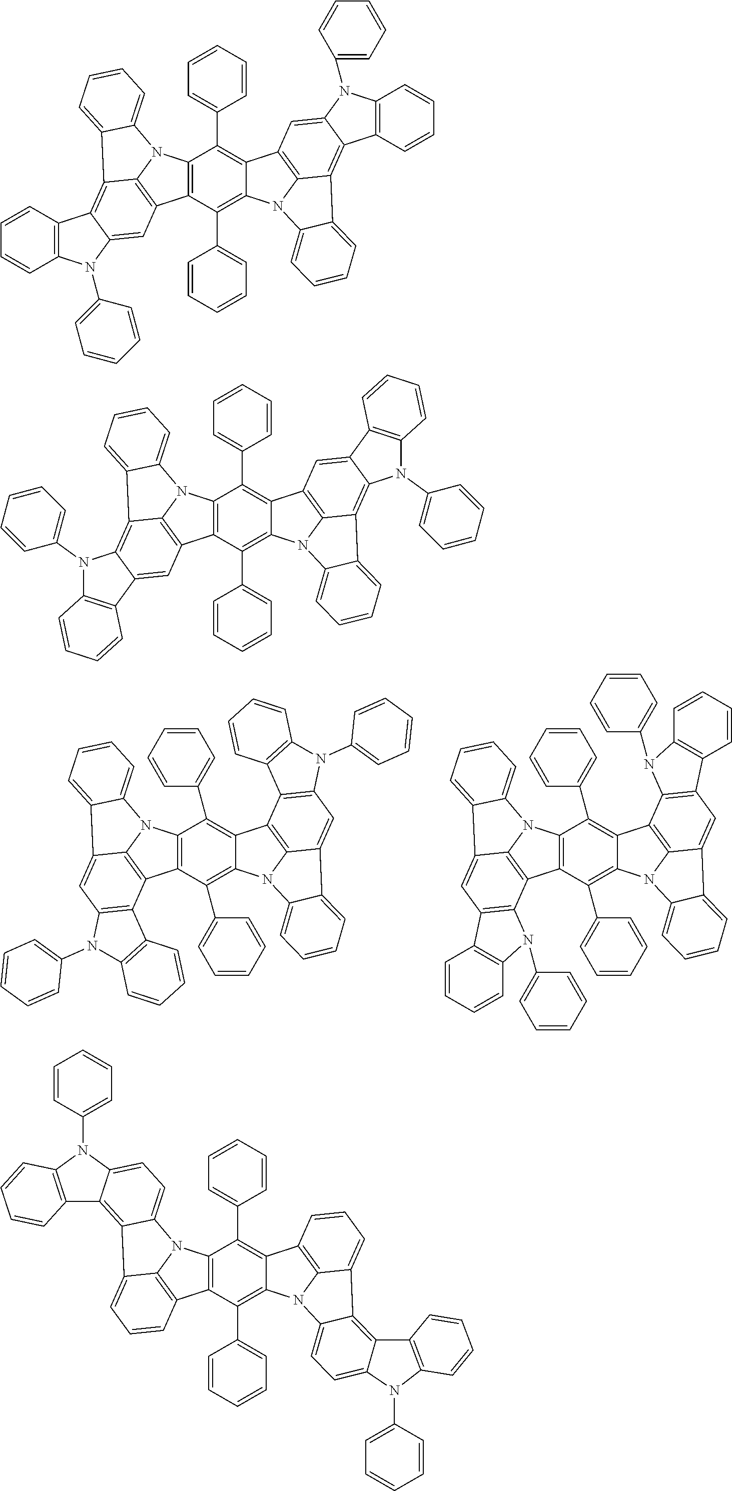

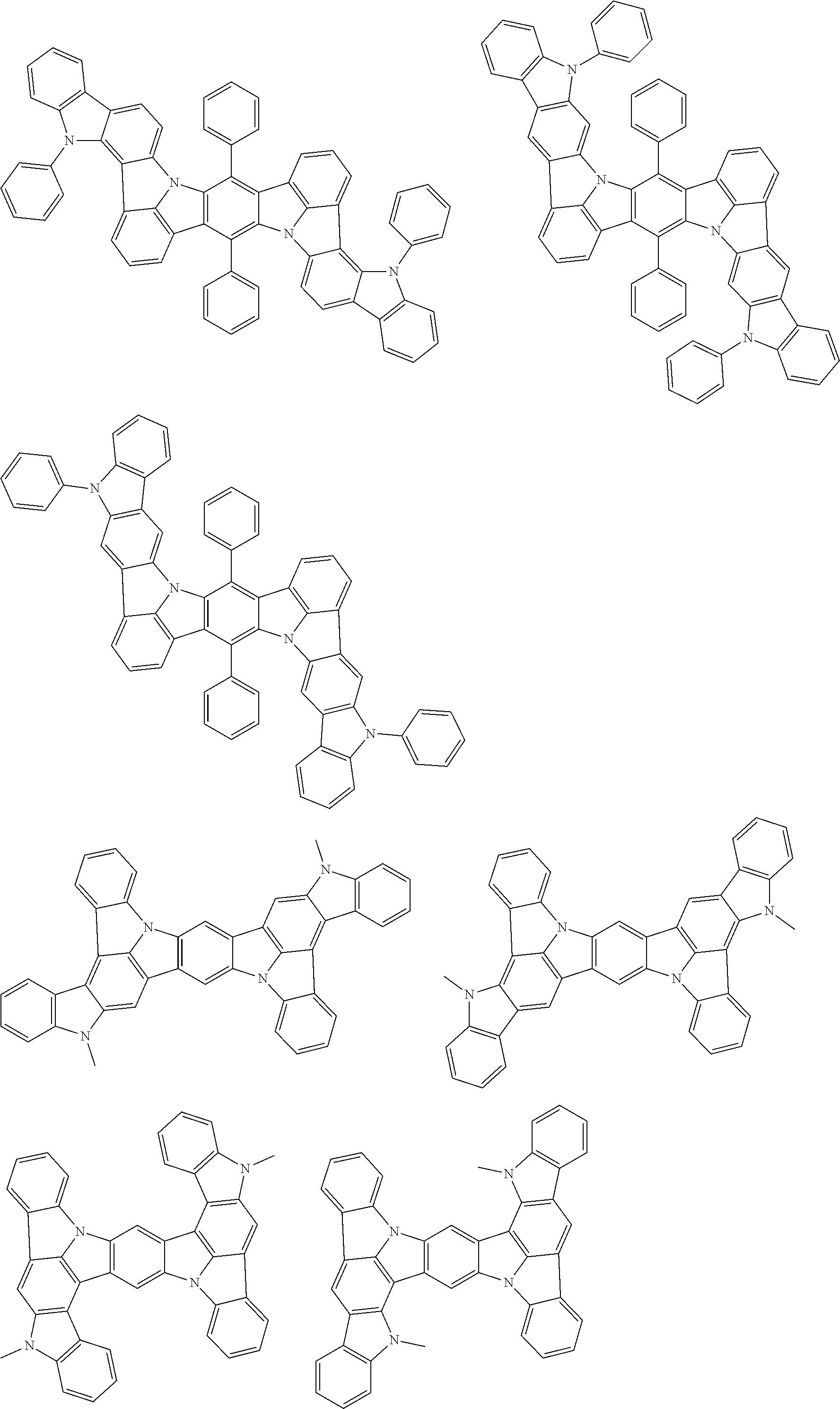

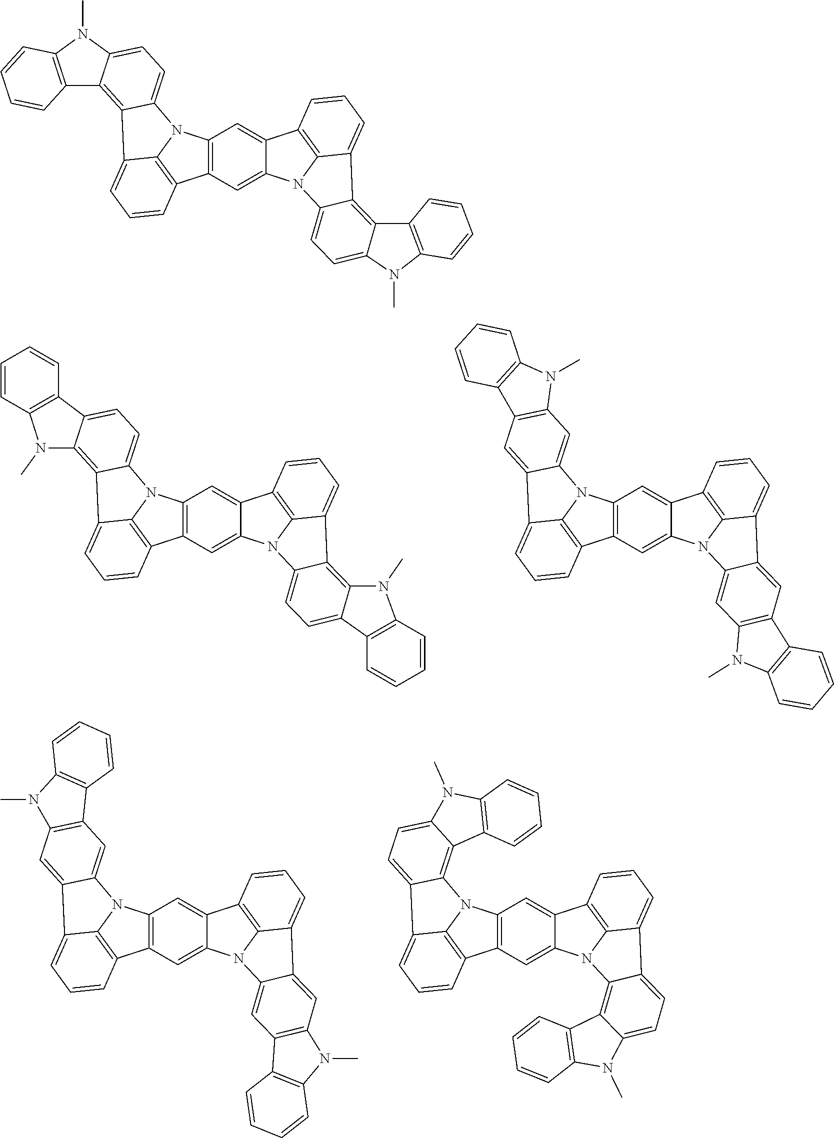

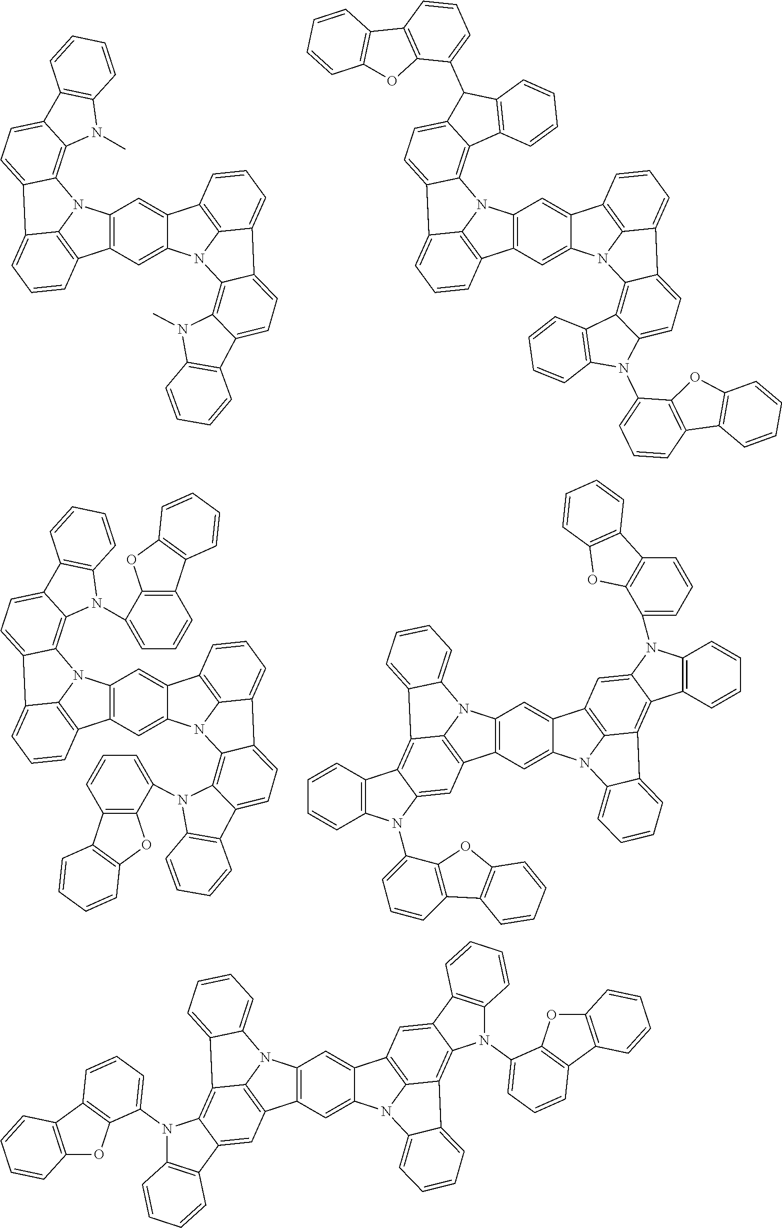

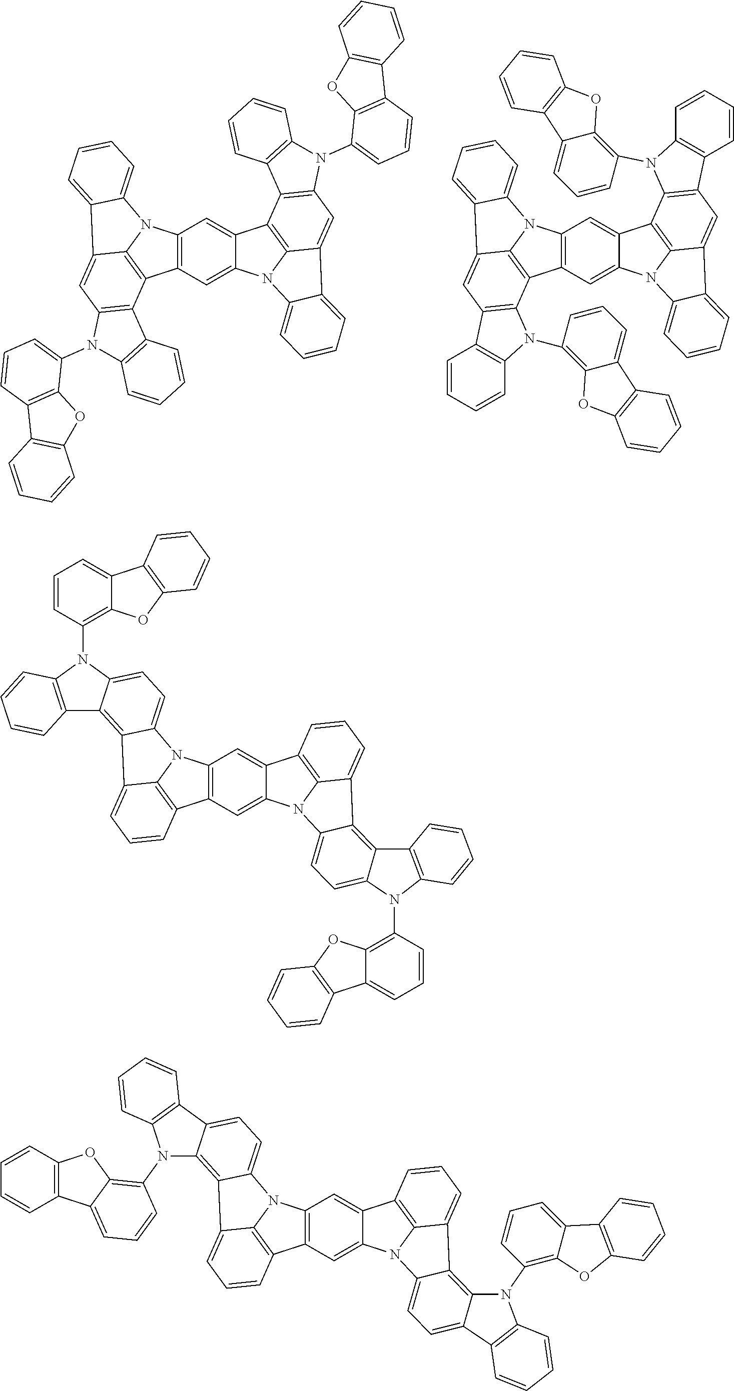

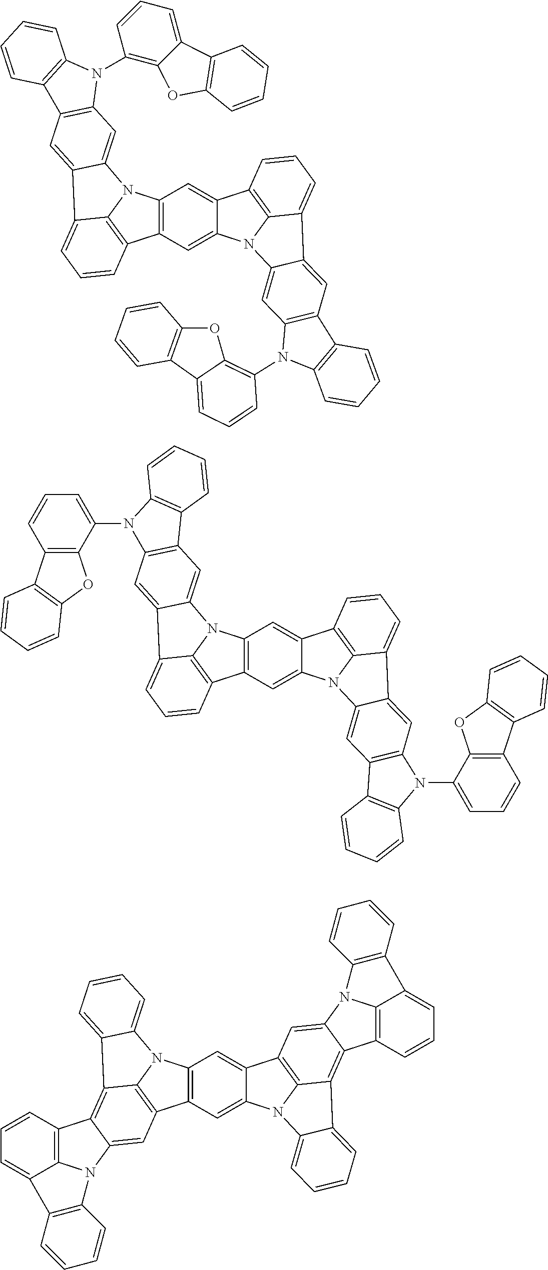

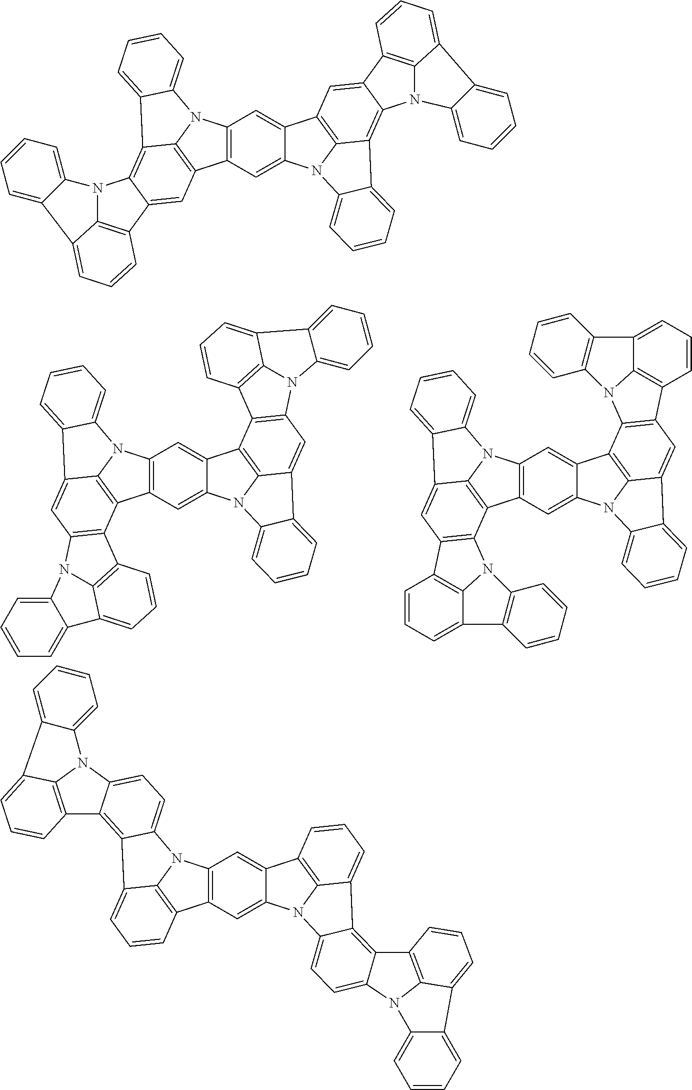

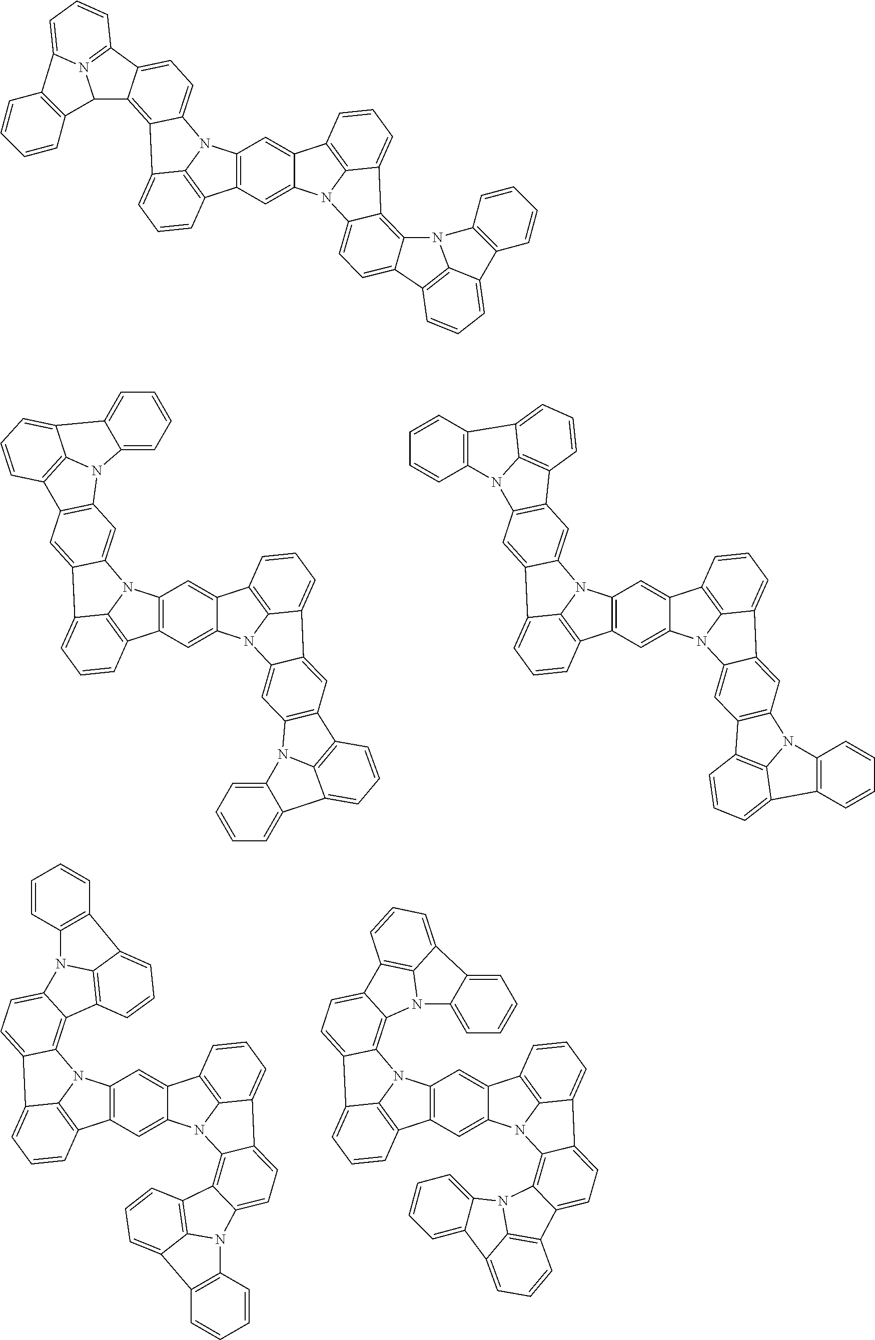

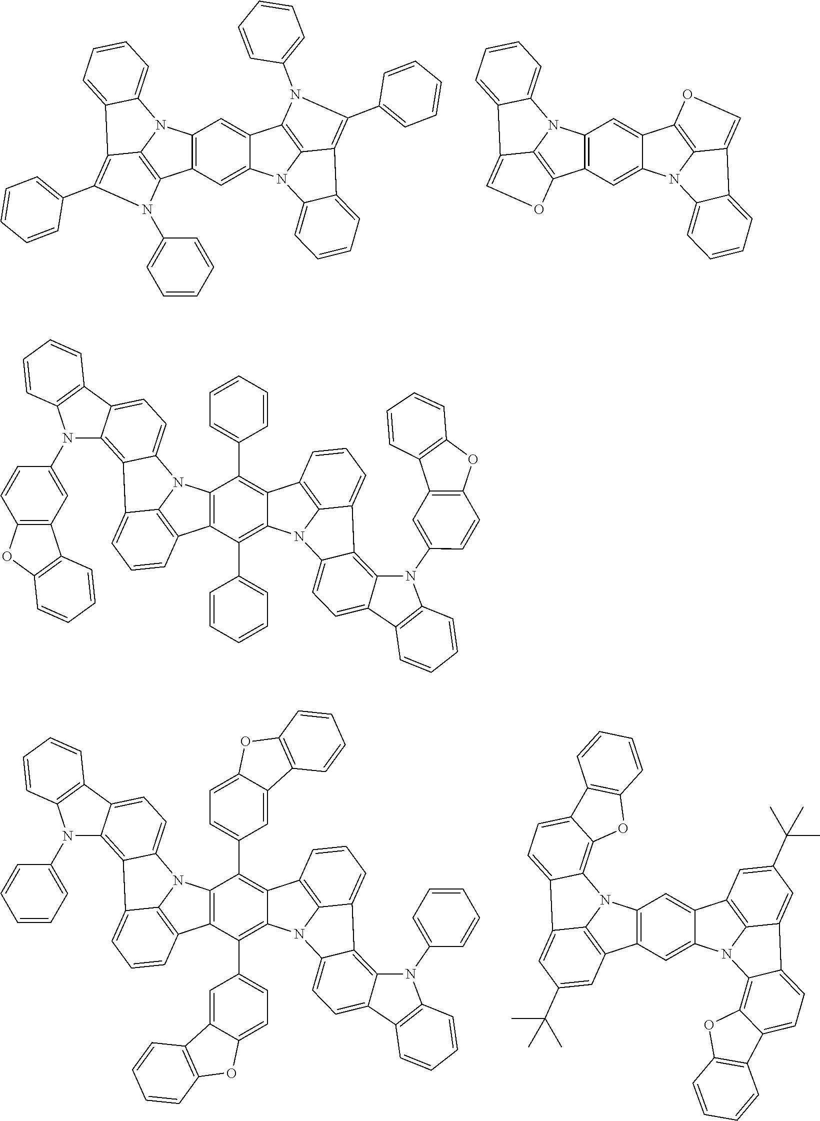

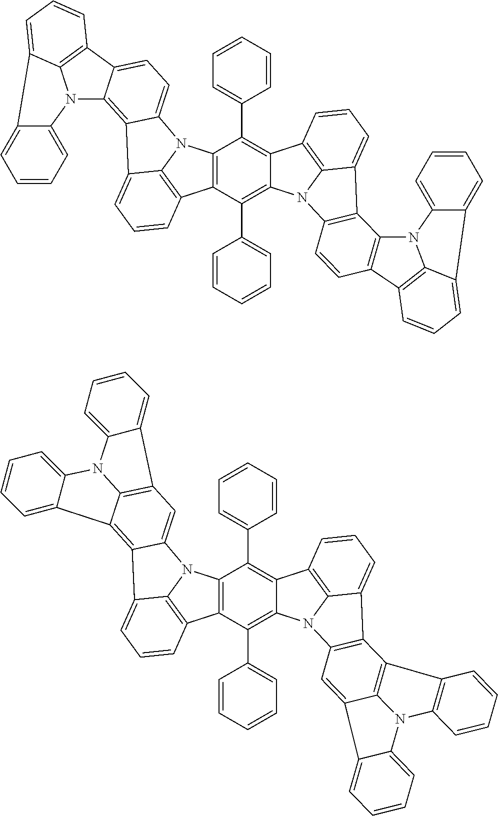 [SMILES: c1ccc(-c2c3c4cccc5c6c(ccc7c8cccc9c%10ccccc%10n(c98)c76)n(c3c(-c3ccccc3)c3c6cccc7c8c(ccc9c%10cccc%11c%12ccccc%12n(c%11%10)c98)n(c23)c67)c45)cc1 ; c1ccc(-c2c3c4cccc5c6c7c8ccccc8n8c9ccccc9c(cc6n(c3c(-c3ccccc3)c3c6cccc9c%10c%11c%12ccccc%12n%12c%13ccccc%13c(cc%10n(c23)c69)c%11%12)c45)c78)cc1]

### (Compound Represented by the Formula (B1))

A compound represented by the following formula (B1) is contained in the first layer.

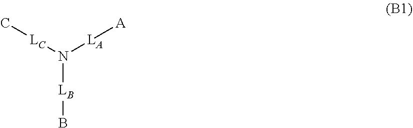

In the formula (B1),

L_{A}, L_{B} and L_{C} are independently a single bond, a substituted or unsubstituted arylene group including 6 to 18 ring carbon atoms, or a substituted or unsubstituted divalent heterocyclic group including 5 to 13 ring atoms.

A, B and C are independently

a substituted or unsubstituted aryl group including 6 to 30 ring carbon atoms,
a substituted or unsubstituted monovalent heterocyclic group including 5 to 30 ring atoms, or
—Si(R′₉₀₁)(R′₉₀₂)(R′₉₀₃).

R′₉₀₁ to R′₉₀₃ are independently a substituted or unsubstituted aryl group including 6 to 30 ring carbon atoms.

When two or more of each of one or more among R′₉₀₁ to R′₉₀₃ are present, each of the two or more R′₉₀₁ to R′₉₀₃ may be the same or different.

In one embodiment, the compound represented by the formula (B1) is a compound represented by the following formula (B11).

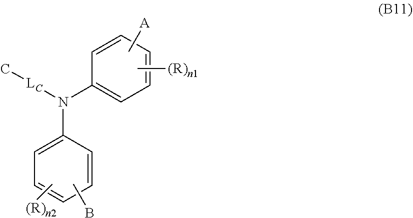

In the formula (B11), L_{C}, A, B and C are as defined in the formula (B1).

When a plurality of R's is present, one or more sets of adjacent two or more among the plurality of R's form a substituted or unsubstituted, saturated or unsaturated ring by bonding with each other, or do not form a substituted or unsubstituted, saturated or unsaturated ring.

R which does not form the substituted or unsubstituted, saturated or unsaturated ring is

a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

a substituted or unsubstituted aryl group including 6 to 50 ring carbon atoms, or
a substituted or unsubstituted monovalent heterocyclic group including 5 to 50 ring atoms.

R₉₀₁ to R₉₀₄ are as defined in the formula (A1).

n1 and n2 are independently an integer of 0 to 4.

When a plurality of R's is present, the plurality of R's may be the same with or different from each other.

In one embodiment, two among A to C in the formula (B1) or (B11) are groups represented by the following formula (Y), and the two groups represented by the formula (Y) may be the same or different.

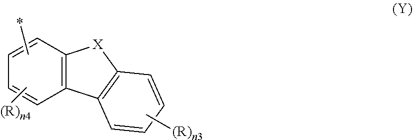

In the formula (Y), X is CR₅₁R₅₂, NR₅₃, an oxygen atom, or a sulfur atom.

When X is CR₅₁R₅₂, R₅₁ and R₅₂ form a substituted or unsubstituted, saturated or unsaturated ring by bonding with each other, or do not form a substituted or unsubstituted, saturated or unsaturated ring.

When a plurality of R's is present, one or more sets of adjacent two or more among the plurality of R's form a substituted or unsubstituted, saturated or unsaturated ring by bonding with each other, or do not form a substituted or unsubstituted, saturated or unsaturated ring.

R which does not form the substituted or unsubstituted, saturated or unsaturated ring is independently

a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

a substituted or unsubstituted aryl group including 6 to 50 ring carbon atoms, or
a substituted or unsubstituted monovalent heterocyclic group including 5 to 50 ring atoms.

R₅₃, and R₅₁ and R₅₂ which do not form the substituted or unsubstituted, saturated or unsaturated ring are independently

a hydrogen atom,
a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

a substituted or unsubstituted aryl group including 6 to 50 ring carbon atoms, or
a substituted or unsubstituted monovalent heterocyclic group including 5 to 50 ring atoms.

R₉₀₁ to R₉₀₄ are as defined in the formula (A1).

n3 is an integer of 0 to 4, and n4 is an integer of 0 to 3.

When a plurality of R's is present, the plurality of R's may be the same as or different from each other.

“*” is bonded to any of L_{A} to L_{C} in the formula (B1), or L_{C}, a benzene ring which is bonded to A, or a benzene ring which is bonded to B in the formula (B11).

In one embodiment, at least one of A to C in the formula (B1) or (B11) is a group represented by the following formula (Y1) or a group represented by the following formula (Y2).

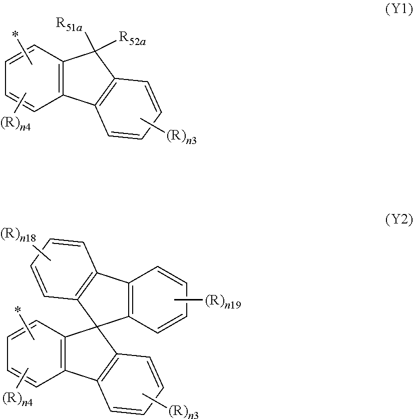

In the formulas (Y1) and (Y2),

“*” is bonded to any of L_{A} to L_{C} in the formula (B1), or L_{C}, a benzene ring which is bonded to A, or a benzene ring which is bonded to B in the formula (B11).

R₅₁ₐ and R₅₂ₐ do not form a substituted or unsubstituted, saturated or unsaturated ring by bonding with each other.

R₅₁ₐ and R₅₂ₐ are independently

a hydrogen atom,
a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

a substituted or unsubstituted aryl group including 6 to 50 ring carbon atoms, or
a substituted or unsubstituted monovalent heterocyclic group including 5 to 50 ring atoms.

R₉₀₁ to R₉₀₄ are as defined in the formula (A1).

n4 is an integer of 0 and 3.

n3, n18, and n19 are independently an integer of 0 to 4.

When n3, n4, n18 or n19 is 2 or more, one or more sets of adjacent two or more among a plurality of R's form a substituted or unsubstituted, saturated or unsaturated ring by bonding with each other, or do not form a substituted or unsubstituted, saturated or unsaturated ring.

R which does not form the substituted or unsubstituted, saturated or unsaturated ring is

a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

a substituted or unsubstituted aryl group including 6 to 50 ring carbon atoms, or
a substituted or unsubstituted monovalent heterocyclic group including 5 to 50 ring atoms.

R₉₀₁ to R₉₀₄ are as defined in the formula (A1).

When a plurality of R's is present, the plurality of R's may be the same with or different from each other.

When two or more groups represented by the formula (Y1) or (Y2) are present, the two or more groups represented by the formula (Y1) or (Y2) may be the same with or different from each other.

In one embodiment, the compound represented by the formula (B1) is a compound represented by the following formula (B12) or (B13).

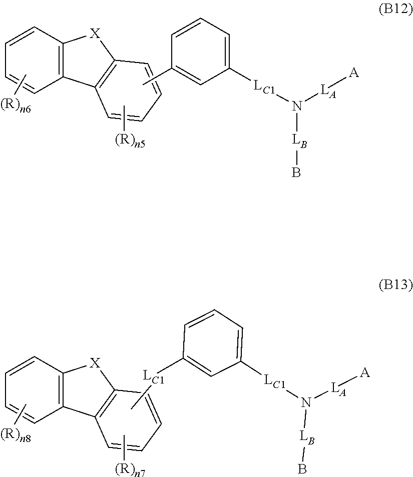

In the formulas (B12) and (B13),

L_{A}, L_{B}, A and B are as defined in the formula (B1).

L_{C1} is an arylene group including 6 to 12 ring carbon atoms.

X is CR₅₁R₅₂, NR₅₃, an oxygen atom, or a sulfur atom.

When X is CR₅₁R₅₂, R₅₁ and R₅₂ form a substituted or unsubstituted, saturated or unsaturated ring by bonding with each other, or do not form a substituted or unsubstituted, saturated or unsaturated ring.

When a plurality of R's is present, one or more sets of adjacent two or more among the plurality of R's form a substituted or unsubstituted, saturated or unsaturated ring by bonding with each other, or do not form a substituted or unsubstituted, saturated or unsaturated ring.

R which does not form the substituted or unsubstituted, saturated or unsaturated ring is independently

a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

a substituted or unsubstituted aryl group including 6 to 50 ring carbon atoms, or
a substituted or unsubstituted monovalent heterocyclic group including 5 to 50 ring atoms.

R₅₃, and R₅₁ and R₅₂ which do not form the substituted or unsubstituted, saturated or unsaturated ring are independently

a hydrogen atom,
a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

a substituted or unsubstituted aryl group including 6 to 50 ring carbon atoms, or
a substituted or unsubstituted monovalent heterocyclic group including 5 to 50 ring atoms.

R₉₀₁ to R₉₀₄ are as defined in the formula (A1).

n5 and n7 are independently an integer of 0 to 3, and n6 and n8 are independently an integer of 0 to 4.

When a plurality of R's is present, the plurality of R's may be the same with or different from each other.

In one embodiment, the compound represented by the formula (B1) is a compound represented by the following formula (B14) or (B15).

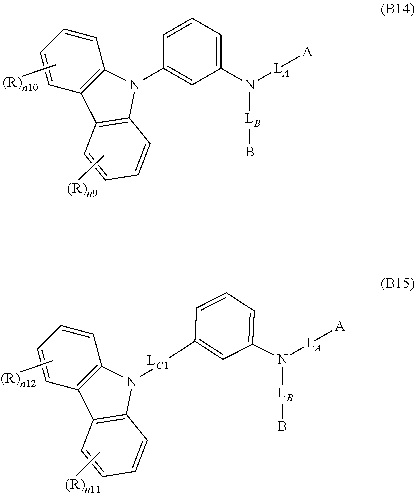

In the formulas (B14) and (B15),

L_{A}, L_{B}, A and B are as defined in the formula (B1).

L_{C1} is an arylene group including 6 to 12 ring carbon atoms.

When a plurality of R's is present, one or more sets of adjacent two or more among the plurality of R's form a substituted or unsubstituted, saturated or unsaturated ring by bonding with each other, or do not form a substituted or unsubstituted, saturated or unsaturated ring.

R which does not form the substituted or unsubstituted, saturated or unsaturated ring is

a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

a substituted or unsubstituted aryl group including 6 to 50 ring carbon atoms, or
a substituted or unsubstituted monovalent heterocyclic group including 5 to 50 ring atoms.

R₉₀₁ to R₉₀₄ are as defined in the formula (A1).

n9 to n12 are independently an integer of 0 to 4.

When a plurality of R's is present, the plurality of R's may be the same with or different from each other.

In one embodiment, the compound represented by the formula (B1) is a compound represented by the following formula (B16) or (B17).

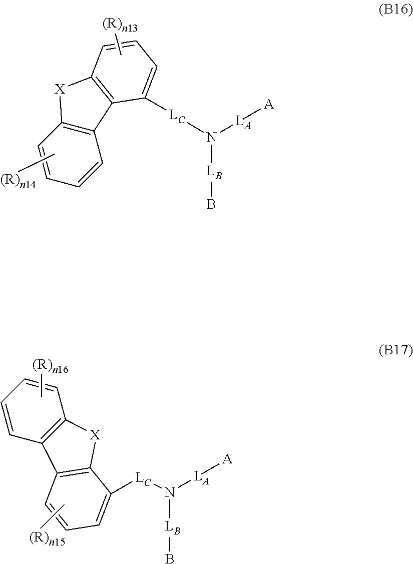

In the formulas (B16) and (B17),

L_{A}, L_{B}, L_{C}, A and B are as defined in the formula (B1).

X is CR₅₁R₅₂, NR₅₃, an oxygen atom, or a sulfur atom.

When X is CR₅₁R₅₂, R₅₁ and R₅₂ form a substituted or unsubstituted, saturated or unsaturated ring by bonding with each other, or do not form a substituted or unsubstituted, saturated or unsaturated ring.

When a plurality of R's is present, one or more sets of adjacent two or more among the plurality of R's form a substituted or unsubstituted, saturated or unsaturated ring by bonding with each other, or do not form a substituted or unsubstituted, saturated or unsaturated ring.

R which does not form the substituted or unsubstituted, saturated or unsaturated ring is independently

a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

a substituted or unsubstituted aryl group including 6 to 50 ring carbon atoms, or
a substituted or unsubstituted monovalent heterocyclic group including 5 to 50 ring atoms.

R₅₃, and R₅₁ and R₅₂ which do not form the substituted or unsubstituted, saturated or unsaturated ring are independently

a hydrogen atom,
a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

a substituted or unsubstituted aryl group including 6 to 50 ring carbon atoms, or
a substituted or unsubstituted monovalent heterocyclic group including 5 to 50 ring atoms.

R₉₀₁ to R₉₀₄ are as defined in the formula (A1).

n13 and n15 are independently an integer of 0 to 3, and n14 and n16 are independently an integer of 0 to 4.

When a plurality of R's is present, the plurality of R's may be the same with or different from each other.

In one embodiment, the compound represented by the formula (B1) is a compound represented by the following formula (B18).

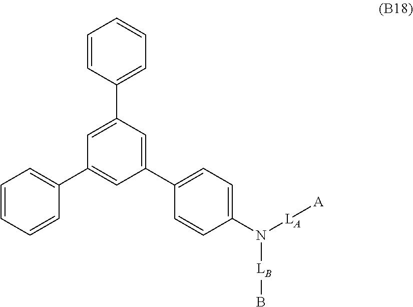

In the formula (B18), L_{A}, L_{B}, A, and B are as defined in the formula (B1).

In one embodiment, the compound represented by the formula (B1) is a compound represented by the following formula (B19).

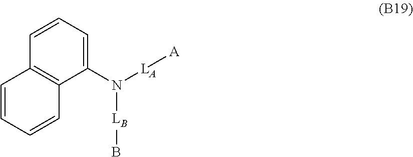

In the formula (B19), L_{A}, L_{B}, A, and B are as defined in the formula (B1).

In one embodiment, the compound represented by the formula (B1) is a compound represented by the following formula (B20).

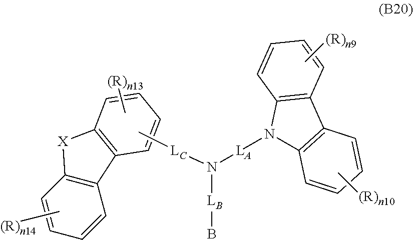

In the formula (B20), L_{A} to L_{C}, and B are as defined in the formula (B1).

X is CR₅₁R₅₂, NR₅₃, an oxygen atom, or a sulfur atom.

When X is CR₅₁R₅₂, R₅₁ and R₅₂ form a substituted or unsubstituted, saturated or unsaturated ring by bonding with each other, or do not form a substituted or unsubstituted, saturated or unsaturated ring.

R₅₃, and R₅₁ and R₅₂ which do not form the substituted or unsubstituted, saturated or unsaturated ring are independently

a hydrogen atom,
a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

a substituted or unsubstituted aryl group including 6 to 50 ring carbon atoms, or
a substituted or unsubstituted monovalent heterocyclic group including 5 to 50 ring atoms.

R₉₀₁ to R₉₀₄ are as defined in the formula (A1).

n9, n10, and n14 are independently an integer of 0 to 4.

n13 is an integer of 0 and 3.

When n9, n10, n13 or n14 is 2 or more, one or more sets of adjacent two or more among a plurality of R's form a substituted or unsubstituted, saturated or unsaturated ring by bonding with each other, or do not form a substituted or unsubstituted, saturated or unsaturated ring.

R which does not form the substituted or unsubstituted, saturated or unsaturated ring is independently

a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

a substituted or unsubstituted aryl group including 6 to 50 ring carbon atoms, or
a substituted or unsubstituted monovalent heterocyclic group including 5 to 50 ring atoms.

R₉₀₁ to R₉₀₄ are as defined in the formula (A1).

When a plurality of R's is present, the plurality of R's may be the same with or different from each other.

In one embodiment, the compound represented by the formula (B1) is a compound represented by the following formula (B21).

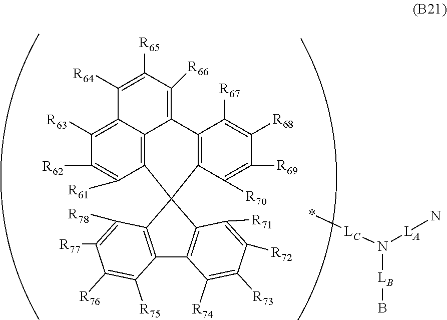

In the formula (B21), L_{A} to L_{C}, A and B are as defined in the formula (B1).

One of R₆₁ to R₇₈ is a single bond which is bonded with “*”.

One or more sets of adjacent two or more among R₆₁ to R₇₈ which are not a single bond bonding with “*” do not form a substituted or unsubstituted, saturated or unsaturated ring by bonding with each other.

R₆₁ to R₇₈ which are not a single bond bonding with “*” are independently a hydrogen atom, or a substituent.

The substituent is

a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

### —S—(R₉₀₅),

—N(R₉₀₆)(R₉₀₇),
a halogen atom, a cyano group, a nitro group,
a substituted or unsubstituted aryl group including 6 to 50 ring carbon atoms, or
a substituted or unsubstituted monovalent heterocyclic group including 5 to 50 ring atoms.

R₉₀₁ to R₉₀₇ are as defined in the formula (A1).

In one embodiment, the compound represented by the formula (B1) is a compound represented by the following formula (B22).

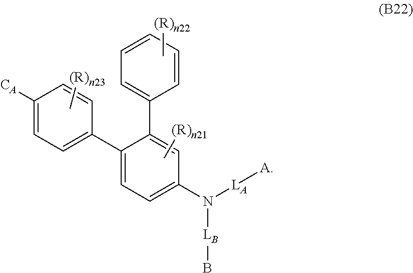

In the formula (B22), L_{A}, L_{B}, A and B are as defined in the formula (B1).

C_{A} is

a substituted or unsubstituted aryl group including 6 to 30 ring carbon atoms, or
a substituted or unsubstituted monovalent heterocyclic group including 5 to 30 ring atoms.

n21 is an integer of 0 to 3.

n22 is an integer of 0 to 5.

n23 is an integer of 0 to 4.

When n21 to n23 are 2 or more, one or more sets of adjacent two or more among two or more R's do not form a substituted or unsubstituted, saturated or unsaturated ring by bonding with each other.

R is

a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

a substituted or unsubstituted aryl group including 6 to 50 ring carbon atoms, or
a substituted or unsubstituted monovalent heterocyclic group including 5 to 50 ring atoms.

R₉₀₁ to R₉₀₄ are as defined in the formula (A1).

When a plurality of R's is present, the plurality of R's may be the same with or different from each other.

In one embodiment, L_{A}, L_{B} and L_{C} are independently an aromatic hydrocarbon ring group represented by the following formula (L1) or (L2).

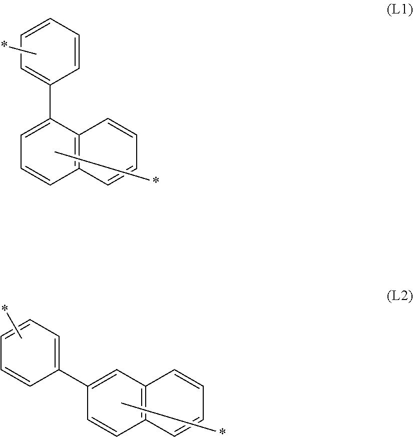

In the formula (L1) or (L2), one of two “*”'s is bonded to nitrogen atom in the formula (B1) and the other is bonded to one of A to C in the formula (B1).

In one embodiment, L_{A}, L_{B} and L_{C} are independently a single bond, or a substituted or unsubstituted arylene group including 6 to 12 ring carbon atoms.

In one embodiment, L_{C1} is a single bond.

In one embodiment, L_{C} is a single bond.

In one embodiment, L_{C} is a phenylene group.

In one embodiment, in the compounds represented by the formulas (B1), (B11) to (B19), A is preferably a substituted or unsubstituted aryl group including 6 to 12 ring carbon atoms, is more preferably a substituted or unsubstituted phenyl group, a substituted or unsubstituted biphenyl group, or a substituted or unsubstituted naphthyl group, and is still more preferably a phenyl group, a biphenyl group, or a naphthyl group.

In one embodiment, in the compounds represented by the formulas (B1), (B11) to (B19), B is preferably a substituted or unsubstituted aryl group including 6 to 12 ring carbon atoms, is more preferably a substituted or unsubstituted phenyl group, a substituted or unsubstituted biphenyl group, or a substituted or unsubstituted naphthyl group, and is still more preferably a phenyl group, a biphenyl group, or a naphthyl group.

Specific examples of the compound represented by the formula (B1) include the following compounds.

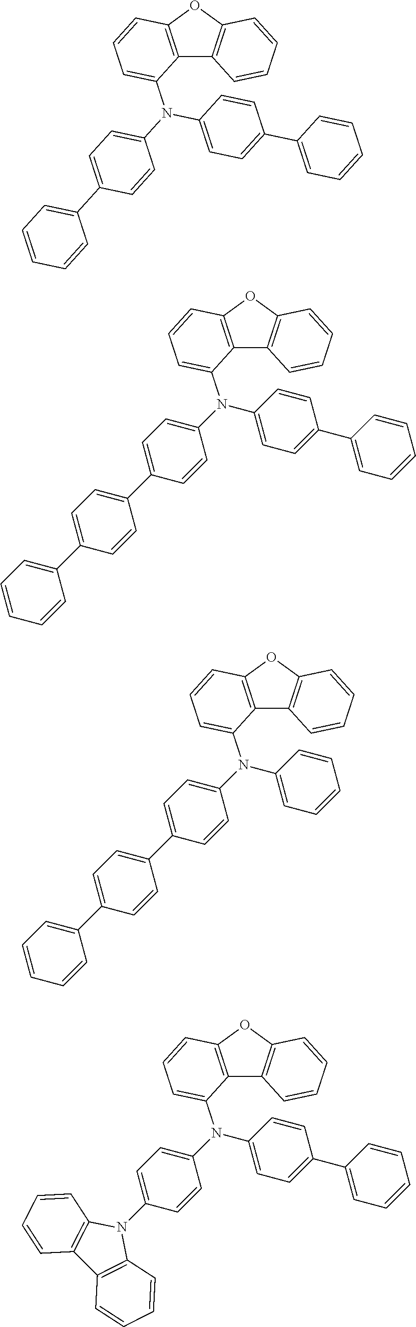 [SMILES: c1ccc(-c2ccc(N(c3ccc(-c4ccccc4)cc3)c3cccc4oc5ccccc5c34)cc2)cc1 ; c1ccc(-c2ccc(-c3ccc(N(c4ccc(-c5ccccc5)cc4)c4cccc5oc6ccccc6c45)cc3)cc2)cc1 ; c1ccc(-c2ccc(-c3ccc(N(c4ccccc4)c4cccc5oc6ccccc6c45)cc3)cc2)cc1 ; c1ccc(-c2ccc(N(c3ccc(-n4c5ccccc5c5ccccc54)cc3)c3cccc4oc5ccccc5c34)cc2)cc1]

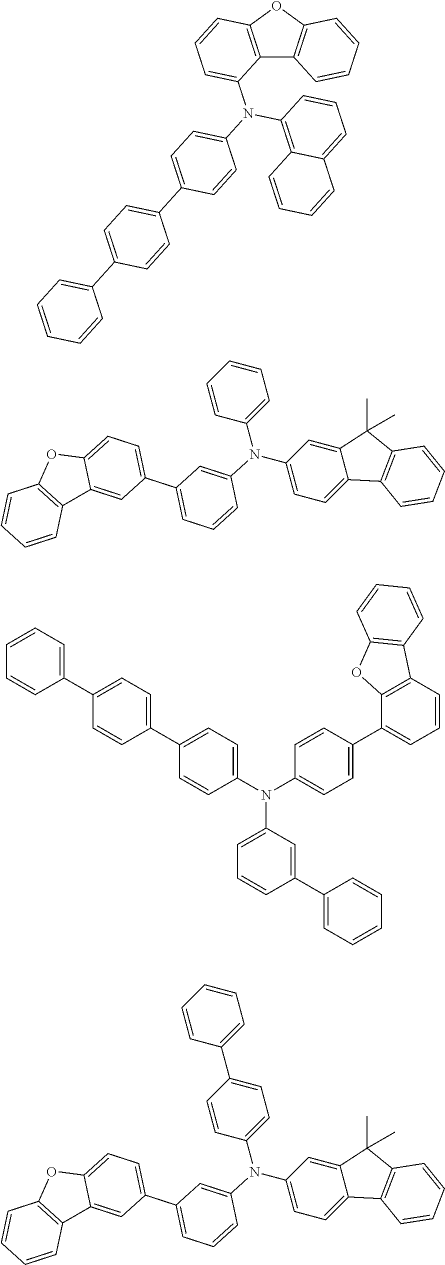 [SMILES: c1ccc(-c2ccc(-c3ccc(N(c4cccc5ccccc45)c4cccc5oc6ccccc6c45)cc3)cc2)cc1 ; CC1(C)c2ccccc2-c2ccc(N(c3ccccc3)c3cccc(-c4ccc5oc6ccccc6c5c4)c3)cc21 ; c1ccc(-c2ccc(-c3ccc(N(c4ccc(-c5cccc6c5oc5ccccc56)cc4)c4cccc(-c5ccccc5)c4)cc3)cc2)cc1 ; CC1(C)c2ccccc2-c2ccc(N(c3ccc(-c4ccccc4)cc3)c3cccc(-c4ccc5oc6ccccc6c5c4)c3)cc21]

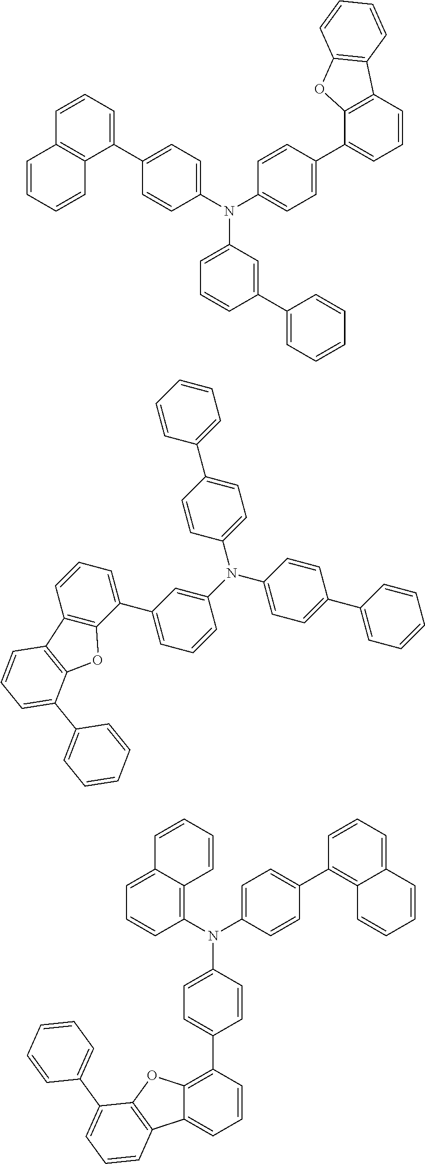 [SMILES: c1ccc(-c2cccc(N(c3ccc(-c4cccc5ccccc45)cc3)c3ccc(-c4cccc5c4oc4ccccc45)cc3)c2)cc1 ; c1ccc(-c2ccc(N(c3ccc(-c4ccccc4)cc3)c3cccc(-c4cccc5c4oc4c(-c6ccccc6)cccc45)c3)cc2)cc1 ; c1ccc(-c2cccc3c2oc2c(-c4ccc(N(c5ccc(-c6cccc7ccccc67)cc5)c5cccc6ccccc56)cc4)cccc23)cc1]

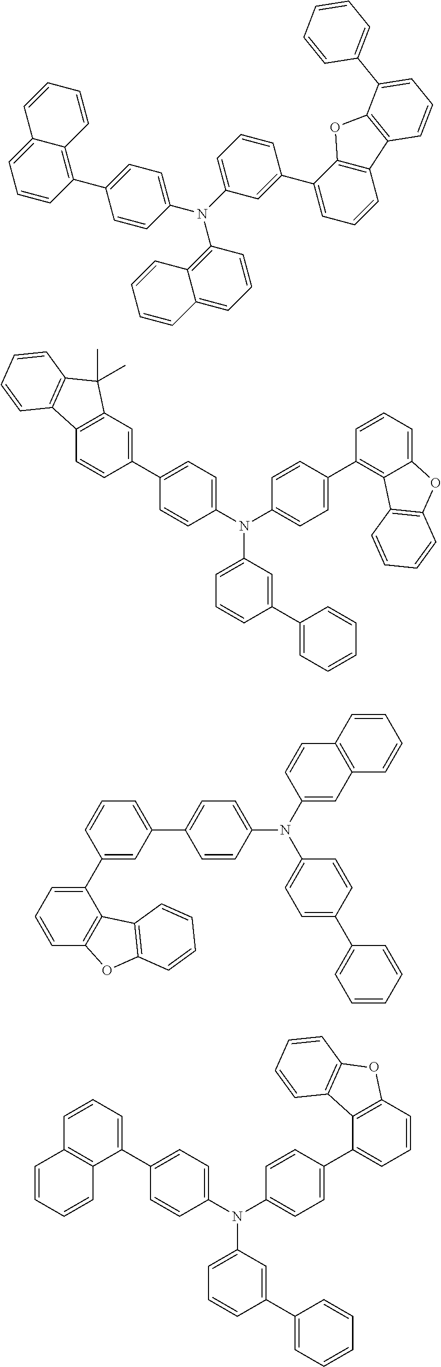

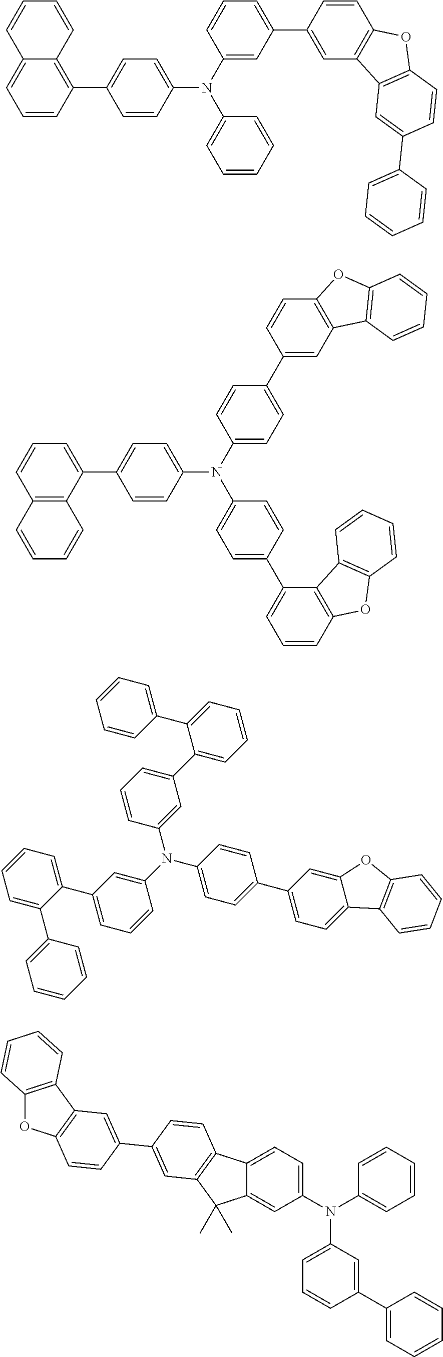

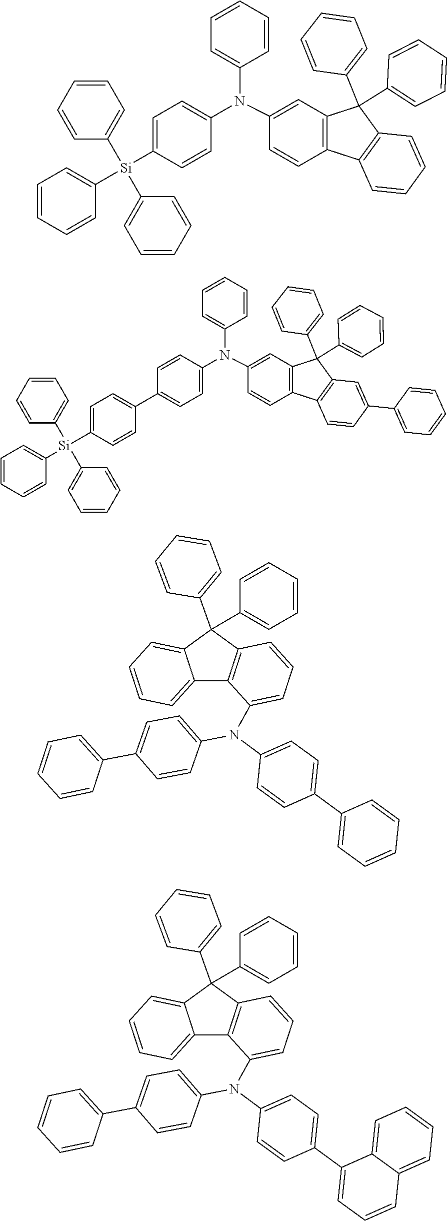

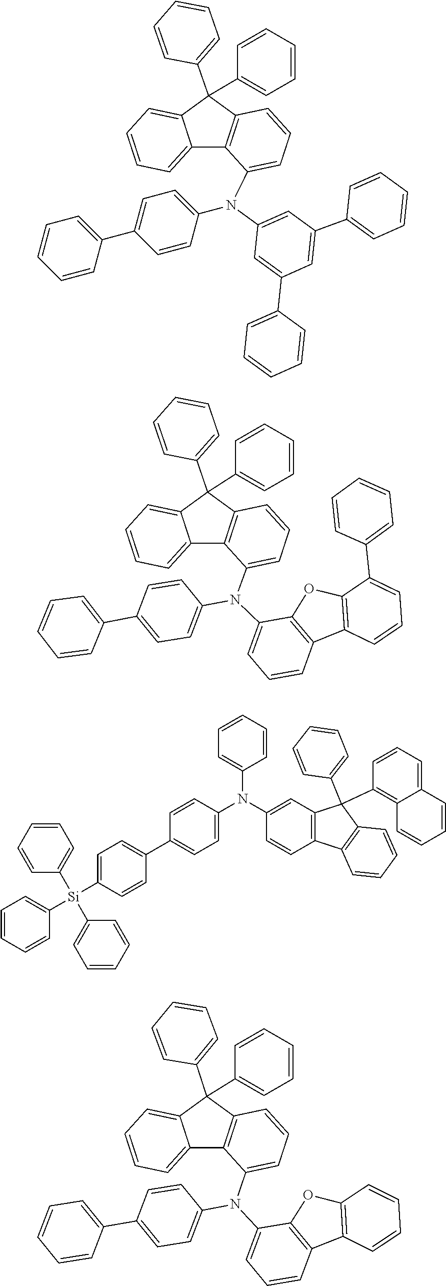

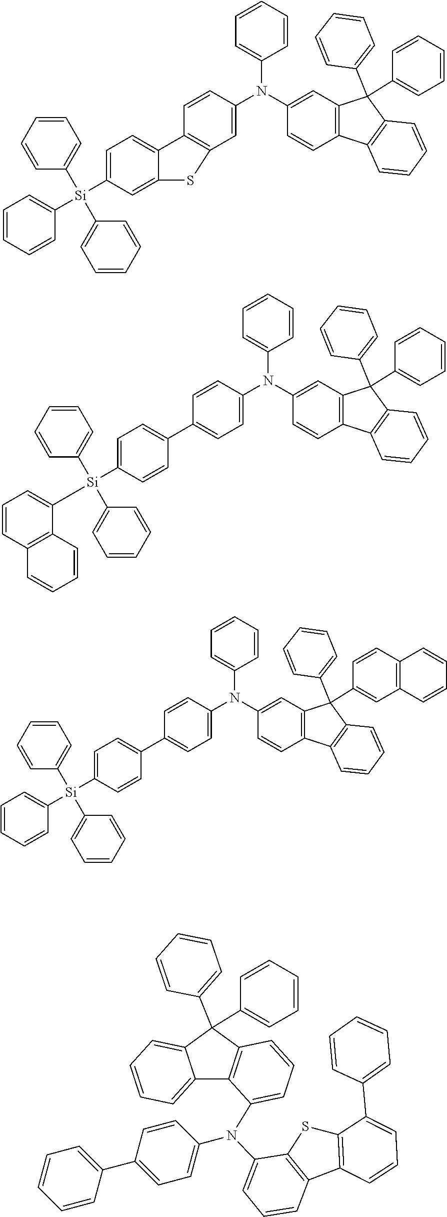 [SMILES: c1ccc(N(c2ccc3c(c2)C(c2ccccc2)(c2ccccc2)c2ccccc2-3)c2ccc3c(c2)sc2cc([Si](c4ccccc4)(c4ccccc4)c4ccccc4)ccc23)cc1 ; c1ccc(N(c2ccc(-c3ccc([Si](c4ccccc4)(c4ccccc4)c4cccc5ccccc45)cc3)cc2)c2ccc3c(c2)C(c2ccccc2)(c2ccccc2)c2ccccc2-3)cc1 ; c1ccc(N(c2ccc(-c3ccc([Si](c4ccccc4)(c4ccccc4)c4ccccc4)cc3)cc2)c2ccc3c(c2)C(c2ccccc2)(c2ccc4ccccc4c2)c2ccccc2-3)cc1 ; c1ccc(-c2ccc(N(c3cccc4c3-c3ccccc3C4(c3ccccc3)c3ccccc3)c3cccc4c3sc3c(-c5ccccc5)cccc34)cc2)cc1]

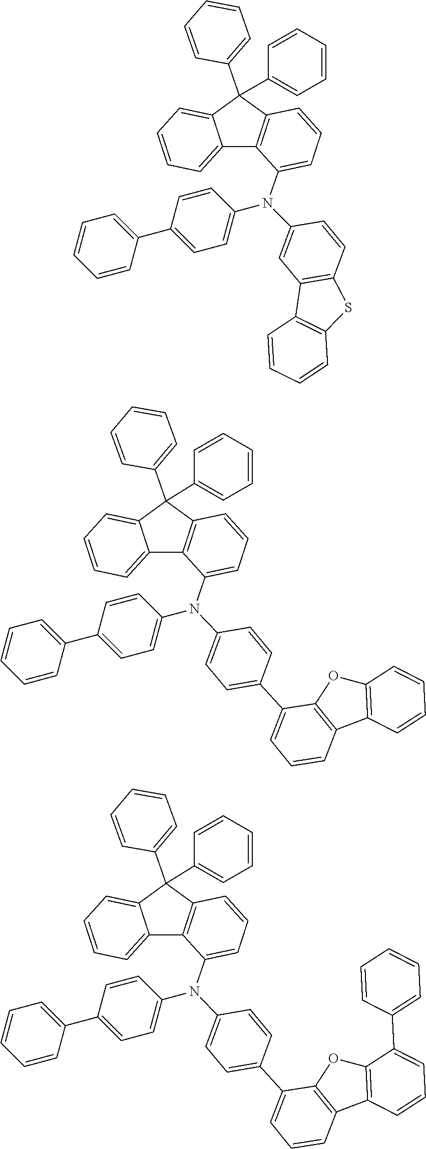 [SMILES: c1ccc(-c2ccc(N(c3ccc4sc5ccccc5c4c3)c3cccc4c3-c3ccccc3C4(c3ccccc3)c3ccccc3)cc2)cc1 ; c1ccc(-c2ccc(N(c3ccc(-c4cccc5c4oc4ccccc45)cc3)c3cccc4c3-c3ccccc3C4(c3ccccc3)c3ccccc3)cc2)cc1 ; c1ccc(-c2ccc(N(c3ccc(-c4cccc5c4oc4c(-c6ccccc6)cccc45)cc3)c3cccc4c3-c3ccccc3C4(c3ccccc3)c3ccccc3)cc2)cc1]

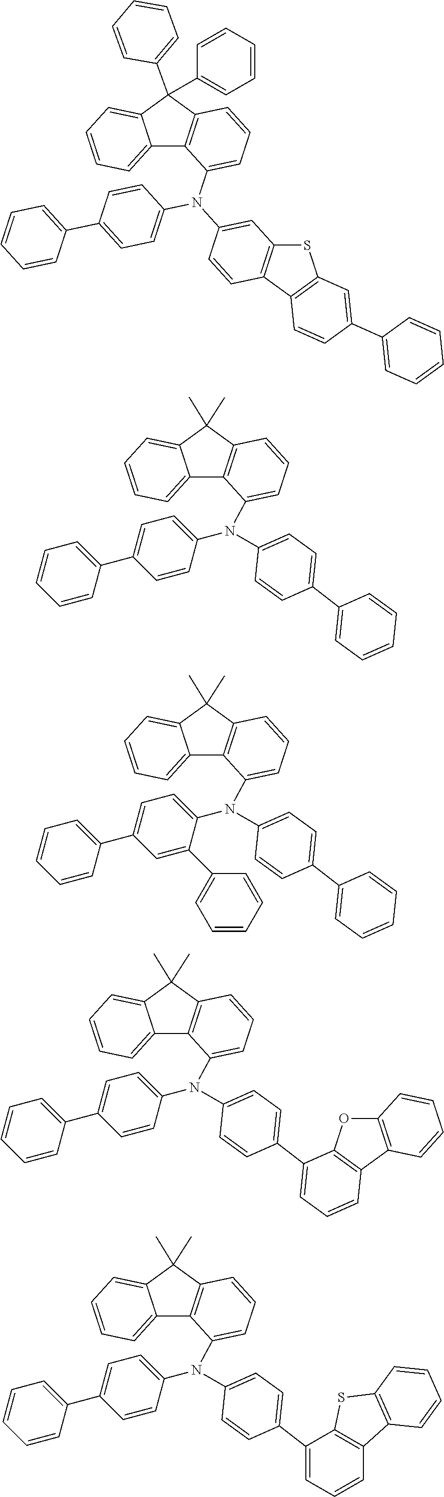

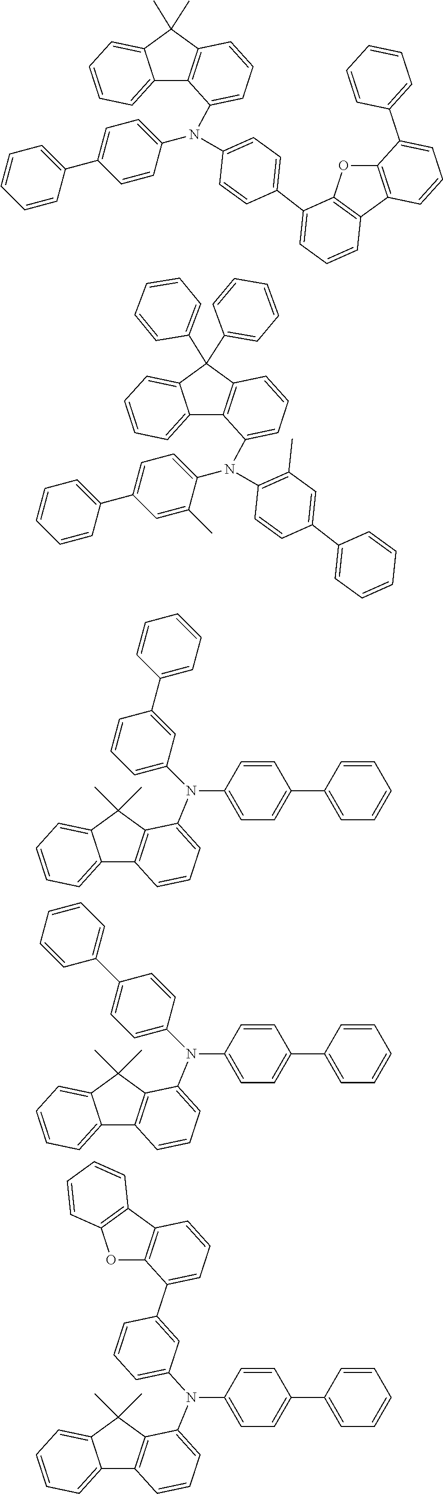

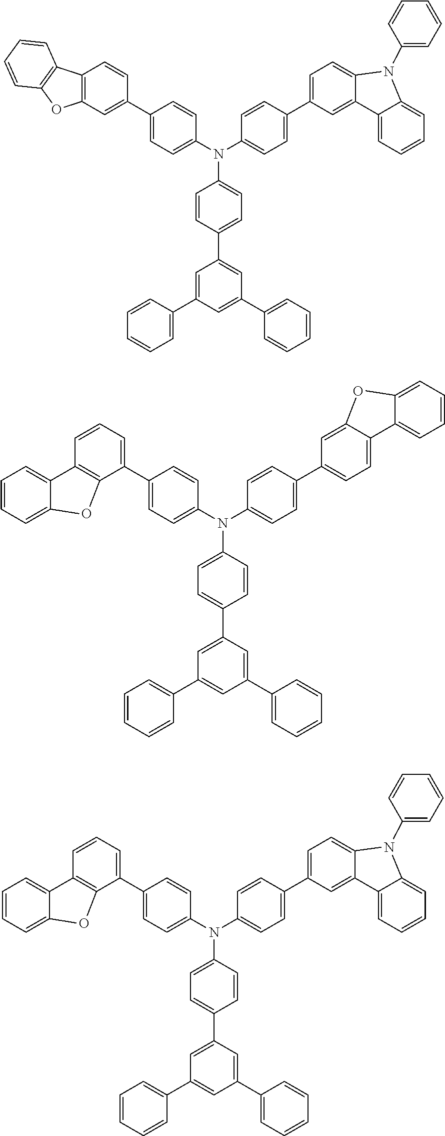 [SMILES: c1ccc(-c2cc(-c3ccccc3)cc(-c3ccc(N(c4ccc(-c5ccc6c(c5)oc5ccccc56)cc4)c4ccc(-c5ccc6c(c5)c5ccccc5n6-c5ccccc5)cc4)cc3)c2)cc1 ; c1ccc(-c2cc(-c3ccccc3)cc(-c3ccc(N(c4ccc(-c5ccc6c(c5)oc5ccccc56)cc4)c4ccc(-c5cccc6c5oc5ccccc56)cc4)cc3)c2)cc1 ; c1ccc(-c2cc(-c3ccccc3)cc(-c3ccc(N(c4ccc(-c5ccc6c(c5)c5ccccc5n6-c5ccccc5)cc4)c4ccc(-c5cccc6c5oc5ccccc56)cc4)cc3)c2)cc1]

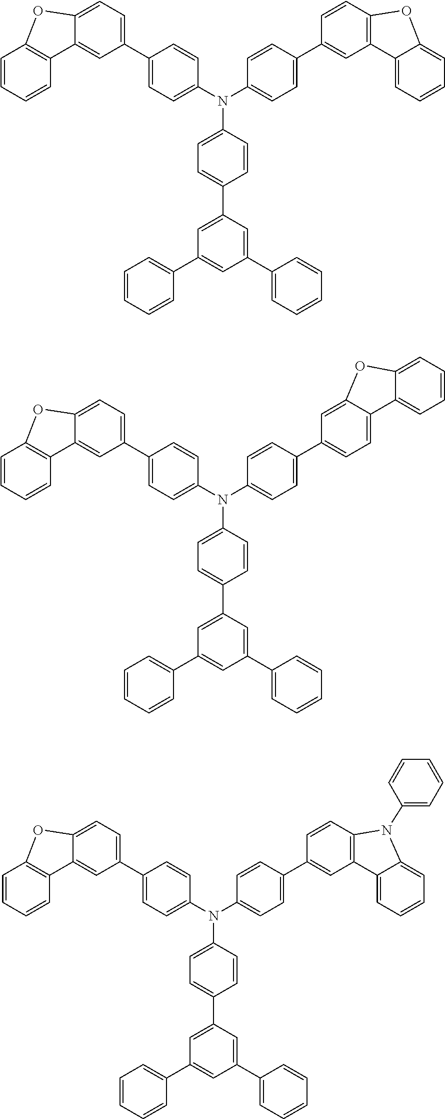 [SMILES: c1ccc(-c2cc(-c3ccccc3)cc(-c3ccc(N(c4ccc(-c5ccc6oc7ccccc7c6c5)cc4)c4ccc(-c5ccc6oc7ccccc7c6c5)cc4)cc3)c2)cc1 ; c1ccc(-c2cc(-c3ccccc3)cc(-c3ccc(N(c4ccc(-c5ccc6c(c5)oc5ccccc56)cc4)c4ccc(-c5ccc6oc7ccccc7c6c5)cc4)cc3)c2)cc1 ; c1ccc(-c2cc(-c3ccccc3)cc(-c3ccc(N(c4ccc(-c5ccc6oc7ccccc7c6c5)cc4)c4ccc(-c5ccc6c(c5)c5ccccc5n6-c5ccccc5)cc4)cc3)c2)cc1]

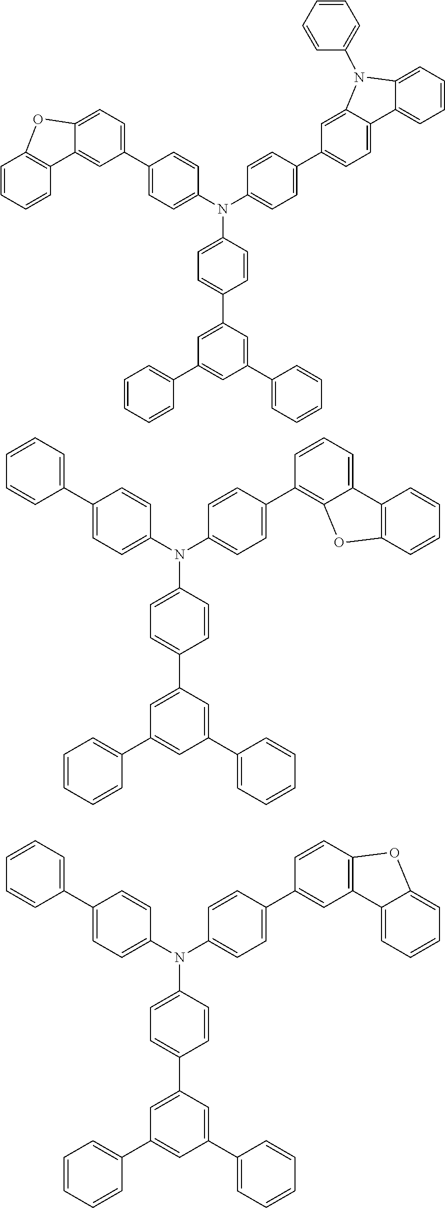

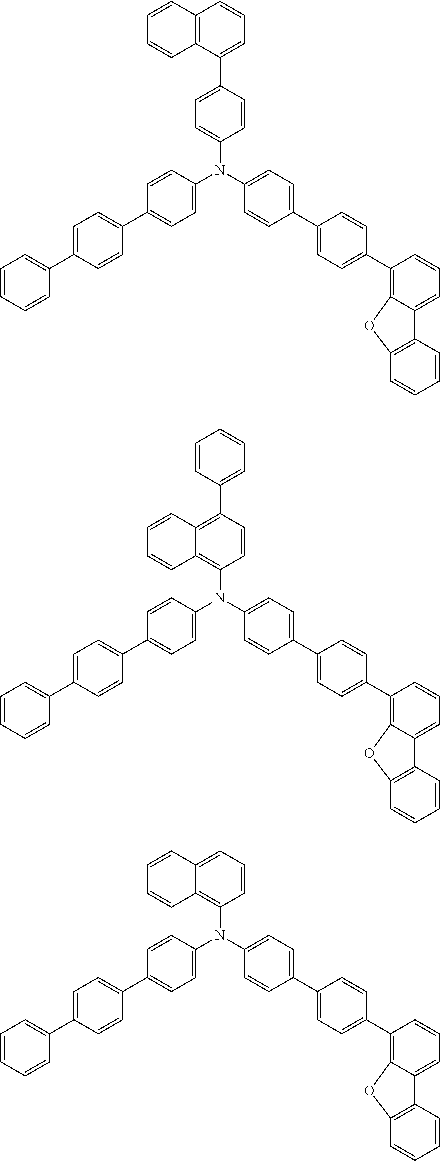 [SMILES: c1ccc(-c2ccc(-c3ccc(N(c4ccc(-c5ccc(-c6cccc7c6oc6ccccc67)cc5)cc4)c4ccc(-c5cccc6ccccc56)cc4)cc3)cc2)cc1 ; c1ccc(-c2ccc(-c3ccc(N(c4ccc(-c5ccc(-c6cccc7c6oc6ccccc67)cc5)cc4)c4ccc(-c5ccccc5)c5ccccc45)cc3)cc2)cc1 ; c1ccc(-c2ccc(-c3ccc(N(c4ccc(-c5ccc(-c6cccc7c6oc6ccccc67)cc5)cc4)c4cccc5ccccc45)cc3)cc2)cc1]

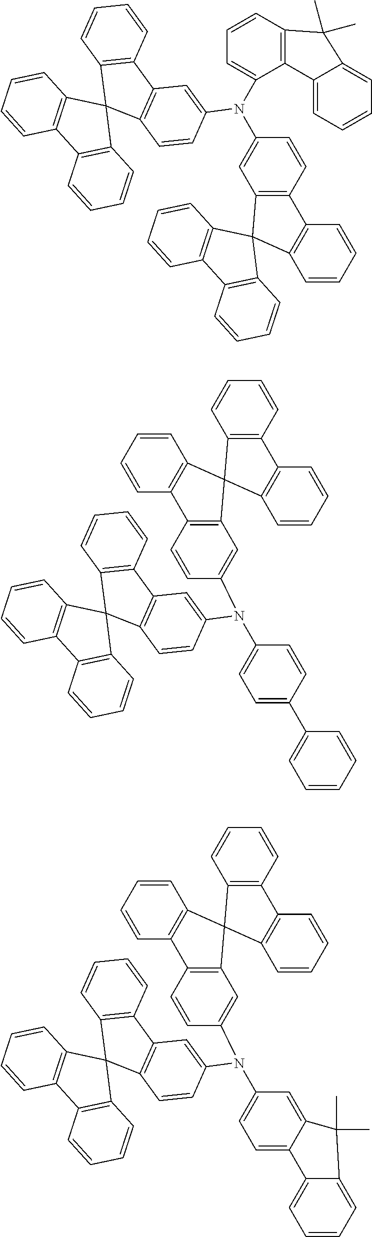

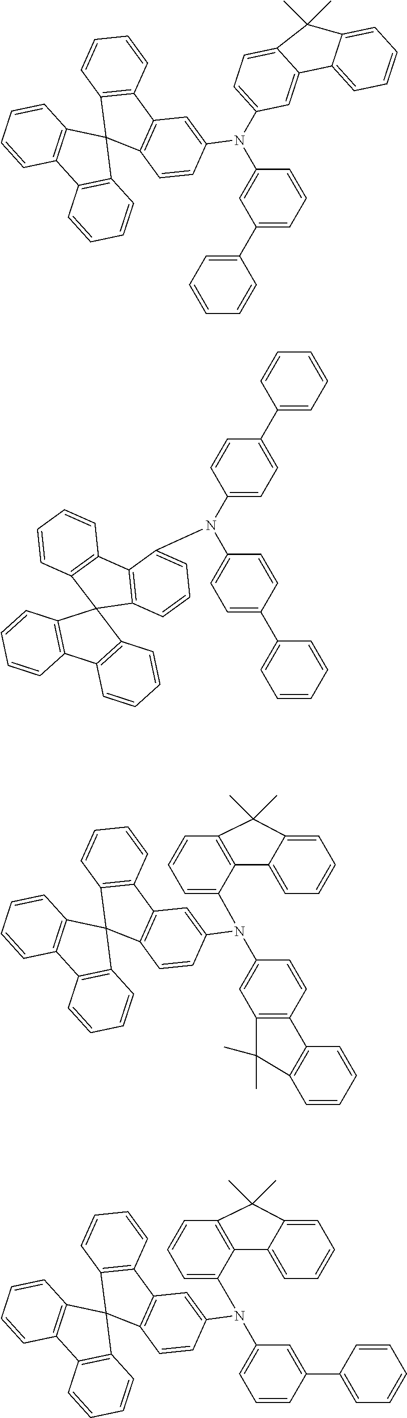 [SMILES: CC1(C)c2ccccc2-c2cc(N(c3cccc(-c4ccccc4)c3)c3ccc4c(c3)-c3ccccc3C43c4ccccc4-c4ccccc43)ccc21 ; c1ccc(-c2ccc(N(c3ccc(-c4ccccc4)cc3)c3cccc4c3-c3ccccc3C43c4ccccc4-c4ccccc43)cc2)cc1 ; CC1(C)c2ccccc2-c2ccc(N(c3ccc4c(c3)-c3ccccc3C43c4ccccc4-c4ccccc43)c3cccc4c3-c3ccccc3C4(C)C)cc21 ; CC1(C)c2ccccc2-c2c(N(c3cccc(-c4ccccc4)c3)c3ccc4c(c3)-c3ccccc3C43c4ccccc4-c4ccccc43)cccc21]

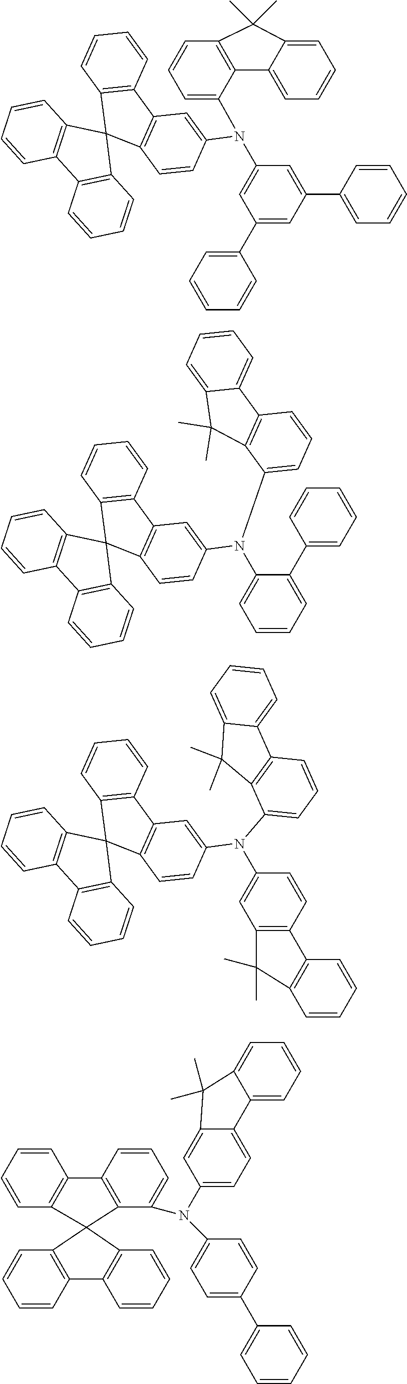

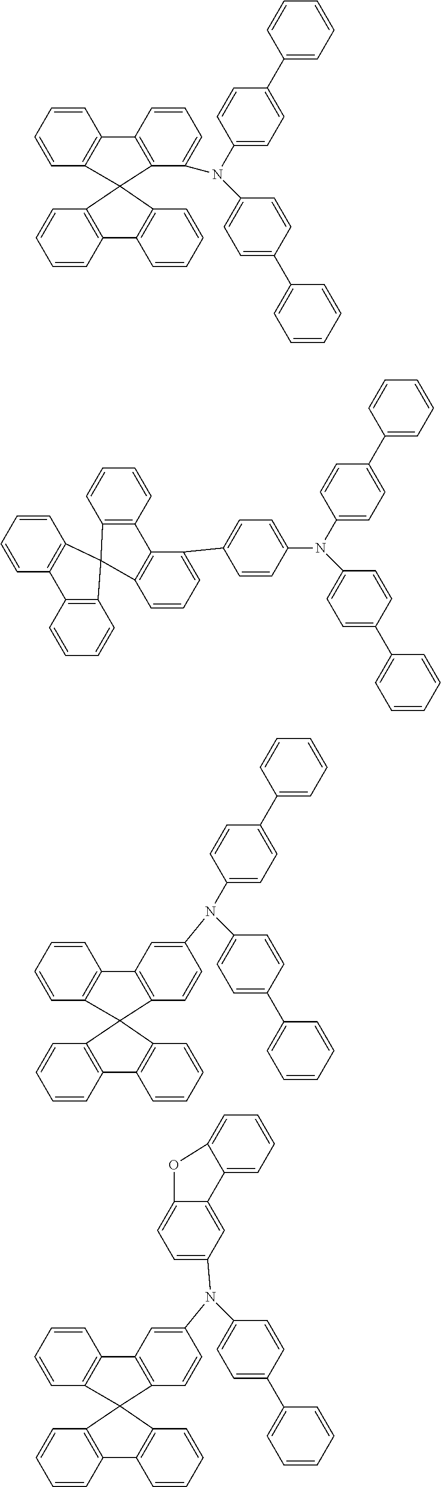

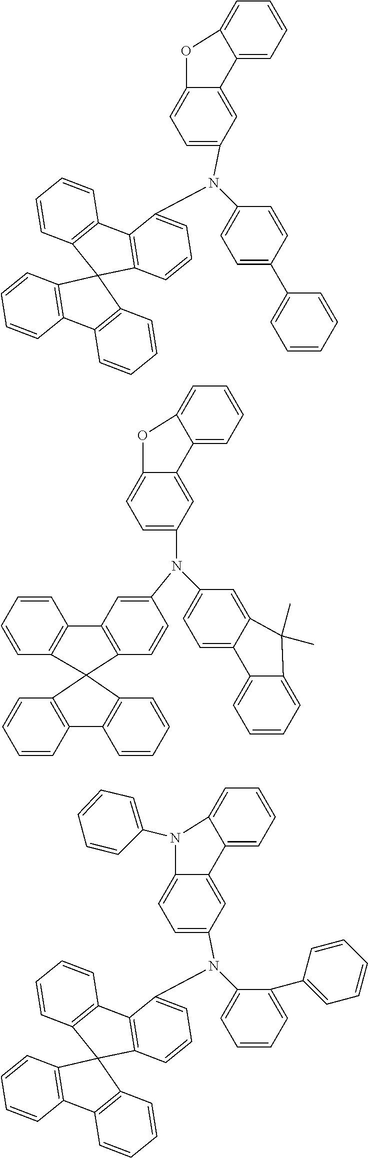

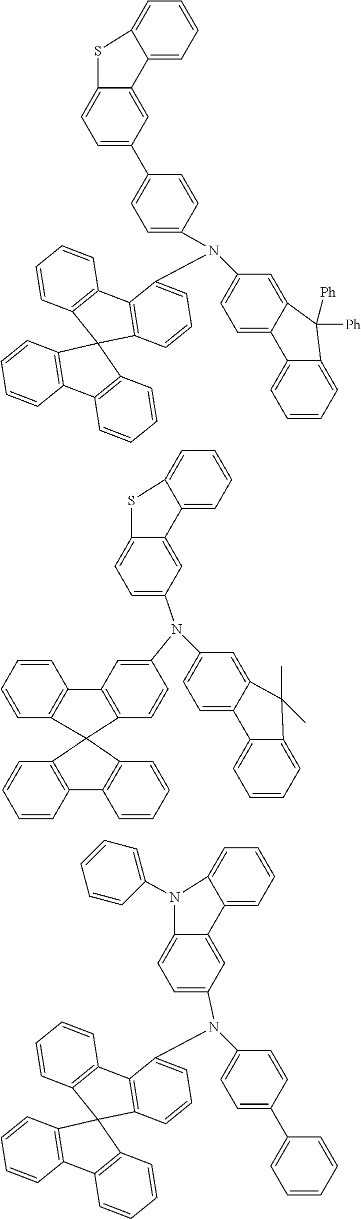

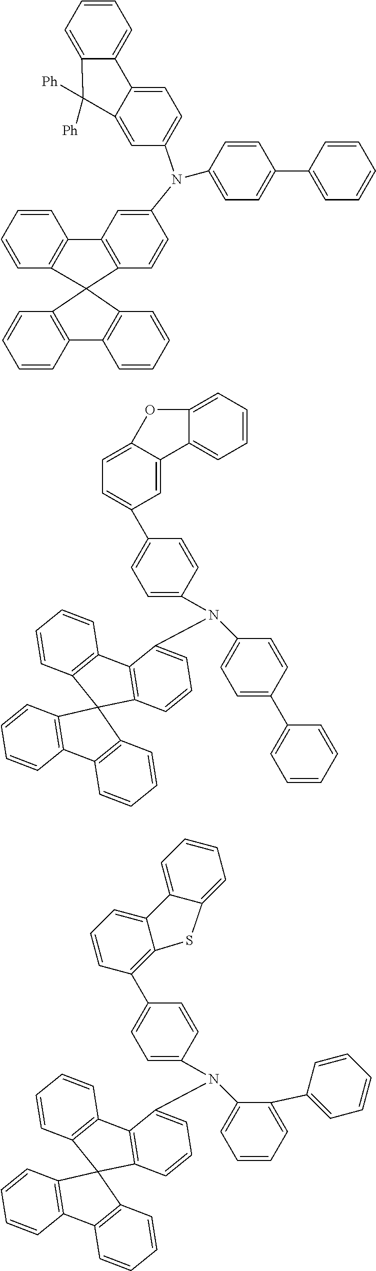

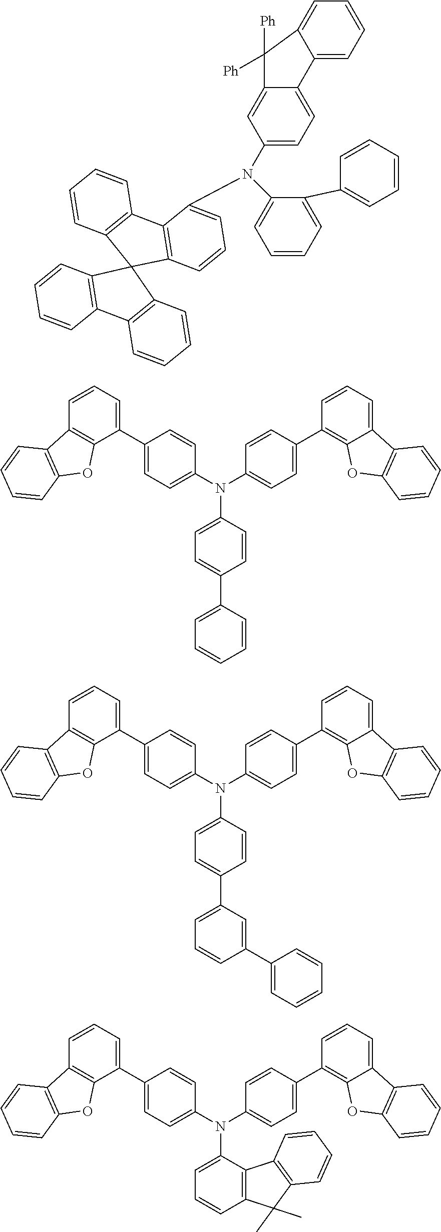

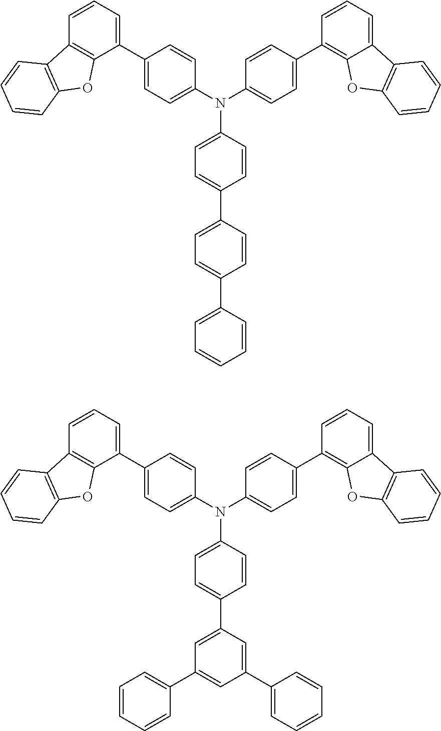 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2cc(-c3ccccc3)cc(-c3ccc(N(c4ccc(-c5cccc6c5oc5ccccc56)cc4)c4ccc(-c5cccc6c5oc5ccccc56)cc4)cc3)c2)cc1]

### (Compound Represented by the Formula (C1))

In one embodiment, the first layer contains a compound represented by the following formula (C1).

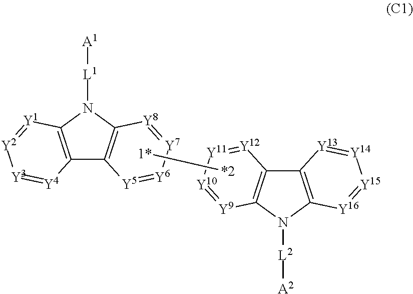

In the formula (c1),

A¹ and A² are independently a substituted or unsubstituted aryl group including 6 to 30 ring carbon atoms or a substituted or unsubstituted monovalent heterocyclic group including 5 to 30 ring atoms.

One of Y⁵ to Y⁸ is a carbon atom which is bonded to *1.

One of Y⁹ to Y¹² is a carbon atom which is bonded to *2.

Y¹ to Y⁴, Y¹³ to Y¹⁶, Y⁵ to Y⁸ which are not bonded to *1, and Y⁹ to Y¹² which are bonded to *2 are independently CR.

When a plurality of R's is present, one or more sets of adjacent two or more among the plurality of R's form a substituted or unsubstituted, saturated or unsaturated ring by bonding with each other, or do not form a substituted or unsubstituted, saturated or unsaturated ring.

R which does not form the substituted or unsubstituted, saturated or unsaturated ring is

a hydrogen atom,
a cyano group,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

a halogen atom, a nitro group,
a substituted or unsubstituted aryl group including 6 to 50 ring carbon atoms, or
a substituted or unsubstituted monovalent heterocyclic group including 5 to 50 ring atoms.

R₉₀₁ to R₉₀₄ are as defined in the formula (A1).

When a plurality of R's is present, the plurality of R's may be the same with or different from each other.

L¹ and L² are independently a single bond, a substituted or unsubstituted arylene group including 6 to 30 ring carbon atoms, or a substituted or unsubstituted divalent heterocyclic group including 5 to 30 ring atoms.

In one embodiment, the compound represented by the formula (C1) is a compound represented by the following formula (C10), (C11), or (C12).

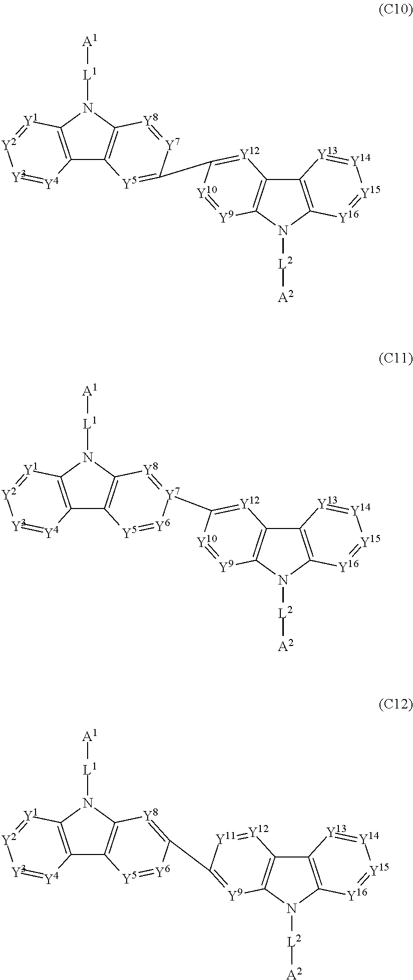

In the formulas (C10), (C11), and (C12), Y¹ to Y¹⁶, A¹, A², L¹ and L² are as defined in the formula (C1).

In the formula (C1), (C10), (C11) or (C12),

it is preferable that one of A¹ and A² be a substituted or unsubstituted aryl group including 6 to 30 ring carbon atoms, and the other of A¹ and A² be a substituted or unsubstituted phenyl group, a substituted or unsubstituted biphenyl group, a substituted or unsubstituted terphenyl group, a substituted or unsubstituted naphthyl group, a naphthylphenyl group, a triphenylenyl group, or a 9,9-biphenylfluorenyl group.

In the formula (C1), (C10), (C11) or (C12),

it is preferable that one of A¹ and A² be a substituted or unsubstituted aryl group including 6 to 30 ring carbon atoms, and the other of A¹ and A² be a substituted or unsubstituted phenyl group, a substituted or unsubstituted p-biphenyl group group, a substituted or unsubstituted m-biphenyl group, a substituted or unsubstituted o-biphenyl group, a substituted or unsubstituted 3-naphthylphenyl group, a triphenylenyl group, or a 9,9-biphenylfluorenyl group.

Specific examples of the compound represented by the formula (C1) include the following compounds.

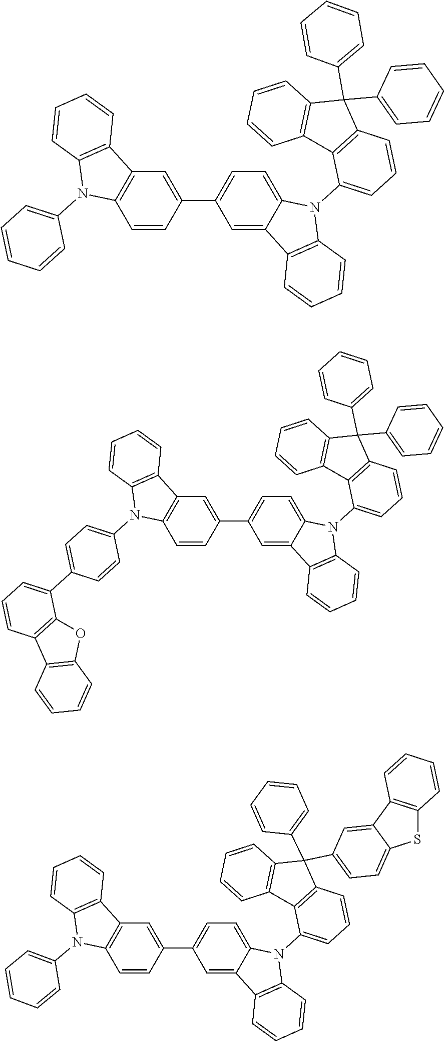 [SMILES: c1ccc(-n2c3ccccc3c3cc(-c4ccc5c(c4)c4ccccc4n5-c4cccc5c4-c4ccccc4C5(c4ccccc4)c4ccccc4)ccc32)cc1 ; c1ccc(C2(c3ccccc3)c3ccccc3-c3c(-n4c5ccccc5c5cc(-c6ccc7c(c6)c6ccccc6n7-c6ccc(-c7cccc8c7oc7ccccc78)cc6)ccc54)cccc32)cc1 ; c1ccc(-n2c3ccccc3c3cc(-c4ccc5c(c4)c4ccccc4n5-c4cccc5c4-c4ccccc4C5(c4ccccc4)c4ccc5sc6ccccc6c5c4)ccc32)cc1]

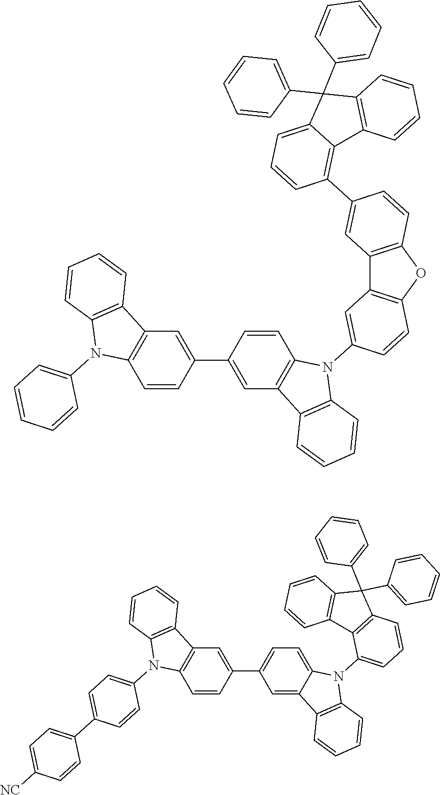 [SMILES: c1ccc(-n2c3ccccc3c3cc(-c4ccc5c(c4)c4ccccc4n5-c4ccc5oc6ccc(-c7cccc8c7-c7ccccc7C8(c7ccccc7)c7ccccc7)cc6c5c4)ccc32)cc1 ; N#Cc1ccc(-c2ccc(-n3c4ccccc4c4cc(-c5ccc6c(c5)c5ccccc5n6-c5cccc6c5-c5ccccc5C6(c5ccccc5)c5ccccc5)ccc43)cc2)cc1]

### (Compound Represented by the Formula (D1))

In one embodiment, a compound represented by the following formula (D1) is contained in the second layer.

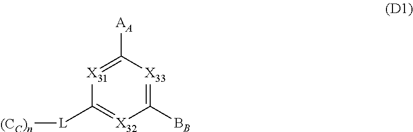

In the formula (D1),

one or more among X₃₁ to X₃₃ are a nitrogen atom, and the others which are not a nitrogen atom are CR.

R is

a hydrogen atom, a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

a substituted or unsubstituted aryl group including 6 to 50 ring carbon atoms, or
a substituted or unsubstituted monovalent heterocyclic group including 5 to 50 ring atoms.

R₉₀₁ to R₉₀₄ are as defined in the formula (A1).

A_{A} is a substituted or unsubstituted aryl group including 6 to 18 ring carbon atoms, or a substituted or unsubstituted monovalent heterocyclic group including 5 to 13 ring atoms.

B_{B} is a substituted or unsubstituted aryl group including 6 to 18 ring carbon atoms, or a substituted or unsubstituted monovalent heterocyclic group including 5 to 13 ring atoms.

L is a single bond, a substituted or unsubstituted (n+1)-valent aromatic hydrocarbon ring group including 6 to 18 ring carbon atoms, or a substituted or unsubstituted (n+1)-valent heterocyclic group including 5 to 13 ring atoms.

C_{C} is independently a substituted or unsubstituted aryl group including 6 to 30 ring carbon atoms, or a substituted or unsubstituted monovalent heterocyclic group including 5 to 60 ring atoms.

n is an integer of 1 to 3. When n is 2 or more, L is not a single bond.

In one embodiment, it is preferable that two among X₃₁ to X₃₃ in the formula (B1) be nitrogen atoms, and is more preferable that X₃₁ to X₃₃ be nitrogen atoms. In other words, a compound represented by the following formula (D10) is preferable.

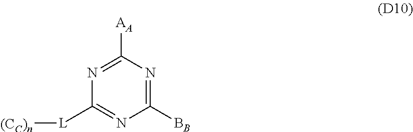

In the formula (D10), A_{A}, B_{B}, C_{C}, L and n are as defined in the formula (D1).

In one embodiment, the compound represented by the formula (D1) is a compound represented by the following formula (D11a).

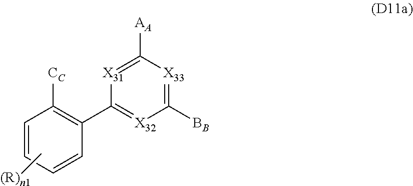

In the formula (D11a), A_{A}, B_{B}, C_{C}, X₃₁, X₃₂ and X₃₃ are as defined in the formula (D1) and the formula (D10).

When a plurality of R's is present, one or more sets of adjacent two or more among the plurality of R's form a substituted or unsubstituted, saturated or unsaturated ring by bonding with each other, or do not form a substituted or unsubstituted, saturated or unsaturated ring.

R which does not form the substituted or unsubstituted, saturated or unsaturated ring is

a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

a substituted or unsubstituted aryl group including 6 to 50 ring carbon atoms, or
a substituted or unsubstituted monovalent heterocyclic group including 5 to 50 ring atoms.

R₉₀₁ to R₉₀₄ are as defined in the formula (A1).

n1 is an integer of 0 to 4.

When a plurality of R's is present, the plurality of R's may be the same with or different from each other.

In one embodiment, it is preferable that two among X₃₁ to X₃₃ in the formula (D11a) be nitrogen atoms, and is more preferable that X₃₁ to X₃₃ be nitrogen atoms, as shown in the following formula (D11).

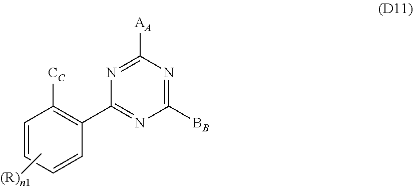

In the formula (D11), A_{A}, B_{B}, C_{C}, R and n1 are as defined in the formula (D11a).

In one embodiment, the compound represented by the formula (D1) is a compound represented by the following formula (D12a).

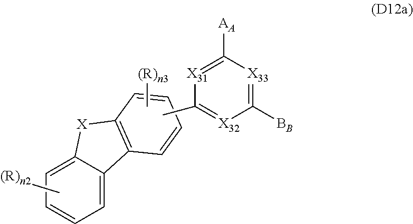

In the formula (D12a), A_{A}, B_{B}, X₃₁, X₃₂ and X₃₃ are as defined in the formula (D1).

X is CR₅₁R₅₂, NR₅₃, an oxygen atom or a sulfur atom.

When X is CR₅₁R₅₂, R₅₁ and R₅₂ form a substituted or unsubstituted, saturated or unsaturated ring by bonding with each other, or do not form a substituted or unsubstituted, saturated or unsaturated ring.

When a plurality of R's is present, one or more sets of adjacent two or more among the plurality of R's form a substituted or unsubstituted, saturated or unsaturated ring by bonding with each other, or do not form a substituted or unsubstituted, saturated or unsaturated ring.

R₅₃; and R, R₅₁ and R₅₂ which do not form the substituted or unsubstituted, saturated or unsaturated ring are independently,
a hydrogen atom,
a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃)

### —O—(R₉₀₄),

a substituted or unsubstituted aryl group including 6 to 50 ring carbon atoms, or
a substituted or unsubstituted monovalent heterocyclic group including 5 to 50 ring atoms.

R₉₀₁ to R₉₀₄ are as defined in the formula (A1).

n2 is an integer of 0 to 4, and n3 is an integer of 0 to 3.

When a plurality of R's is present, the plurality of R's may be the same with or different from each other.

In one embodiment, it is preferable that two among X₃₁ to X₃₃ in the formula (D12a) be nitrogen atoms, and is more preferable that X₃₁ to X₃₃ be nitrogen atoms, as shown in the following formula (D12).

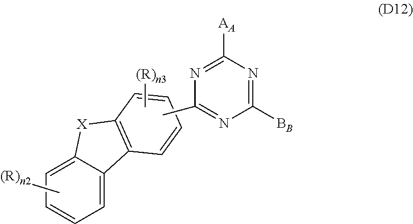

In the formula (D12), A_{A}, B_{B}, X, R, n2 and n3 are as defined in the formula (D12a).

In one embodiment, the compound represented by the formula (D12) is a compound represented by the following formula (D12-1).

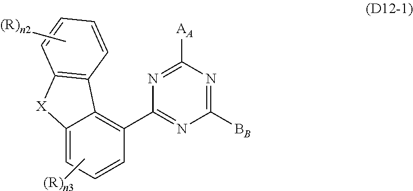

In the formula (D12-1), A_{A}, B_{B}, X, R, n2 and n3 are as defined in the formula (D12).

In one embodiment, the compound represented by the formula (D1) is a compound represented by the following formula (D13a).

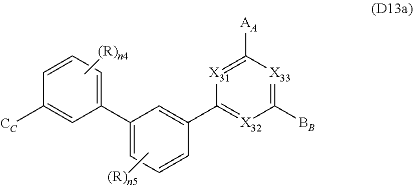

In the formula (D13a), A_{A}, B_{B}, C_{C}, X₃₁, X₃₂ and X₃₃ are as defined in the formula (D1).

When a plurality of R's is present, one or more sets of adjacent two or more among the plurality of R's form a substituted or unsubstituted, saturated or unsaturated ring by bonding with each other, or do not form a substituted or unsubstituted, saturated or unsaturated ring.

R which does not form the substituted or unsubstituted, saturated or unsaturated ring is

a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

a substituted or unsubstituted aryl group including 6 to 50 ring carbon atoms, or
a substituted or unsubstituted monovalent heterocyclic group including 5 to 50 ring atoms.

R₉₀₁ to R₉₀₄ are as defined in the formula (A1).

n4 and n5 are independently, an integer of 0 to 4.

When a plurality of R's is present, the plurality of R's may be the same with or different from each other.

In one embodiment, it is preferable that two among X₃₁ to X₃₃ in the formula (D13a) be nitrogen atoms, and is more preferable that X₃₁ to X₃₃ be nitrogen atoms, as shown in the following formula (D13).

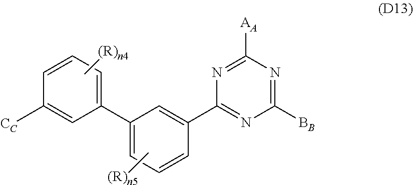

In the formula (D13), A_{A}, B_{B}, C_{C}, R, n4 and n5 are as defined in the formula (D13a).

In one embodiment, C_{C} of the formulas is preferably a substituted or unsubstituted monovalent heterocyclic group including 13 to 35 ring atoms, and is preferably a substituted or unsubstituted aryl group including 14 to 24 ring carbon atoms.

In one embodiment, the compound represented by the formula (D1) is a compound represented by the following formula (D14a).

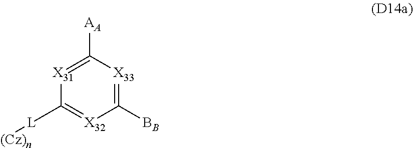

In the formula (D14a), A_{A}, B_{B}, L, X₃₁, X₃₂ and X₃₃ are as defined in the formula (D1). Cz is a group represented by any one of the following formulas (Cz1), (Cz2) and (Cz3). n is an integer of 1 to 3.

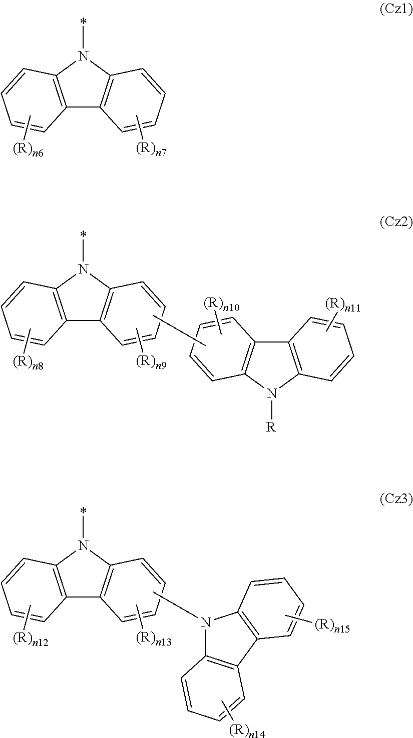

In the formulas (Cz1), (Cz2) and (Cz3),

when a plurality of R's is present, one or more sets of adjacent two or more among the plurality of R's form a substituted or unsubstituted, saturated or unsaturated ring by bonding with each other, or do not form a substituted or unsubstituted, saturated or unsaturated ring.

R which does not form the substituted or unsubstituted, saturated or unsaturated ring is

a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

a substituted or unsubstituted aryl group including 6 to 50 ring carbon atoms, or
a substituted or unsubstituted monovalent heterocyclic group including 5 to 50 ring atoms.
R₉₀₁ to R₉₀₄ are as defined in the formula (A1).

n6 and n7 are independently an integer of 0 to 4.

n8 and n11 are independently an integer of 0 to 4, and n9 and n10 are independently an integer of 0 to 3.

n12, n14 and n15 are independently an integer of 0 to 4, and n13 is an integer of 0 to 3.

When a plurality of R's is present, the plurality of R's may be the same with or different from each other.

* is bonded to L.

In one embodiment, it is preferable that two among X₃₁ to X₃₃ in the formula (D14a) be nitrogen atoms, and is more preferable that X₃₁ to X₃₃ be nitrogen atoms, as shown in the following formula (D14).

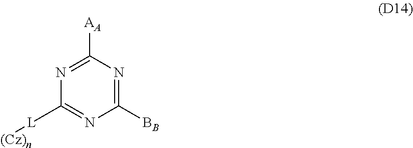

In the formula (D14), A_{A}, B_{B}, L, Cz, and n are as defined in the formula (D14a).

In one embodiment, the compound represented by the formula (D1) is a compound represented by the following formula (D15a).

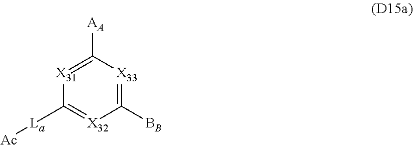

In the formula (D15a), A_{A}, B_{B}, X₃₁, X₃₂ and X₃₃ are as defined in the formula (D1).

Lₐ is a single bond, a substituted or unsubstituted divalent aromatic hydrocarbon ring group including 6 to 18 ring carbon atoms, or a substituted or unsubstituted divalent heterocyclic group including 5 to 13 ring atoms.

Ac is a group represented by any one of the following formulas (Ac1), (Ac2) and (Ac3).

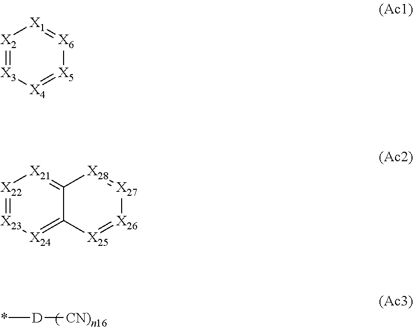

In the formula (Ac1),

one or more among X₁ to X₆ are nitrogen atoms, the other which are not nitrogen atoms are CR's, and one of R is bonded to Lₐ.

R is

a hydrogen atom, a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

a substituted or unsubstituted aryl group including 6 to 50 ring carbon atoms, or
a substituted or unsubstituted monovalent heterocyclic group including 5 to 50 ring atoms.
R₉₀₁ to R₉₀₄ are as defined in the formula (A1).

When a plurality of R's is present, the plurality of R's may be the same with or different from each other.

In the formula (Ac2),

one or more among X₂₁ to X₂₈ is nitrogen atom, the other which are not nitrogen atoms are CR's, and one of R is bonded to Lₐ.

When a plurality of R's is present, one or more sets of adjacent two or more among the plurality of R's form a substituted or unsubstituted, saturated or unsaturated ring by bonding with each other, or do not form a substituted or unsubstituted, saturated or unsaturated ring.

R which does not form the substituted or unsubstituted, saturated or unsaturated ring is

a hydrogen atom,
a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

a substituted or unsubstituted aryl group including 6 to 50 ring carbon atoms, or
a substituted or unsubstituted monovalent heterocyclic group including 5 to 50 ring atoms.
R₉₀₁ to R₉₀₄ are as defined in the formula (A1).

When a plurality of R's is present, the plurality of R's may be the same with or different from each other.

In the formula (Ac3),

D is an aryl group including 6 to 18 ring carbon atoms which is substituted by n16 cyano groups, or a heteroaryl group including 5 to 13 ring atoms which is substituted by n16 cyano groups. Provided that D may have a substituent other than a cyano group.

n16 represents the number of cyano groups substituted to D and is integer of 1 to 9. “*” is bonded to Lₐ.

In one embodiment, it is preferable that two among X₃₁ to X₃₃ in the formula (D15a) be nitrogen atoms, and is more preferable that X₃₁ to X₃₃ be nitrogen atoms, as shown in the following formula (D15).

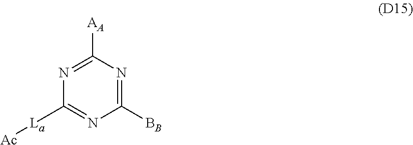

In the formula (D15), A_{A}, B_{B}, La and Ac are as defined in the formula (D15a).

In one embodiment, the compound represented by the formula (D1) is a compound represented by the following formula (D16a).

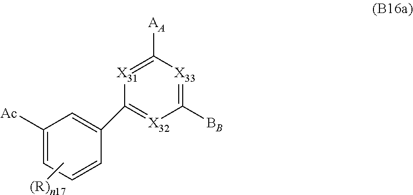

In the formula (D16a),

A_{A}, B_{B}, Ac, X₃₁, X₃₂ and X₃₃ are as defined in the formula (D15a).

n17 is an integer of 0 to 4.

When a plurality of R's is present, one or more sets of adjacent two or more among the plurality of R's form a substituted or unsubstituted, saturated or unsaturated ring by bonding with each other, or do not form a substituted or unsubstituted, saturated or unsaturated ring.

R which does not form the ring is

a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),

### —O—(R₉₀₄),

a substituted or unsubstituted aryl group including 6 to 50 ring carbon atoms, or
a substituted or unsubstituted monovalent heterocyclic group including 5 to 50 ring atoms.
R₉₀₁ to R₉₀₄ is as defined in the formula (A1).

When a plurality of R's is present, the plurality of R's may be the same with or different from each other.

In one embodiment, it is preferable that two among X₃₁ to X₃₃ in the formula (D16a) be nitrogen atoms, and is more preferable that X₃₁ to X₃₃ be nitrogen atoms, as shown in the following formula (D16).

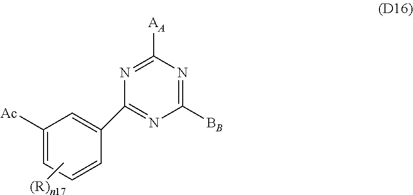

In the formula (D16), A_{A}, B_{B}, Ac, R and n17 are as defined in the formula (D16a).

In one embodiment, a compound represented by the following formula (D16-1) is preferred.

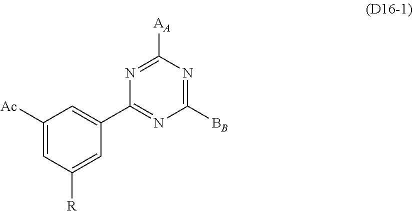

In the formula (D16-1), A_{A}, B_{B}, Ac and R are as defined in the formula (D16a).

In one embodiment, L or Lₐ of each of the formulas is an aromatic hydrocarbon ring group represented by the following formula (L1) or (L2).

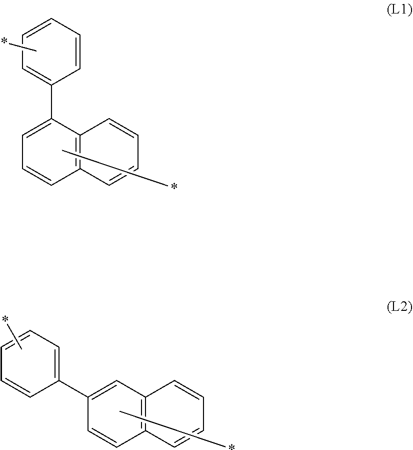

In the formula (L1) or (L2), either one of two “*”'s is bonded to the nitrogen-containing six-membered ring and the other is bonded to (C)n, (Cz)n or Ac. When n is an integer of 1 to 3, one to three bondings to (C)n or (Cz)n are present, respectively.

In one embodiment, L of each of the formulas is a single bond, or a substituted or unsubstituted (n+1)-valent aromatic hydrocarbon ring group including 6 to 12 ring carbon atoms.

In one embodiment, L or Lₐ of each of the formulas is a single bond.

In one embodiment, A_{A} of each of the formulas is preferably a substituted or unsubstituted aryl group including 6 to 12 ring carbon atoms, and is more preferably a substituted or unsubstituted phenyl group, a substituted or unsubstituted biphenyl group, or a substituted or unsubstituted naphthyl group, and is still more preferably a phenyl group, a biphenyl group, or a naphthyl group.

In one embodiment, B_{B} of each of the formulas is preferably a substituted or unsubstituted aryl group including 6 to 12 ring carbon atoms, and is more preferably a substituted or unsubstituted phenyl group, a substituted or unsubstituted biphenyl group, or a substituted or unsubstituted naphthyl group, and is still more preferably a phenyl group, a biphenyl group, or a naphthyl group.

Specific examples of the compound represented by the formula (D1) include the following compounds.

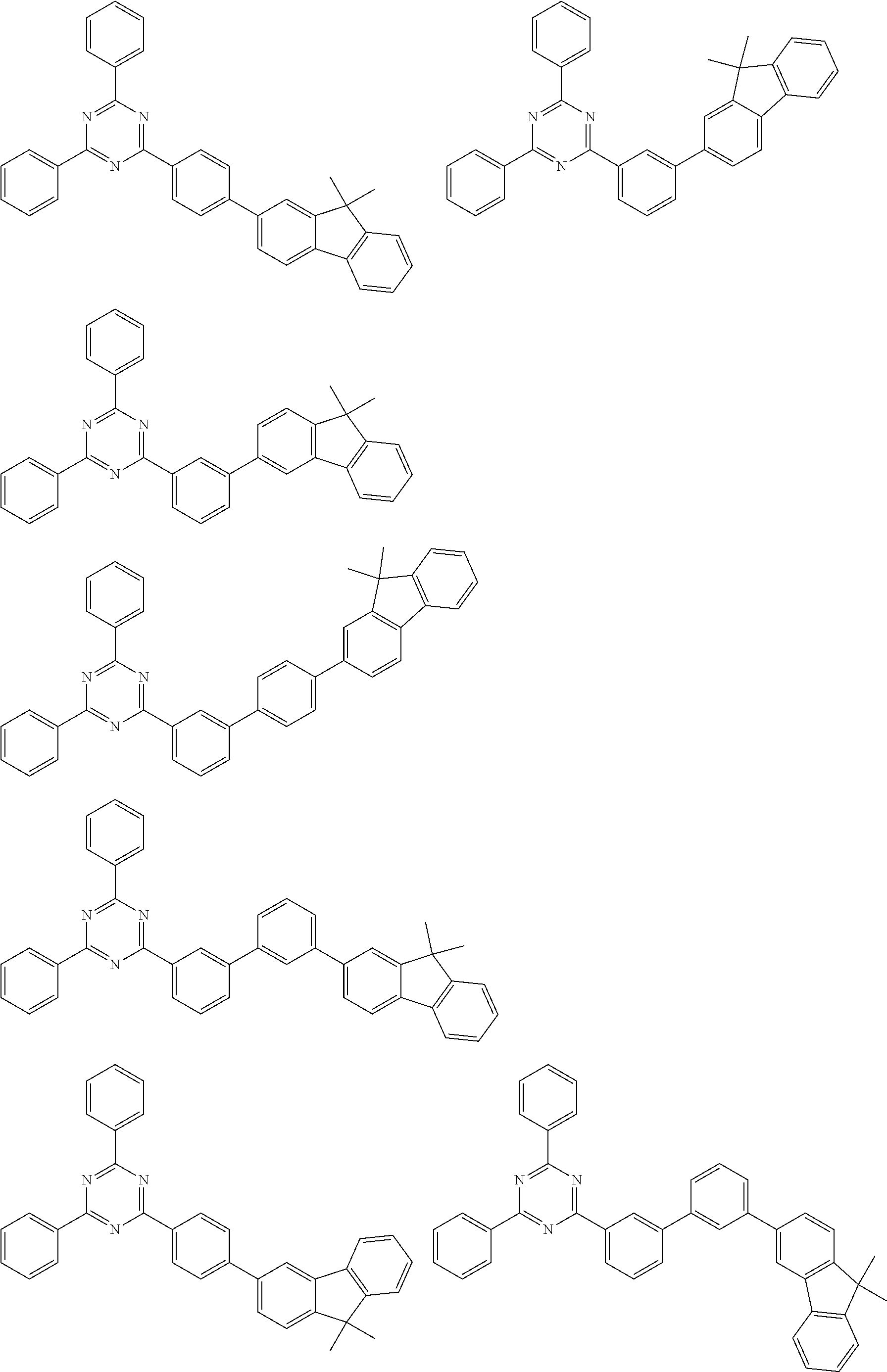

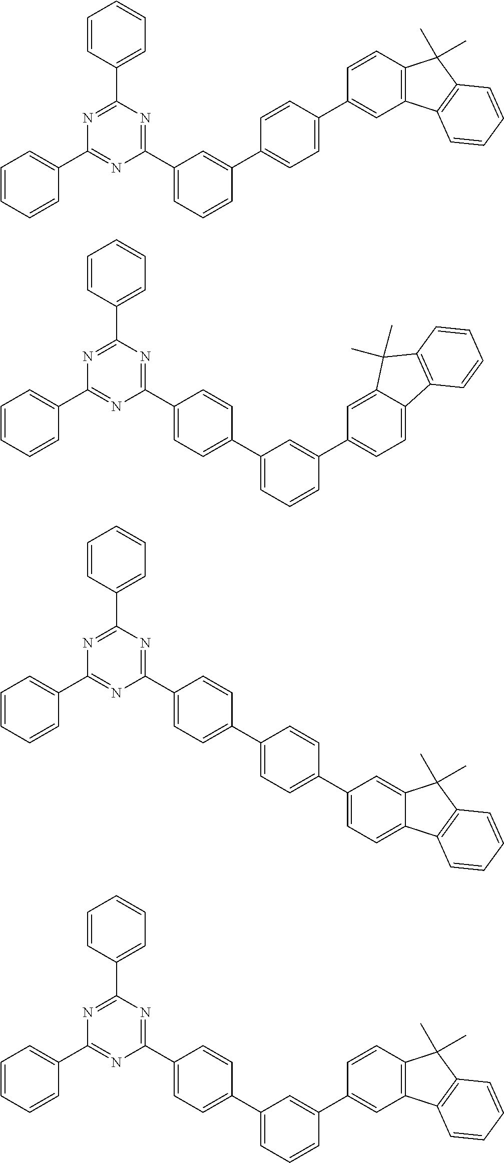 [SMILES: CC1(C)c2ccccc2-c2cc(-c3ccc(-c4cccc(-c5nc(-c6ccccc6)nc(-c6ccccc6)n5)c4)cc3)ccc21 ; CC1(C)c2ccccc2-c2ccc(-c3cccc(-c4ccc(-c5nc(-c6ccccc6)nc(-c6ccccc6)n5)cc4)c3)cc21 ; CC1(C)c2ccccc2-c2ccc(-c3ccc(-c4ccc(-c5nc(-c6ccccc6)nc(-c6ccccc6)n5)cc4)cc3)cc21 ; CC1(C)c2ccccc2-c2cc(-c3cccc(-c4ccc(-c5nc(-c6ccccc6)nc(-c6ccccc6)n5)cc4)c3)ccc21]

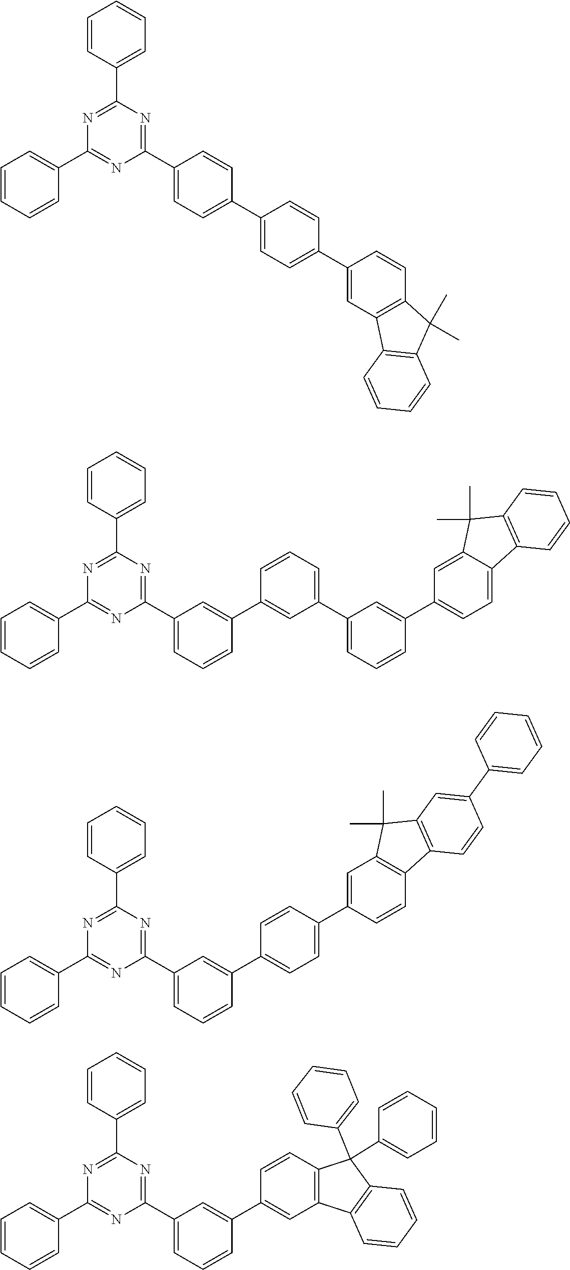 [SMILES: CC1(C)c2ccccc2-c2cc(-c3ccc(-c4ccc(-c5nc(-c6ccccc6)nc(-c6ccccc6)n5)cc4)cc3)ccc21 ; CC1(C)c2ccccc2-c2ccc(-c3cccc(-c4cccc(-c5cccc(-c6nc(-c7ccccc7)nc(-c7ccccc7)n6)c5)c4)c3)cc21 ; CC1(C)c2cc(-c3ccccc3)ccc2-c2ccc(-c3ccc(-c4cccc(-c5nc(-c6ccccc6)nc(-c6ccccc6)n5)c4)cc3)cc21 ; c1ccc(-c2nc(-c3ccccc3)nc(-c3cccc(-c4ccc5c(c4)-c4ccccc4C5(c4ccccc4)c4ccccc4)c3)n2)cc1]

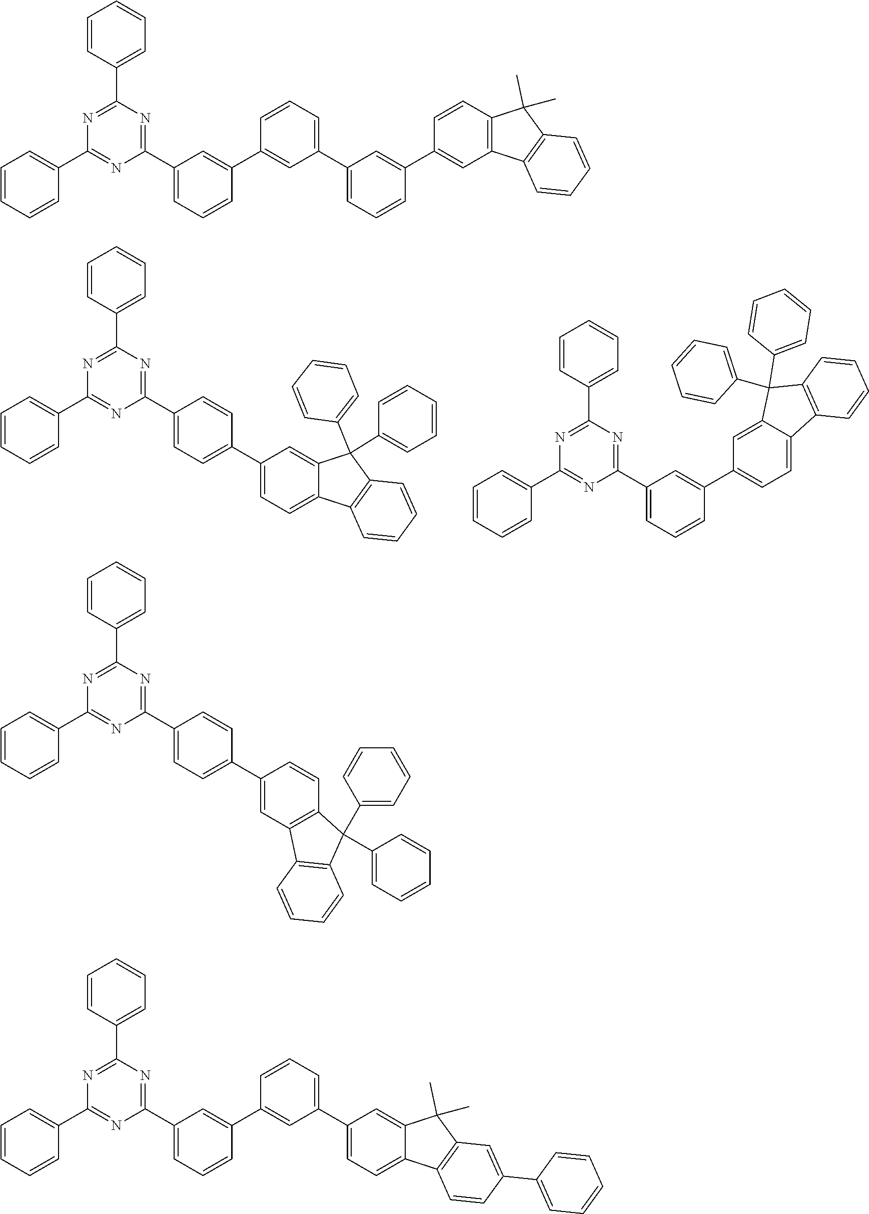 [SMILES: CC1(C)c2ccccc2-c2cc(-c3cccc(-c4cccc(-c5cccc(-c6nc(-c7ccccc7)nc(-c7ccccc7)n6)c5)c4)c3)ccc21 ; c1ccc(-c2nc(-c3ccccc3)nc(-c3ccc(-c4ccc5c(c4)C(c4ccccc4)(c4ccccc4)c4ccccc4-5)cc3)n2)cc1 ; c1ccc(-c2nc(-c3ccccc3)nc(-c3cccc(-c4ccc5c(c4)C(c4ccccc4)(c4ccccc4)c4ccccc4-5)c3)n2)cc1 ; c1ccc(-c2nc(-c3ccccc3)nc(-c3ccc(-c4ccc5c(c4)-c4ccccc4C5(c4ccccc4)c4ccccc4)cc3)n2)cc1 ; CC1(C)c2cc(-c3ccccc3)ccc2-c2ccc(-c3cccc(-c4cccc(-c5nc(-c6ccccc6)nc(-c6ccccc6)n5)c4)c3)cc21]

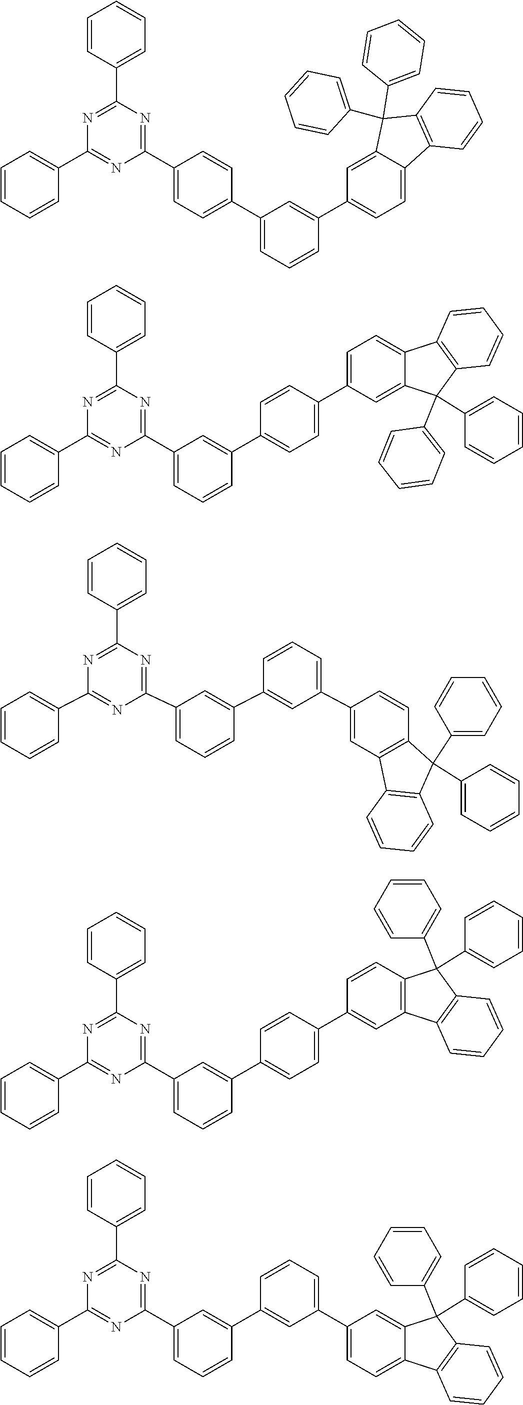

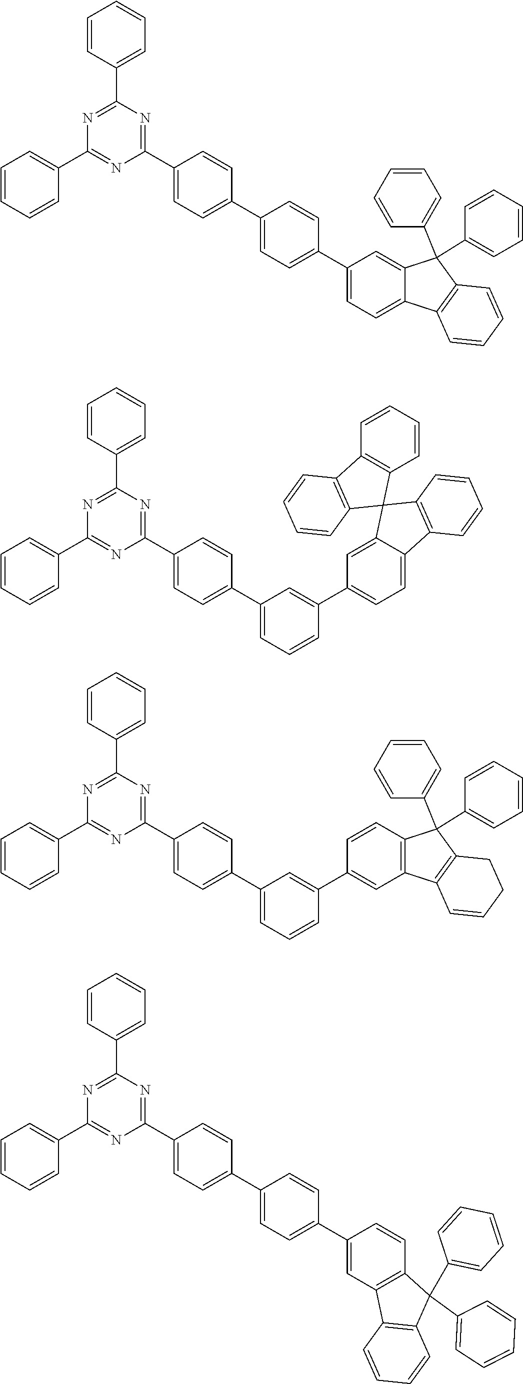 [SMILES: c1ccc(-c2nc(-c3ccccc3)nc(-c3ccc(-c4ccc(-c5ccc6c(c5)C(c5ccccc5)(c5ccccc5)c5ccccc5-6)cc4)cc3)n2)cc1 ; c1ccc(-c2nc(-c3ccccc3)nc(-c3ccc(-c4cccc(-c5ccc6c(c5)C5(c7ccccc7-c7ccccc75)c5ccccc5-6)c4)cc3)n2)cc1 ; C1=CC2=C(CC1)C(c1ccccc1)(c1ccccc1)c1ccc(-c3cccc(-c4ccc(-c5nc(-c6ccccc6)nc(-c6ccccc6)n5)cc4)c3)cc12 ; c1ccc(-c2nc(-c3ccccc3)nc(-c3ccc(-c4ccc(-c5ccc6c(c5)-c5ccccc5C6(c5ccccc5)c5ccccc5)cc4)cc3)n2)cc1]

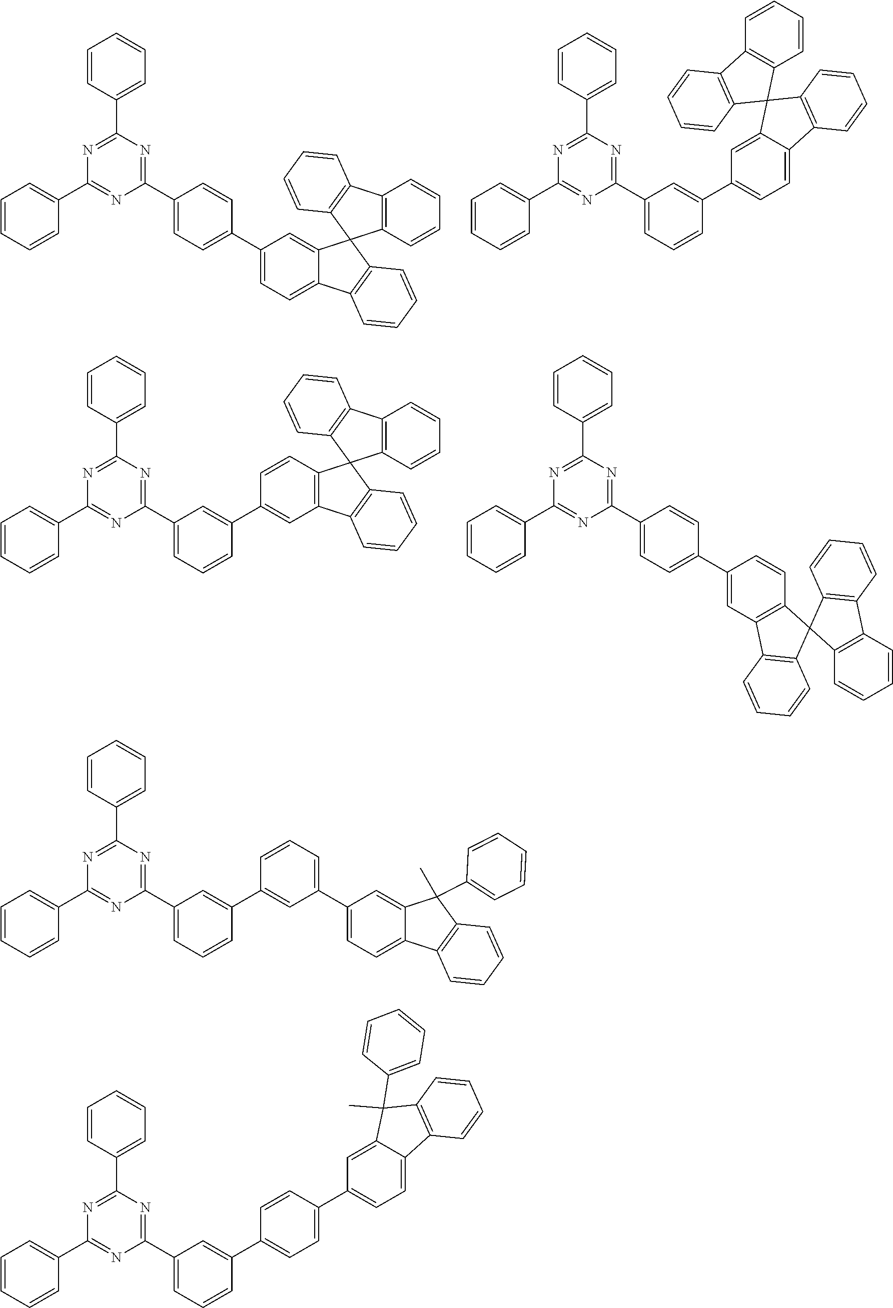

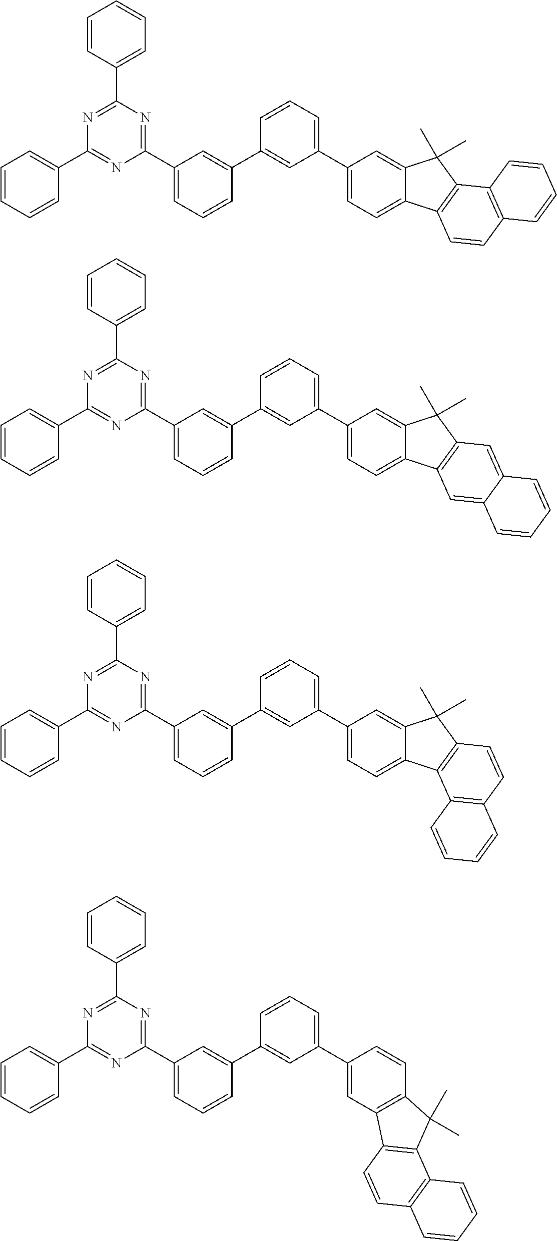 [SMILES: CC1(C)c2cc(-c3cccc(-c4cccc(-c5nc(-c6ccccc6)nc(-c6ccccc6)n5)c4)c3)ccc2-c2ccc3ccccc3c21 ; CC1(C)c2cc(-c3cccc(-c4cccc(-c5nc(-c6ccccc6)nc(-c6ccccc6)n5)c4)c3)ccc2-c2cc3ccccc3cc21 ; CC1(C)c2cc(-c3cccc(-c4cccc(-c5nc(-c6ccccc6)nc(-c6ccccc6)n5)c4)c3)ccc2-c2c1ccc1ccccc21 ; CC1(C)c2ccc(-c3cccc(-c4cccc(-c5nc(-c6ccccc6)nc(-c6ccccc6)n5)c4)c3)cc2-c2ccc3ccccc3c21]

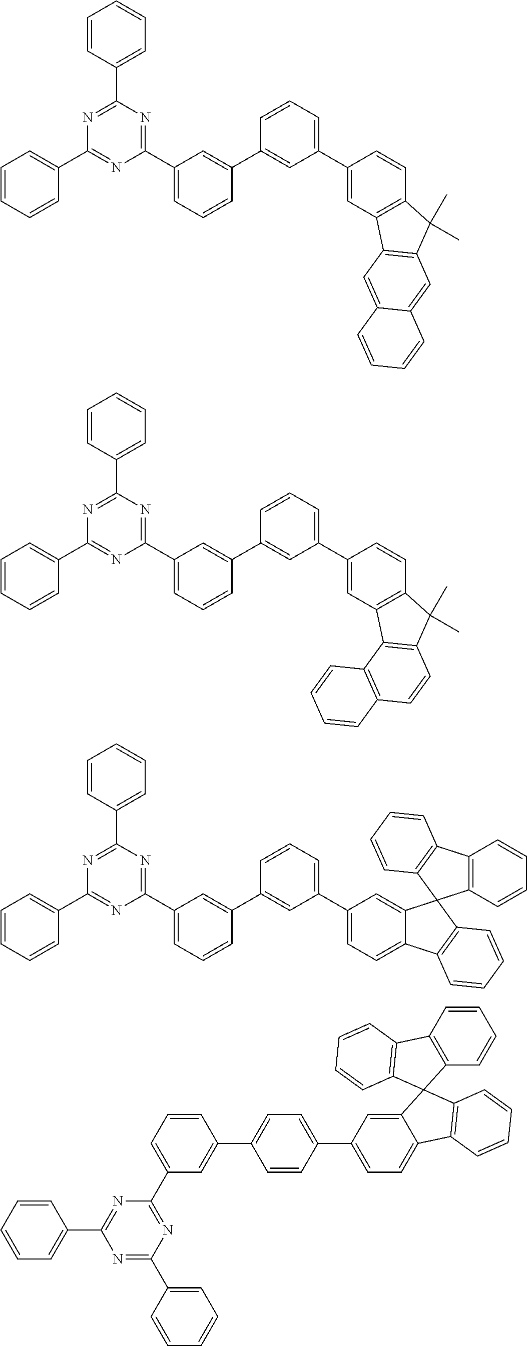

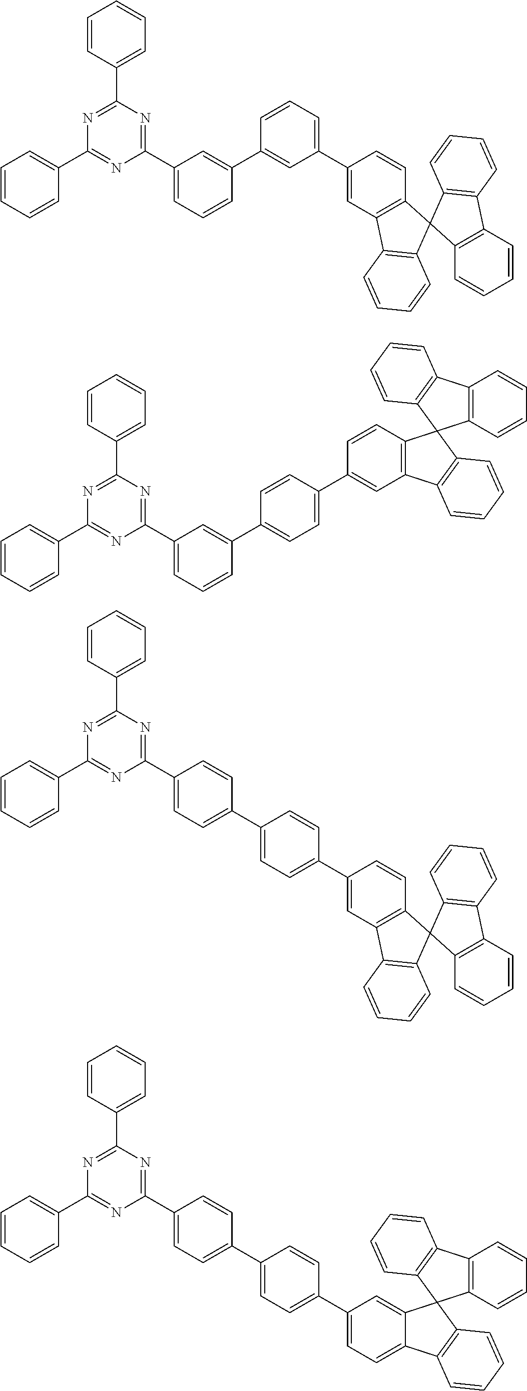

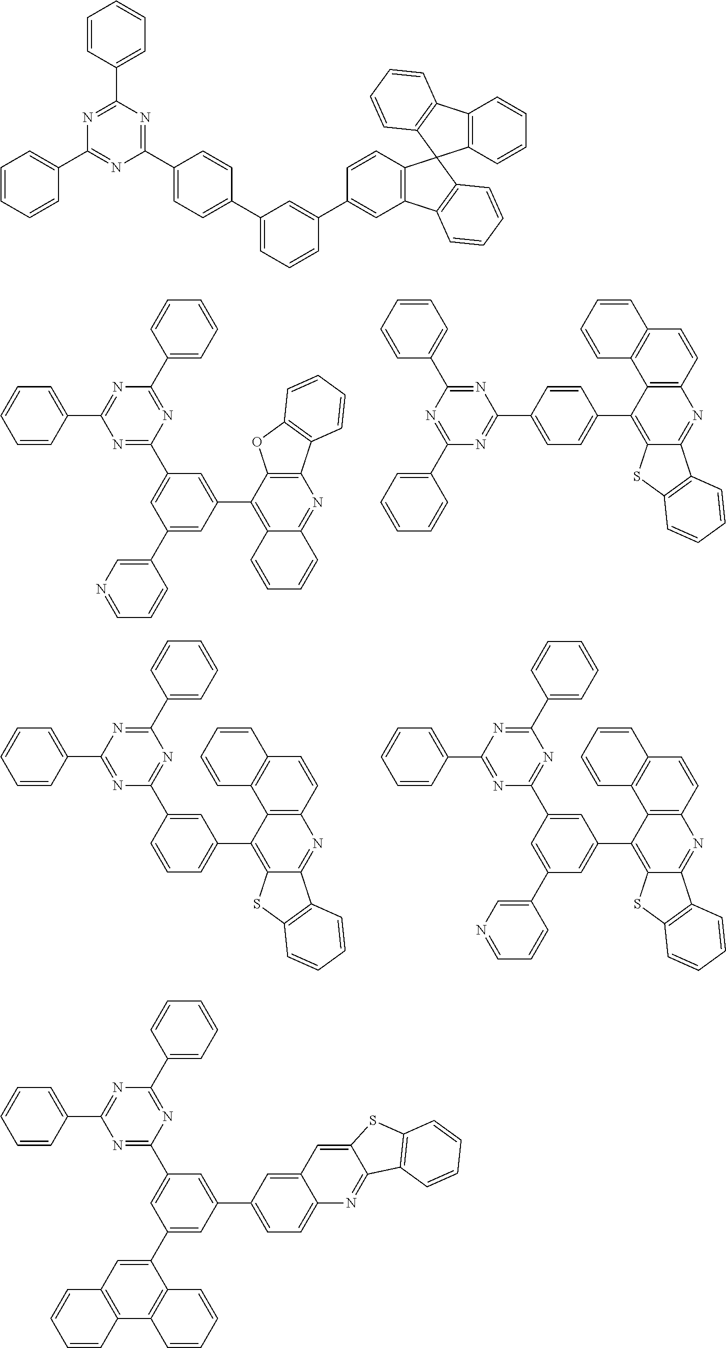

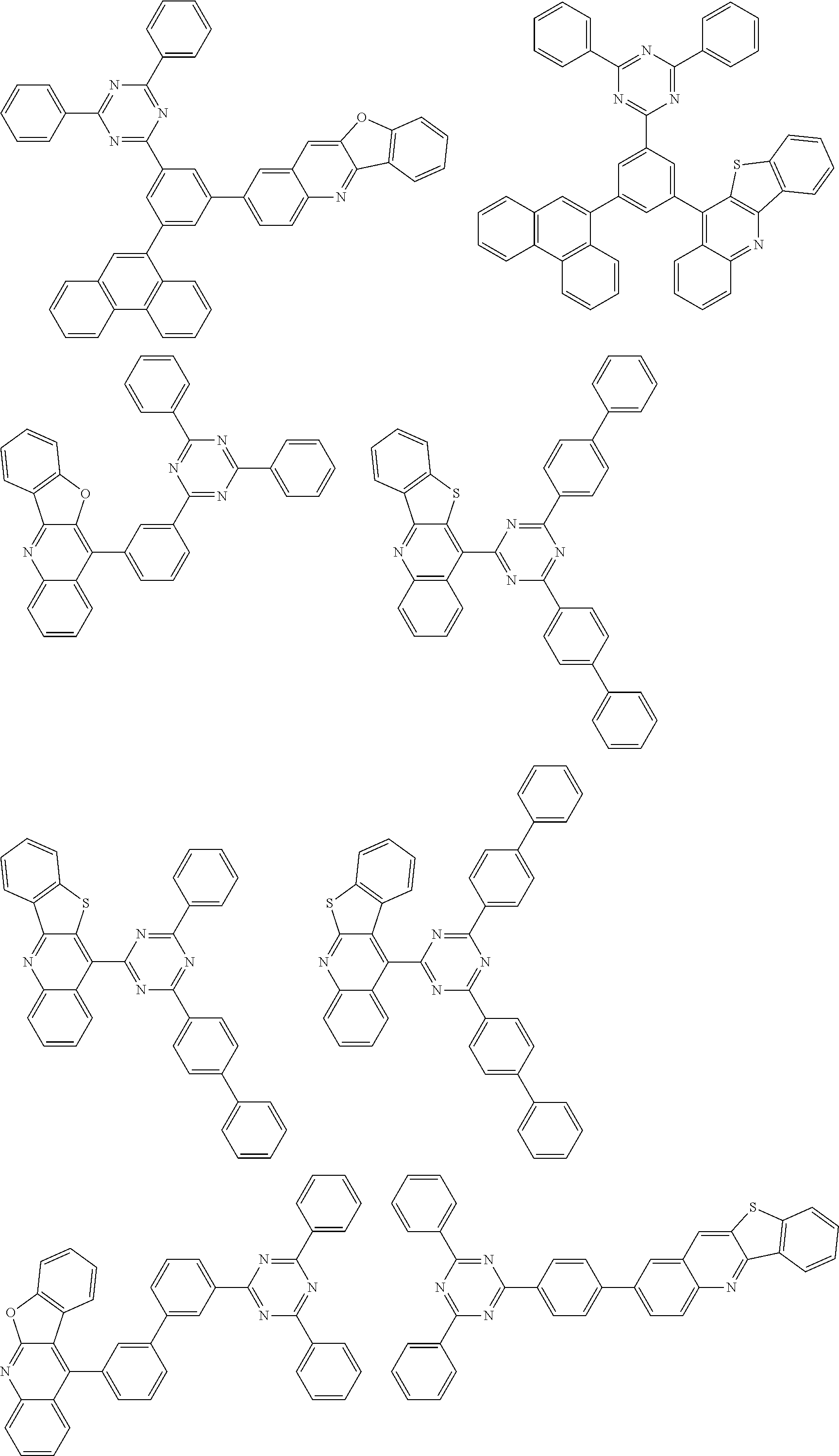

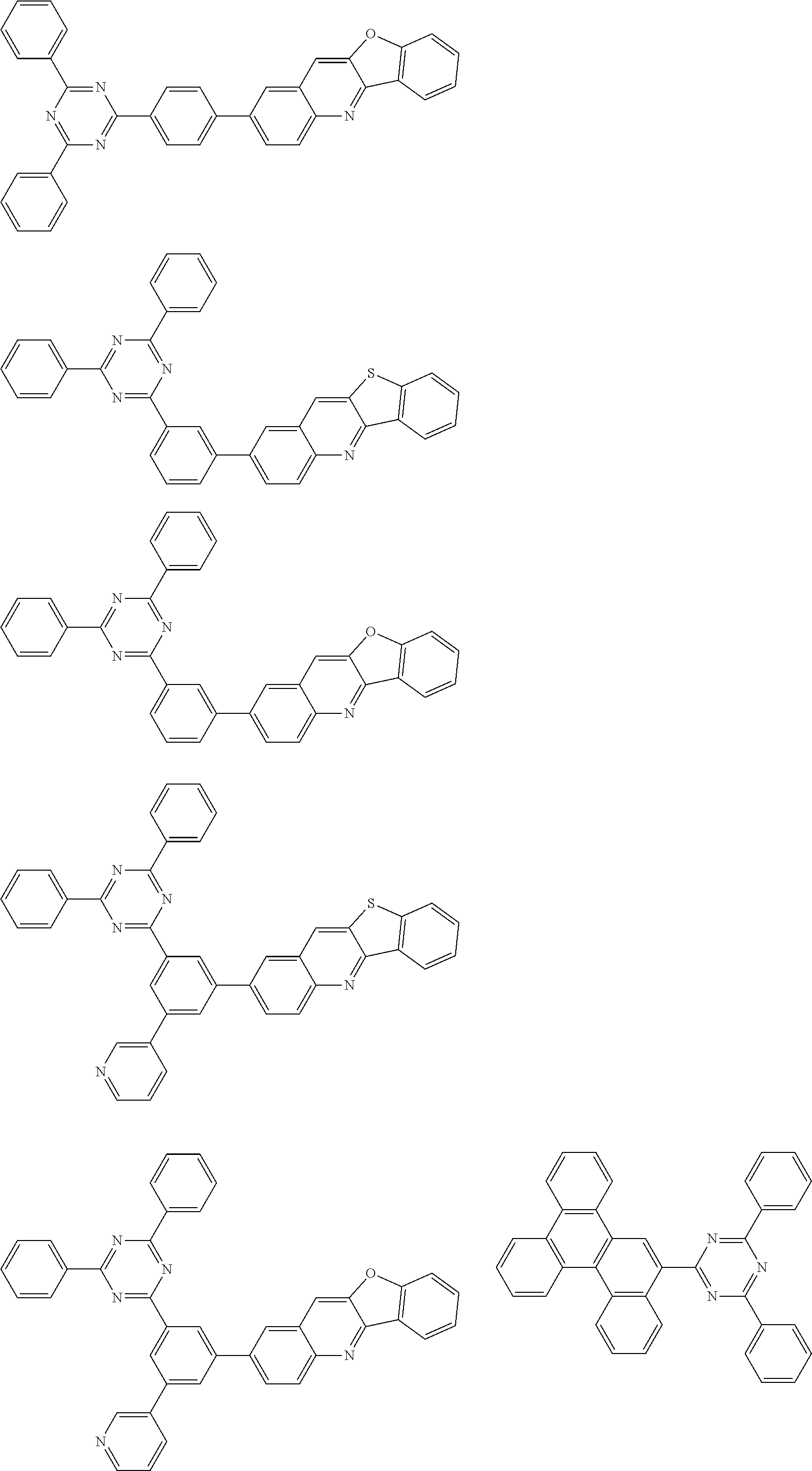

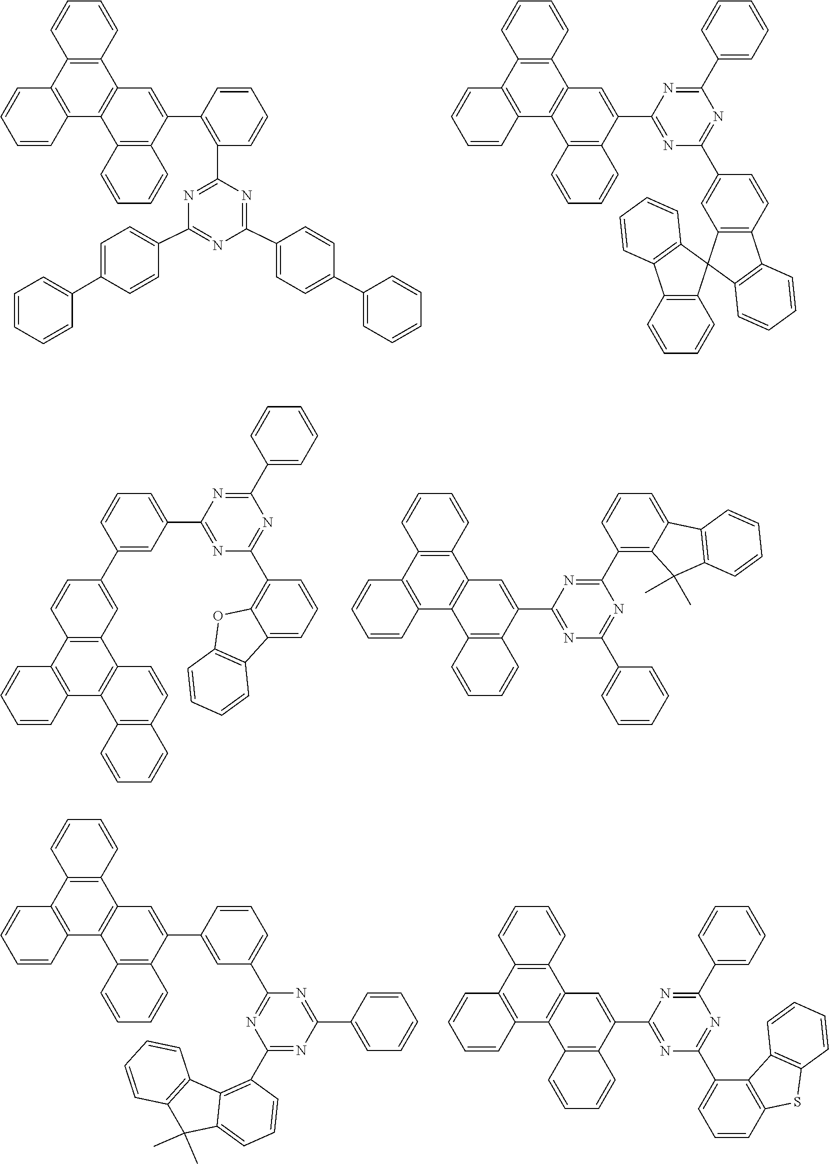

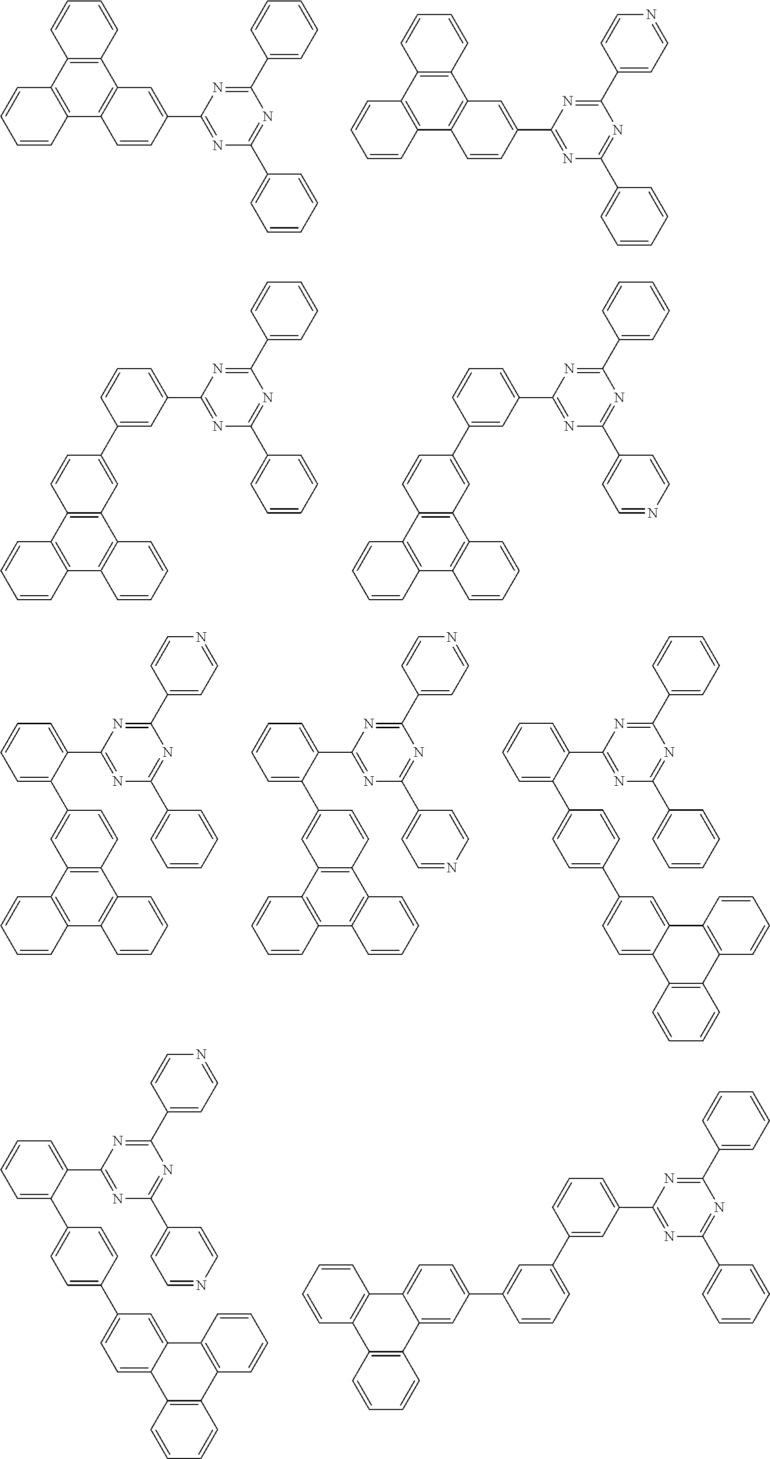

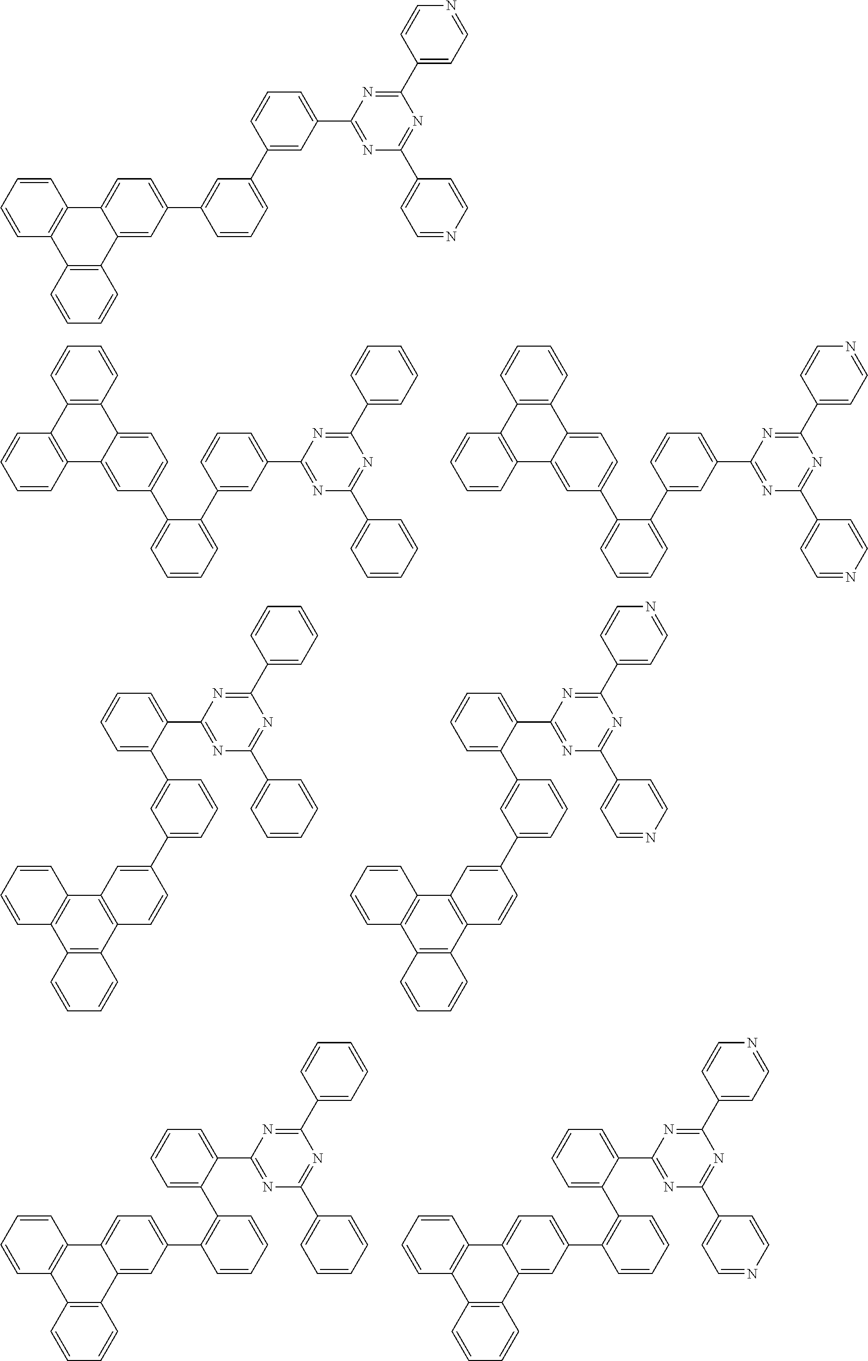

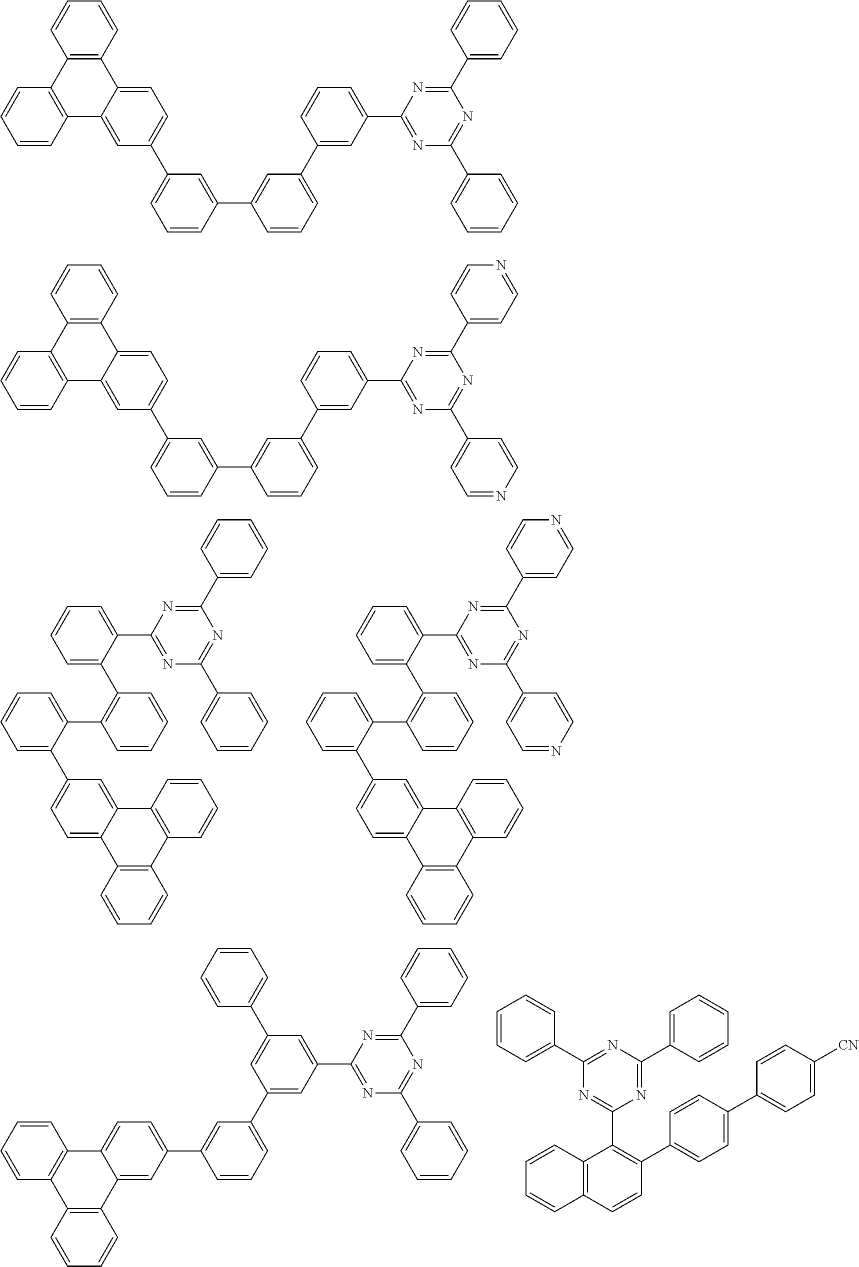

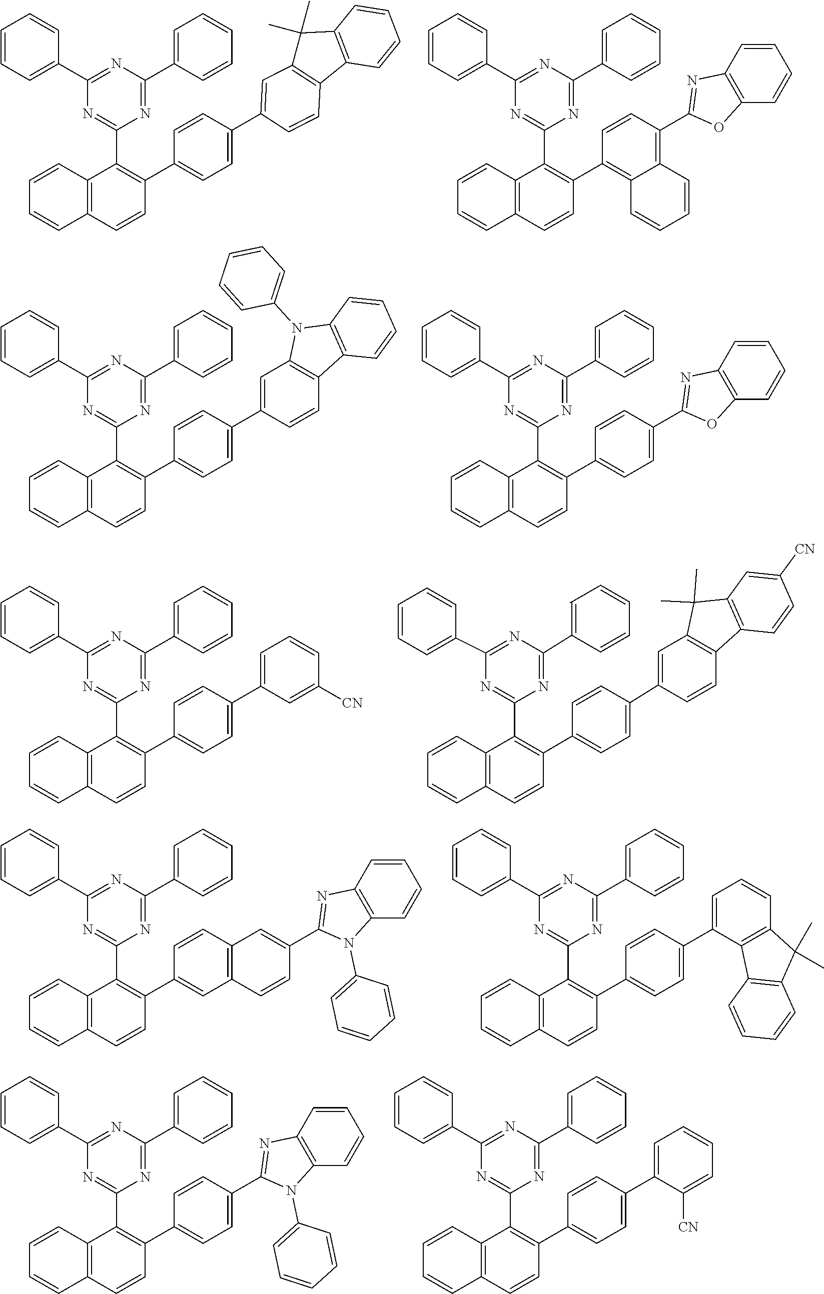

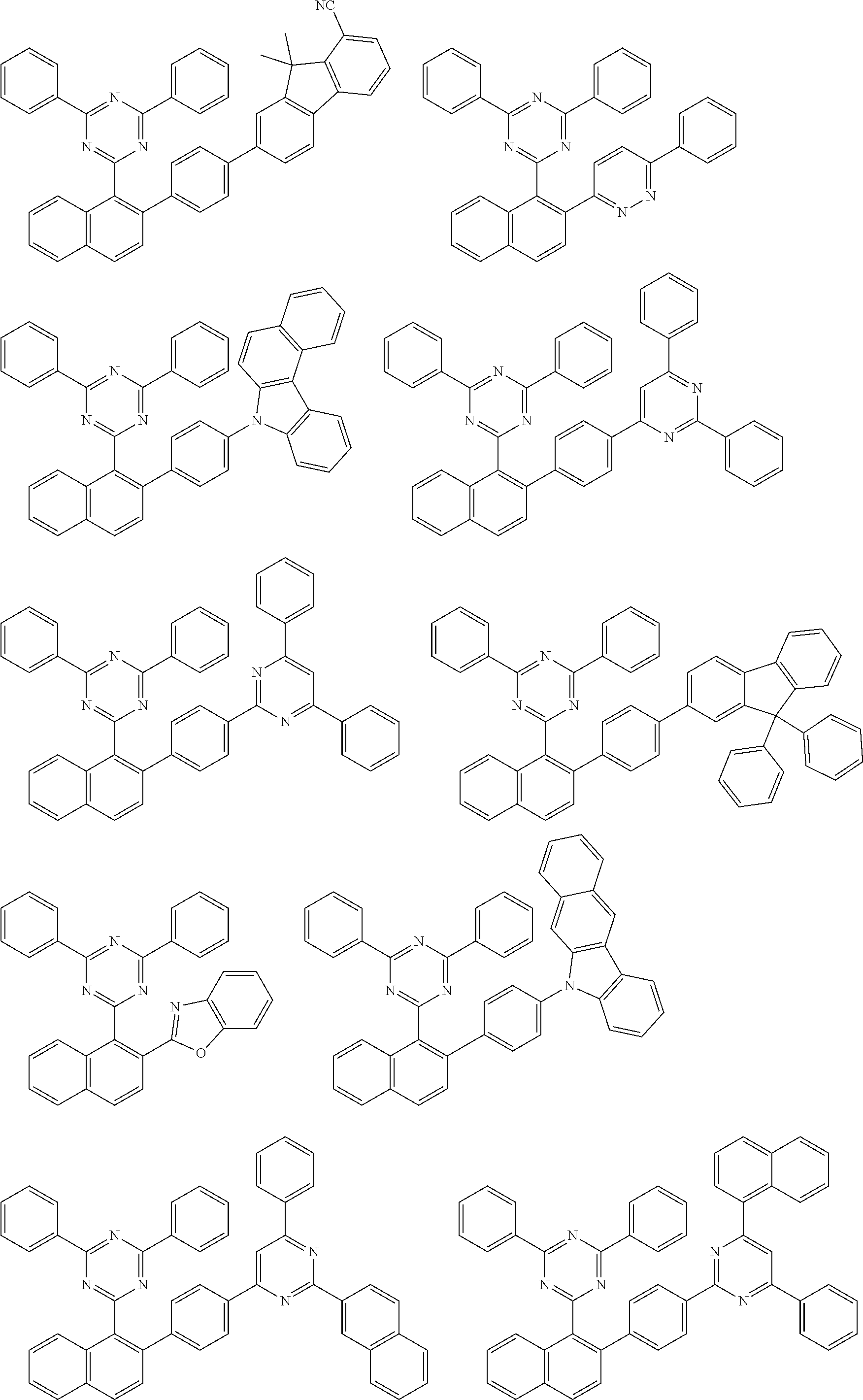

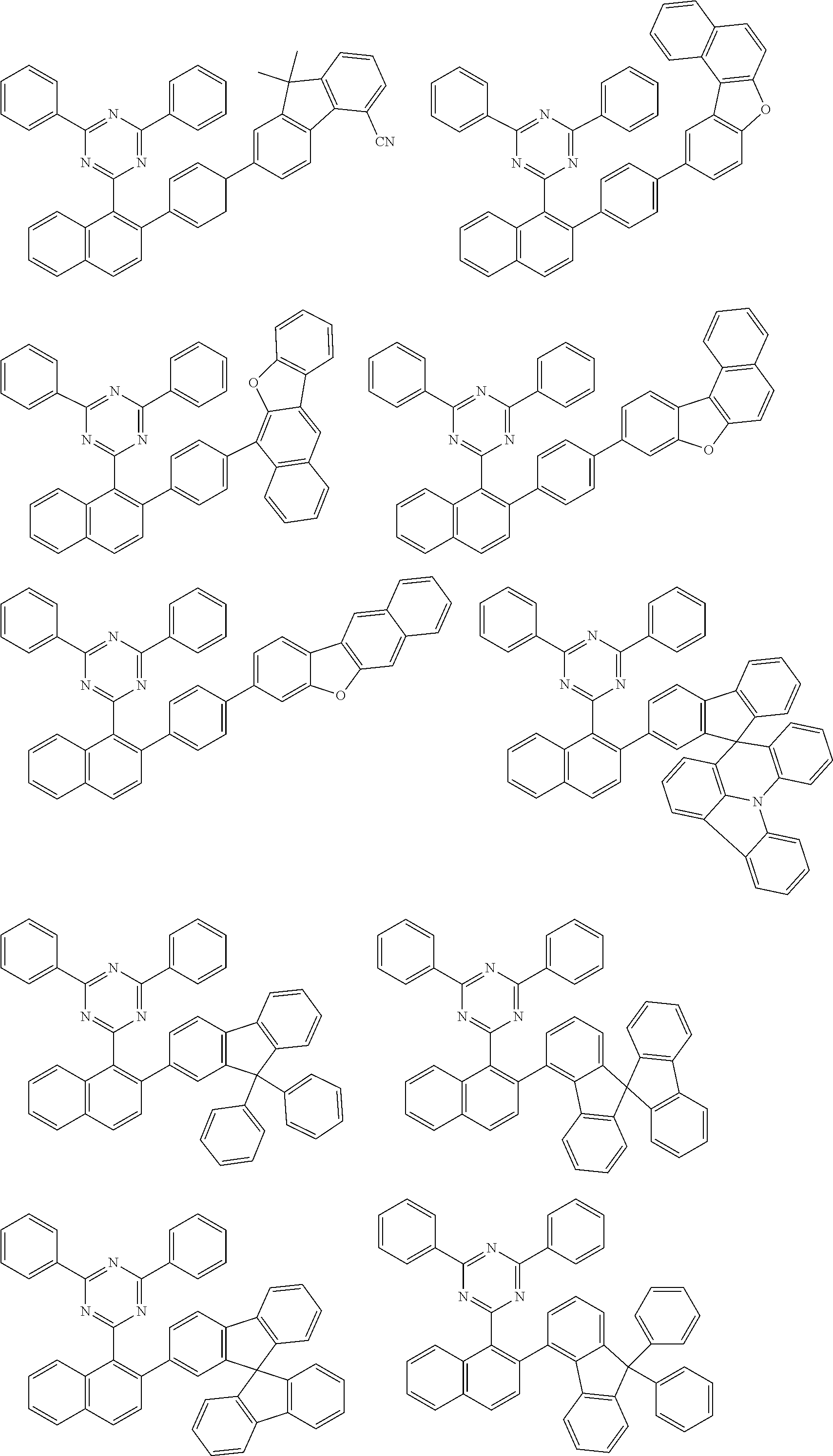

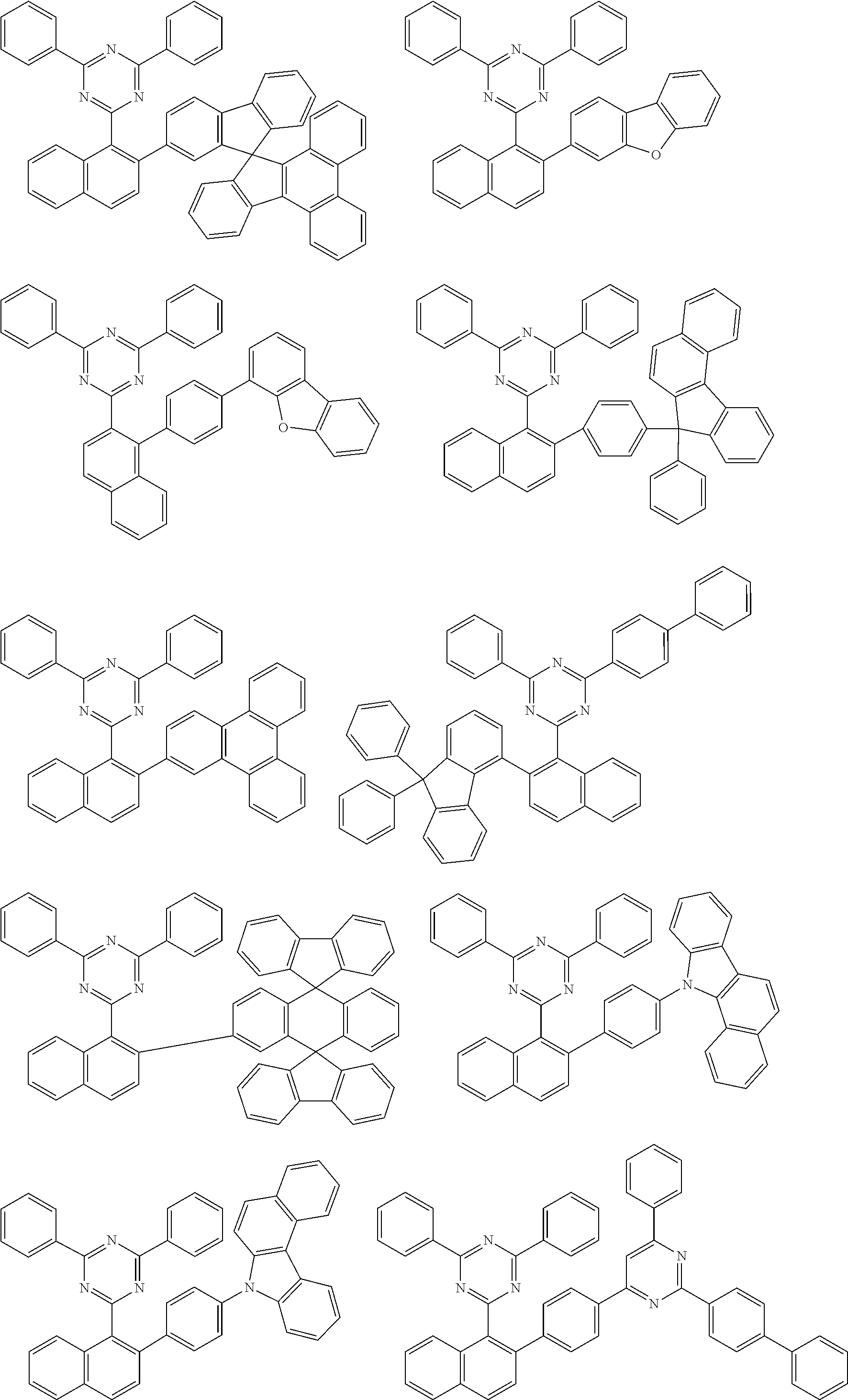

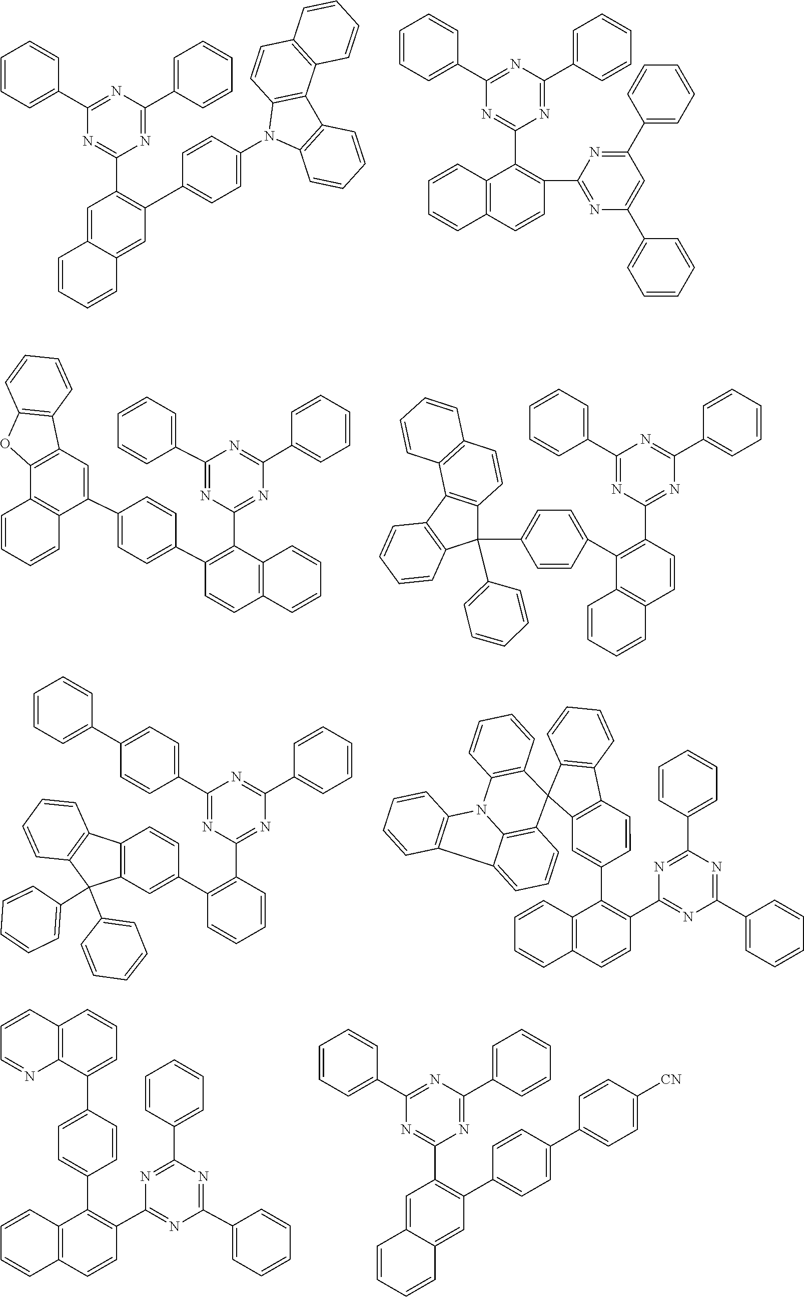

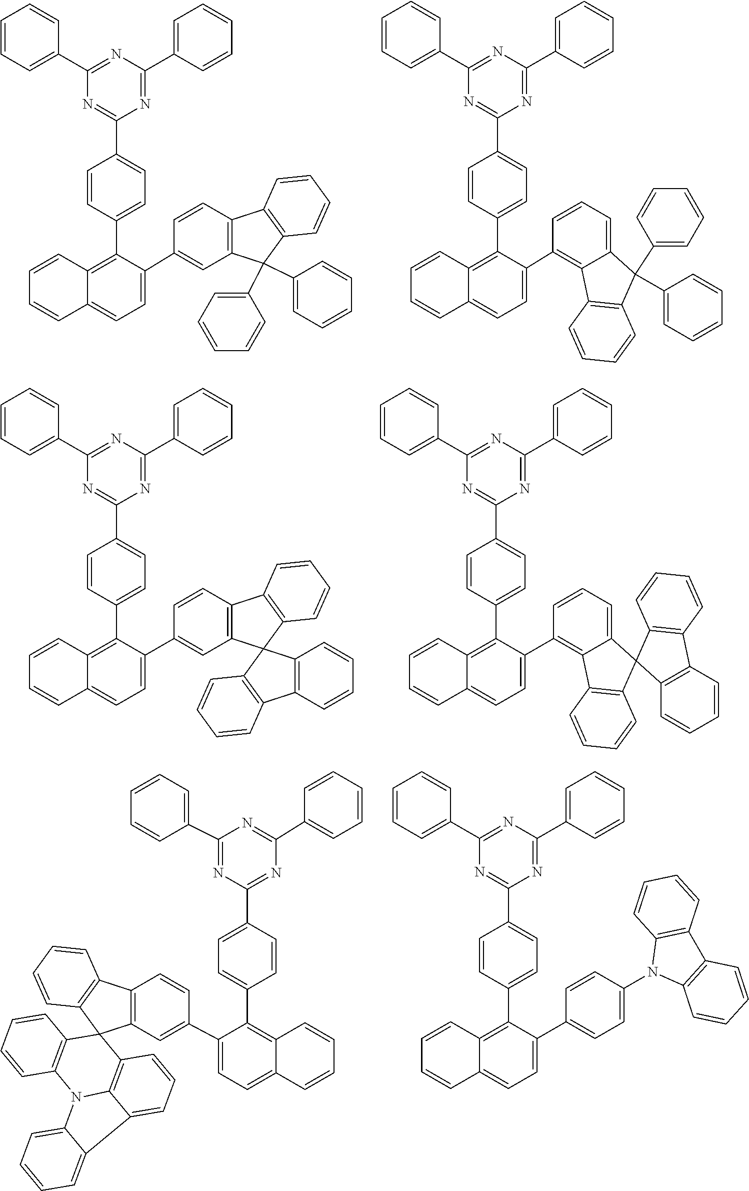

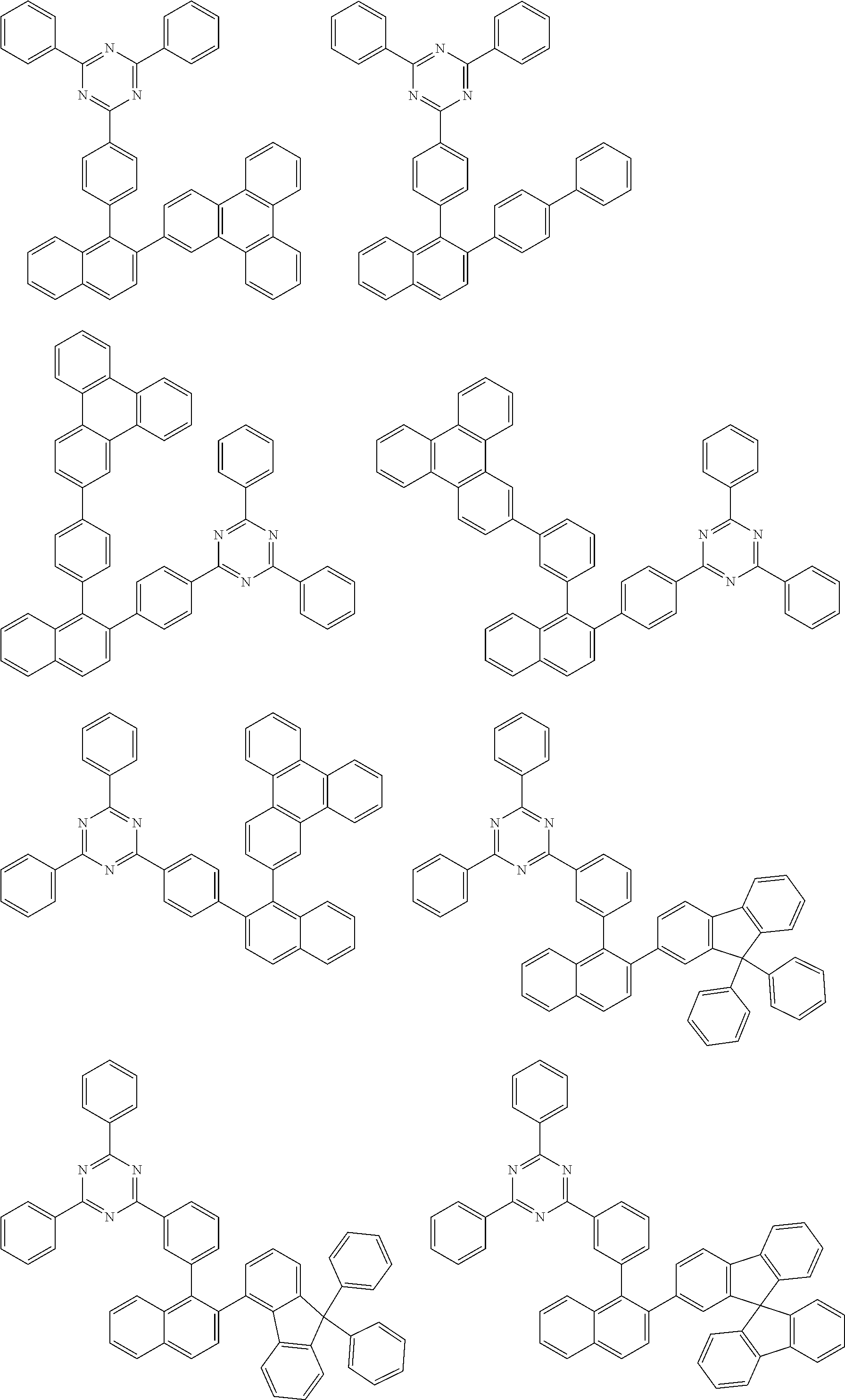

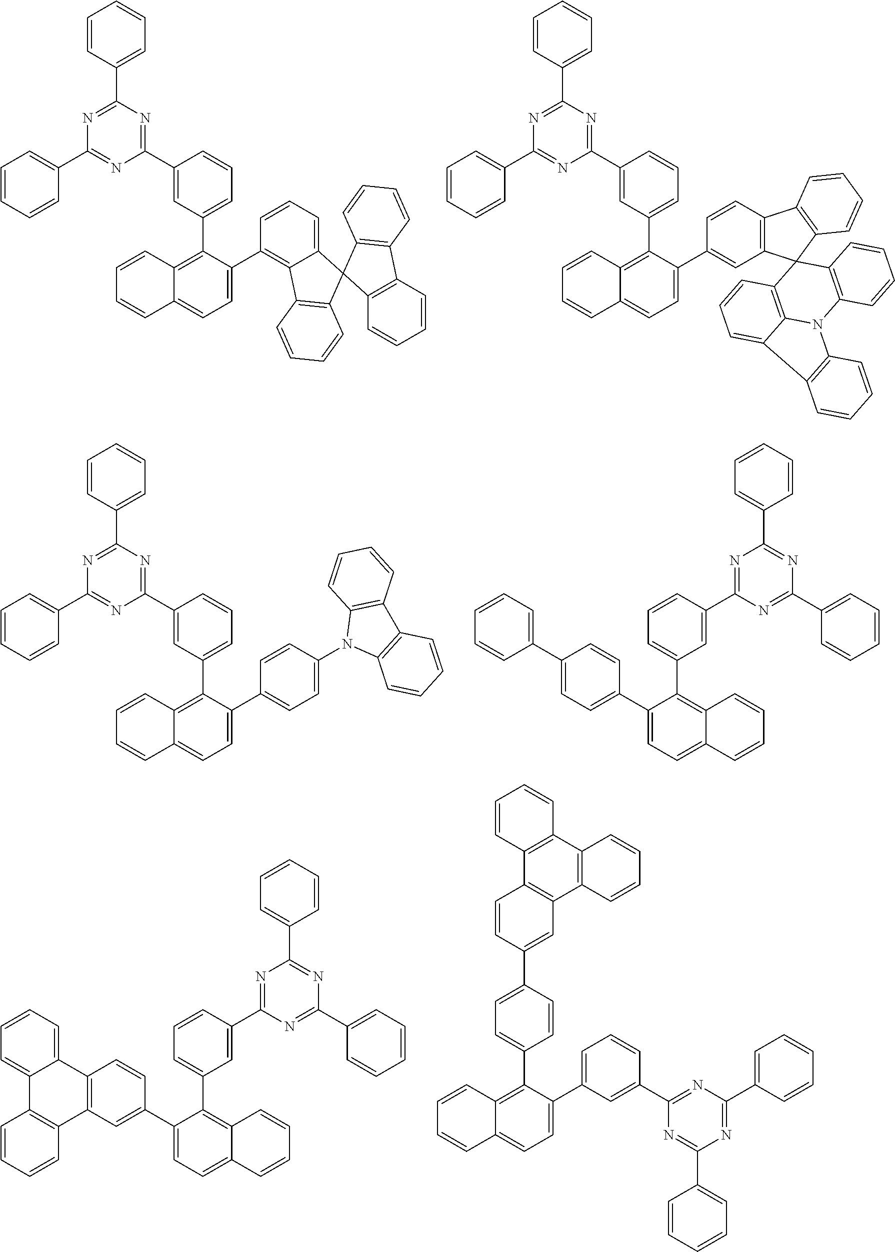

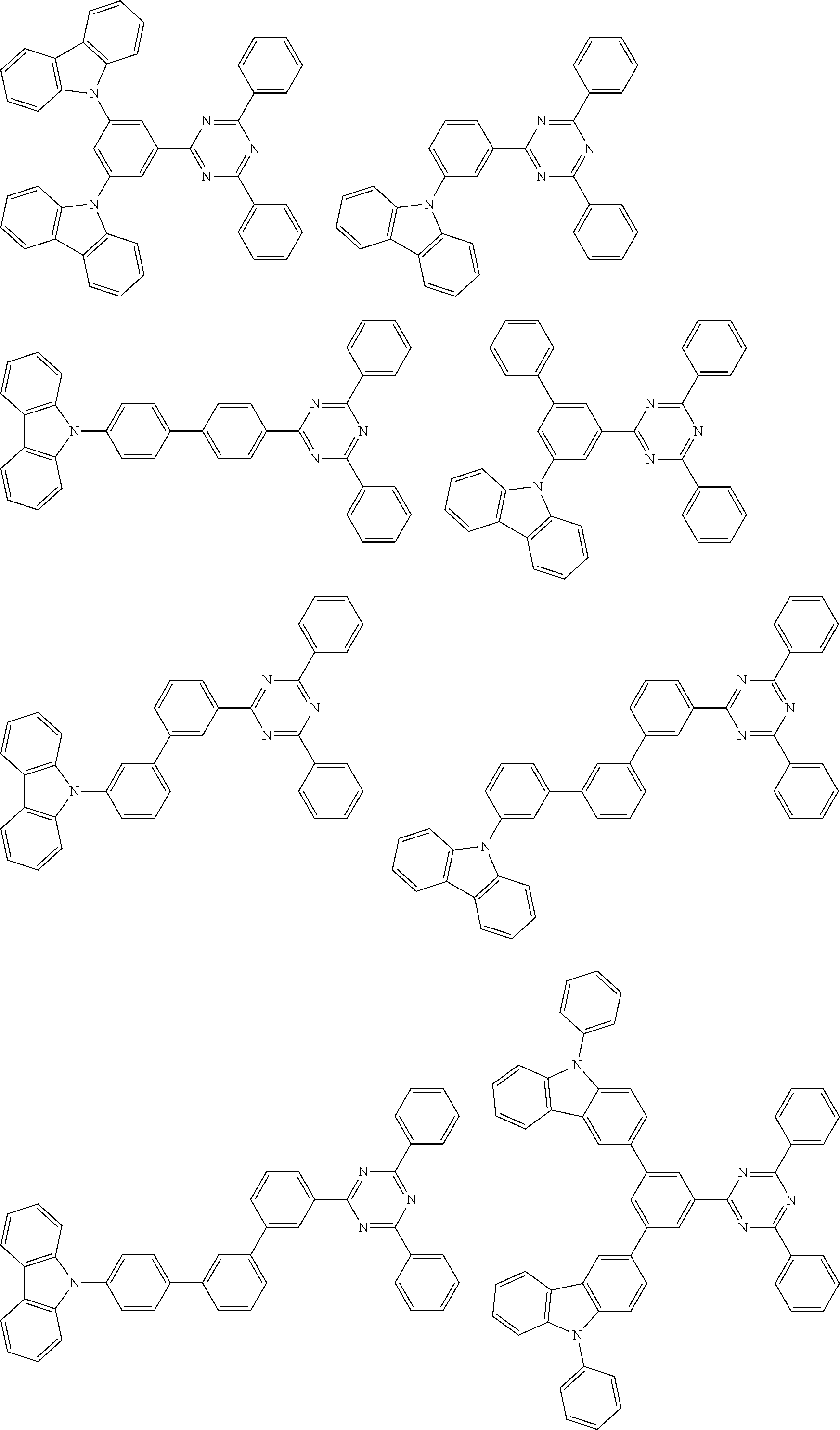

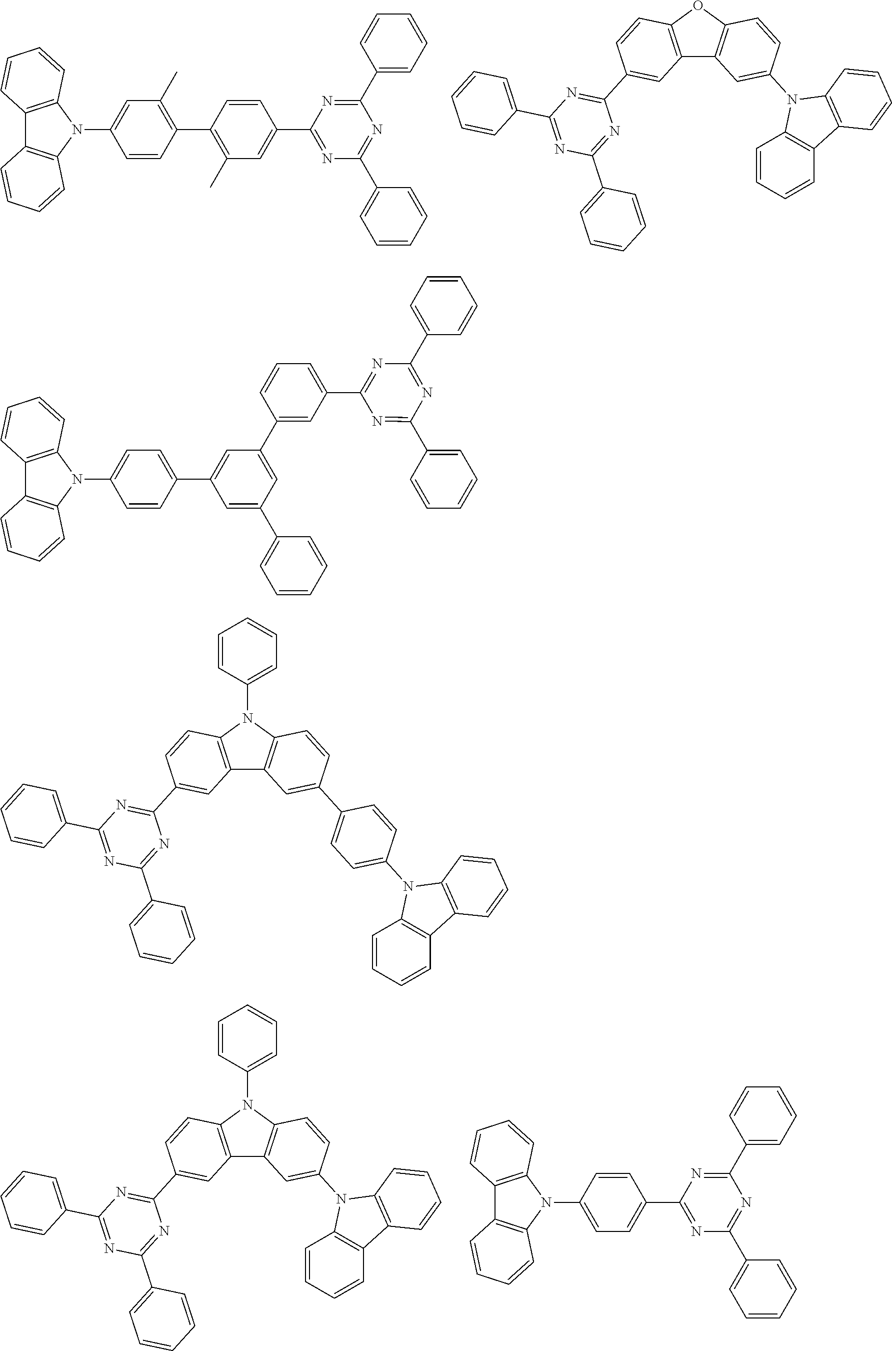

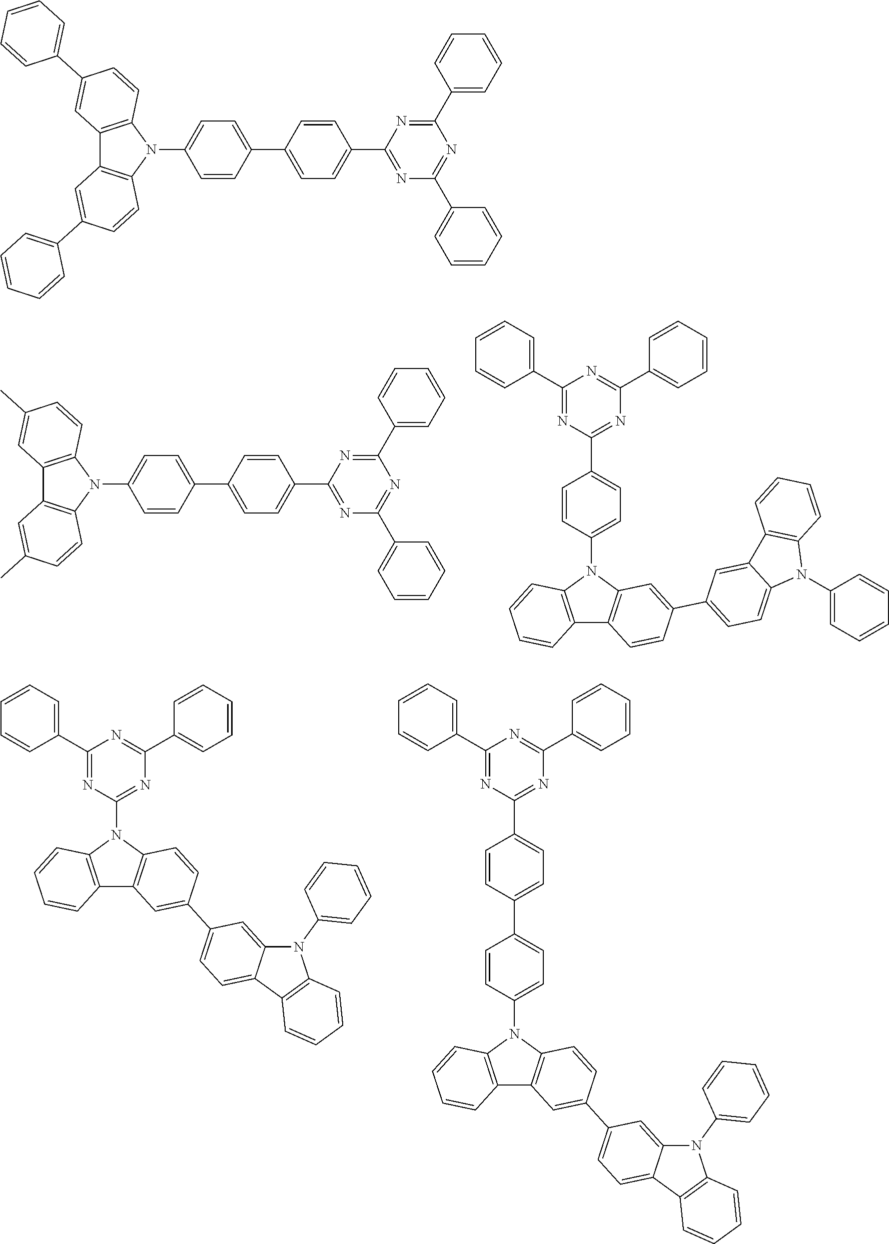

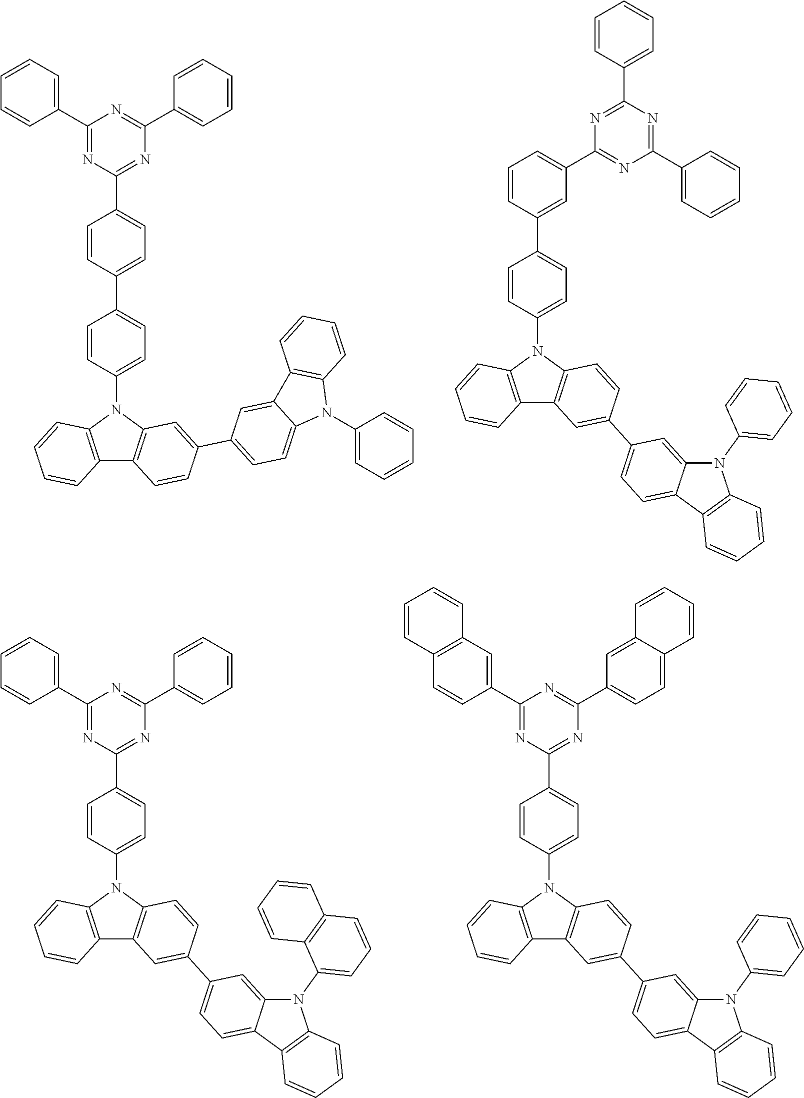 [SMILES: c1ccc(-c2nc(-c3ccccc3)nc(-c3ccc(-c4ccc(-n5c6ccccc6c6ccc(-c7ccc8c(c7)c7ccccc7n8-c7ccccc7)cc65)cc4)cc3)n2)cc1 ; c1ccc(-c2nc(-c3ccccc3)nc(-c3cccc(-c4ccc(-n5c6ccccc6c6cc(-c7ccc8c9ccccc9n(-c9ccccc9)c8c7)ccc65)cc4)c3)n2)cc1 ; c1ccc(-c2nc(-c3ccccc3)nc(-c3ccc(-n4c5ccccc5c5cc(-c6ccc7c8ccccc8n(-c8cccc9ccccc89)c7c6)ccc54)cc3)n2)cc1 ; c1ccc(-n2c3ccccc3c3ccc(-c4ccc5c(c4)c4ccccc4n5-c4ccc(-c5nc(-c6ccc7ccccc7c6)nc(-c6ccc7ccccc7c6)n5)cc4)cc32)cc1]

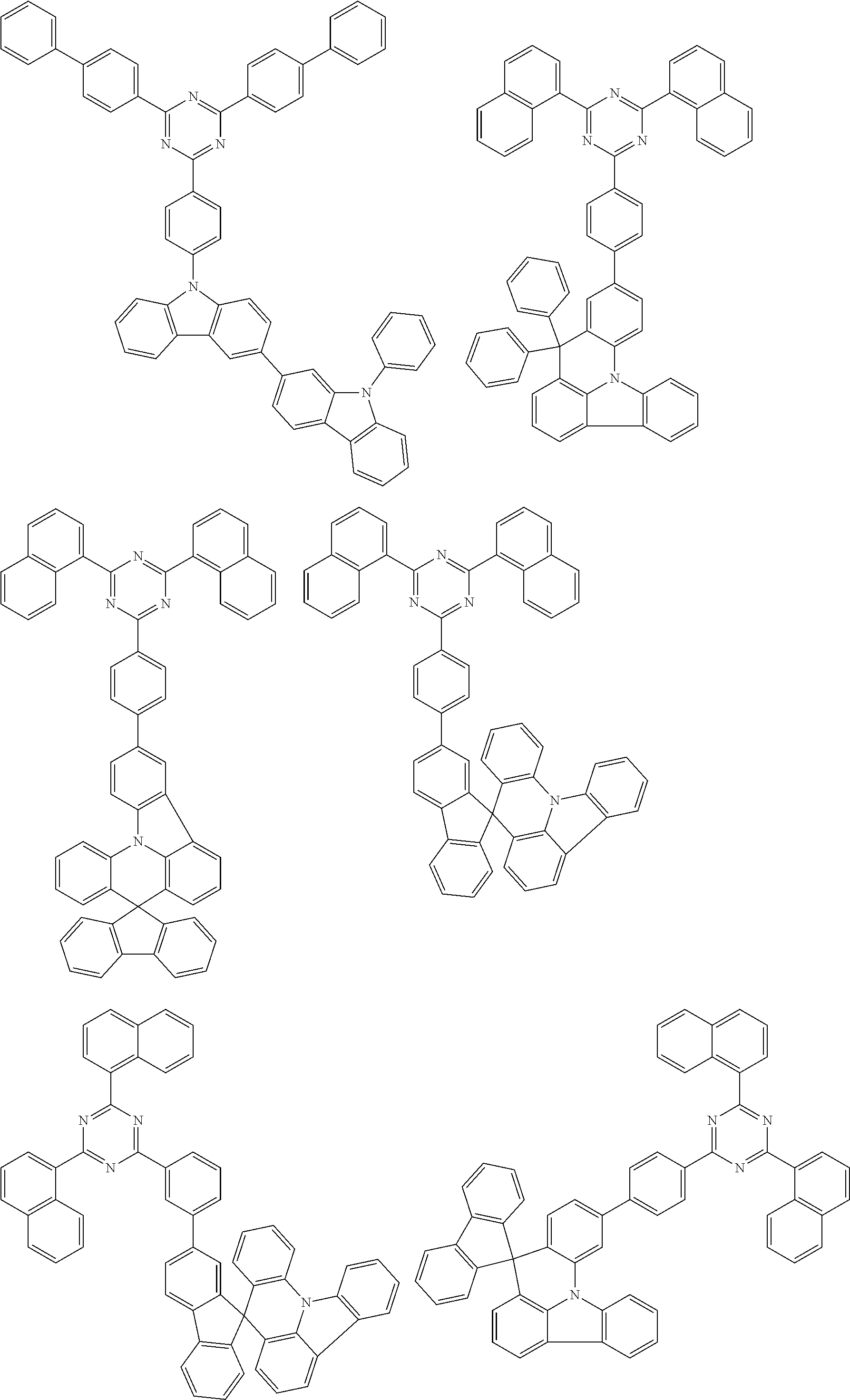

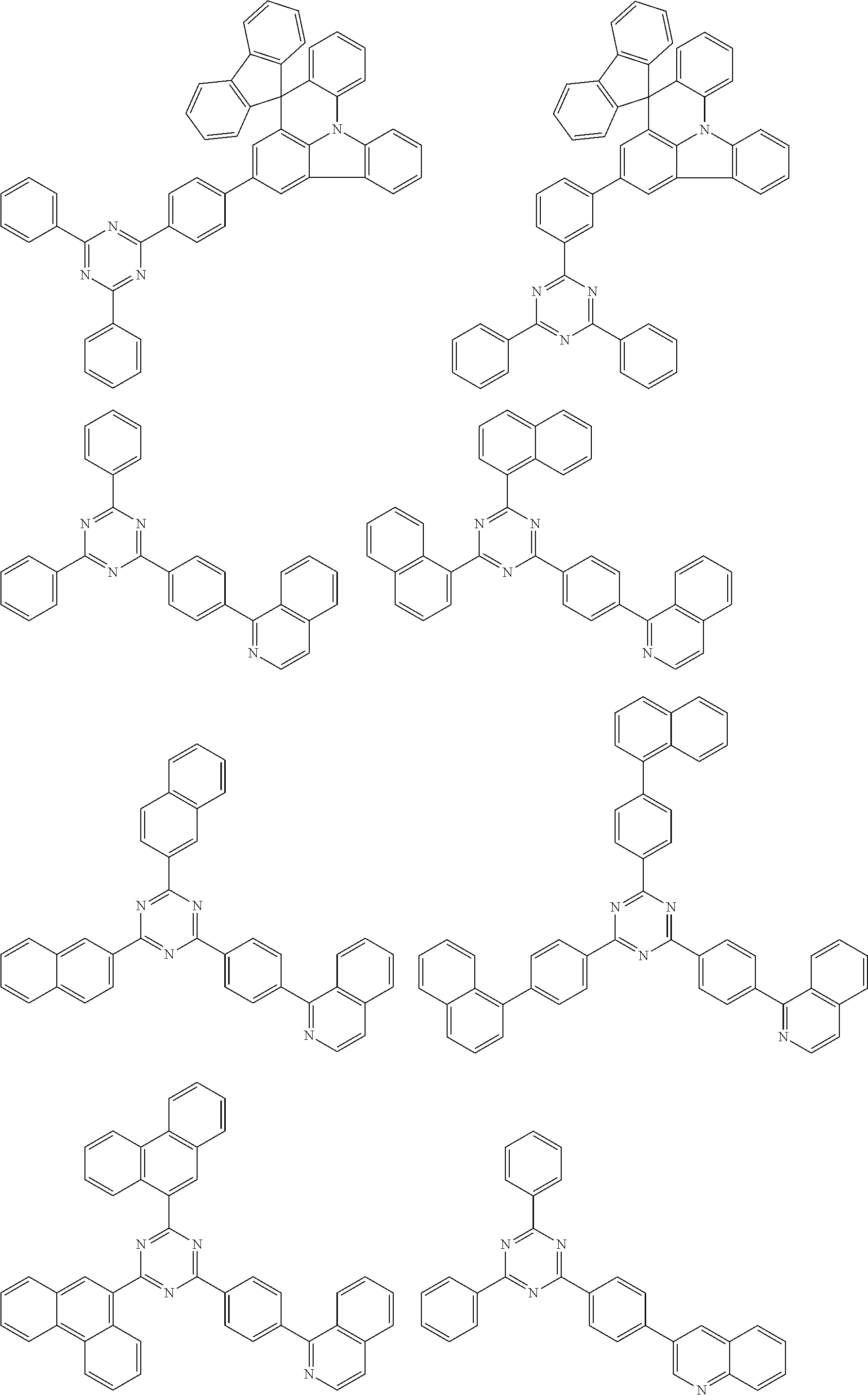

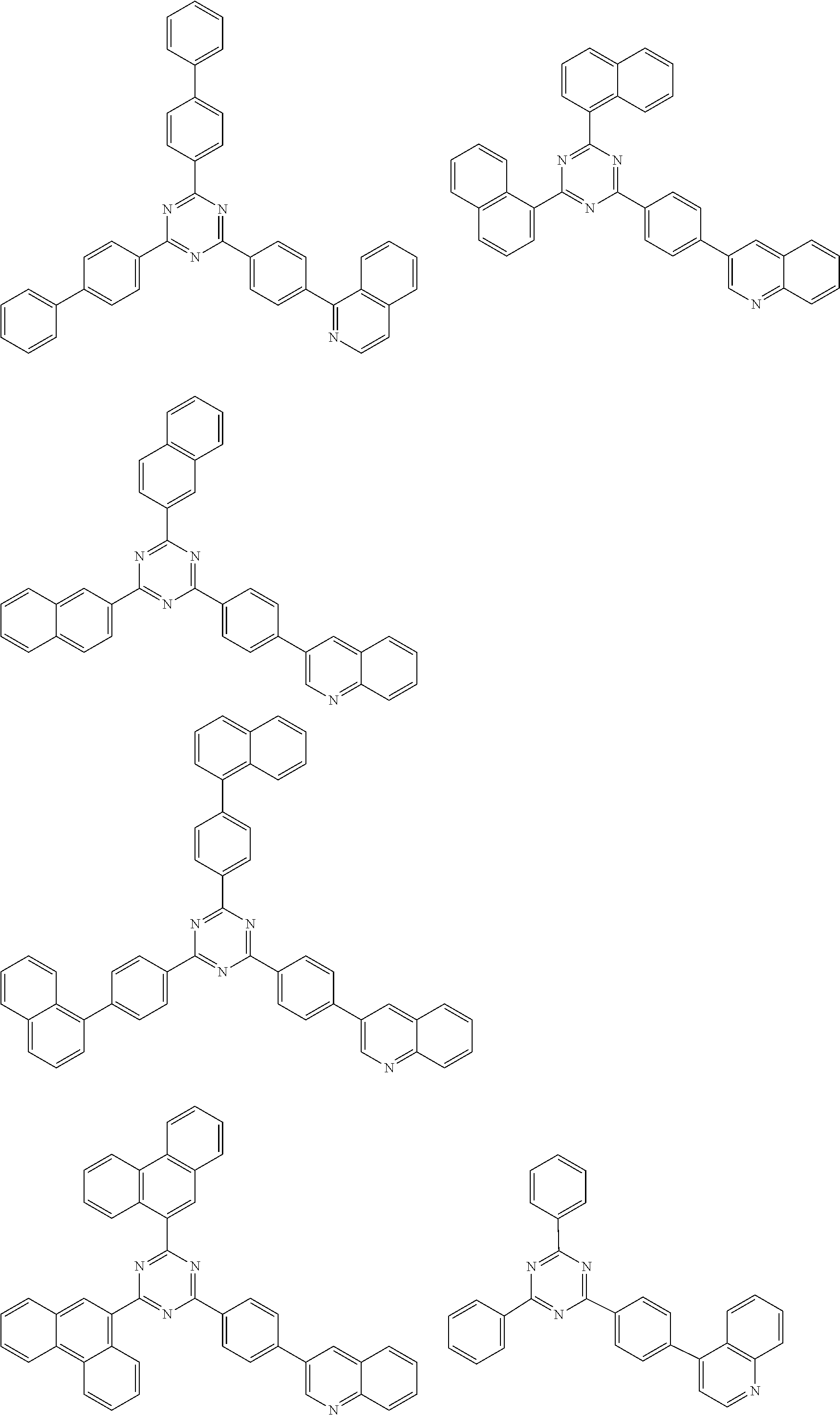

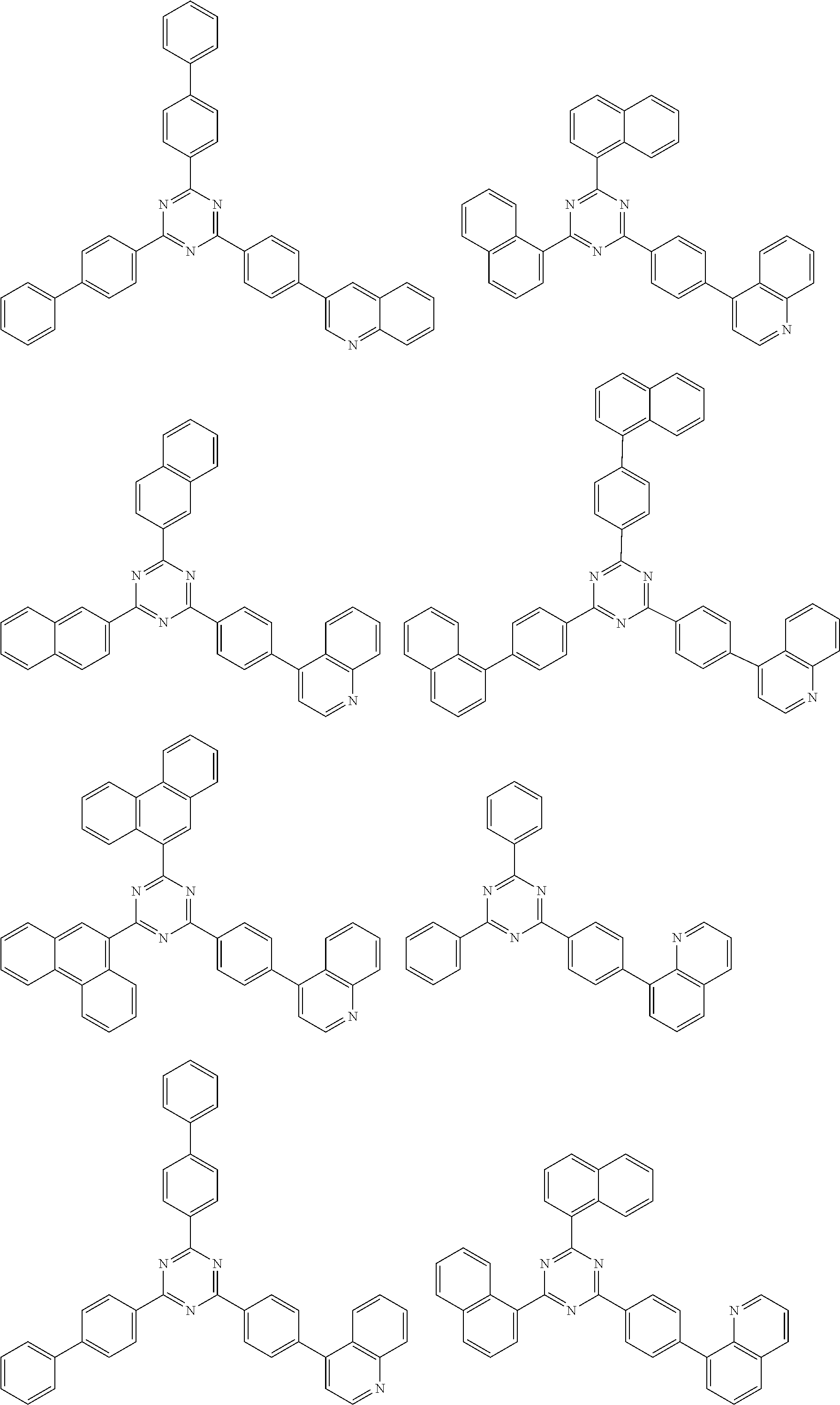

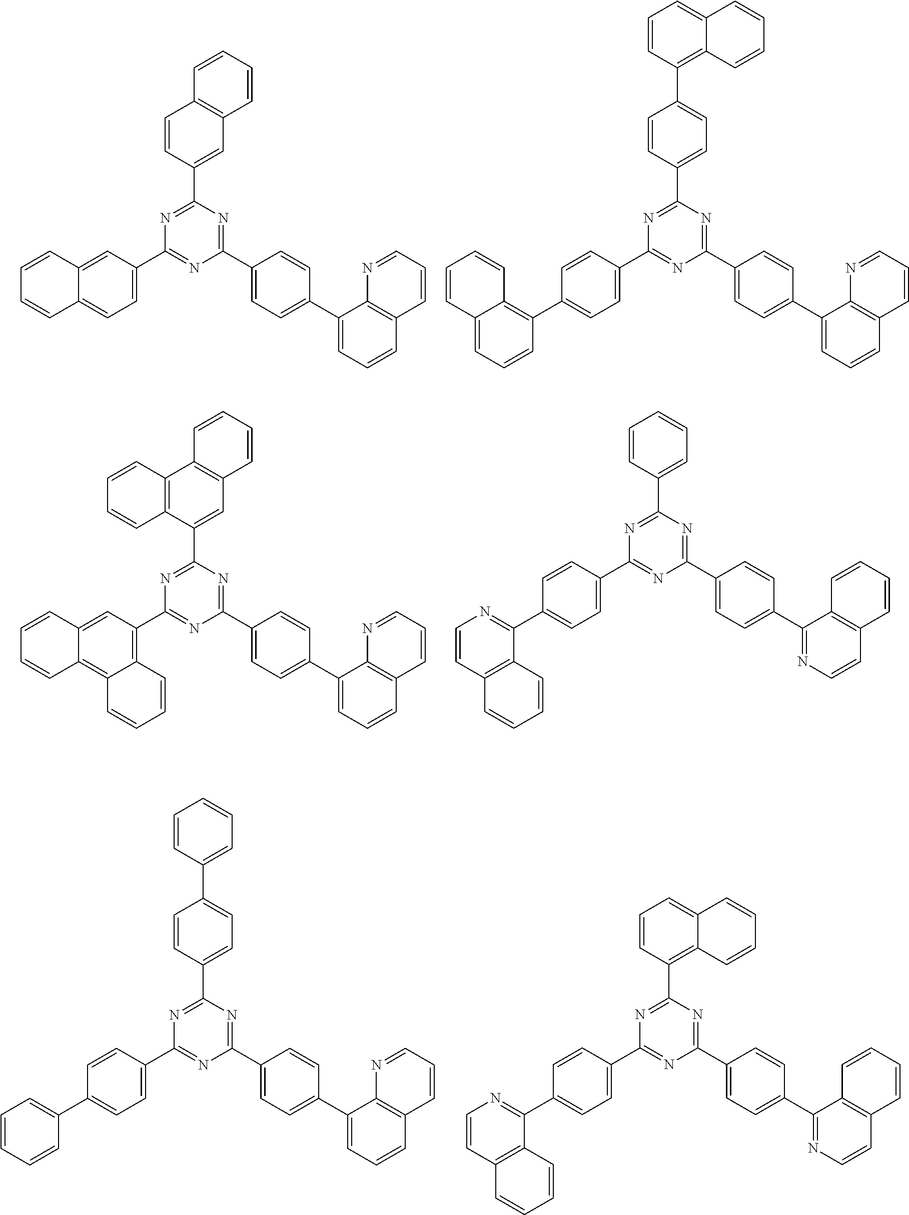

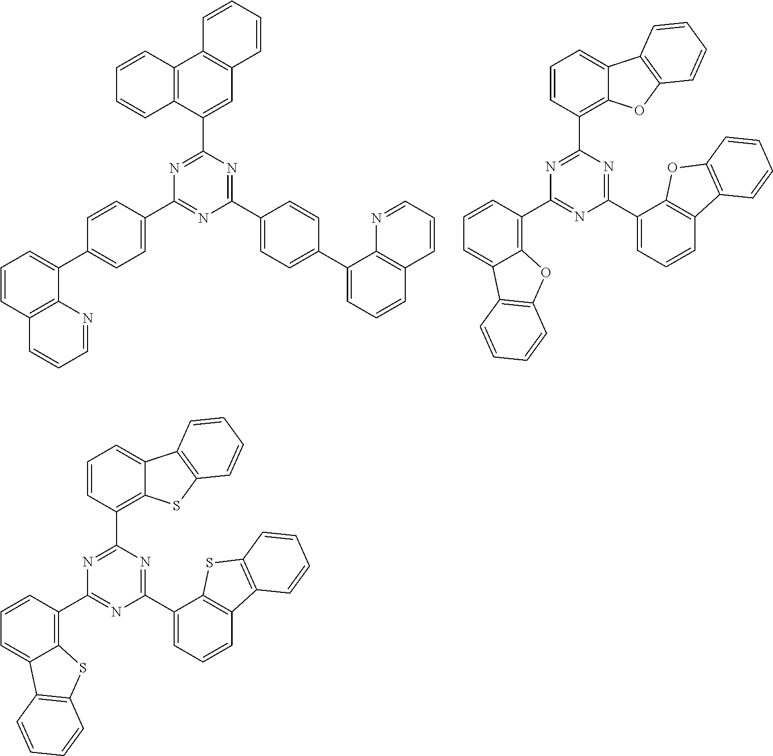

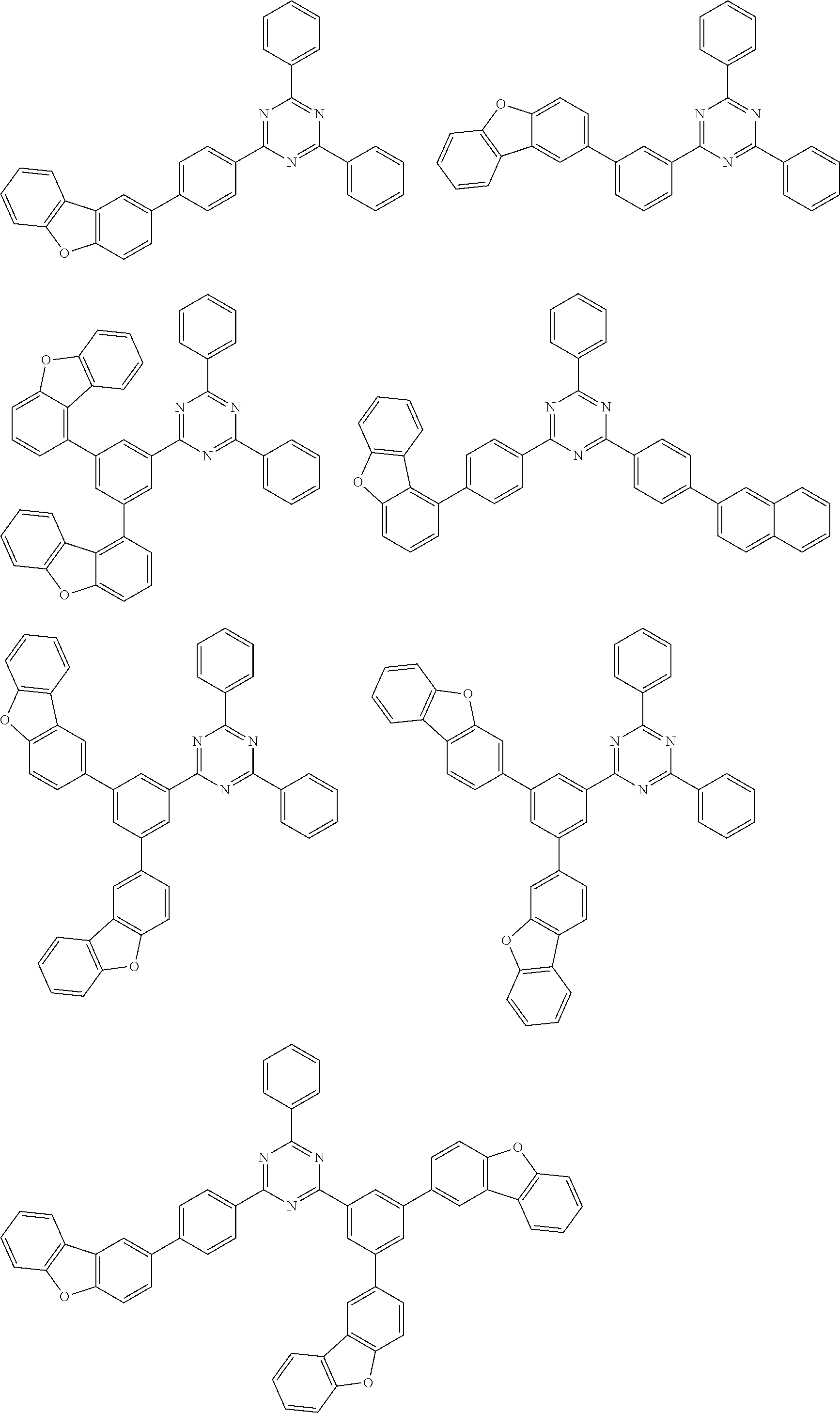

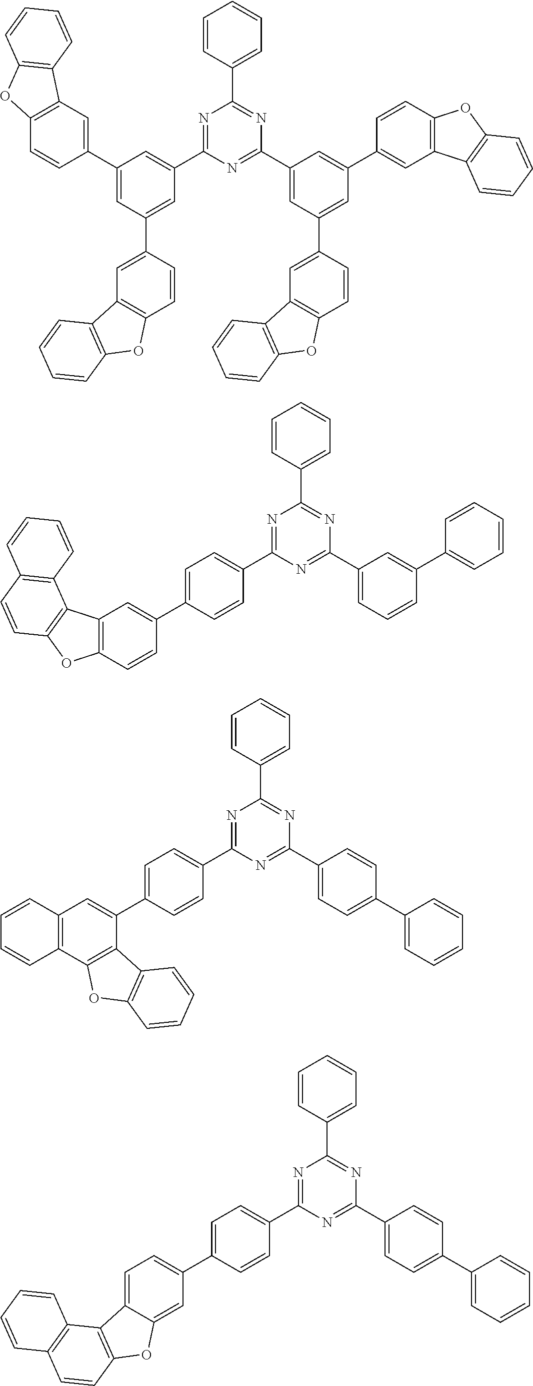

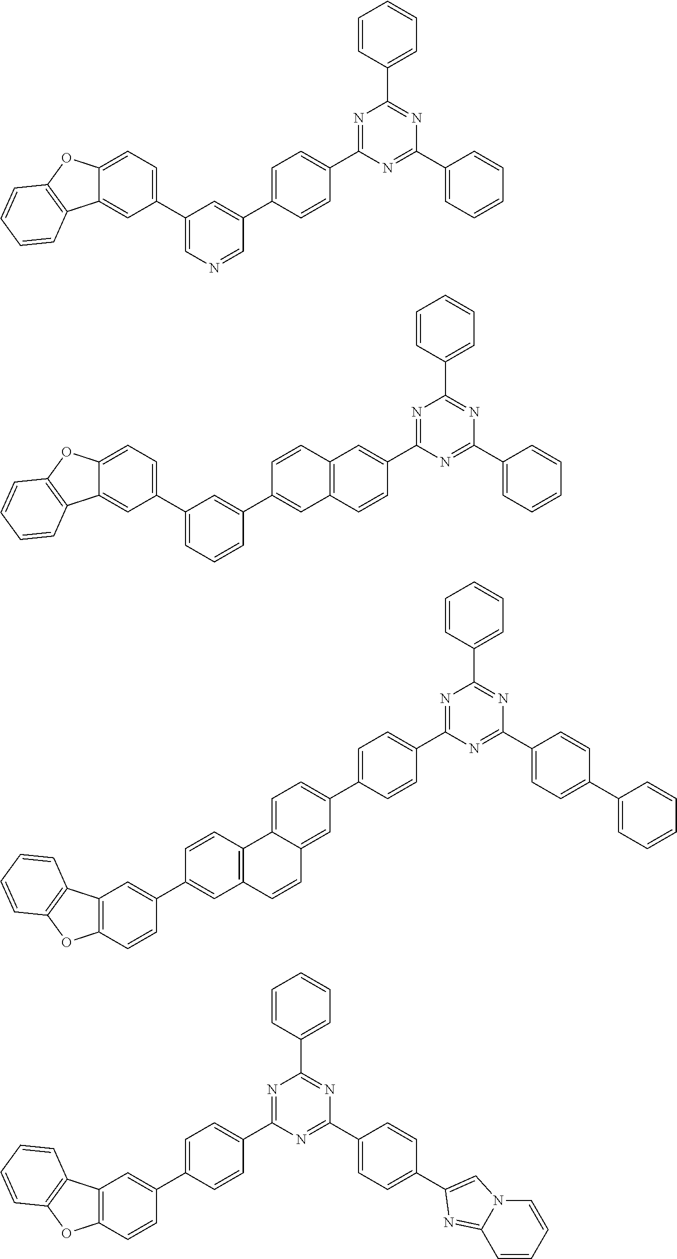 [SMILES: c1ccc(-c2nc(-c3ccccc3)nc(-c3ccc(-c4cncc(-c5ccc6oc7ccccc7c6c5)c4)cc3)n2)cc1 ; c1ccc(-c2nc(-c3ccccc3)nc(-c3ccc4cc(-c5cccc(-c6ccc7oc8ccccc8c7c6)c5)ccc4c3)n2)cc1 ; c1ccc(-c2ccc(-c3nc(-c4ccccc4)nc(-c4ccc(-c5ccc6c(ccc7cc(-c8ccc9oc%10ccccc%10c9c8)ccc76)c5)cc4)n3)cc2)cc1 ; c1ccc(-c2nc(-c3ccc(-c4ccc5oc6ccccc6c5c4)cc3)nc(-c3ccc(-c4cn5ccccc5n4)cc3)n2)cc1]

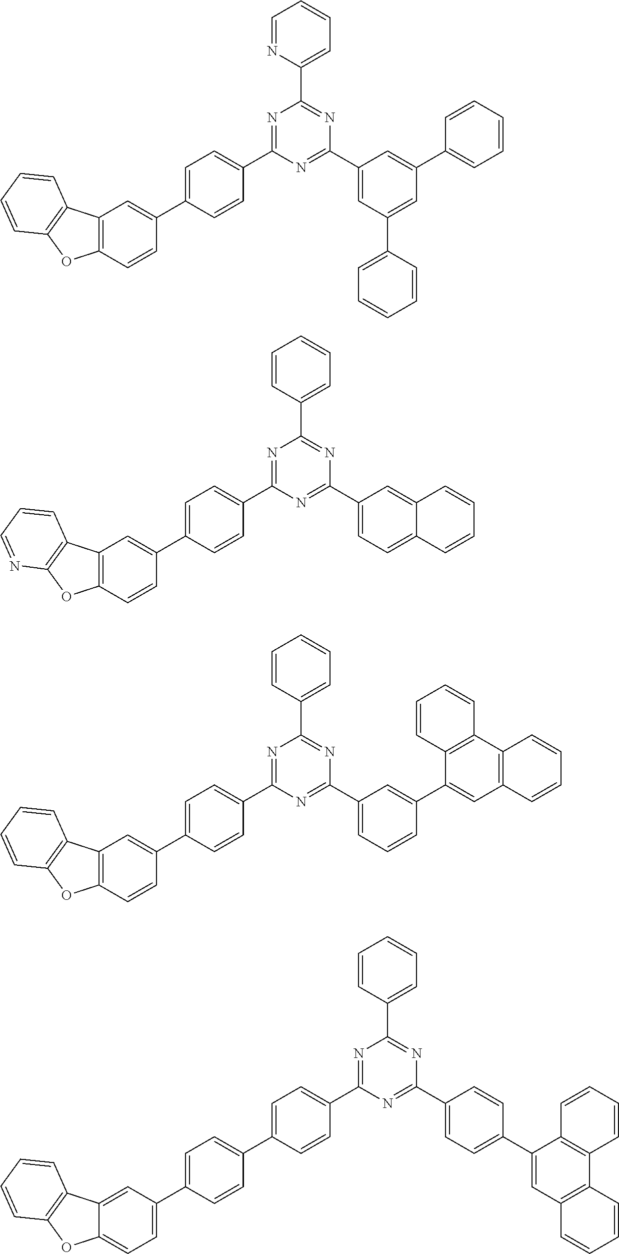 [SMILES: c1ccc(-c2cc(-c3ccccc3)cc(-c3nc(-c4ccc(-c5ccc6oc7ccccc7c6c5)cc4)nc(-c4ccccn4)n3)c2)cc1 ; c1ccc(-c2nc(-c3ccc(-c4ccc5oc6ncccc6c5c4)cc3)nc(-c3ccc4ccccc4c3)n2)cc1 ; c1ccc(-c2nc(-c3ccc(-c4ccc5oc6ccccc6c5c4)cc3)nc(-c3cccc(-c4cc5ccccc5c5ccccc45)c3)n2)cc1 ; c1ccc(-c2nc(-c3ccc(-c4ccc(-c5ccc6oc7ccccc7c6c5)cc4)cc3)nc(-c3ccc(-c4cc5ccccc5c5ccccc45)cc3)n2)cc1]

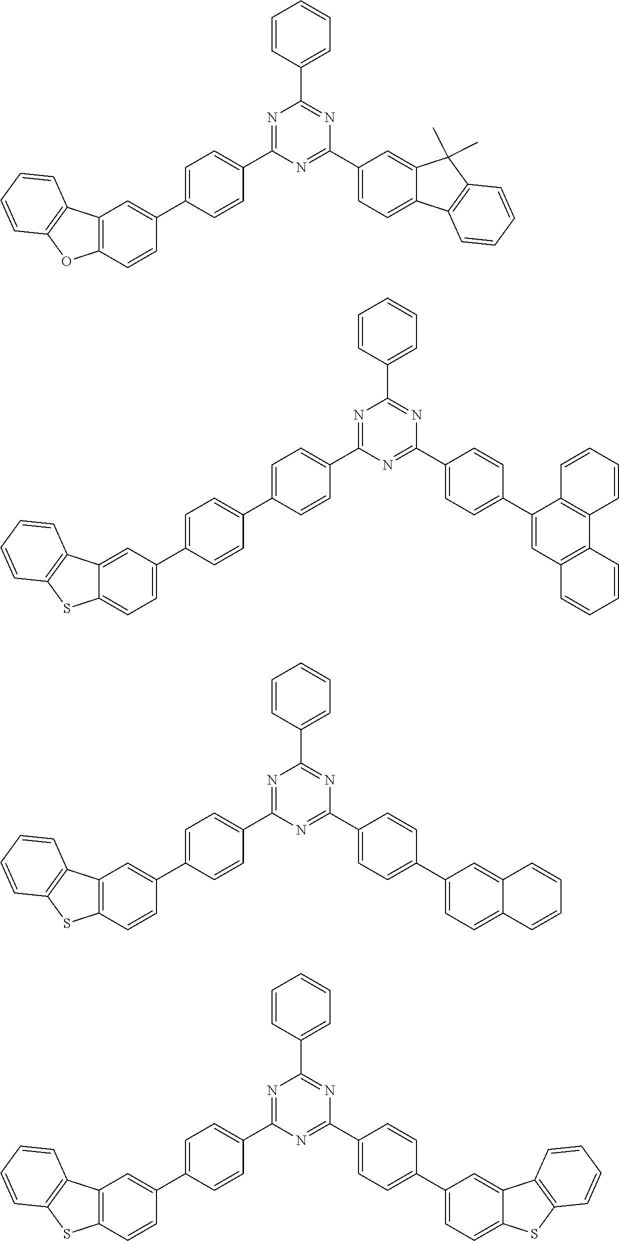 [SMILES: CC1(C)c2ccccc2-c2ccc(-c3nc(-c4ccccc4)nc(-c4ccc(-c5ccc6oc7ccccc7c6c5)cc4)n3)cc21 ; c1ccc(-c2nc(-c3ccc(-c4ccc(-c5ccc6sc7ccccc7c6c5)cc4)cc3)nc(-c3ccc(-c4cc5ccccc5c5ccccc45)cc3)n2)cc1 ; c1ccc(-c2nc(-c3ccc(-c4ccc5ccccc5c4)cc3)nc(-c3ccc(-c4ccc5sc6ccccc6c5c4)cc3)n2)cc1 ; c1ccc(-c2nc(-c3ccc(-c4ccc5sc6ccccc6c5c4)cc3)nc(-c3ccc(-c4ccc5sc6ccccc6c5c4)cc3)n2)cc1]

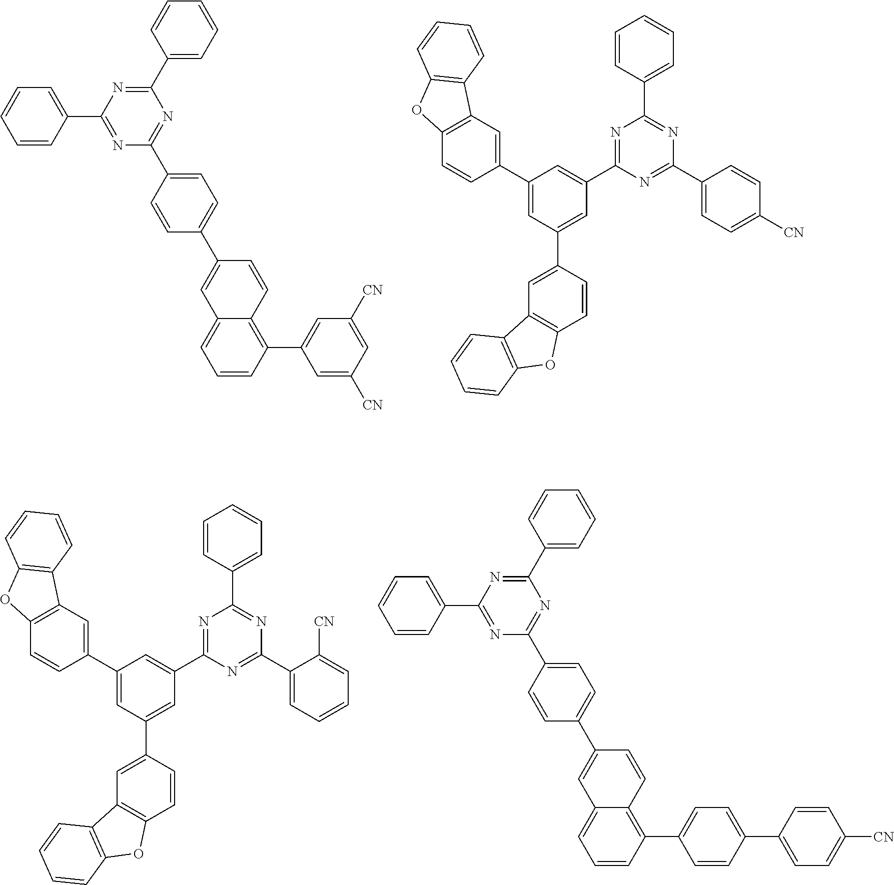 [SMILES: N#Cc1cc(C#N)cc(-c2cccc3cc(-c4ccc(-c5nc(-c6ccccc6)nc(-c6ccccc6)n5)cc4)ccc23)c1 ; N#Cc1ccc(-c2nc(-c3ccccc3)nc(-c3cc(-c4ccc5oc6ccccc6c5c4)cc(-c4ccc5oc6ccccc6c5c4)c3)n2)cc1 ; N#Cc1ccccc1-c1nc(-c2ccccc2)nc(-c2cc(-c3ccc4oc5ccccc5c4c3)cc(-c3ccc4oc5ccccc5c4c3)c2)n1 ; N#Cc1ccc(-c2ccc(-c3cccc4cc(-c5ccc(-c6nc(-c7ccccc7)nc(-c7ccccc7)n6)cc5)ccc34)cc2)cc1]

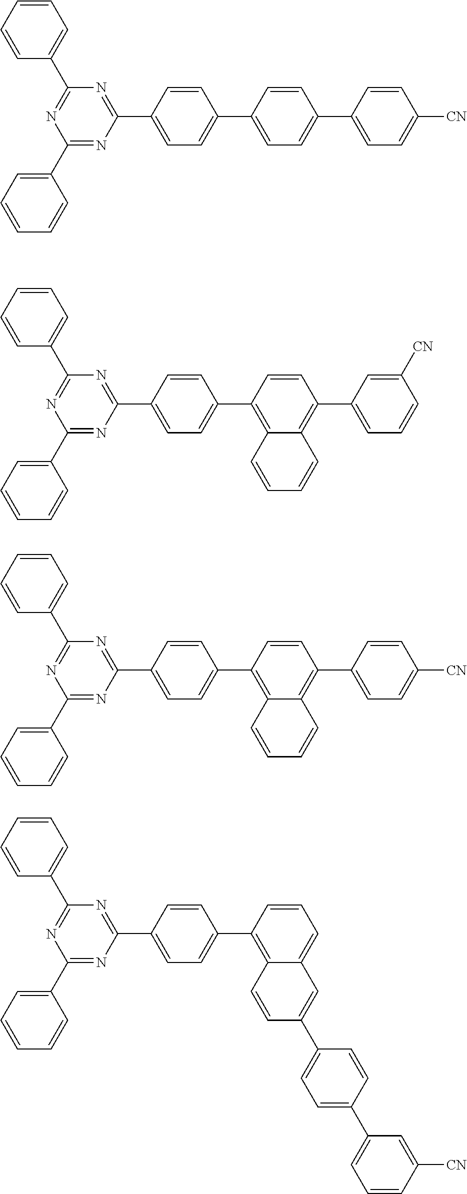

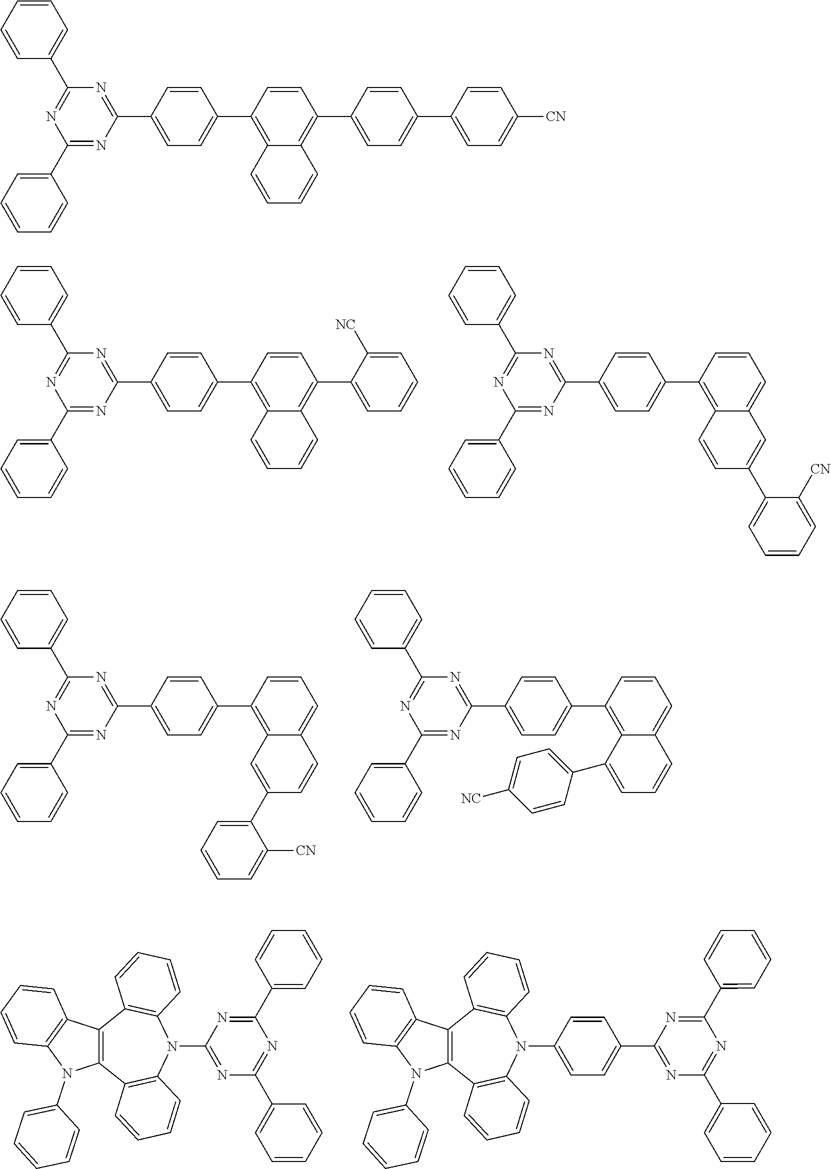

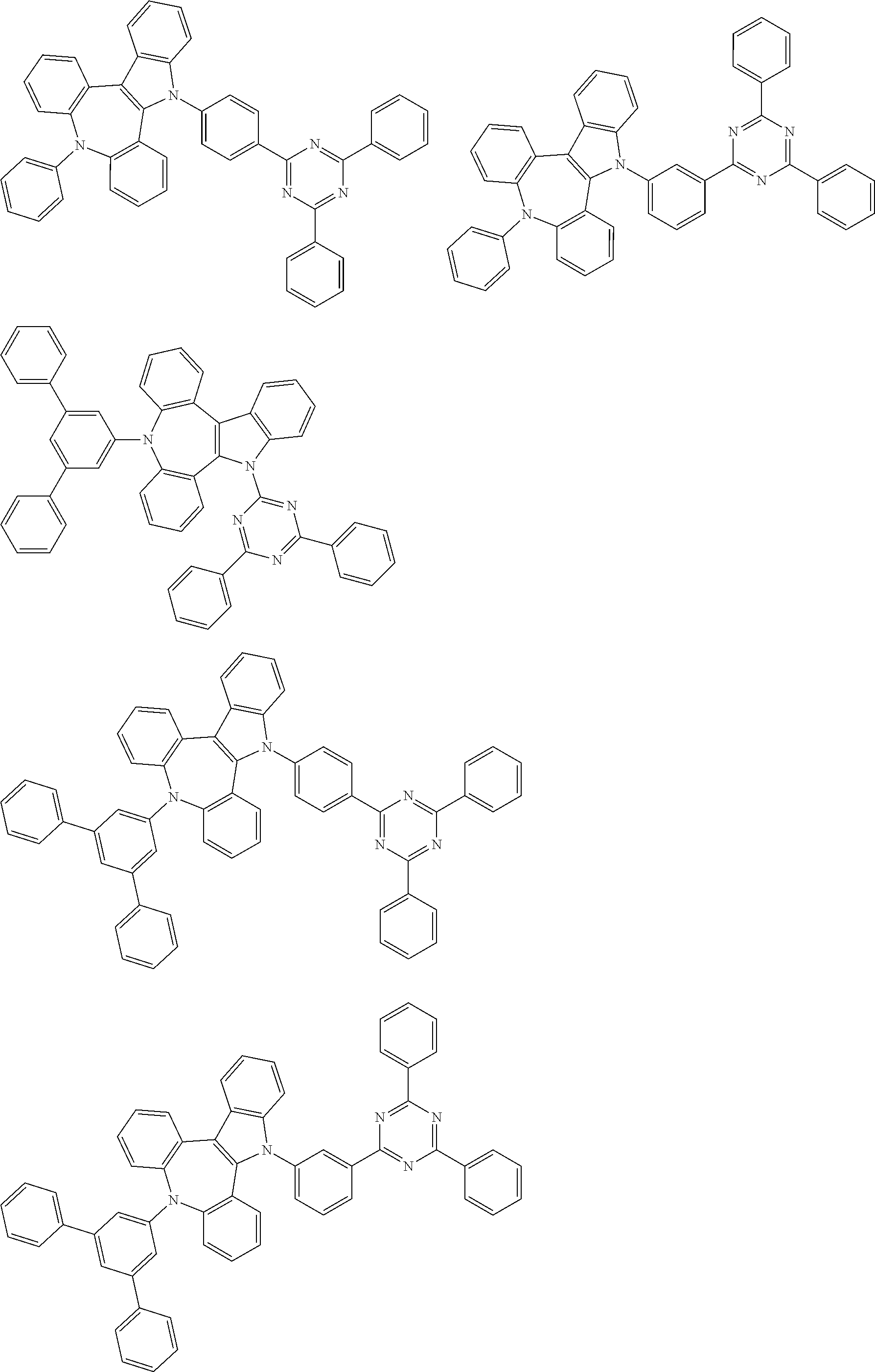

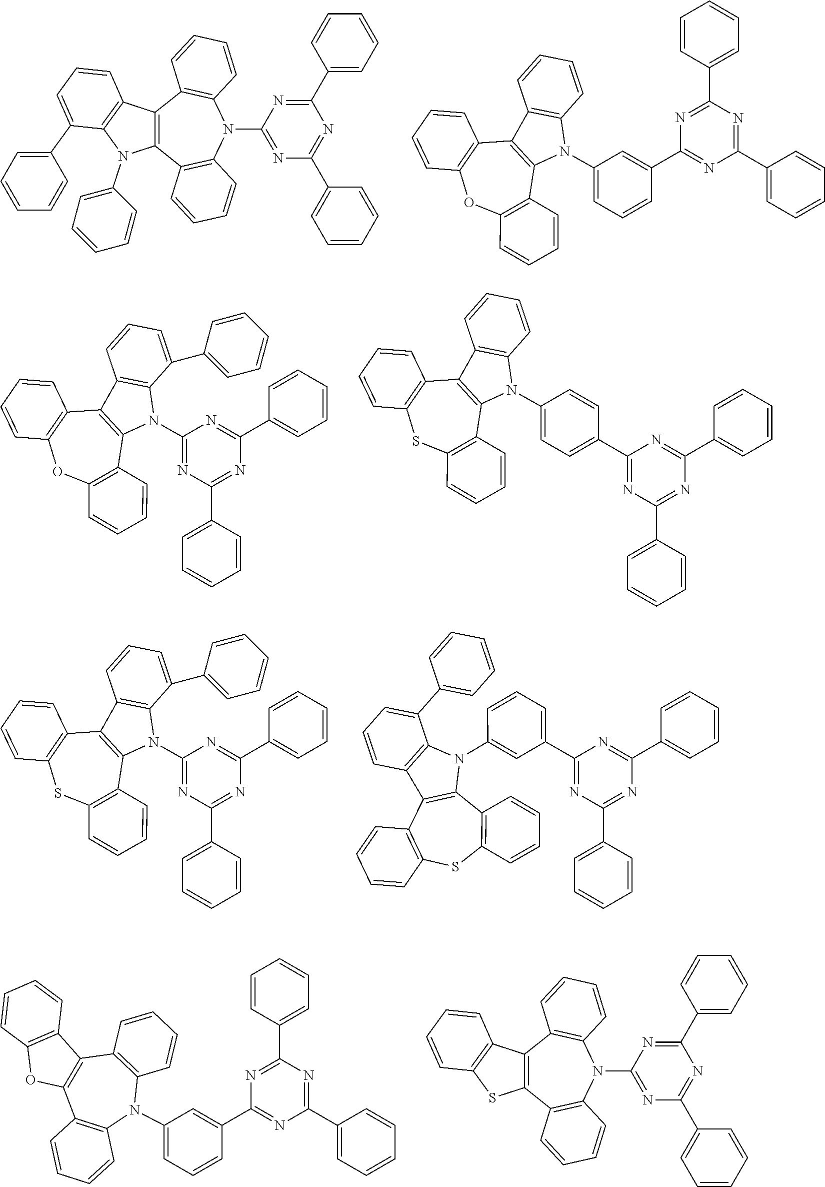

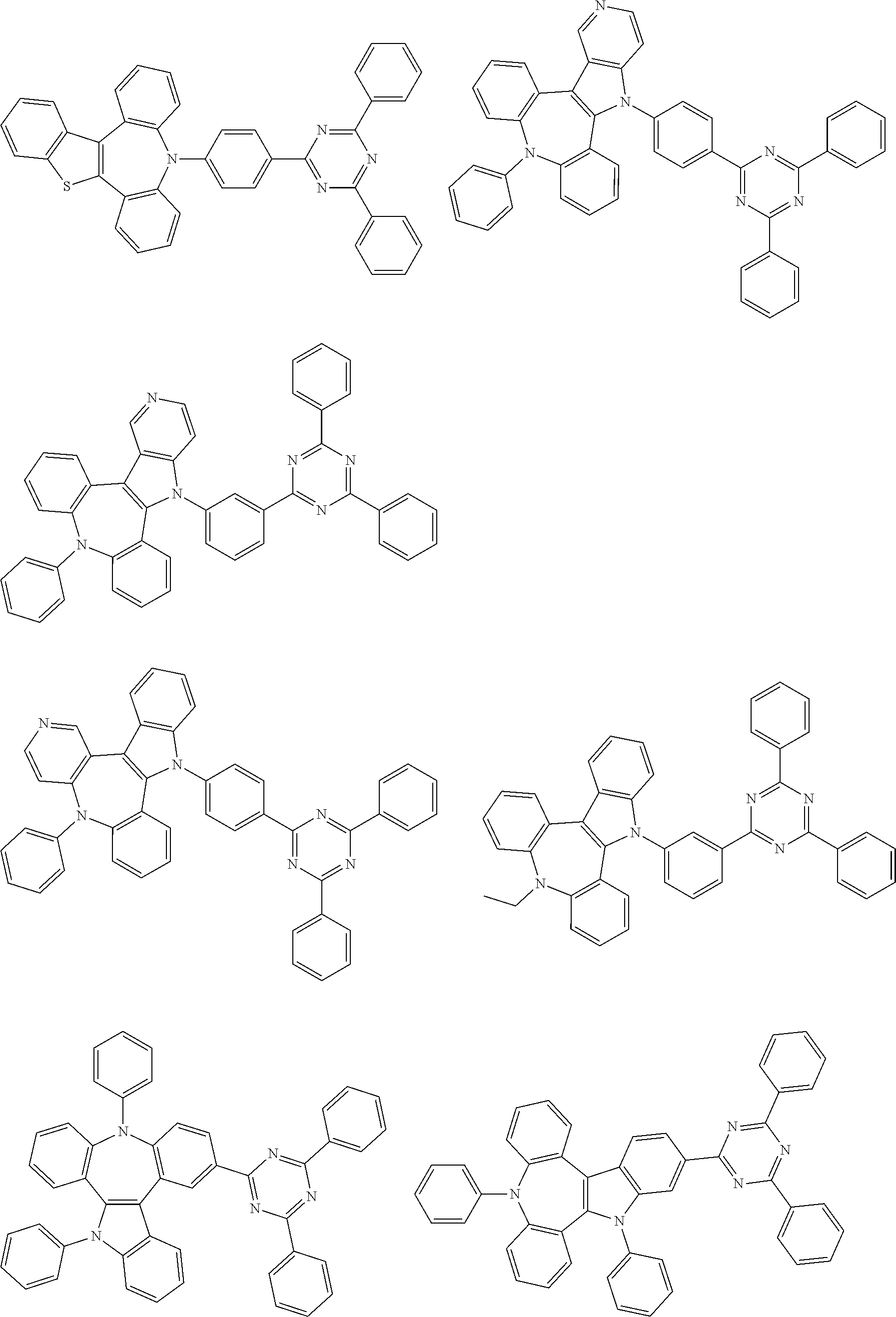

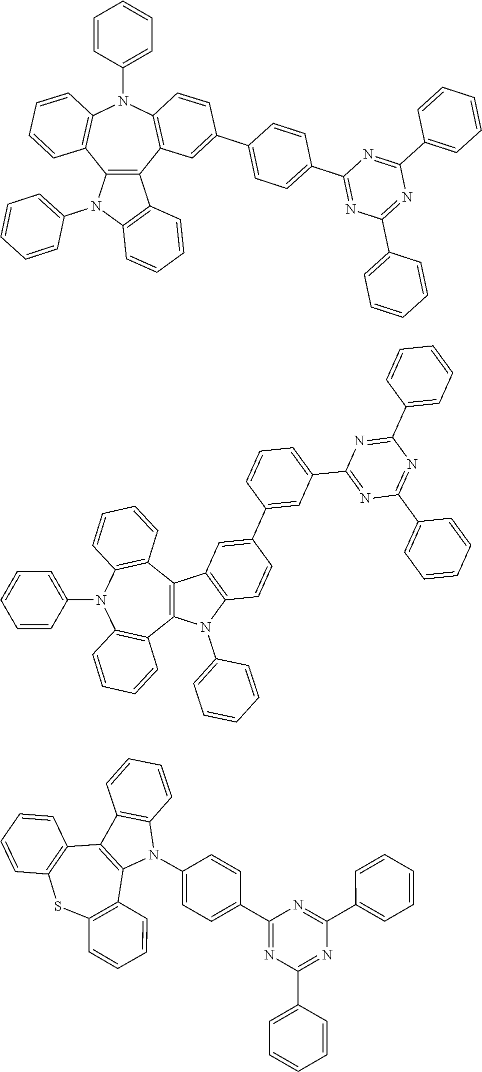

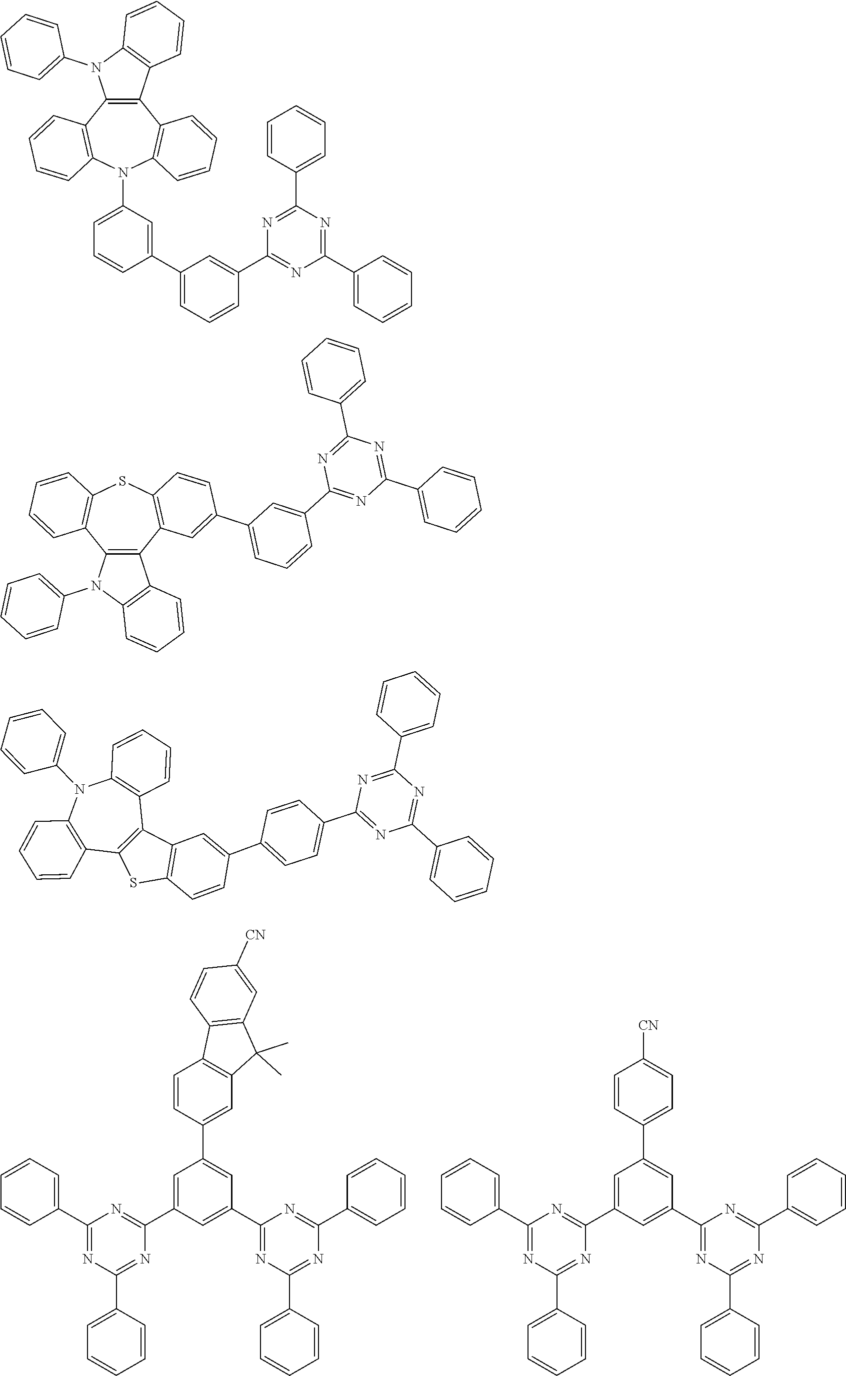

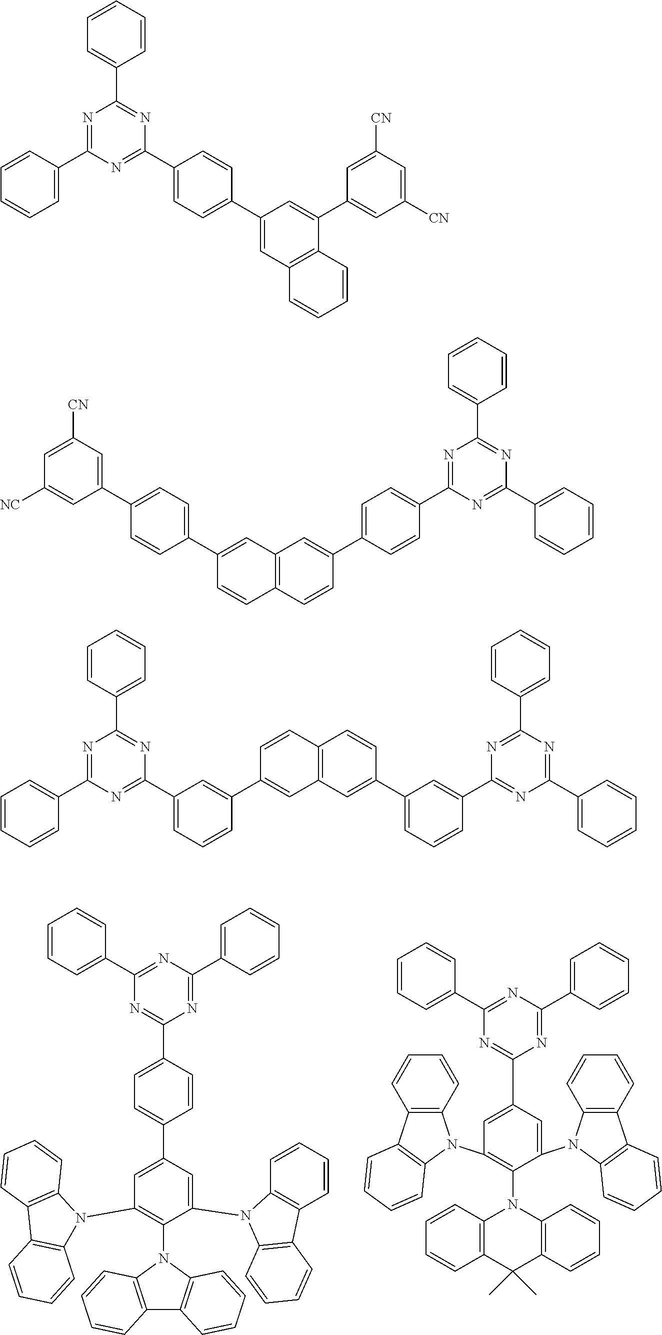

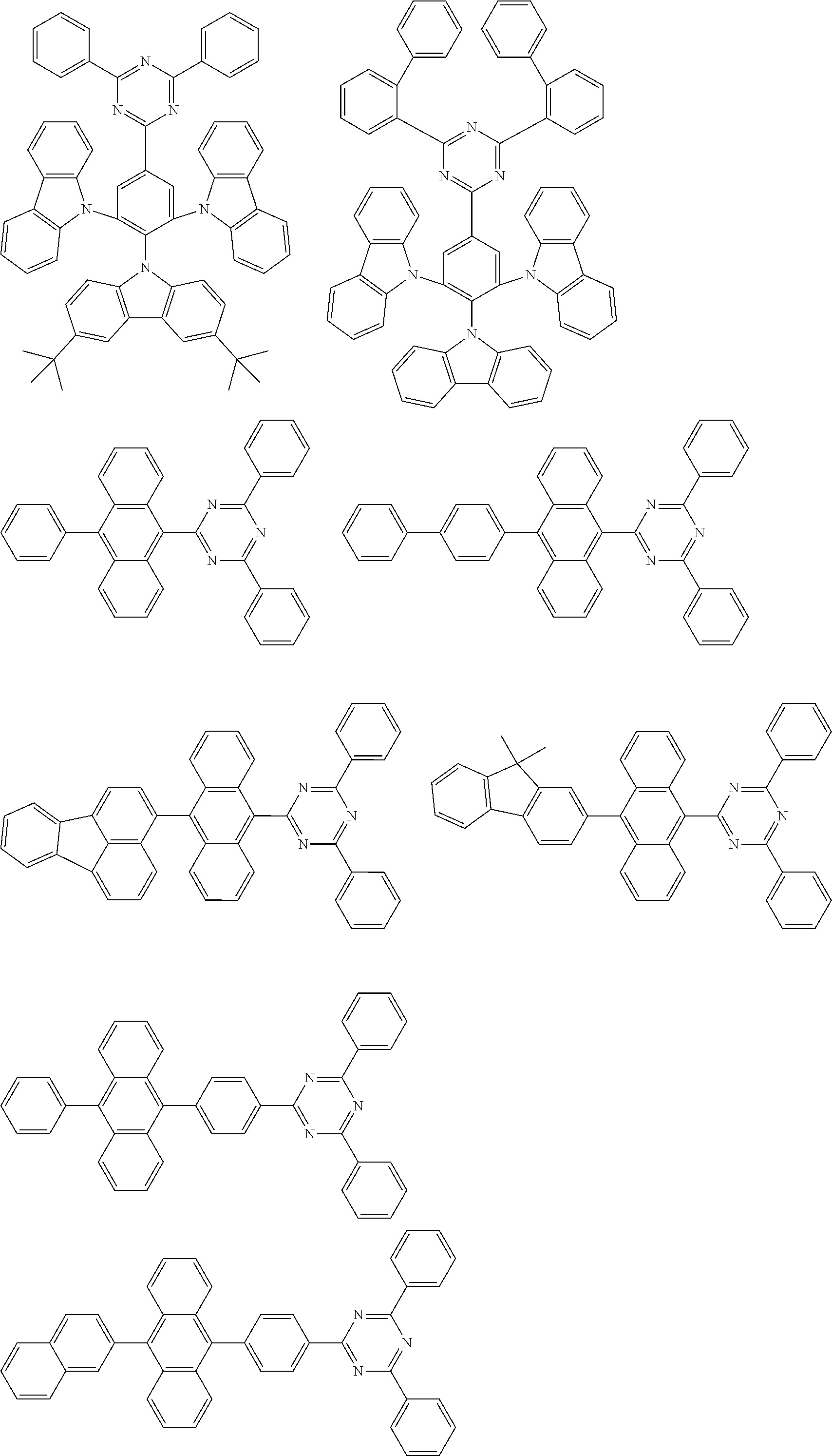

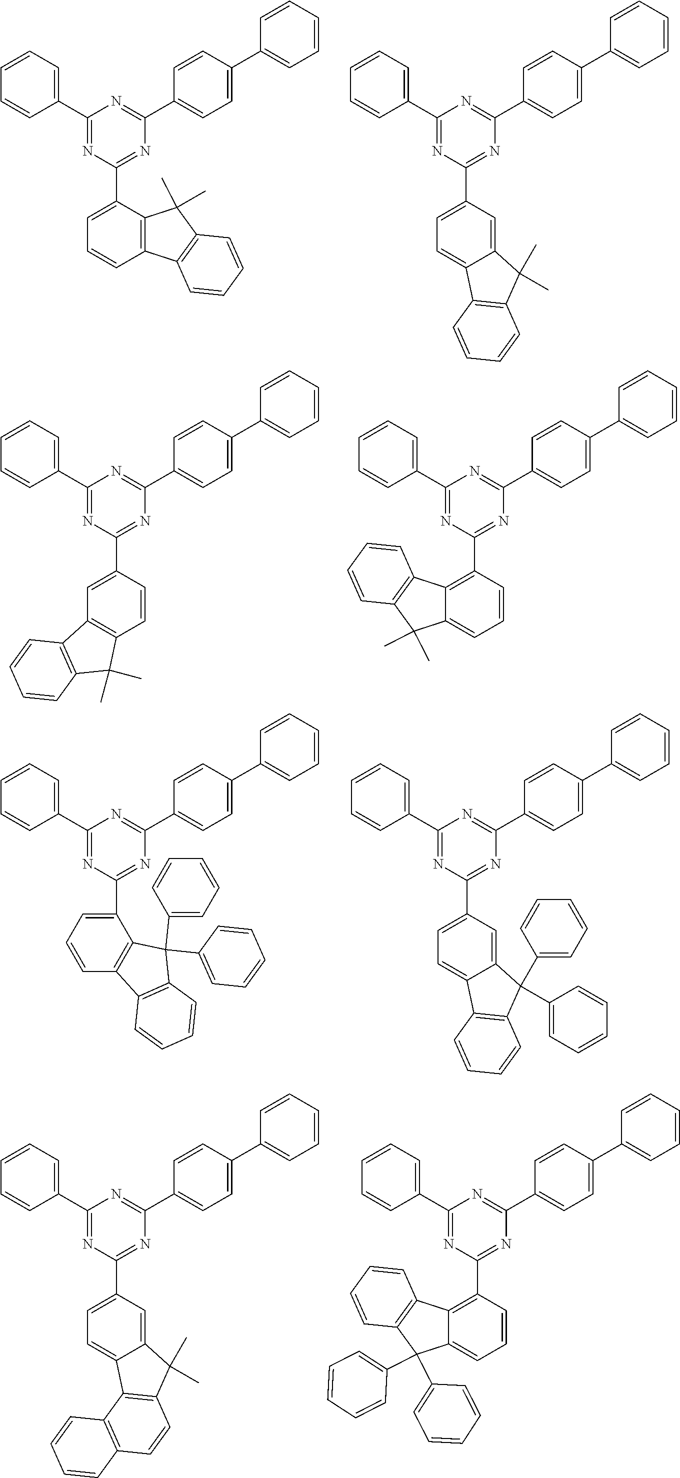

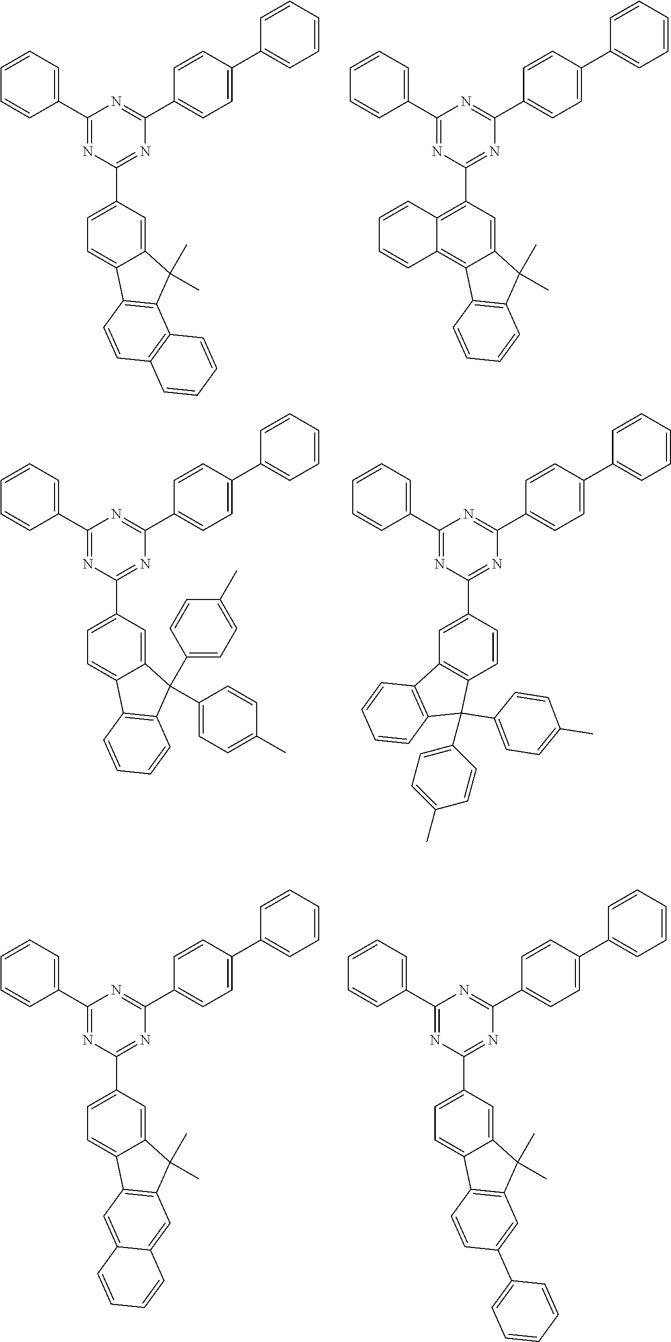

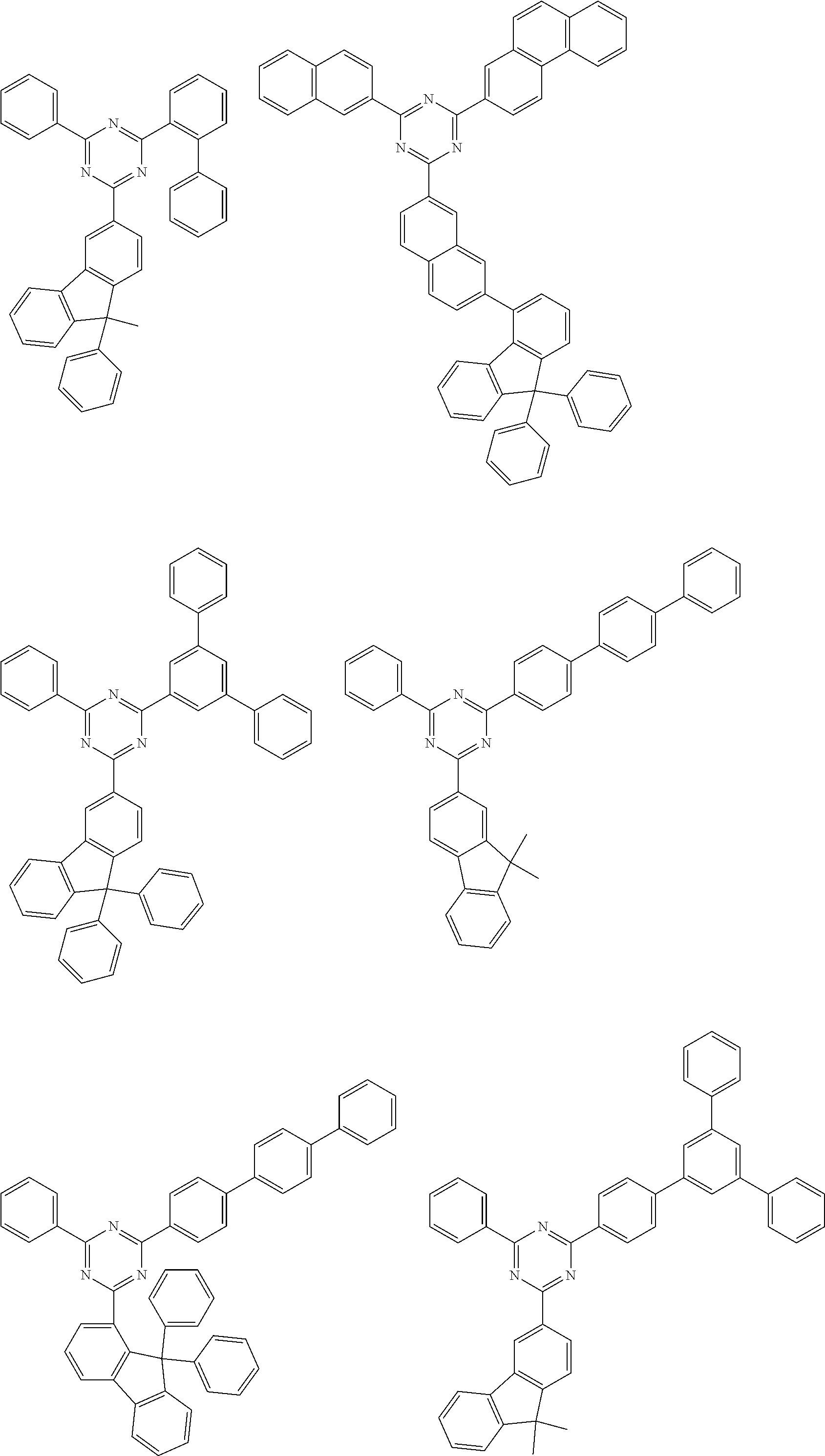

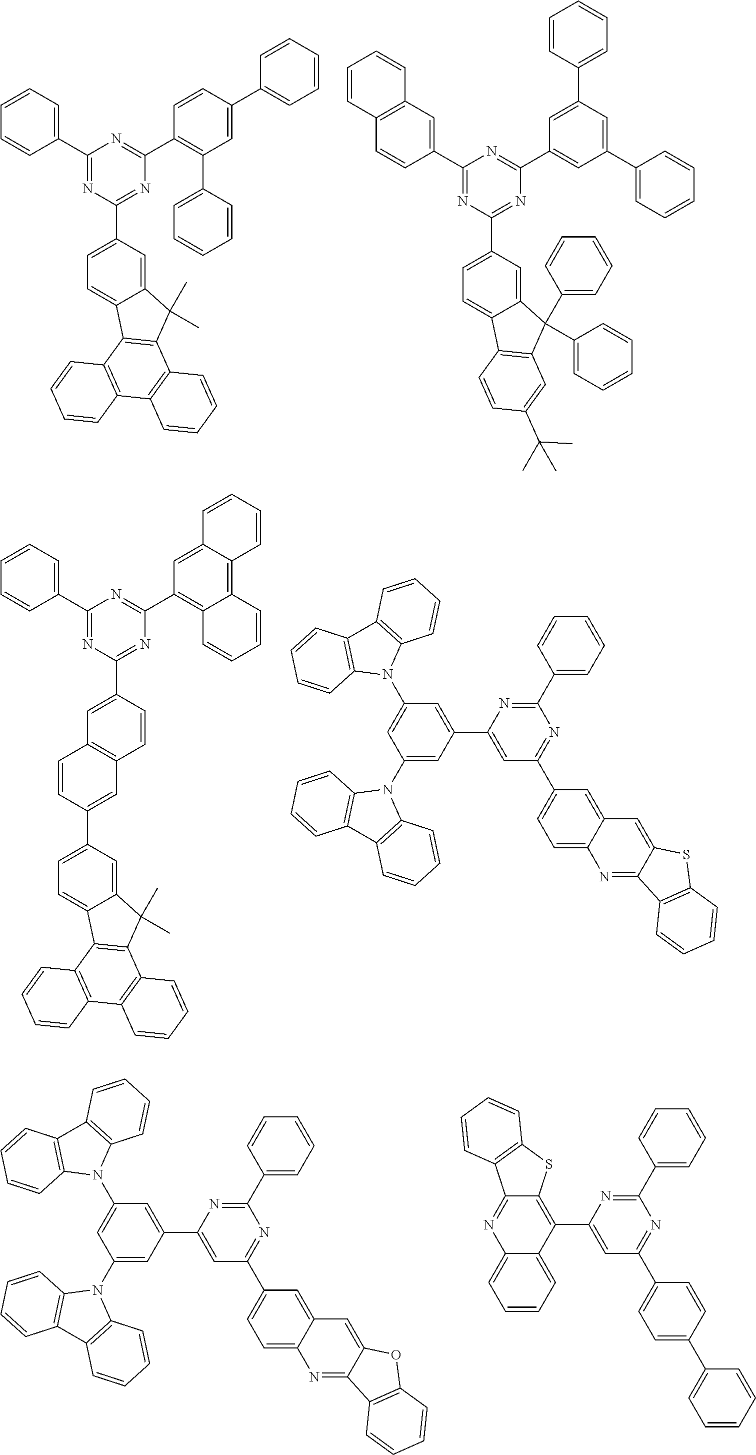

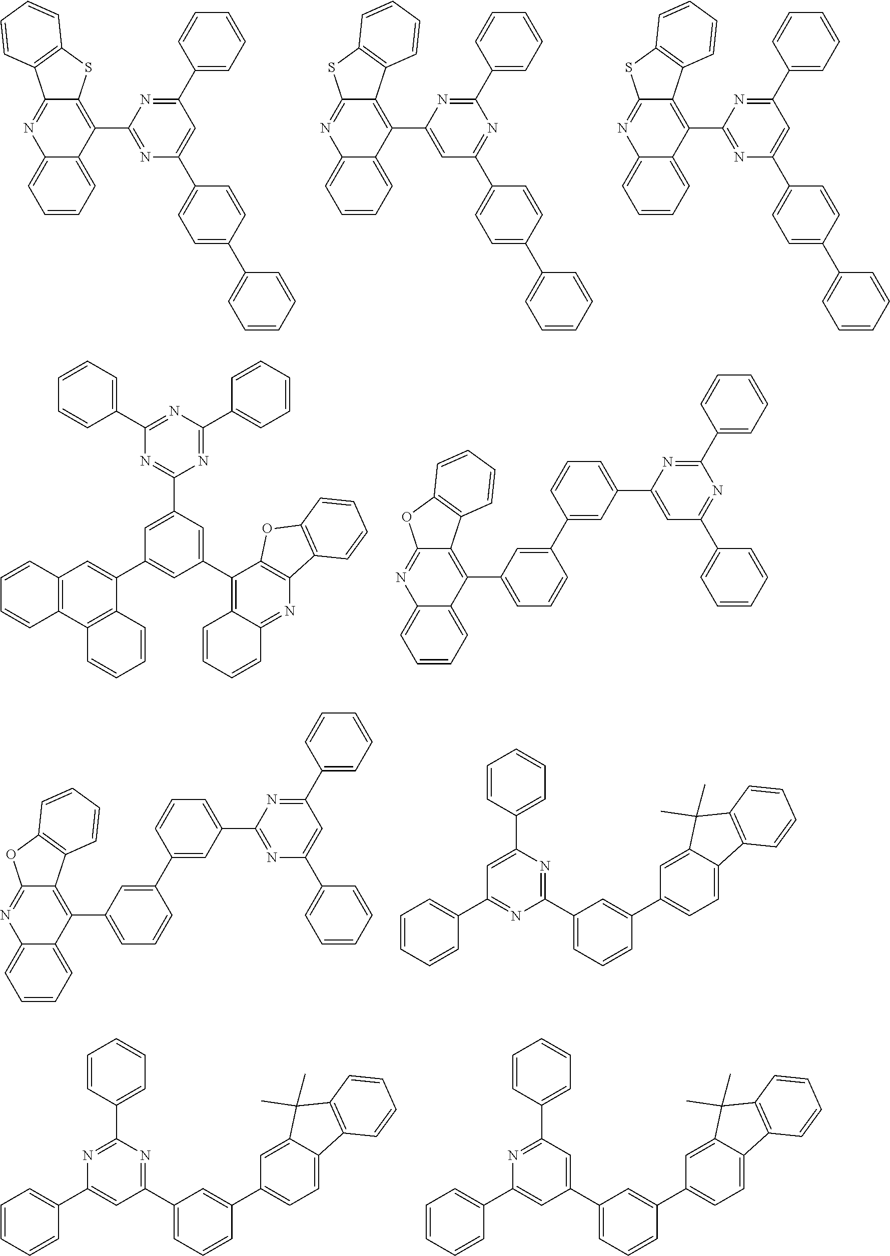

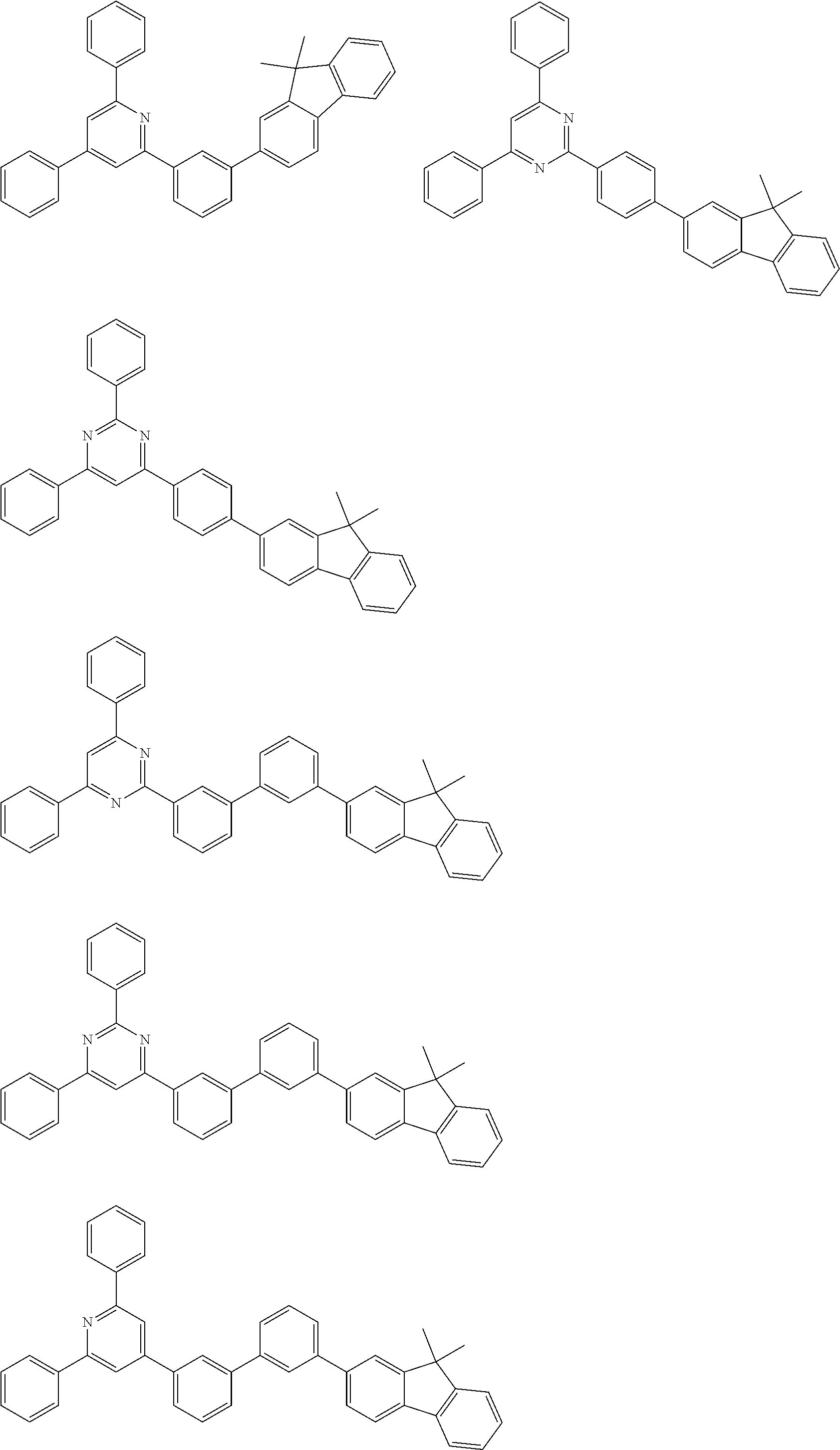 [SMILES: CC1(C)c2ccccc2-c2ccc(-c3cccc(-c4cc(-c5ccccc5)cc(-c5ccccc5)n4)c3)cc21 ; CC1(C)c2ccccc2-c2ccc(-c3ccc(-c4nc(-c5ccccc5)cc(-c5ccccc5)n4)cc3)cc21 ; CC1(C)c2ccccc2-c2ccc(-c3ccc(-c4cc(-c5ccccc5)nc(-c5ccccc5)n4)cc3)cc21 ; CC1(C)c2ccccc2-c2ccc(-c3cccc(-c4cccc(-c5nc(-c6ccccc6)cc(-c6ccccc6)n5)c4)c3)cc21 ; CC1(C)c2ccccc2-c2ccc(-c3cccc(-c4cccc(-c5cc(-c6ccccc6)nc(-c6ccccc6)n5)c4)c3)cc21 ; CC1(C)c2ccccc2-c2ccc(-c3cccc(-c4cccc(-c5cc(-c6ccccc6)nc(-c6ccccc6)c5)c4)c3)cc21]

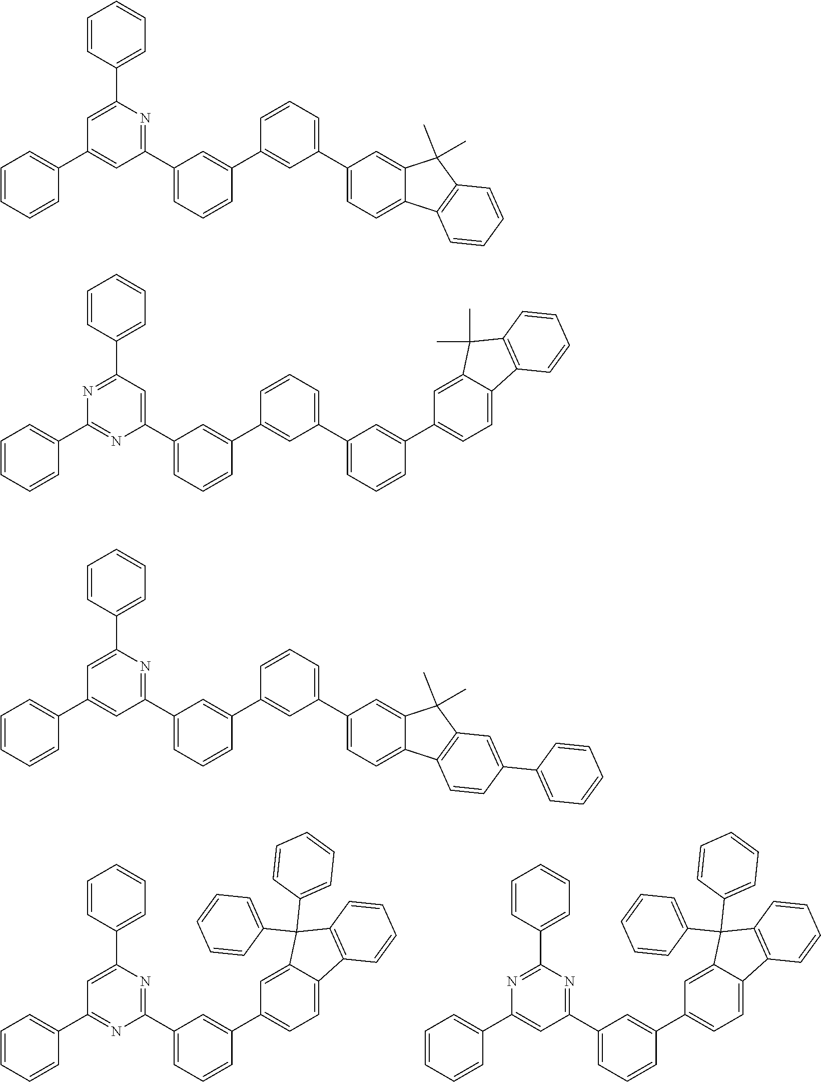 [SMILES: CC1(C)c2ccccc2-c2ccc(-c3cccc(-c4cccc(-c5cc(-c6ccccc6)cc(-c6ccccc6)n5)c4)c3)cc21 ; CC1(C)c2ccccc2-c2ccc(-c3cccc(-c4cccc(-c5cccc(-c6cc(-c7ccccc7)nc(-c7ccccc7)n6)c5)c4)c3)cc21 ; CC1(C)c2cc(-c3ccccc3)ccc2-c2ccc(-c3cccc(-c4cccc(-c5cc(-c6ccccc6)cc(-c6ccccc6)n5)c4)c3)cc21 ; c1ccc(-c2cc(-c3ccccc3)nc(-c3cccc(-c4ccc5c(c4)C(c4ccccc4)(c4ccccc4)c4ccccc4-5)c3)n2)cc1 ; c1ccc(-c2cc(-c3cccc(-c4ccc5c(c4)C(c4ccccc4)(c4ccccc4)c4ccccc4-5)c3)nc(-c3ccccc3)n2)cc1]

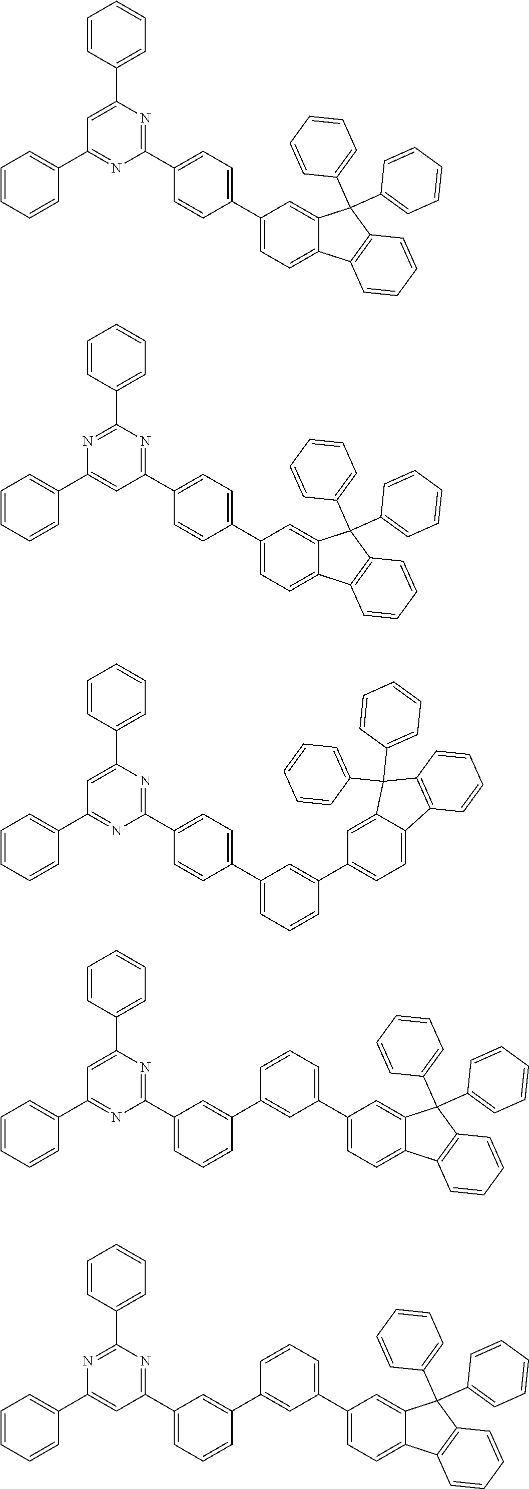 [SMILES: c1ccc(-c2cc(-c3ccccc3)nc(-c3ccc(-c4ccc5c(c4)C(c4ccccc4)(c4ccccc4)c4ccccc4-5)cc3)n2)cc1 ; c1ccc(-c2cc(-c3ccc(-c4ccc5c(c4)C(c4ccccc4)(c4ccccc4)c4ccccc4-5)cc3)nc(-c3ccccc3)n2)cc1 ; c1ccc(-c2cc(-c3ccccc3)nc(-c3ccc(-c4cccc(-c5ccc6c(c5)C(c5ccccc5)(c5ccccc5)c5ccccc5-6)c4)cc3)n2)cc1 ; c1ccc(-c2cc(-c3ccccc3)nc(-c3cccc(-c4cccc(-c5ccc6c(c5)C(c5ccccc5)(c5ccccc5)c5ccccc5-6)c4)c3)n2)cc1 ; c1ccc(-c2cc(-c3cccc(-c4cccc(-c5ccc6c(c5)C(c5ccccc5)(c5ccccc5)c5ccccc5-6)c4)c3)nc(-c3ccccc3)n2)cc1]

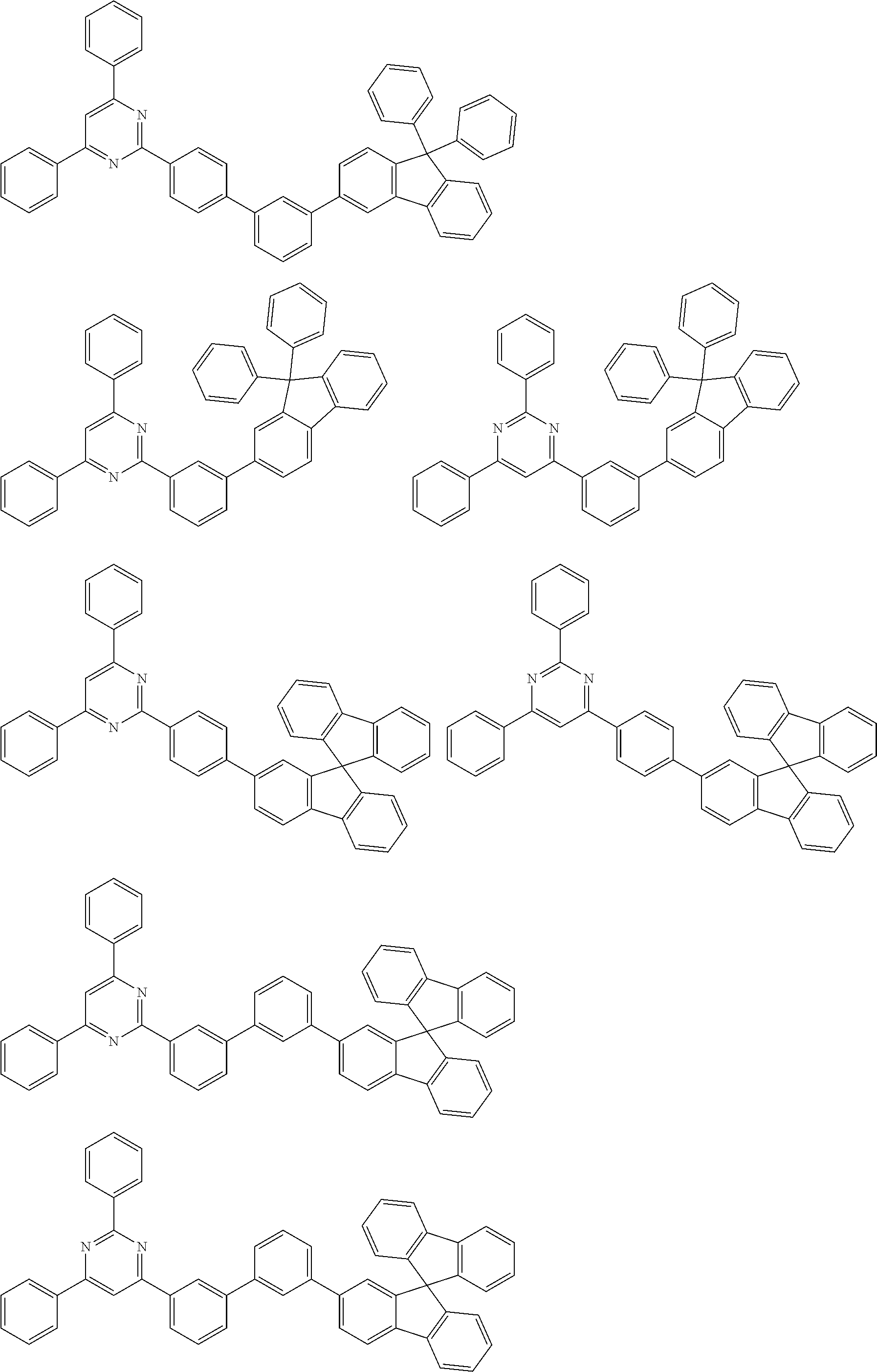

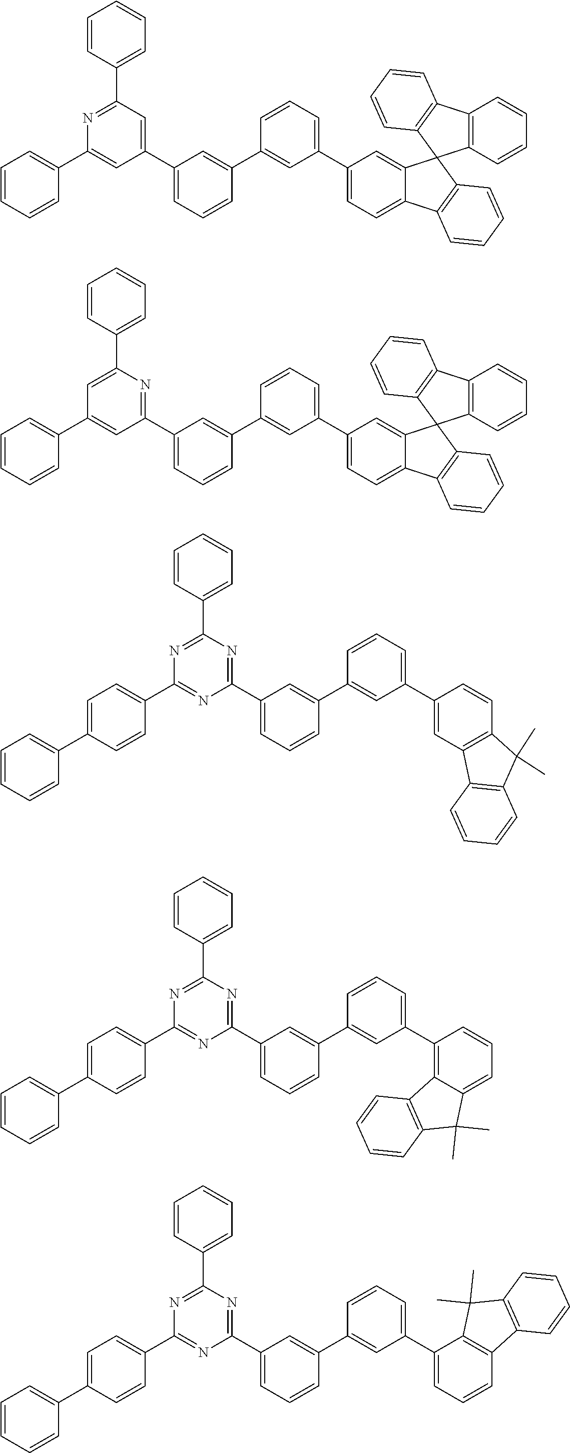 [SMILES: c1ccc(-c2cc(-c3cccc(-c4cccc(-c5ccc6c(c5)C5(c7ccccc7-c7ccccc75)c5ccccc5-6)c4)c3)cc(-c3ccccc3)n2)cc1 ; c1ccc(-c2cc(-c3ccccc3)nc(-c3cccc(-c4cccc(-c5ccc6c(c5)C5(c7ccccc7-c7ccccc75)c5ccccc5-6)c4)c3)c2)cc1 ; CC1(C)c2ccccc2-c2cc(-c3cccc(-c4cccc(-c5nc(-c6ccccc6)nc(-c6ccc(-c7ccccc7)cc6)n5)c4)c3)ccc21 ; CC1(C)c2ccccc2-c2c(-c3cccc(-c4cccc(-c5nc(-c6ccccc6)nc(-c6ccc(-c7ccccc7)cc6)n5)c4)c3)cccc21 ; CC1(C)c2ccccc2-c2cccc(-c3cccc(-c4cccc(-c5nc(-c6ccccc6)nc(-c6ccc(-c7ccccc7)cc6)n5)c4)c3)c21]

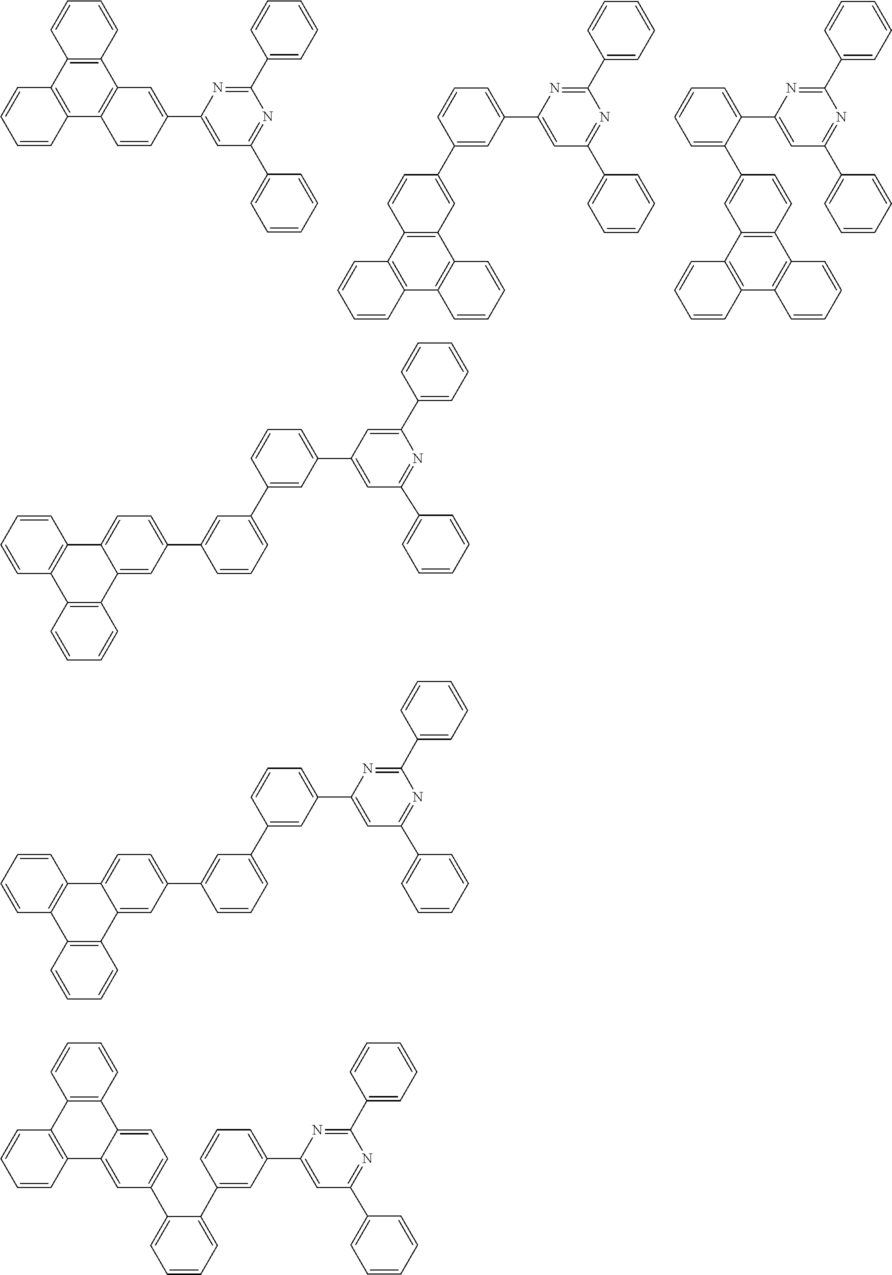

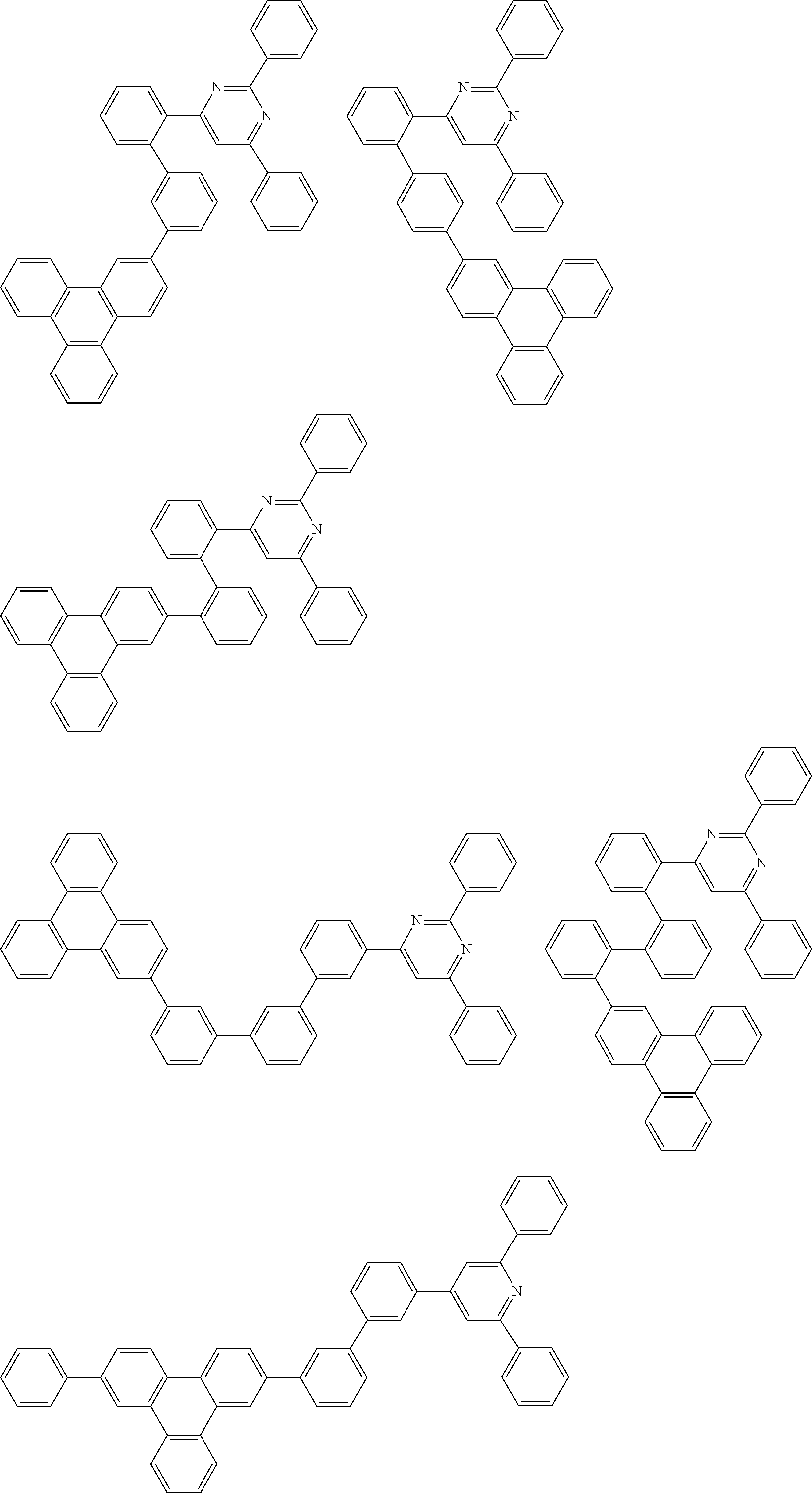

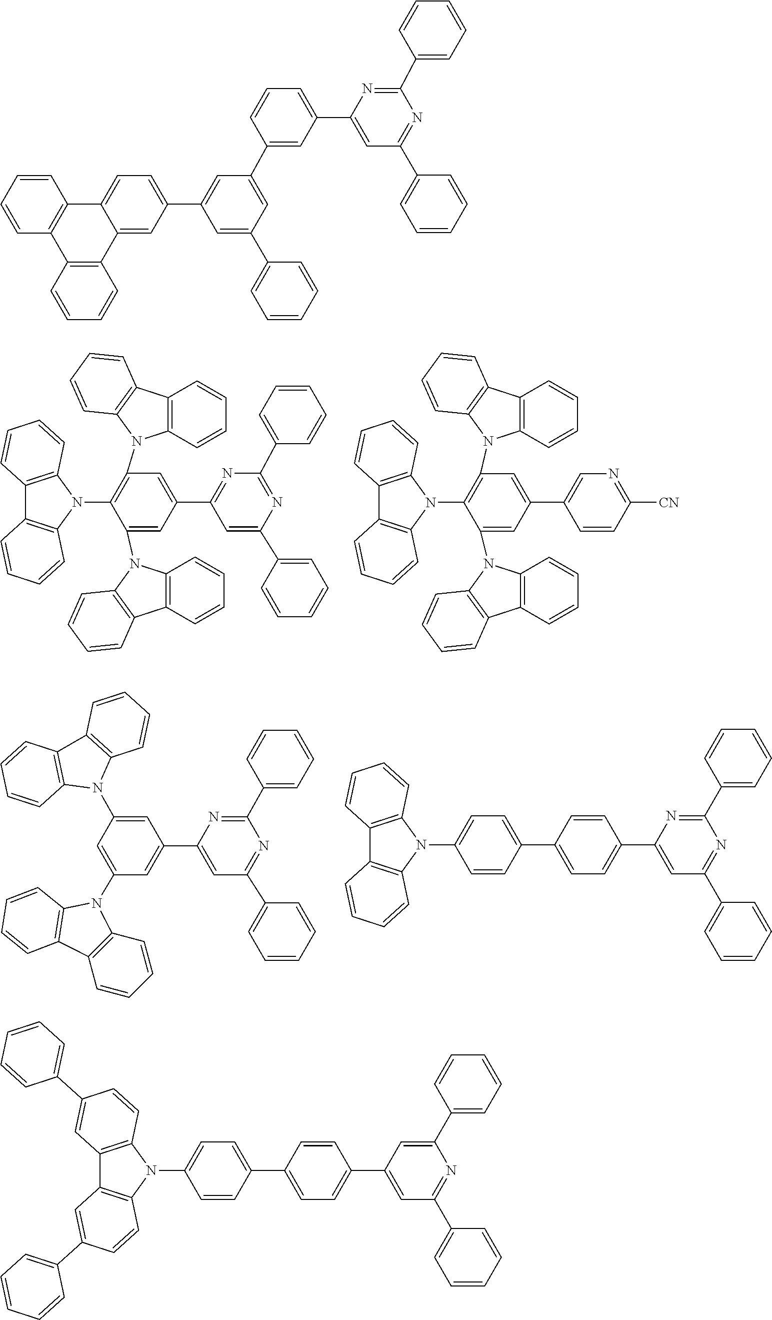

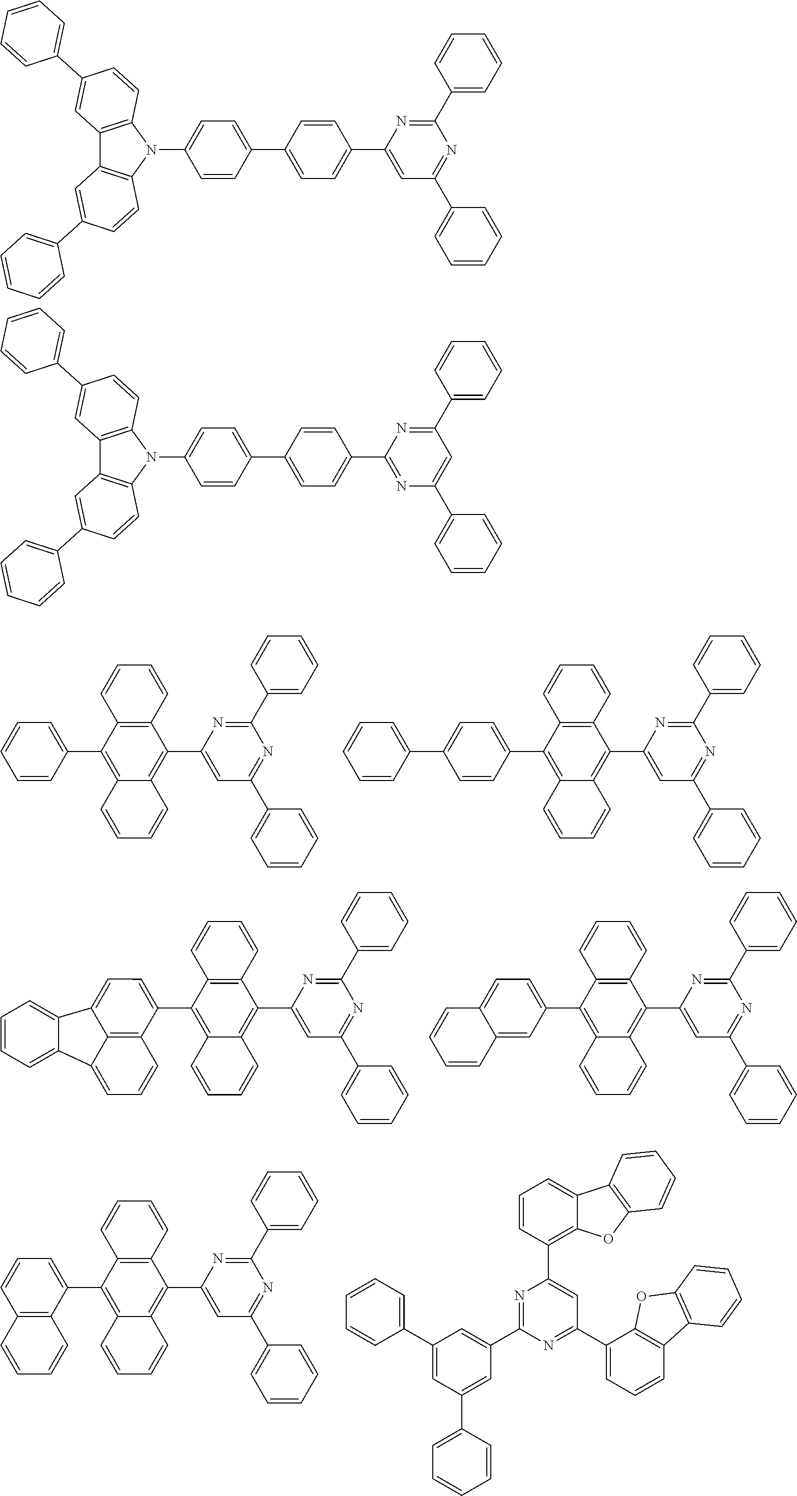

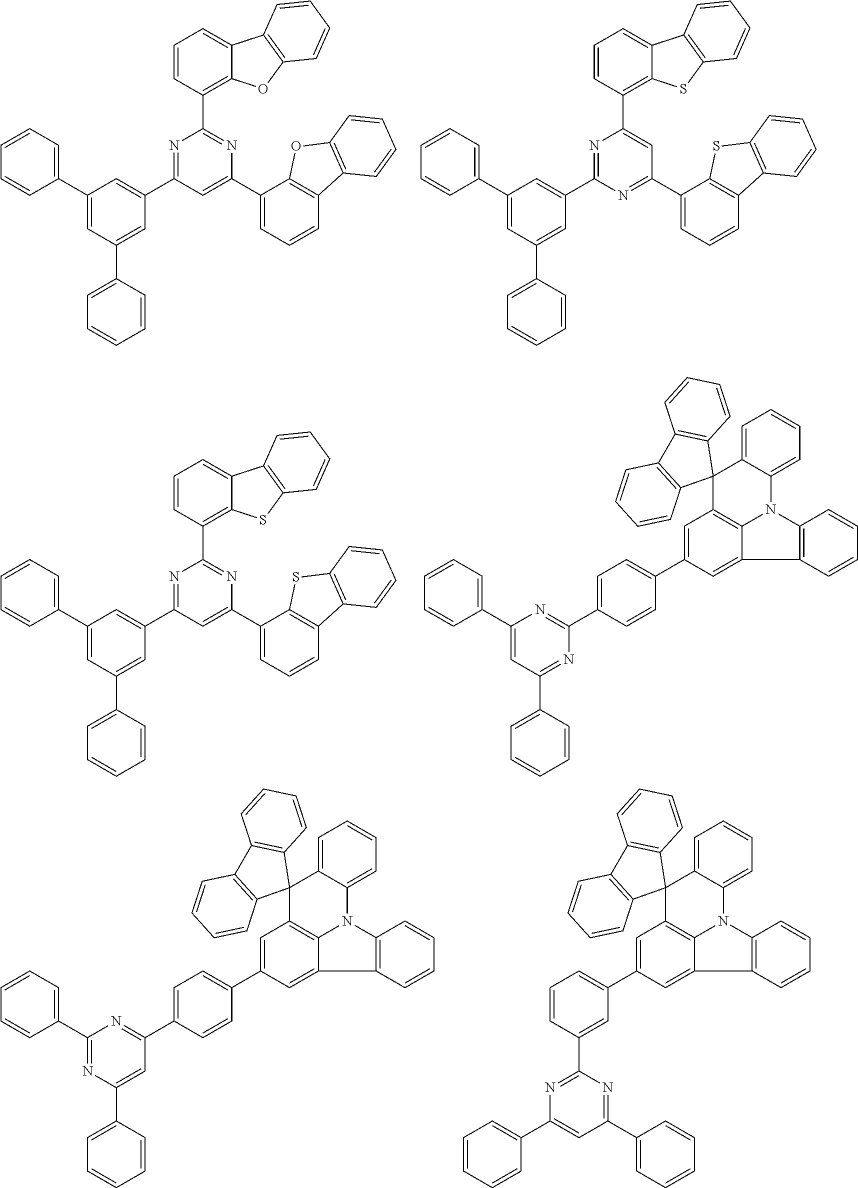

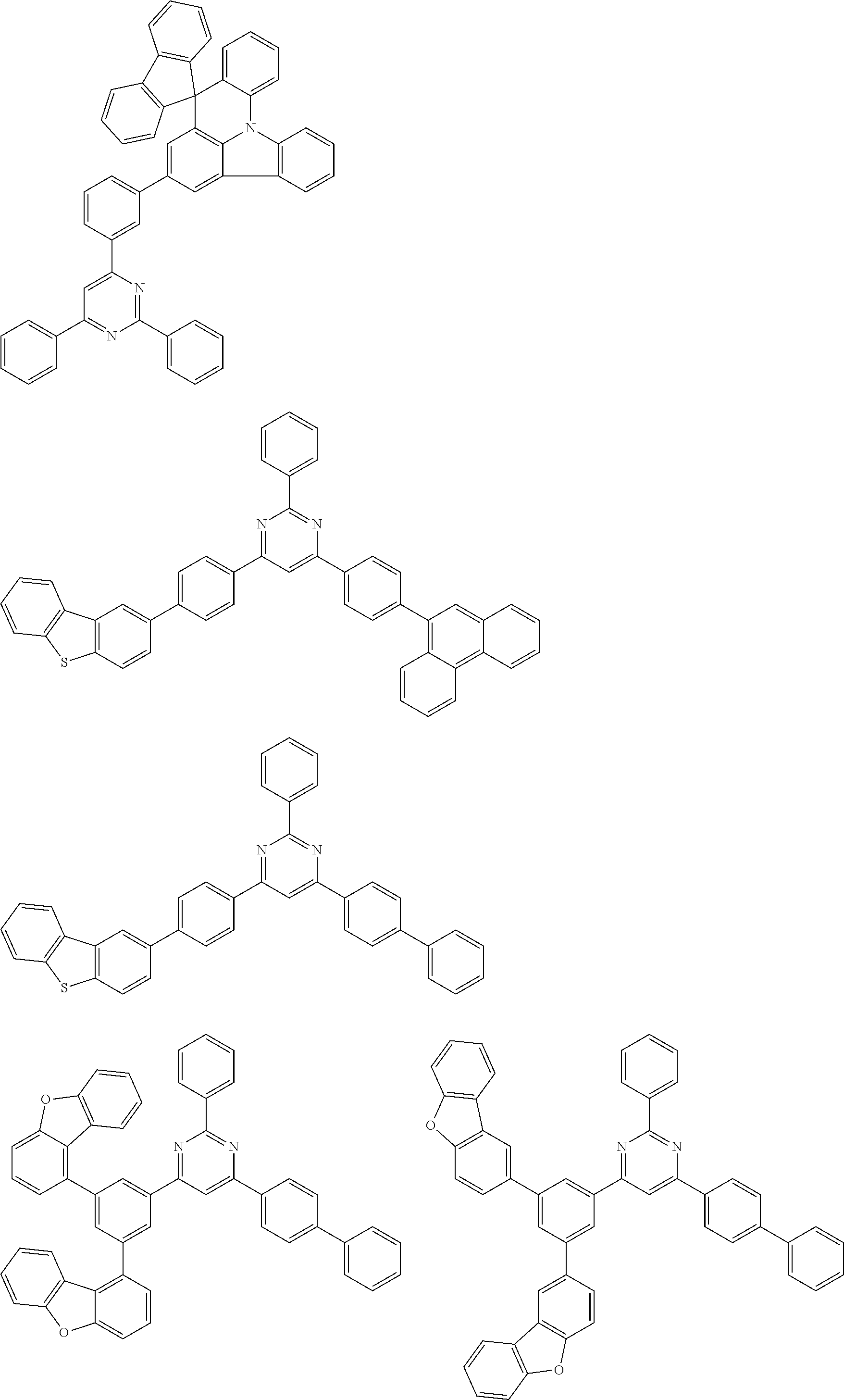 [SMILES: c1ccc(-c2cc(-c3cccc(-c4cc5c6c(c4)c4ccccc4n6-c4ccccc4C54c5ccccc5-c5ccccc54)c3)nc(-c3ccccc3)n2)cc1 ; c1ccc(-c2nc(-c3ccc(-c4ccc5sc6ccccc6c5c4)cc3)cc(-c3ccc(-c4cc5ccccc5c5ccccc45)cc3)n2)cc1 ; c1ccc(-c2ccc(-c3cc(-c4ccc(-c5ccc6sc7ccccc7c6c5)cc4)nc(-c4ccccc4)n3)cc2)cc1 ; c1ccc(-c2ccc(-c3cc(-c4cc(-c5cccc6oc7ccccc7c56)cc(-c5cccc6oc7ccccc7c56)c4)nc(-c4ccccc4)n3)cc2)cc1 ; c1ccc(-c2ccc(-c3cc(-c4cc(-c5ccc6oc7ccccc7c6c5)cc(-c5ccc6oc7ccccc7c6c5)c4)nc(-c4ccccc4)n3)cc2)cc1]

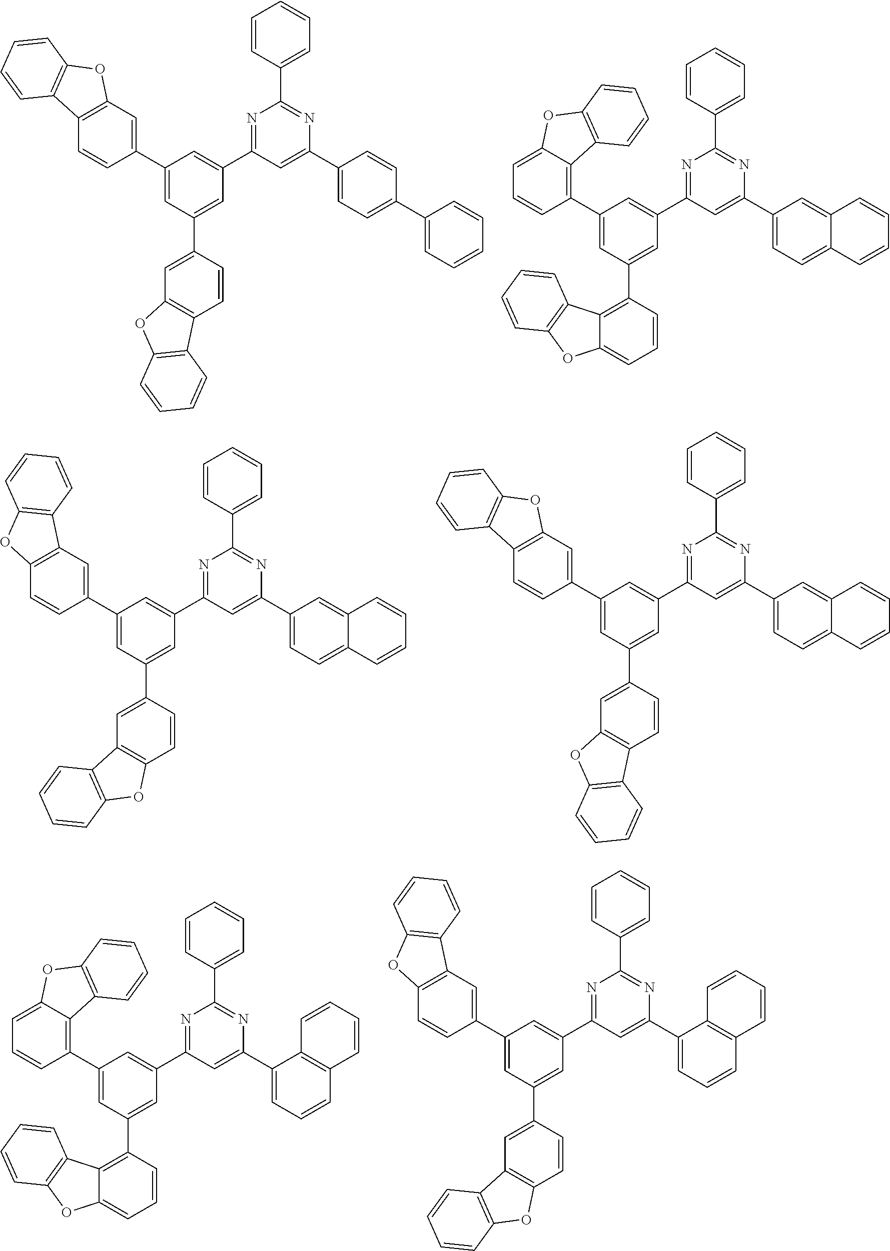

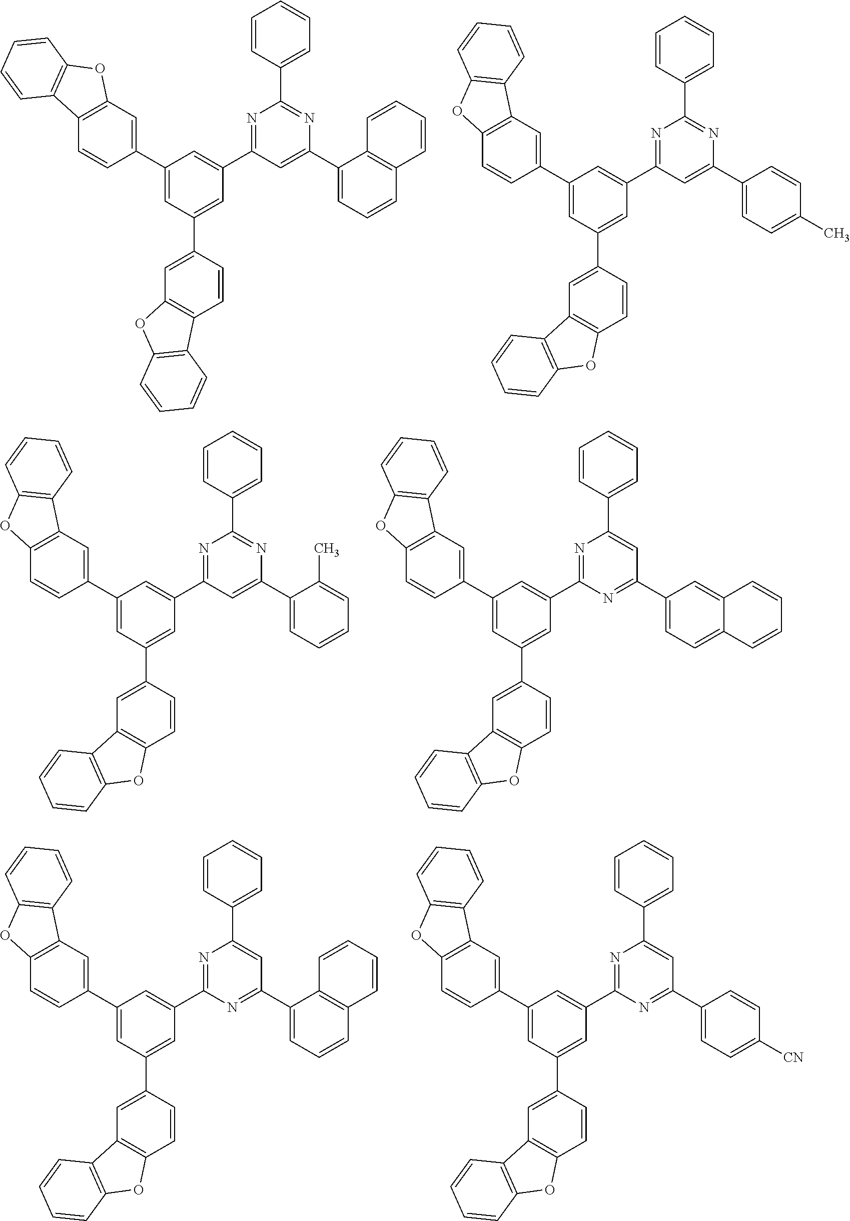

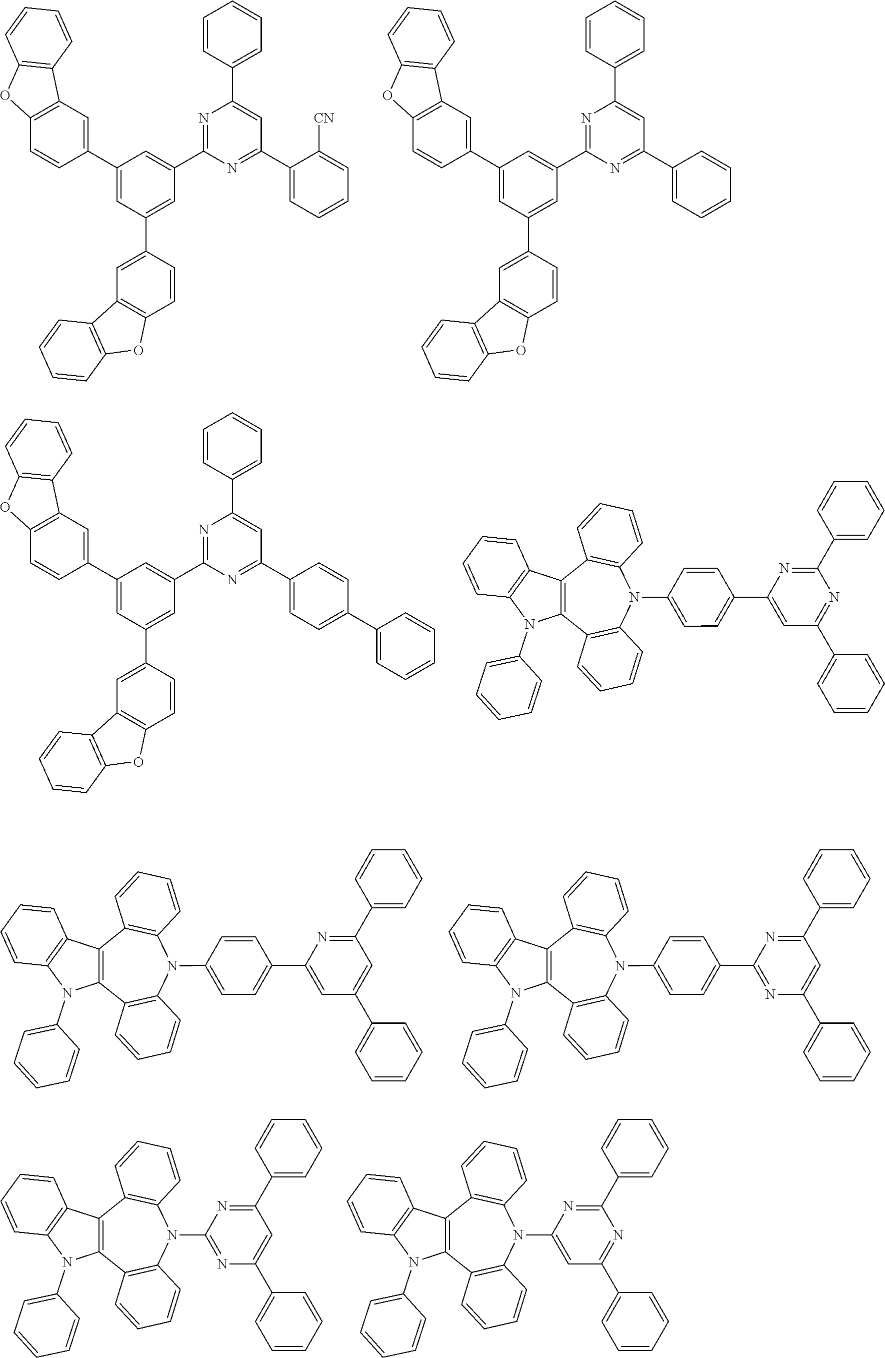

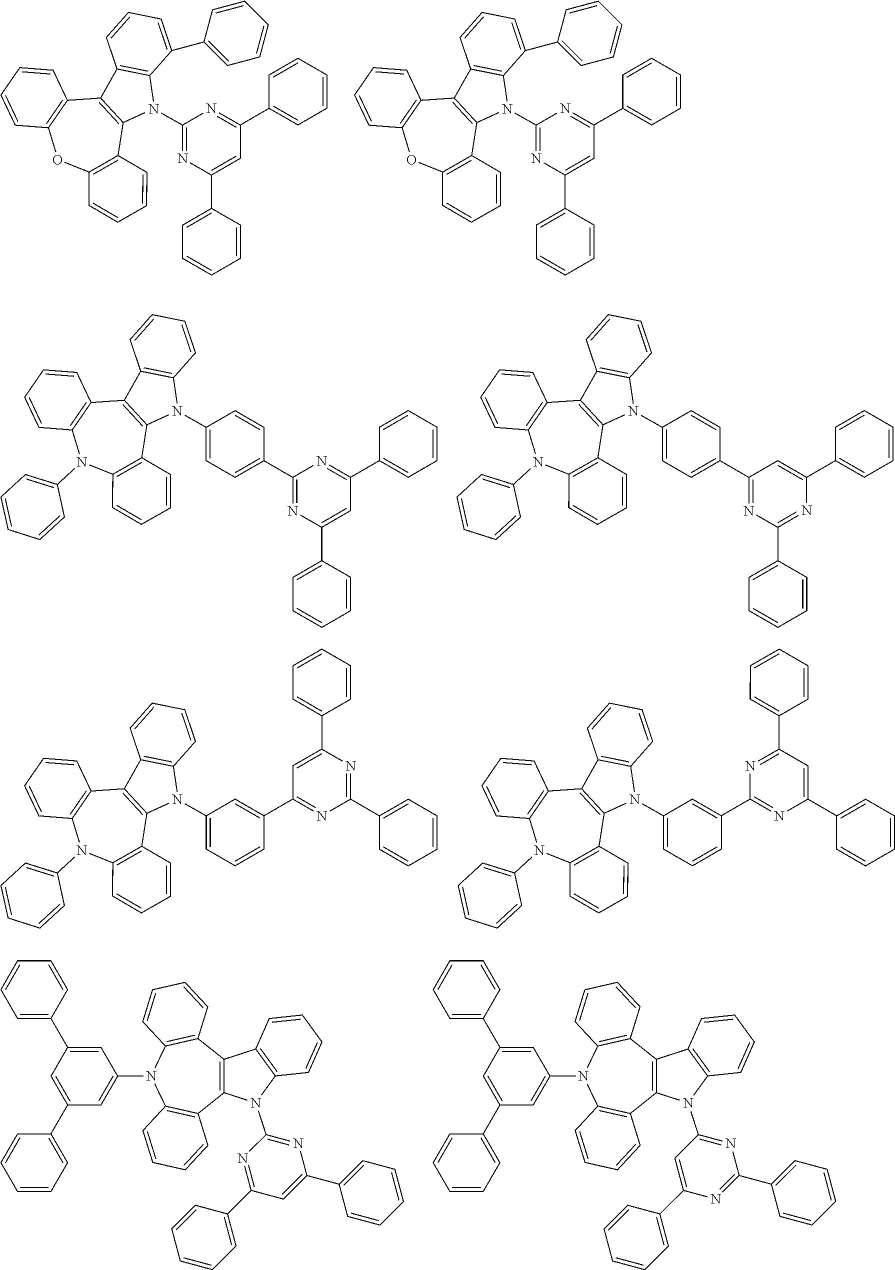

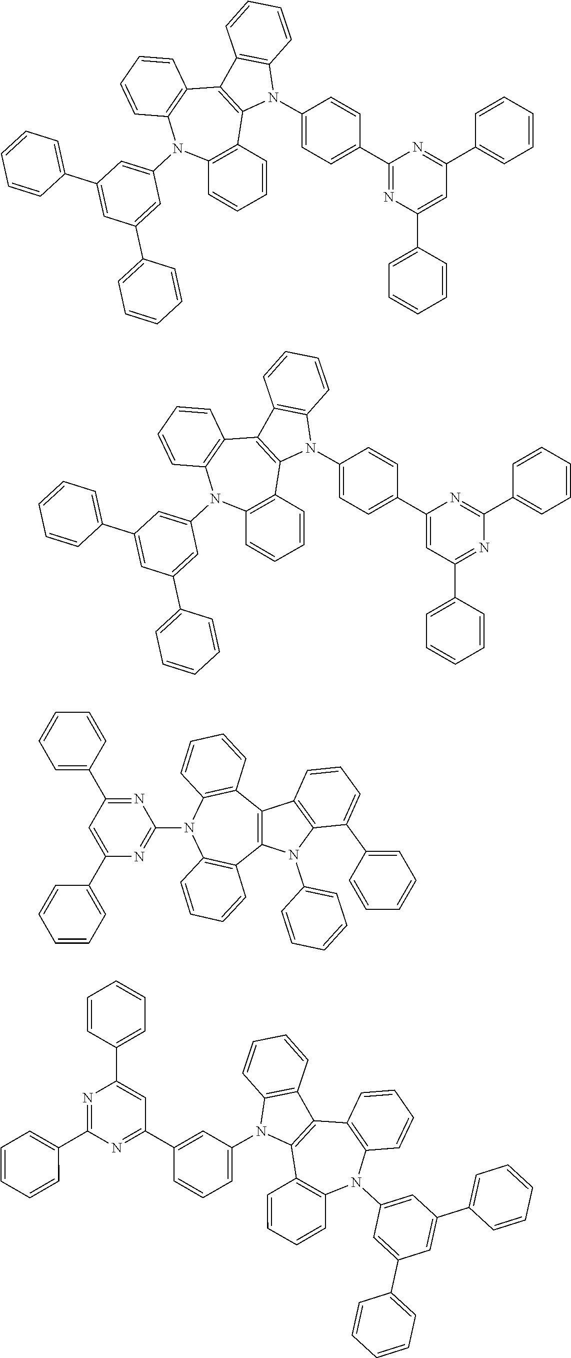

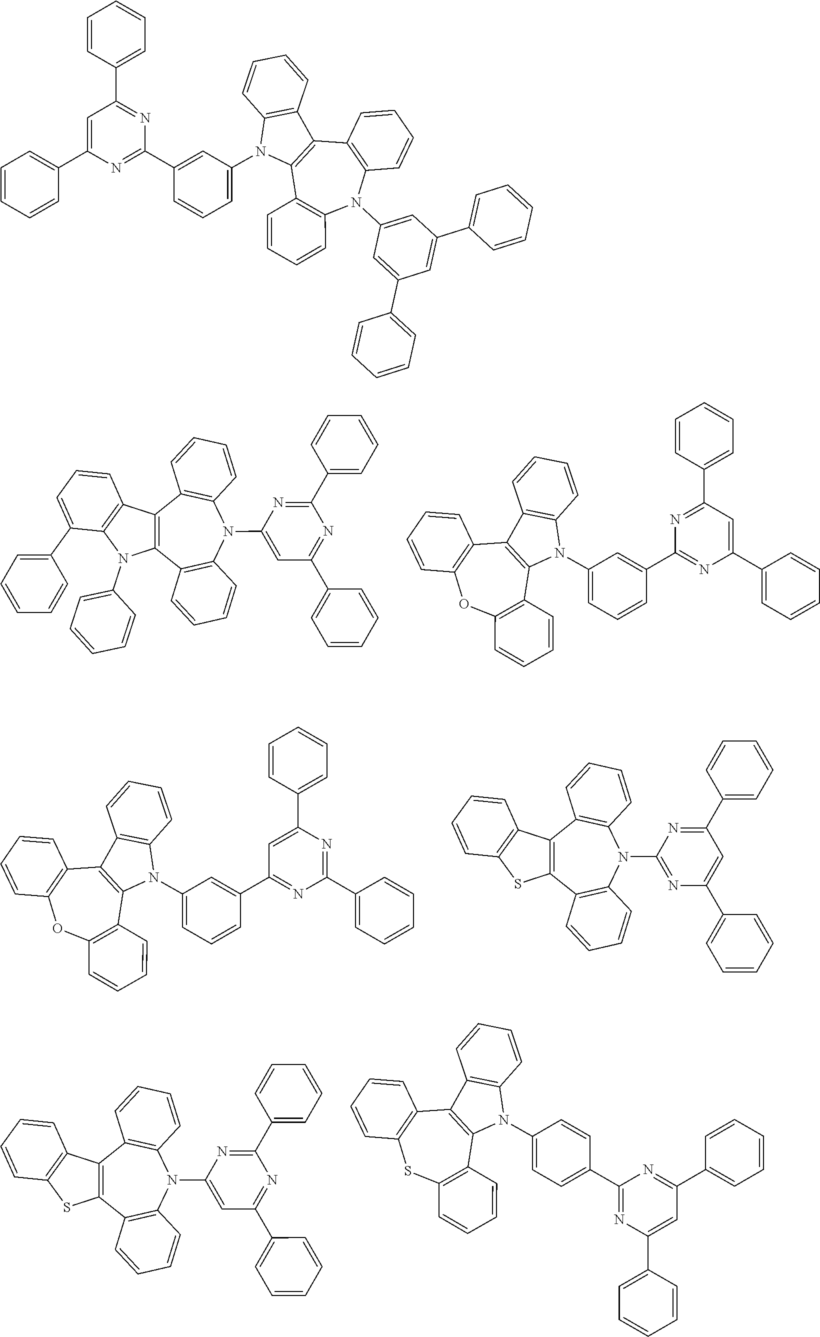

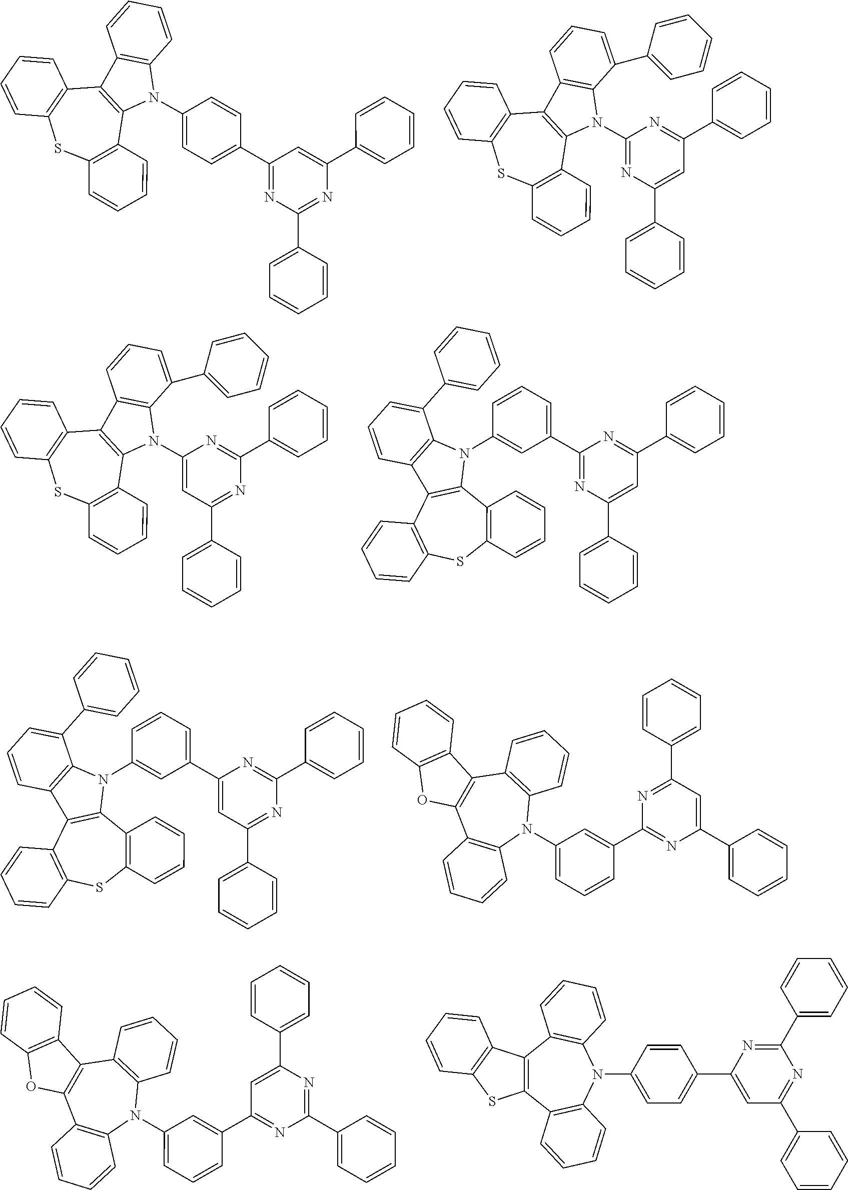

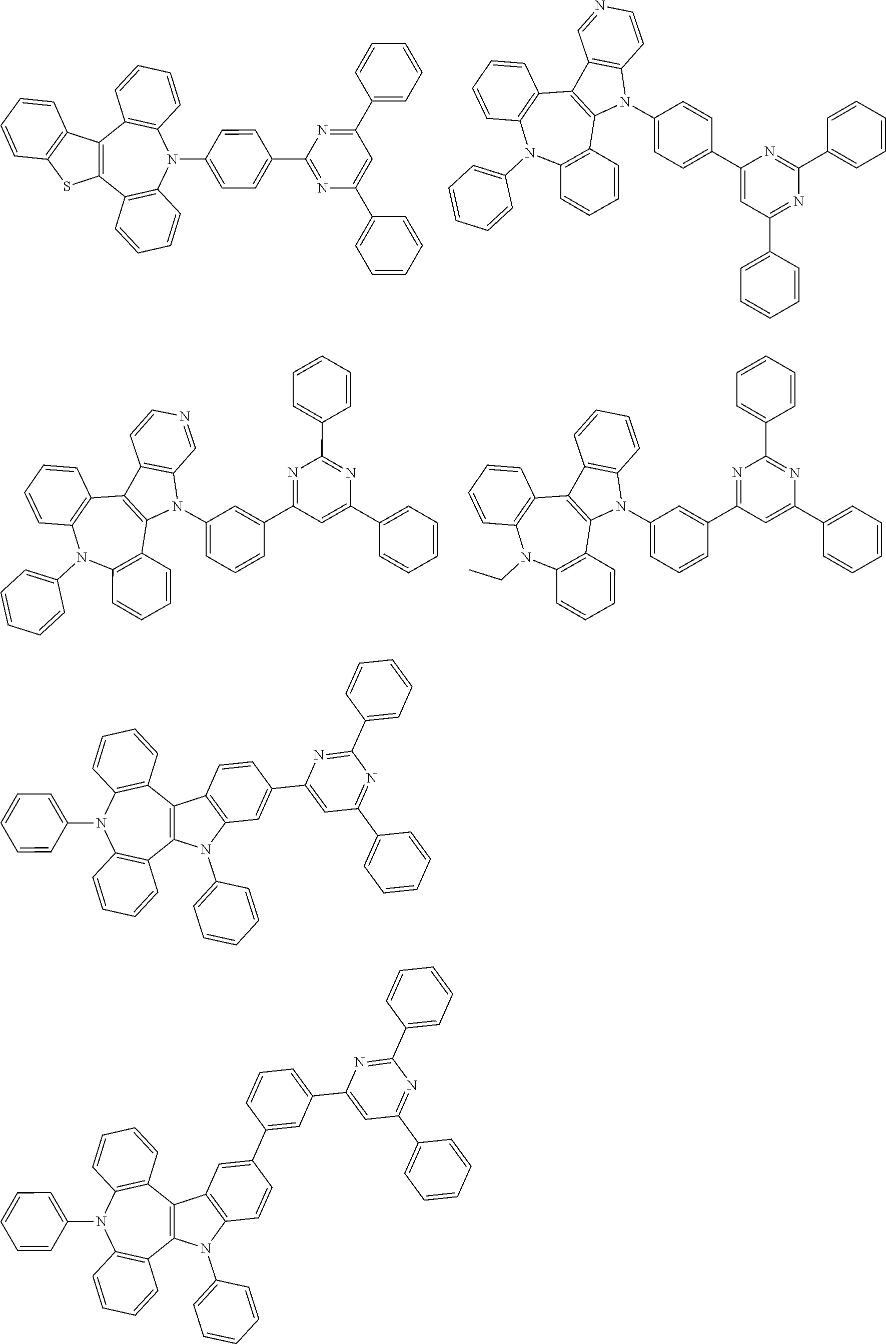

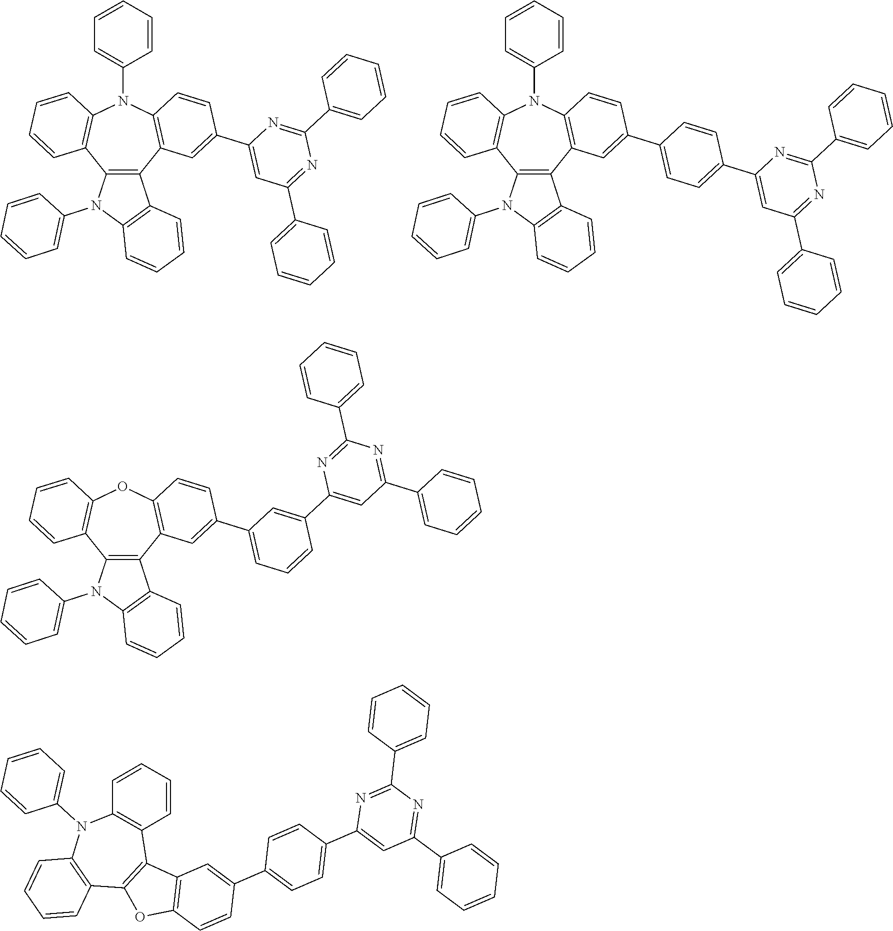 [SMILES: c1ccc(-c2cc(-c3ccc4c(c3)-c3c(n(-c5ccccc5)c5ccccc35)-c3ccccc3N4c3ccccc3)nc(-c3ccccc3)n2)cc1 ; c1ccc(-c2cc(-c3ccc(-c4ccc5c(c4)-c4c(n(-c6ccccc6)c6ccccc46)-c4ccccc4N5c4ccccc4)cc3)nc(-c3ccccc3)n2)cc1 ; c1ccc(-c2cc(-c3cccc(-c4ccc5c(c4)-c4c(n(-c6ccccc6)c6ccccc46)-c4ccccc4O5)c3)nc(-c3ccccc3)n2)cc1 ; c1ccc(-c2cc(-c3ccc(-c4ccc5oc6c(c5c4)-c4ccccc4N(c4ccccc4)c4ccccc4-6)cc3)nc(-c3ccccc3)n2)cc1]

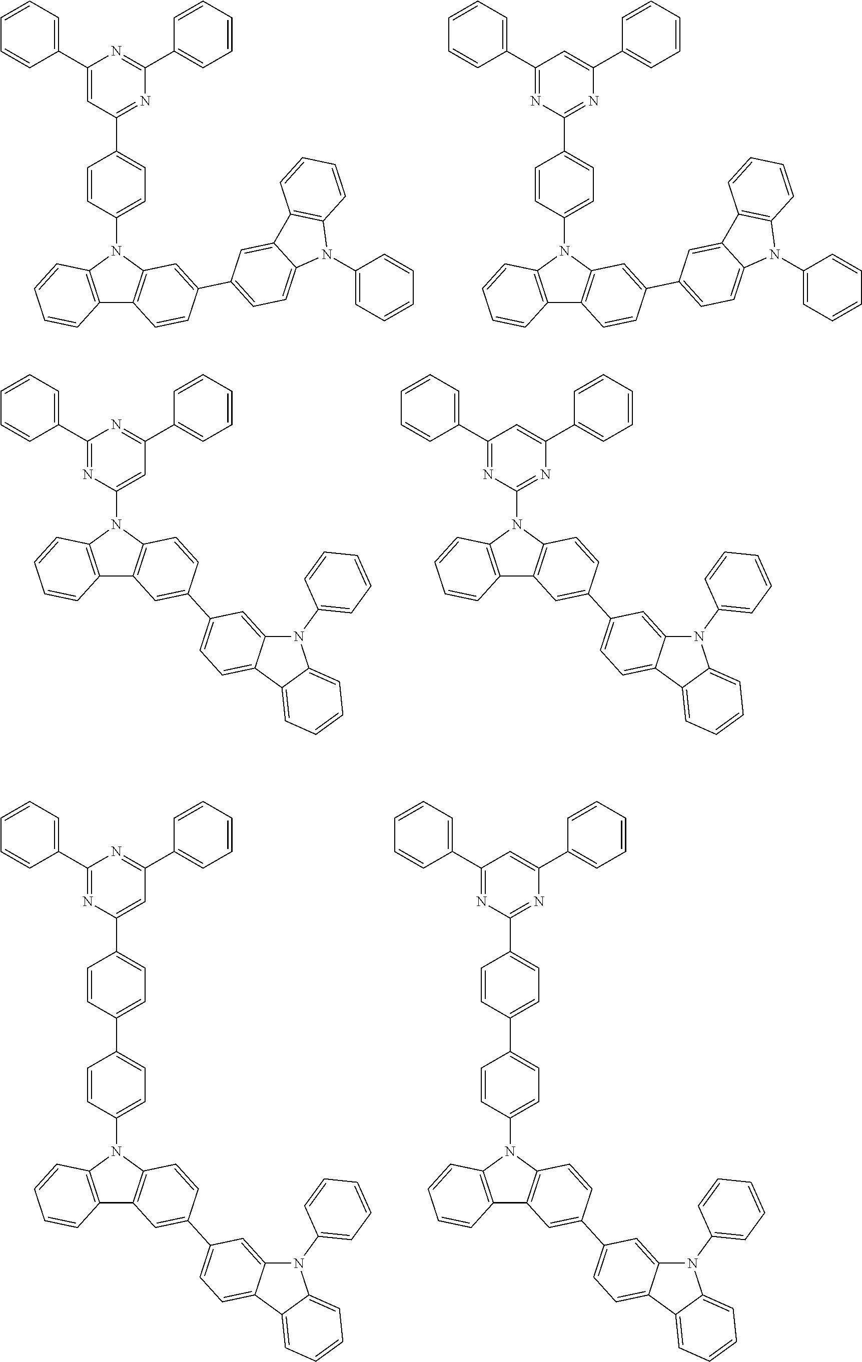

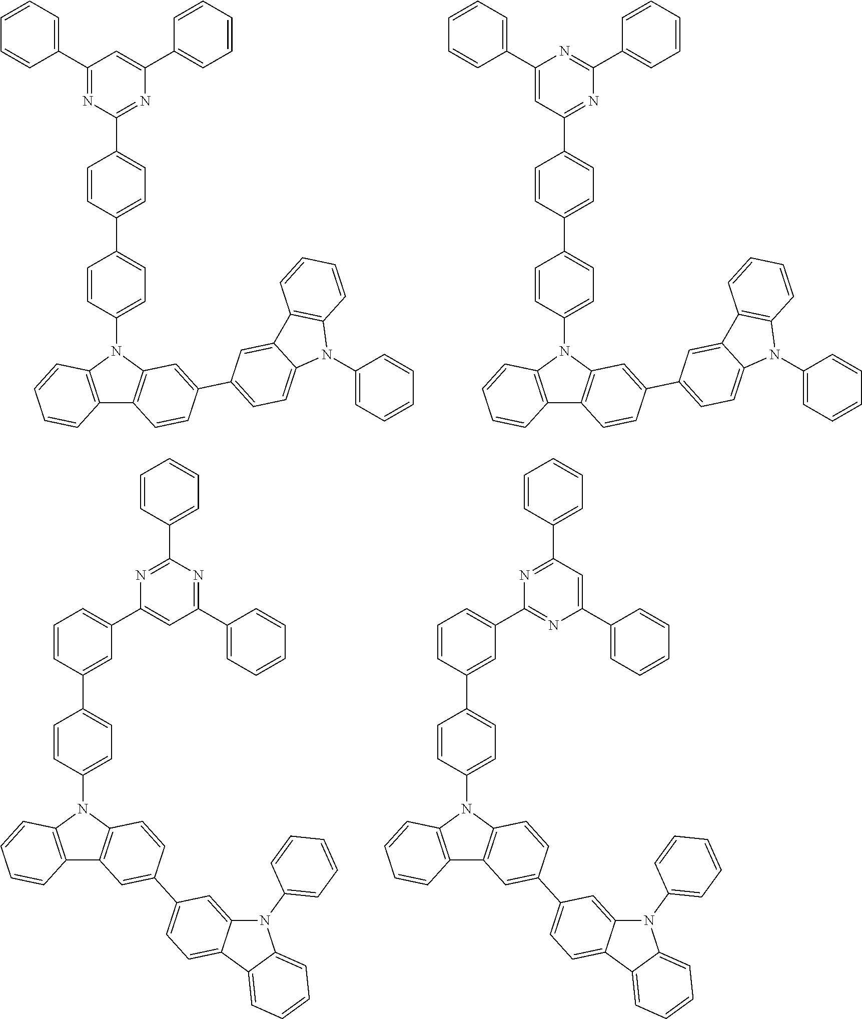

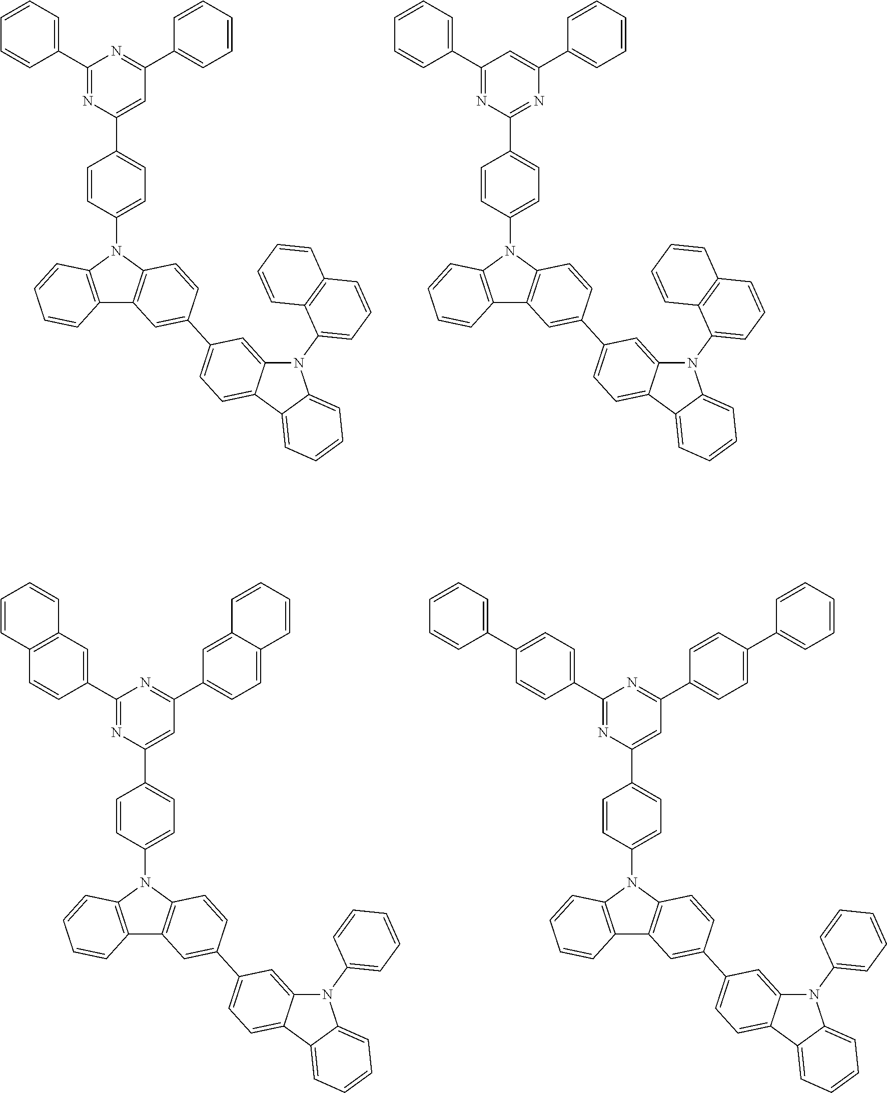

In one embodiment, in the compound represented by the formulas (A1) to (D1), the substituent in the case of “substituted or unsubstituted” is a group selected from the group consisting of:

an alkyl group including 1 to 50 carbon atoms,
an aryl group including 6 to 50 ring carbon atoms, and
a monovalent heterocyclic group including 5 to 50 ring atoms.

In one embodiment, in the compound represented by the formulas (A1) to (D1), the substituent in the case of “substituted or unsubstituted” is a group selected from the group consisting of:

an alkyl group including 1 to 18 carbon atoms,
an aryl group including 6 to 18 ring carbon atoms, and
a monovalent heterocyclic group including 5 to 18 ring atoms.

Specific examples of each group in the formulas (A1) to (D1) are as described in the section of [Definition] of this specification.

As described above, known materials and device configurations may be applied to the organic EL device according to an aspect of the invention, as long as the device contains a cathode; an anode; and an organic layer between the cathode and the anode, wherein the organic layer contains an emitting layer and a first layer; the first layer is disposed between the anode and the emitting layer and is directly adjacent to the emitting layer; the emitting layer contains a compound represented by the formula (A1); and the first layer contains a compound represented by the formula (B1) or the formula (C1); and the effect of the invention is not impaired.

Parts which can be used in the organic EL device according to an aspect of the invention, materials for forming respective layers, other than the above-mentioned compounds, and the like, will be described later.

### (Substrate)

A substrate is used as a support of an emitting device. As the substrate, glass, quartz, plastic or the like can be used, for example. Further, a flexible substrate may be used. The “flexible substrate” means a bendable (flexible) substrate, and specific examples thereof include a plastic substrate formed of polycarbonate, polyvinyl chloride, or the like.

### (Anode)

For the anode formed on the substrate, metals, alloys, electrically conductive compounds, mixtures thereof, and the like, which have a large work function (specifically 4.0 eV or more) are preferably used. Specific examples include indium oxide-tin oxide (ITO: Indium Tin Oxide), indium oxide-tin oxide containing silicon or silicon oxide; indium oxide-zinc oxide, tungsten oxide, indium oxide containing zinc oxide, graphene, and the like. In addition thereto, specific examples thereof include gold (Au), platinum (Pt), a nitride of a metallic material (for example, titanium nitride), and the like.

### (Hole-Injecting Layer)

The hole-injecting layer is a layer containing a substance having high hole-injecting property. As such a substance having high hole-injecting property molybdenum oxide, titanium oxide, vanadium oxide, rhenium oxide, ruthenium oxide, chromium oxide, zirconium oxide, hafnium oxide, tantalum oxide, silver oxide, tungsten oxide, manganese oxide, an aromatic amine compound, or a polymer compound (oligomers, dendrimers, polymers, etc.) can be given.

### (Hole-Transporting Layer)

The hole-transporting layer is a layer containing a substance having high hole-transporting property. For the hole-transporting layer, an aromatic amine compound, a carbazole derivative, an anthracene derivative, or the like can be used. A polymer compound such as poly(N-vinylcarbazole) (abbreviation: PVK) and poly(4-vinyltriphenylamine) (abbreviation: PVTPA) can also be used. However, a substance other than the above-described substances may be used as long as the substance has higher hole-transporting property in comparison with an electron-transporting property. It should be noted that the layer containing the substance having high hole-transporting property may be not only a single layer, but also a layer in which two or more layers formed of the above-described substances are stacked.

In the invention, the electron barrier layer may contain the above-described materials.

### (Guest Material for Emitting Layer)

The emitting layer is a layer containing a substance having a high emitting property, and various materials can be used for forming it. For example, as the substance having a high emitting property, a fluorescent compound which emits fluorescence or a phosphorescent compound which emits phosphorescence can be used. The fluorescent compound is a compound which can emit from a singlet excited state, and the phosphorescent compound is a compound which can emit from a triplet excited state.

As a blue fluorescent emitting material which can be used for an emitting layer, pyrene derivatives, styrylamine derivatives, chrysene derivatives, fluoranthene derivatives, fluorene derivatives, diamine derivatives, triarylamine derivatives, and the like can be used. As a green fluorescent emitting material which can be used for an emitting layer, aromatic amine derivatives and the like can be used. As a red fluorescent emitting material which can be used for an emitting layer, tetracene derivatives, diamine derivatives and the like can be used.

As a blue phosphorescent emitting material which can be used for an emitting layer, metal complexes such as iridium complexes, osmium complexes, platinum complexes and the like are used. As a green phosphorescent emitting material which can be used for an emitting layer, iridium complexes and the like are used. As a red phosphorescent emitting material which can be used for an emitting layer, metal complexes such as iridium complexes, platinum complexes, terbium complexes, europium complexes and the like are used.

### (Host Material for Emitting Layer)

The emitting layer may have a constitution in which the substance having a high emitting property (guest material) is dispersed in another substance (host material). As a substance for dispersing the substance having a high emitting property, a variety of substances can be used, and it is preferable to use a substance having a higher lowest unoccupied orbital level (LUMO level) and a lower highest occupied orbital level (HOMO level) than the substance having a high emitting property.

As a substance for dispersing the substance having a high emitting property (host material), 1) metal complexes such as aluminum complexes, beryllium complexes, zinc complexes, or the like; 2) heterocyclic compounds such as oxadiazole derivatives, benzimidazole derivatives, phenanthroline derivatives, or the like; 3) fused aromatic compounds such as carbazole derivatives, anthracene derivatives, phenanthrene derivatives, pyrene derivatives, chrysene derivatives, or the like; and 3) aromatic amine compound such as triarylamine derivatives, aromatic amine derivatives, or the like are used.

### (Electron-Transporting Layer)

An electron-transporting layer is a layer which contains a substance having high electron-transporting property. For the electron-transporting layer, 1) metal complexes such as aluminum complexes, beryllium complexes, zinc complexes, or the like; 2) heteroaromatic complexes such as imidazole derivatives, benzimidazole derivatives, azine derivatives, carbazole derivatives, phenanthroline derivatives, or the like; and 3) polymer compounds can be used.

### (Electron-Injecting Layer)

An electron-injecting layer is a layer which contains a substance having a high electron-injecting property. For the electron-injecting layer, metal complex compounds such as lithium (Li), ytterbium (Yb), lithium fluoride (LiF), cesium fluoride (CsF), calcium fluoride (CaF₂), 8-hydroxyquinolinolato-lithium (Liq); alkali metals such as lithium oxide (LiOₓ); alkaline earth metals; or a compound thereof can be used.

### (Cathode)

For the cathode, metals, alloys, electrically conductive compounds, mixtures thereof, and the like having a small work function (specifically, 3.8 eV or less) are preferably used. Specific examples of such a cathode material include elements belonging to Group 1 or Group 2 of the Periodic Table of the Elements, i.e., alkali metals such as lithium (Li) and cesium (Cs), alkaline earth metals such as magnesium (Mg), calcium (Ca) and strontium (Sr), and alloys containing these metals (e.g., MgAg and AlLi); rare earth metals such as europium (Eu) and ytterbium (Yb), and alloys containing these metals.

In the organic EL device according to an aspect of the invention, the methods for forming the respective layers are not particularly limited. A conventionally-known method for forming each layer according to a vacuum deposition process, a spin coating process or the like can be used. Each layer such as the emitting layer can be formed by a known method such as a vacuum deposition process, a molecular beam deposition process (MBE process), or an application process such as a dipping process, a spin coating process, a casting process, a bar coating process and a roll coating process, using a solution prepared by dissolving the material in a solvent.

In the organic EL device according to an aspect of the invention, the thickness of each layer is not particularly limited, but is generally preferable that the thickness be in the range of several nm to 1 μm in order to suppress defects such as pinholes, to suppress applied voltages to be low, and to improve luminous efficiency.

### [Electronic Appliance]

The electronic appliance according to an aspect of the invention is characterized in that the organic EL device according to an aspect of the invention is provided.

Specific examples of the electronic appliance include a display component such as an organic EL panel module, and the like; a display device such as a television, a cellular phone, a personal computer, and the like; and an emitting device such as a light, a vehicular lamp, and the like.

### EXAMPLES

Next, the invention will be explained in more detail with reference to Examples and Comparative Examples. The invention is not limited to the description of these Examples in any way.

### <Compound>

The compounds represented by the formula (A1) used for fabricating the organic EL device of Examples 1 to 164 are shown below.

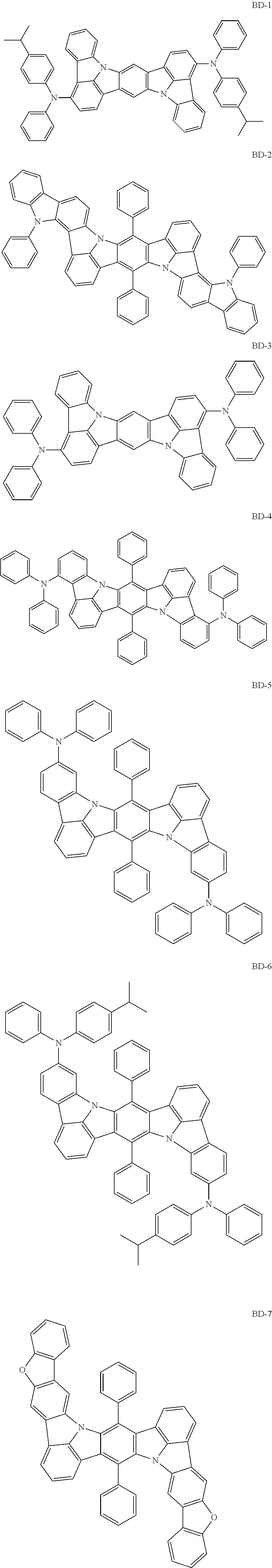

The compounds represented by the formula (B1) or (C1) used for fabricating the organic EL device of Examples 1 to 164 are shown below.

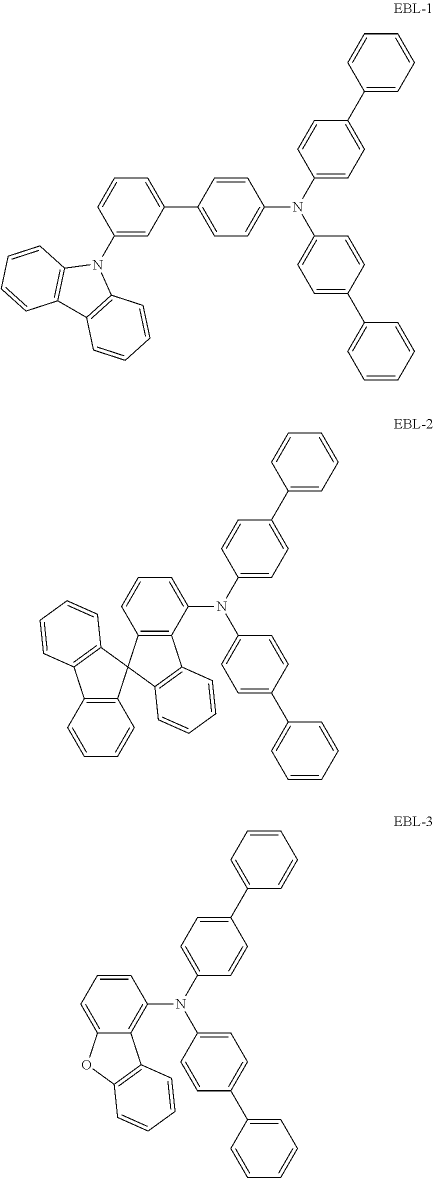 [SMILES: c1ccc(-c2ccc(N(c3ccc(-c4ccccc4)cc3)c3ccc(-c4cccc(-n5c6ccccc6c6ccccc65)c4)cc3)cc2)cc1 ; c1ccc(-c2ccc(N(c3ccc(-c4ccccc4)cc3)c3cccc4c3-c3ccccc3C43c4ccccc4-c4ccccc43)cc2)cc1 ; c1ccc(-c2ccc(N(c3ccc(-c4ccccc4)cc3)c3cccc4oc5ccccc5c34)cc2)cc1]

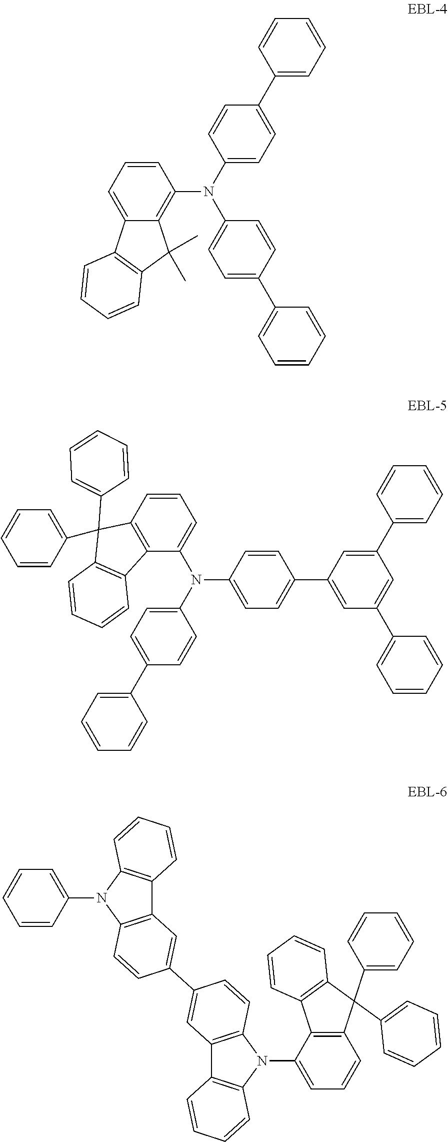 [SMILES: CC1(C)c2ccccc2-c2cccc(N(c3ccc(-c4ccccc4)cc3)c3ccc(-c4ccccc4)cc3)c21 ; c1ccc(-c2ccc(N(c3ccc(-c4cc(-c5ccccc5)cc(-c5ccccc5)c4)cc3)c3cccc4c3-c3ccccc3C4(c3ccccc3)c3ccccc3)cc2)cc1 ; c1ccc(-n2c3ccccc3c3cc(-c4ccc5c(c4)c4ccccc4n5-c4cccc5c4-c4ccccc4C5(c4ccccc4)c4ccccc4)ccc32)cc1]

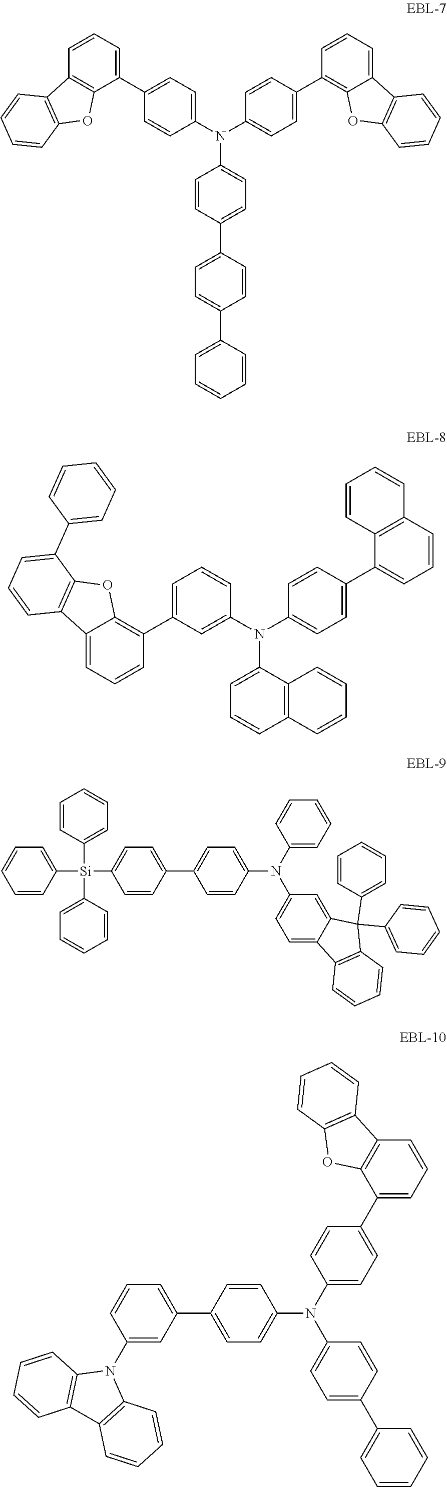 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2cccc3c2oc2c(-c4cccc(N(c5ccc(-c6cccc7ccccc67)cc5)c5cccc6ccccc56)c4)cccc23)cc1 ; c1ccc(N(c2ccc(-c3ccc([Si](c4ccccc4)(c4ccccc4)c4ccccc4)cc3)cc2)c2ccc3c(c2)C(c2ccccc2)(c2ccccc2)c2ccccc2-3)cc1 ; c1ccc(-c2ccc(N(c3ccc(-c4cccc(-n5c6ccccc6c6ccccc65)c4)cc3)c3ccc(-c4cccc5c4oc4ccccc45)cc3)cc2)cc1]

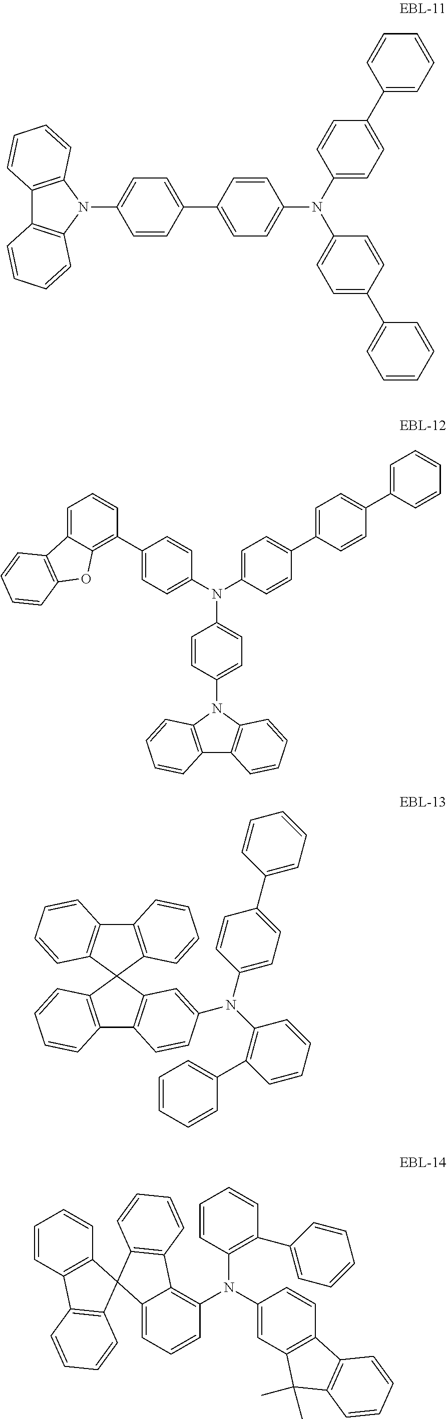

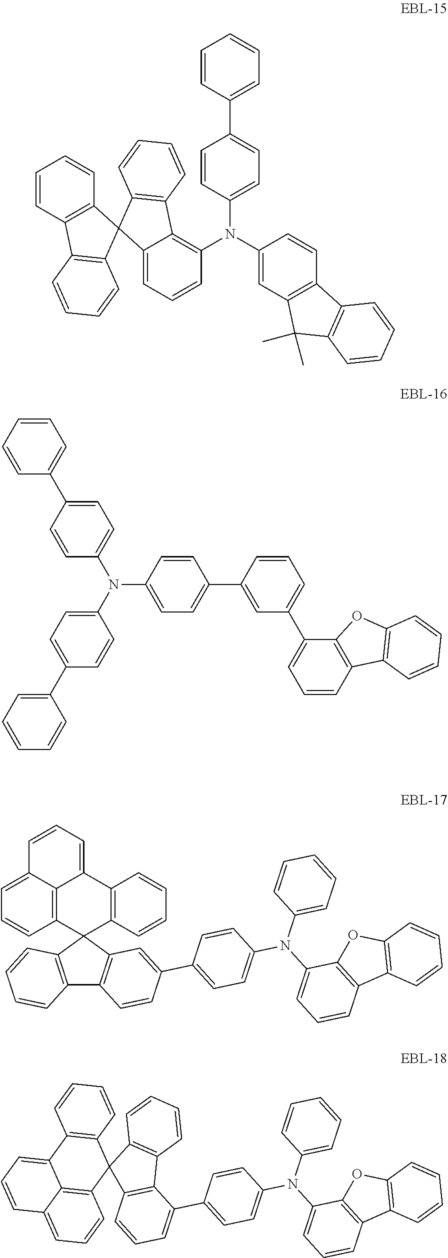 [SMILES: CC1(C)c2ccccc2-c2ccc(N(c3ccc(-c4ccccc4)cc3)c3cccc4c3-c3ccccc3C43c4ccccc4-c4ccccc43)cc21 ; c1ccc(-c2ccc(N(c3ccc(-c4ccccc4)cc3)c3ccc(-c4cccc(-c5cccc6c5oc5ccccc56)c4)cc3)cc2)cc1 ; c1ccc(N(c2ccc(-c3ccc4c(c3)C3(c5ccccc5-4)c4ccccc4-c4cccc5cccc3c45)cc2)c2cccc3c2oc2ccccc23)cc1 ; c1ccc(N(c2ccc(-c3cccc4c3-c3ccccc3C43c4ccccc4-c4cccc5cccc3c45)cc2)c2cccc3c2oc2ccccc23)cc1]

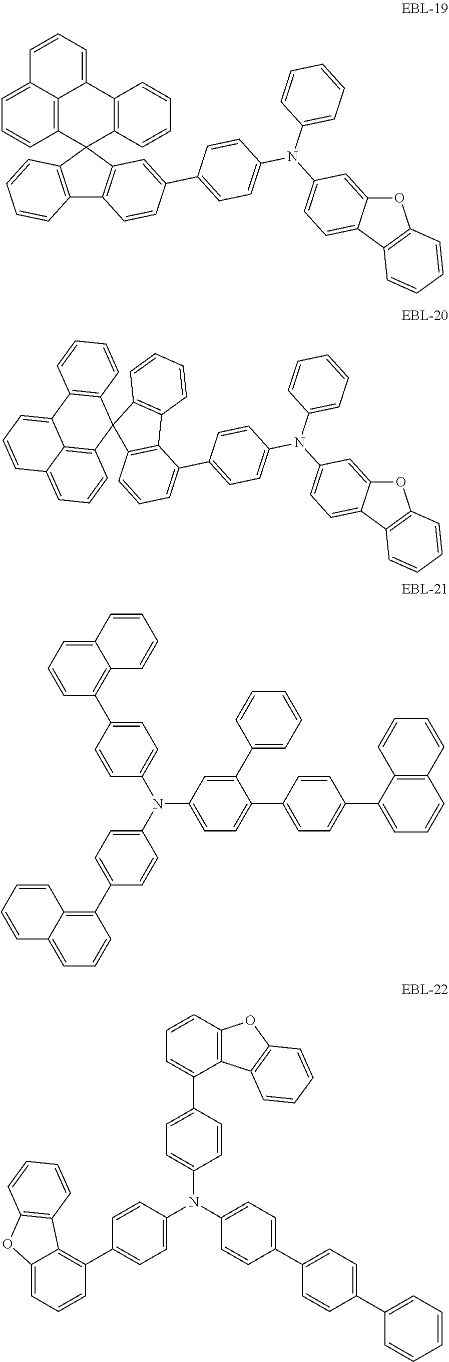 [SMILES: c1ccc(N(c2ccc(-c3ccc4c(c3)C3(c5ccccc5-4)c4ccccc4-c4cccc5cccc3c45)cc2)c2ccc3c(c2)oc2ccccc23)cc1 ; c1ccc(N(c2ccc(-c3cccc4c3-c3ccccc3C43c4ccccc4-c4cccc5cccc3c45)cc2)c2ccc3c(c2)oc2ccccc23)cc1 ; c1ccc(-c2cc(N(c3ccc(-c4cccc5ccccc45)cc3)c3ccc(-c4cccc5ccccc45)cc3)ccc2-c2ccc(-c3cccc4ccccc34)cc2)cc1 ; c1ccc(-c2ccc(-c3ccc(N(c4ccc(-c5cccc6oc7ccccc7c56)cc4)c4ccc(-c5cccc6oc7ccccc7c56)cc4)cc3)cc2)cc1]

The compound represented by the formula (D1) used for fabricating the organic EL device of Examples 1 to 20 and Comparative Examples 1 to 20 is shown below.

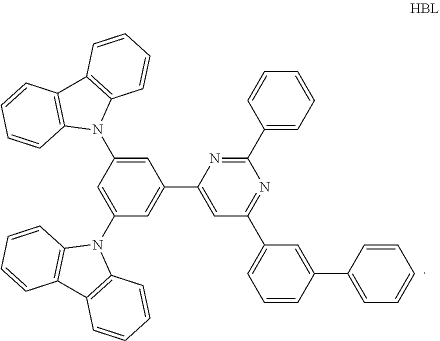 [SMILES: c1ccc(-c2cccc(-c3cc(-c4cc(-n5c6ccccc6c6ccccc65)cc(-n5c6ccccc6c6ccccc65)c4)nc(-c4ccccc4)n3)c2)cc1]

Comparative compounds used for fabricating the organic EL device of Comparative Examples 1 to 61 are shown below.

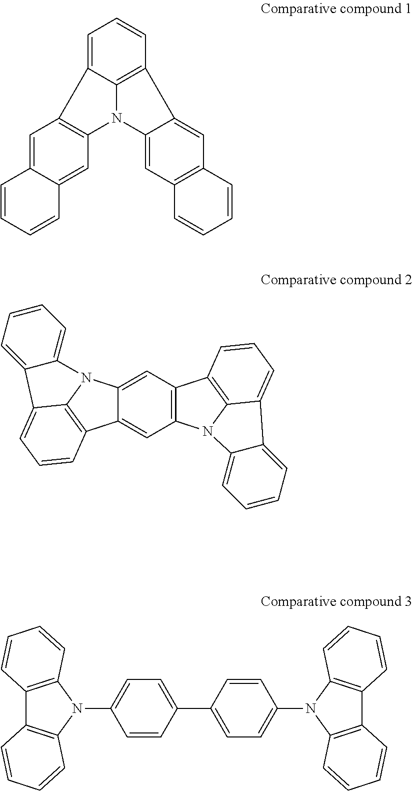 [SMILES: c1ccc2cc3c(cc2c1)c1cccc2c4cc5ccccc5cc4n3c12 ; c1ccc2c(c1)c1cccc3c4cc5c(cc4n2c13)c1cccc2c3ccccc3n5c21 ; c1ccc2c(c1)c1ccccc1n2-c1ccc(-c2ccc(-n3c4ccccc4c4ccccc43)cc2)cc1]

The structure of the other compounds used for fabricating the organic EL device of Examples 1 to 20 and Comparative Examples 1 to 20 are shown below.

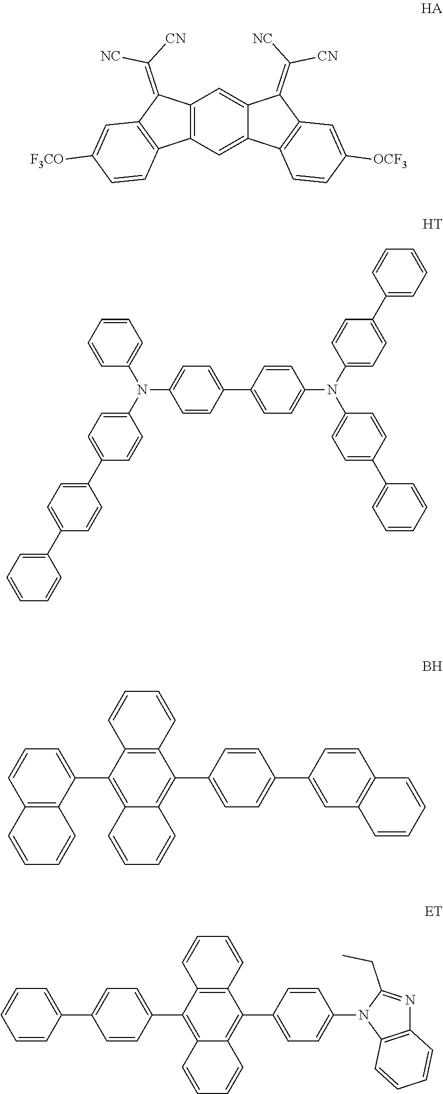

### <Fabrication of Organic EL Device>

An organic EL device was fabricated and evaluated as follows.

### Example 1

A 25 mm×75 mm×1.1 mm-thick glass substrate with an ITO transparent electrode (anode) (manufactured by GEOMATEC Co., Ltd.) was subjected to ultrasonic cleaning in isopropyl alcohol for 5 minutes, and then subjected to UV-ozone cleaning for 30 minutes. The thickness of the ITO film was 130 nm.

The glass substrate with the transparent electrode after being cleaned was mounted onto a substrate holder in a vacuum vapor deposition apparatus. First, a compound HA was deposited on a surface on the side on which the transparent electrode was formed so as to cover the transparent electrode to form an HA film having a thickness of 5 nm. The HA film functions as a hole-injecting layer.

A compound HT was deposited on the HA film to form an HT film having a thickness of 80 nm. The HT film functions as a hole-transporting layer (hereinafter, also referred to as an HT layer).

A compound EBL-1 was deposited on the HT film to form an EBL-1 film (a first layer) having a thickness of 10 nm. The EBL-1 film functions as an electron barrier layer (hereinafter, also referred to as an EBL layer).

A compound BH (host material) and a compound BD-1 (dopant material) were co-deposited on the EBL-1 film such that the proportion of the compound BD-1 became 4 mass %, and a BH:BD-1 film having a thickness of 25 nm was formed. The BH:BD-1 film functions as an emitting layer.

A compound HBL was deposited on the emitting layer to form an HBL film having a thickness of 10 nm. The HBL film functions as a first electron-transporting layer.

A compound ET was deposited on the HBL film to form an ET film having a thickness of 15 nm. The ET film functions as a second electron-transporting layer.

LiF was deposited on the ET film to form a LiF film having a thickness of 1 nm.

Al metal was deposited on the LiF film to form a metal cathode having a thickness of 80 nm to obtain an organic EL device.

The layer configuration of the obtained organic EL device is as follows. ITO(130)/HA(5)/HT(80)/EBL-1(10)/BH:BD-1(25:4 mass %)/HBL(10)/ET(15)/LiF(1)/Al(80)

Numerical values in parentheses indicate film thickness (unit: nm).

### (Evaluation of Organic EL Device)

Voltage was applied to the obtained organic EL device to be 10 mA/cm² in current density, thereby measuring an EL emission spectrum by using Spectroradiometer CS-1000 (manufactured by Konica Minolta, Inc.). External quantum efficiency (EQE (unit:%)) was calculated from the resulting spectral radiance spectrum. The results are shown in Table 1.

### Examples 2 to 10

The organic EL devices were fabricated and evaluated in the same manner as in Example 1 except that the compounds shown in Table 1 were used as materials of the EBL layer (the first layer). The results are shown in Table 1.

### Comparative Examples 1 to 10

The organic EL devices were fabricated and evaluated in the same manner as in Example 1 except that the comparative compound 1 was used in place of the compound BD-1 (dopant material) and the compounds shown in Table 1 were used as materials of the first layer. The results are shown in Table 1.

### Examples 11 to 20

The organic EL devices were fabricated and evaluated in the same manner as in Example 1 except that the compound BD-2 was used in place of the compound BD-1 (dopant material) and the compounds shown in Table 1 were used as materials of the first layer. The results are shown in Table 1.

### Comparative Examples 11 to 20

The organic EL devices were fabricated and evaluated in the same manner as in Example 1 except that the comparative compound 2 was used in place of the compound BD-1 (dopant material) and the compounds shown in Table 1 were used as materials of the first layer. The results are shown in Table 1.

TABLE 1

| | | First | First electron- | |
|---|---|---|---|---|
| | Dopant | layer | transporting layer | EQE(%) |
| Ex. 1 | BD-1 | EBL-1 | HBL | 10.1 |
| Ex. 2 | BD-1 | EBL-2 | HBL | 10.6 |
| Ex. 3 | BD-1 | EBL-3 | HBL | 10.5 |
| Ex. 4 | BD-1 | EBL-4 | HBL | 10.7 |
| Ex. 5 | BD-1 | EBL-5 | HBL | 11.2 |
| Ex. 6 | BD-1 | EBL-6 | HBL | 10.8 |
| Ex. 7 | BD-1 | EBL-7 | HBL | 9.5 |
| Ex. 8 | BD-1 | EBL-8 | HBL | 10.3 |
| Ex. 9 | BD-1 | EBL-9 | HBL | 10.8 |
| Ex. 10 | BD-1 | EBL-10 | HBL | 10.5 |
| Ex. 11 | BD-2 | EBL-1 | HBL | 8.0 |
| Ex. 12 | BD-2 | EBL-2 | HBL | 8.4 |
| Ex. 13 | BD-2 | EBL-3 | HBL | 8.3 |
| Ex. 14 | BD-2 | EBL-4 | HBL | 8.4 |
| Ex. 15 | BD-2 | EBL-5 | HBL | 8.9 |
| Ex. 16 | BD-2 | EBL-6 | HBL | 8.5 |
| Ex. 17 | BD-2 | EBL-7 | HBL | 7.5 |
| Ex. 18 | BD-2 | EBL-8 | HBL | 8.1 |
| Ex. 19 | BD-2 | EBL-9 | HBL | 8.5 |
| Ex. 20 | BD-2 | EBL-10 | HBL | 8.3 |
| Comp. | Comp. | EBL-1 | HBL | 4.8 |
| Ex. 1 | compound-1 | | | |
| Comp. | Comp. | EBL-2 | HBL | 5.1 |
| Ex. 2 | compound-1 | | | |
| Comp. | Comp. | EBL-3 | HBL | 5.0 |
| Ex. 3 | compound-1 | | | |
| Comp. | Comp. | EBL-4 | HBL | 5.1 |
| Ex. 4 | compound-1 | | | |
| Comp. | Comp. | EBL-5 | HBL | 5.4 |
| Ex. 5 | compound-1 | | | |
| Comp. | Comp. | EBL-6 | HBL | 5.1 |
| Ex. 6 | compound-1 | | | |
| Comp. | Comp. | EBL-7 | HBL | 4.5 |
| Ex. 7 | compound-1 | | | |
| Comp. | Comp. | EBL-8 | HBL | 4.9 |
| Ex. 8 | compound-1 | | | |
| Comp. | Comp. | EBL-9 | HBL | 5.1 |
| Ex. 9 | compound-1 | | | |
| Comp. | Comp. | EBL-10 | HBL | 5.0 |
| Ex. 10 | compound-1 | | | |
| Comp. | Comp. | EBL-1 | HBL | 5.4 |
| Ex. 11 | compound-2 | | | |
| Comp. | Comp. | EBL-2 | HBL | 5.7 |
| Ex. 12 | compound-2 | | | |
| Comp. | Comp. | EBL-3 | HBL | 5.6 |
| Ex. 13 | compound-2 | | | |
| Comp. | Comp. | EBL-4 | HBL | 5.7 |
| Ex. 14 | compound-2 | | | |
| Comp. | Comp. | EBL-5 | HBL | 6.0 |
| Ex. 15 | compound-2 | | | |
| Comp. | Comp. | EBL-6 | HBL | 5.8 |
| Ex. 16 | compound-2 | | | |
| Comp. | Comp. | EBL-7 | HBL | 5.1 |
| Ex. 17 | compound-2 | | | |
| Comp. | Comp. | EBL-8 | HBL | 5.5 |
| Ex. 18 | compound-2 | | | |
| Comp. | Comp. | EBL-9 | HBL | 5.8 |
| Ex. 19 | compound-2 | | | |
| Comp. | Comp. | EBL-10 | HBL | 5.6 |
| Ex. 20 | compound-2 | | | |

From the results shown in Table 1, it can be seen that the organic EL devices of Examples 1 to 20, which contain a particular dopant material in the emitting layer and a particular material in the first layer, are highly efficient.

### Example 21

The organic EL device was fabricated as follows and evaluated in the same manner as in Example 1. The results are shown in Table 2.

A 25 mm×75 mm×1.1 mm-thick glass substrate with an ITO transparent electrode (anode) (manufactured by GEOMATEC Co., Ltd.) was subjected to ultrasonic cleaning in isopropyl alcohol for 5 minutes, and then subjected to UV-ozone cleaning for 30 minutes. The thickness of the ITO film was 130 nm.

The glass substrate with the transparent electrode after being cleaned was mounted onto a substrate holder in a vacuum vapor deposition apparatus. First, the following compound HT-2 and the following compound HA-2 were co-deposited such that the proportion of the compound HA-2 became 3 mass % on a surface on the side on which the transparent electrode was formed so as to cover the transparent electrode to form a HT-2:HA-2 film having a thickness of 5 nm. The HT-2:HA-2 film functions as a hole-injecting layer.

A compound HT-2 was deposited on the HT-2:HA-2 film to form an HT-2 film having a thickness of 80 nm. The HT-2 film functions as a hole-transporting layer (hereinafter, also referred to as an HT layer).

A compound EBL-2 was deposited on the HT-2 film to form an EBL-2 film (a first layer) having a thickness of 10 nm. The EBL-2 film functions as an electron barrier layer (hereinafter, also referred to as an EBL layer).

The compound BH (host material) and the compound BD-1 (dopant material) were co-deposited on the EBL-2 film such that the proportion of the compound BD-1 became 2 mass % to form a BH:BD-1 film having a thickness of 25 nm. The BH:BD-1 film functions as an emitting layer.

The following compound HBL-2 was deposited on the emitting layer to form an HBL-2 film having a thickness of 10 nm. The HBL-2 film functions as a first electron-transporting layer (hereinafter, also referred to as an HBL layer).

The following compound ET-2 and Li were co-deposited on the HBL-2 film such that the proportion of Li became 4 mass % to form an ET-2:Li film having a thickness of 15 nm. The ET-2:Li film functions as a second electron-transporting layer.

Al metal was deposited on the ET-2:LiF film to form a metal cathode having a thickness of 80 nm to obtain an organic EL device.

The layer configuration of the obtained organic EL device is as follows. ITO (130)/HT-2:HA-2 (5:3 mass %)/HT-2 (80)/EBL-2 (10)/BH:BD-1 (25:2 mass %)/HBL-2 (10)/ET-2:Li (15:4 mass %)/Al (80)

Numerical values in parentheses indicate film thickness (unit: nm).

### Examples 22 to 28

The organic EL devices were fabricated in the same manner as in Example 1 and evaluated in the same manner as in Example 21 except that the compounds shown in Table 2 were used as materials of a hole-injecting layer, a hole-transporting layer, a hole barrier layer (a first layer), a dopant material of an emitting layer, and a first electron-transporting layer. The results are shown in Table 2.

### Comparative Examples 21 to 28

The organic EL devices were fabricated in the same manner as in Example 1 and evaluated in the same manner as in Example 21 except that the compounds shown in Table 2 were used as materials of a hole-injecting layer, a hole-transporting layer, a hole barrier layer (a first layer), a dopant material of an emitting layer, and a first electron-transporting layer. The results are shown in Table 2.

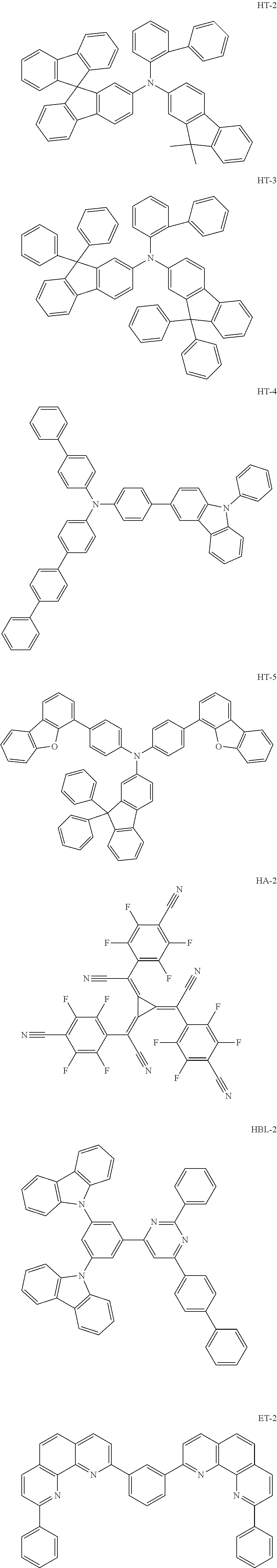

TABLE 2

| | | | | | First | |
|---|---|---|---|---|---|---|
| | | | Hole- | | electron- | |
| | | Hole | trans- | | trans- | |
| | | injecting | porting | First | porting | |
| | Dopant | layer | layer | layer | layer | EQE(%) |
| Ex. 21 | BD-1 | HT-2: | HT-2 | EBL-2 | HBL-2 | 9.5 |
| | | HA-2 | | | | |
| Ex. 22 | BD-1 | HT-3: | HT-3 | EBL-2 | HBL-2 | 8.9 |
| | | HA-2 | | | | |
| Ex. 23 | BD-1 | HT-4: | HT-4 | EBL-2 | HBL-2 | 9 |
| | | HA-2 | | | | |
| Ex. 24 | BD-1 | HT-5: | HT-5 | EBL-2 | HBL-2 | 9.1 |
| | | HA-2 | | | | |
| Ex. 25 | BD-2 | HT-2: | HT-2 | EBL-2 | HBL-2 | 8 |
| | | HA-2 | | | | |
| Ex. 26 | BD-2 | HT-3: | HT-3 | EBL-2 | HBL-2 | 7.7 |
| | | HA-2 | | | | |
| Ex. 27 | BD-2 | HT-4: | HT-4 | EBL-2 | HBL-2 | 7.7 |
| | | HA-2 | | | | |
| Ex. 28 | BD-2 | HT-5: | HT-5 | EBL-2 | HBL-2 | 7.9 |
| | | HA-2 | | | | |
| Comp. | Comp. | HT-2: | HT-2 | EBL-2 | HBL-2 | 4.5 |
| Ex. 21 | compound-1 | HA-2 | | | | |
| Comp. | Comp. | HT-3: | HT-3 | EBL-2 | HBL-2 | 4.2 |
| Ex. 22 | compound-1 | HA-2 | | | | |
| Comp. | Comp. | HT-4: | HT-4 | EBL-2 | HBL-2 | 4.3 |
| Ex. 23 | compound-1 | HA-2 | | | | |
| Comp. | Comp. | HT-5: | HT-5 | EBL-2 | HBL-2 | 4.3 |
| Ex. 24 | compound-1 | HA-2 | | | | |
| Comp. | Comp. | HT-2: | HT-2 | EBL-2 | HBL-2 | 5.1 |
| Ex. 25 | compound-2 | HA-2 | | | | |
| Comp. | Comp. | HT-3: | HT-3 | EBL-2 | HBL-2 | 4.7 |
| Ex. 26 | compound-2 | HA-2 | | | | |
| Comp. | Comp. | HT-4: | HT-4 | EBL-2 | HBL-2 | 4.8 |
| Ex. 27 | compound-2 | HA-2 | | | | |
| Comp. | Comp. | HT-5: | HT-5 | EBL-2 | HBL-2 | 5 |
| Ex. 28 | compound-2 | HA-2 | | | | |

From the results shown in Table 2, it can be seen that the organic EL devices of Examples 21 to 28, which contain a particular dopant material in the emitting layer and a particular material in the first layer, are highly efficient.

Further, it can be seen that if the emitting layer and the first layer contain a particular material, a highly efficient organic EL device can be obtained even if the materials of the hole-injecting layer and the hole-transporting layer are changed.

### Examples 29 to 30 and Comparative Examples 29 to 30

The organic EL devices of Example 29, Example 30, Comparative Example 29, and Comparative Example 30 were fabricated in the same manner as in Example 21, Example 25, Comparative Example 21, and Comparative Example 25, respectively, except that the following compound HBL-3 was used as a material of the first electron-transporting layer. These were evaluated in the same manner as in Example 1. Results are shown in Table 3.

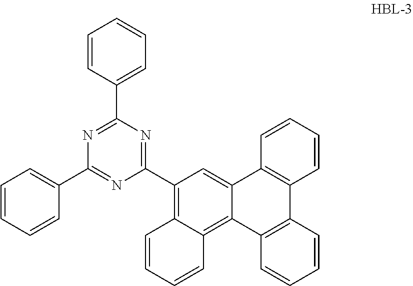 [SMILES: c1ccc(-c2nc(-c3ccccc3)nc(-c3cc4c5ccccc5c5ccccc5c4c4ccccc34)n2)cc1]

TABLE 3

| | | | | | First | |
|---|---|---|---|---|---|---|
| | | | Hole- | | electron- | |
| | | Hole | trans- | | trans - | |
| | | injecting | porting | First | porting | |
| | Dopant | layer | layer | layer | layer | EQE(%) |
| Ex. 29 | BD-1 | HT-2: | HT-2 | EBL-2 | HBL-3 | 8.9 |
| | | HA-2 | | | | |
| Ex. 30 | BD-2 | HT-2: | HT-2 | EBL-2 | HBL-3 | 7.8 |
| | | HA-2 | | | | |
| Comp. | Comp. | HT-2: | HT-2 | EBL-2 | HBL-3 | 4.3 |
| Ex. 29 | compound-1 | HA-2 | | | | |
| Comp. | Comp. | HT-2: | HT-2 | EBL-2 | HBL-3 | 4.6 |
| Ex. 30 | compound-2 | HA-2 | | | | |

From the results shown in Table 3, it can be seen that if the emitting layer contains a particular dopant material and the first layer contains a particular material, a highly efficient organic EL device can be obtained even if the material of the first electron-transporting layer is changed.

### Examples 31 to 42

The organic EL devices were fabricated and evaluated in the same manner as in Example 1 except that the compounds shown in Table 4 were used as materials of the EBL layer (the first layer). The results are shown in Table 4.

### Comparative Examples 31 to 42

The organic EL devices were fabricated and evaluated in the same manner as in Example 1 except that the comparative compound-1 was used as the dopant material and the compounds shown in Table 4 were used as materials of the EBL layer (the first layer). The results are shown in Table 4.

### Comparative Examples 43 to 54

The organic EL devices were fabricated and evaluated in the same manner as in Example 1 except that the comparative compound-2 was used as the dopant material and the compounds shown in Table 4 were used as materials of the EBL layer (the first layer). The results are shown in Table 4.

### Comparative Example 55

The organic EL device was fabricated and evaluated in the same manner as in Example 1 except that the compound BD-1 was used as the dopant material and a comparative compound-3 was used as a material of the EBL layer (the first layer). The results are shown in Table 4.

Table 4 also shows the results of the above-mentioned Examples 1 to 10 and Comparative Examples 1 to 20.

TABLE 4

| Dopant: BD-1 | | | Dopant: Comp. compound-1 | | | Dopant: Comp. compound-2 | | |
|---|---|---|---|---|---|---|---|---|
| | First layer | EQE(%) | | First layer | EQE(%) | | First layer | EQE(%) |
| Ex. 1 | EBL-1 | 10.1 | Comp. | EBL-1 | 4.8 | Comp. | EBL-1 | 5.4 |
| | | | Ex. 1 | | | Ex. 11 | | |
| Ex. 2 | EBL-2 | 10.6 | Comp. | EBL-2 | 5.1 | Comp. | EBL-2 | 5.7 |
| | | | Ex. 2 | | | Ex. 12 | | |
| Ex. 3 | EBL-3 | 10.5 | Comp. | EBL-3 | 5.0 | Comp. | EBL-3 | 5.6 |
| | | | Ex. 3 | | | Ex. 13 | | |
| Ex. 4 | EBL-4 | 10.7 | Comp. | EBL-4 | 5.1 | Comp. | EBL-4 | 5.7 |
| | | | Ex. 4 | | | Ex. 14 | | |
| Ex. 5 | EBL-5 | 11.2 | Comp. | EBL-5 | 5.4 | Comp. | EBL-5 | 6.0 |
| | | | Ex. 5 | | | Ex. 15 | | |
| Ex. 6 | EBL-6 | 10.8 | Comp. | EBL-6 | 5.1 | Comp. | EBL-6 | 5.8 |
| | | | Ex. 6 | | | Ex. 16 | | |
| Ex. 7 | EBL-7 | 9.5 | Comp. | EBL-7 | 4.5 | Comp. | EBL-7 | 5.1 |
| | | | Ex. 7 | | | Ex. 17 | | |
| Ex. 8 | EBL-8 | 10.3 | Comp. | EBL-8 | 4.9 | Comp. | EBL-8 | 5.5 |
| | | | Ex. 8 | | | Ex. 18 | | |
| Ex. 9 | EBL-9 | 10.8 | Comp. | EBL-9 | 5.1 | Comp. | EBL-9 | 5.8 |
| | | | Ex. 9 | | | Ex. 19 | | |
| Ex. 10 | EBL-10 | 10.5 | Comp. | EBL-10 | 5.0 | Comp. | EBL-10 | 5.6 |
| | | | Ex. 10 | | | Ex. 20 | | |
| Ex. 31 | EBL-11 | 10.3 | Comp. | EBL-11 | 4.8 | Comp. | EBL-11 | 5.4 |
| | | | Ex. 31 | | | Ex. 43 | | |
| Ex. 32 | EBL-12 | 10.6 | Comp. | EBL-12 | 5.0 | Comp. | EBL-12 | 5.7 |
| | | | Ex. 32 | | | Ex. 44 | | |
| Ex. 33 | EBL-13 | 9.7 | Comp. | EBL-13 | 4.4 | Comp. | EBL-13 | 5.3 |
| | | | Ex. 33 | | | Ex. 45 | | |
| Ex. 34 | EBL-14 | 10.0 | Comp. | EBL-14 | 4.6 | Comp. | EBL-14 | 5.4 |
| | | | Ex. 34 | | | Ex. 46 | | |
| Ex. 35 | EBL-15 | 10.0 | Comp. | EBL-15 | 5.0 | Comp. | EBL-15 | 5.1 |
| | | | Ex. 35 | | | Ex. 47 | | |
| Ex. 36 | EBL-16 | 10.1 | Comp. | EBL-16 | 5.0 | Comp. | EBL-16 | 5.2 |
| | | | Ex. 36 | | | Ex. 48 | | |
| Ex. 37 | EBL-17 | 9.7 | Comp. | EBL-17 | 4.7 | Comp. | EBL-17 | 5.2 |
| | | | Ex. 37 | | | Ex. 49 | | |
| Ex. 38 | EBL-18 | 10.3 | Comp. | EBL-18 | 5.0 | Comp. | EBL-18 | 5.7 |
| | | | Ex. 38 | | | Ex. 50 | | |
| Ex. 39 | EBL-19 | 9.6 | Comp. | EBL-19 | 4.4 | Comp. | EBL-19 | 4.9 |
| | | | Ex. 39 | | | Ex. 51 | | |
| Ex. 40 | EBL-20 | 10.2 | Comp. | EBL-20 | 4.6 | Comp. | EBL-20 | 5.3 |
| | | | Ex. 40 | | | Ex. 52 | | |
| Ex. 41 | EBL-21 | 10.1 | Comp. | EBL-21 | 4.9 | Comp. | EBL-21 | 5.4 |
| | | | Ex. 41 | | | Ex. 53 | | |
| Ex. 42 | EBL-22 | 11.2 | Comp. | EBL-22 | 5.4 | Comp. | EBL-22 | 6.0 |
| | | | Ex. 42 | | | Ex. 54 | | |
| Comp. | Comp. | 2.4 | | | | | | |
| Ex. 55 | compound- | | | | | | | |
| | 3 | | | | | | | |

### Examples 43 to 54

The organic EL devices were fabricated and evaluated in the same manner as in Example 1 except that the compound BD-2 was used as the dopant material and compounds shown in Table 5 were used as materials of the EBL layer (the first layer). The results are shown in Table 5.

### Comparative Example 56

The organic EL device was fabricated and evaluated in the same manner as in Example 1 except that the compound BD-2 was used as the dopant material and a comparative compound-3 was used as a material of the EBL layer (the first layer). The results are shown in Table 5.

Table 5 also shows the results of the above-mentioned Examples 1 to 10 and Comparative Examples 1 to 20 and 31 to 54.

TABLE 5

| Dopant: BD-2 | | | Dopant: Comp. compound-1 | | | Dopant: Comp. compound-2 | | |
|---|---|---|---|---|---|---|---|---|
| | First layer | EQE(%) | | First layer | EQE(%) | | First layer | EQE(%) |
| Ex. 11 | EBL-1 | 8.0 | Comp. | EBL-1 | 4.8 | Comp. | EBL-1 | 5.4 |
| | | | Ex. 1 | | | Ex. 11 | | |
| Ex. 12 | EBL-2 | 8.4 | Comp. | EBL-2 | 5.1 | Comp. | EBL-2 | 5.7 |
| | | | Ex. 2 | | | Ex. 12 | | |
| Ex. 13 | EBL-3 | 8.3 | Comp. | EBL-3 | 5.0 | Comp. | EBL-3 | 5.6 |
| | | | Ex. 3 | | | Ex. 13 | | |
| Ex. 14 | EBL-4 | 8.4 | Comp. | EBL-4 | 5.1 | Comp. | EBL-4 | 5.7 |
| | | | Ex. 4 | | | Ex. 14 | | |
| Ex. 15 | EBL-5 | 8.9 | Comp. | EBL-5 | 5.4 | Comp. | EBL-5 | 6.0 |
| | | | Ex. 5 | | | Ex. 15 | | |
| Ex. 16 | EBL-6 | 8.5 | Comp. | EBL-6 | 5.1 | Comp. | EBL-6 | 5.8 |
| | | | Ex. 6 | | | Ex. 16 | | |
| Ex. 17 | EBL-7 | 7.5 | Comp. | EBL-7 | 4.5 | Comp. | EBL-7 | 5.1 |
| | | | Ex. 7 | | | Ex. 17 | | |
| Ex. 18 | EBL-8 | 8.1 | Comp. | EBL-8 | 4.9 | Comp. | EBL-8 | 5.5 |
| | | | Ex. 8 | | | Ex. 18 | | |
| Ex. 19 | EBL-9 | 8.5 | Comp. | EBL-9 | 5.1 | Comp. | EBL-9 | 5.8 |
| | | | Ex. 9 | | | Ex. 19 | | |
| Ex. 20 | EBL-10 | 8.3 | Comp. | EBL-10 | 5.0 | Comp. | EBL-10 | 5.6 |
| | | | Ex. 10 | | | Ex. 20 | | |
| Ex. 43 | EBL-11 | 8.4 | Comp. | EBL-11 | 4.8 | Comp. | EBL-11 | 5.4 |
| | | | Ex. 31 | | | Ex. 43 | | |
| Ex. 44 | EBL-12 | 8.5 | Comp. | EBL-12 | 5.0 | Comp. | EBL-12 | 5.7 |
| | | | Ex. 32 | | | Ex. 44 | | |
| Ex. 45 | EBL-13 | 7.9 | Comp. | EBL-13 | 4.4 | Comp. | EBL-13 | 5.3 |
| | | | Ex. 33 | | | Ex. 45 | | |
| Ex. 46 | EBL-14 | 7.9 | Comp. | EBL-14 | 4.6 | Comp. | EBL-14 | 5.4 |
| | | | Ex. 34 | | | Ex. 46 | | |
| Ex. 47 | EBL-15 | 8.0 | Comp. | EBL-15 | 5.0 | Comp. | EBL-15 | 5.1 |
| | | | Ex. 35 | | | Ex. 47 | | |
| Ex. 48 | EBL-16 | 8.1 | Comp. | EBL-16 | 5.0 | Comp. | EBL-16 | 5.2 |
| | | | Ex. 36 | | | Ex. 48 | | |
| Ex. 49 | EBL-17 | 7.7 | Comp. | EBL-17 | 4.7 | Comp. | EBL-17 | 5.2 |
| | | | Ex. 37 | | | Ex. 49 | | |
| Ex. 50 | EBL-18 | 8.2 | Comp. | EBL-18 | 5.0 | Comp. | EBL-18 | 5.7 |
| | | | Ex. 38 | | | Ex. 50 | | |
| Ex. 51 | EBL-19 | 7.5 | Comp. | EBL-19 | 4.4 | Comp. | EBL-19 | 4.9 |
| | | | Ex. 39 | | | Ex. 51 | | |
| Ex. 52 | EBL-20 | 8.3 | Comp. | EBL-20 | 4.6 | Comp. | EBL-20 | 5.3 |
| | | | Ex. 40 | | | Ex. 52 | | |
| Ex. 53 | EBL-21 | 8.3 | Comp. | EBL-21 | 4.9 | Comp. | EBL-21 | 5.4 |
| | | | Ex. 41 | | | Ex. 53 | | |
| Ex. 54 | EBL-22 | 9.0 | Comp. | EBL-22 | 5.4 | Comp. | EBL-22 | 6.0 |
| | | | Ex. 42 | | | Ex. 54 | | |
| Comp. | Comp. | 2.7 | | | | | | |
| Ex. 56 | compound- | | | | | | | |
| | 3 | | | | | | | |

### Examples 55 to 76

The organic EL devices were fabricated and evaluated in the same manner as in Example 1 except that the compound BD-3 was used as the dopant material and compounds shown in Table 6 were used as materials of the EBL layer (the first layer). The results are shown in Table 6.

### Comparative Example 57

The organic EL device was fabricated and evaluated in the same manner as in Example 1 except that the compound BD-3 was used as the dopant material and a comparative compound-3 was used as a material of the EBL layer (the first layer). The results are shown in Table 6.

Table 6 also shows the results of the above-mentioned Comparative Examples 1 to 20 and 31 to 54.

TABLE 6

| Dopant: BD-3 | | | Dopant: Comp. compound-1 | | | Dopant: Comp. compound-2 | | |
|---|---|---|---|---|---|---|---|---|
| | First layer | EQE(%) | | First layer | EQE(%) | | First layer | EQE(%) |
| Ex. 55 | EBL-1 | 9.6 | Comp. | EBL-1 | 4.8 | Comp. | EBL-1 | 5.4 |
| | | | Ex. 1 | | | Ex. 11 | | |
| Ex. 56 | EBL-2 | 10.4 | Comp. | EBL-2 | 5.1 | Comp. | EBL-2 | 5.7 |
| | | | Ex. 2 | | | Ex. 12 | | |
| Ex. 57 | EBL-3 | 10.2 | Comp. | EBL-3 | 5.0 | Comp. | EBL-3 | 5.6 |
| | | | Ex. 3 | | | Ex. 13 | | |
| Ex. 58 | EBL-4 | 10.4 | Comp. | EBL-4 | 5.1 | Comp. | EBL-4 | 5.7 |
| | | | Ex. 4 | | | Ex. 14 | | |
| Ex. 59 | EBL-5 | 10.8 | Comp. | EBL-5 | 5.4 | Comp. | EBL-5 | 6.0 |
| | | | Ex. 5 | | | Ex. 15 | | |
| Ex. 60 | EBL-6 | 10.4 | Comp. | EBL-6 | 5.1 | Comp. | EBL-6 | 5.8 |
| | | | Ex. 6 | | | Ex. 16 | | |
| Ex. 61 | EBL-7 | 9.4 | Comp. | EBL-7 | 4.5 | Comp. | EBL-7 | 5.1 |
| | | | Ex. 7 | | | Ex. 17 | | |
| Ex. 62 | EBL-8 | 9.9 | Comp. | EBL-8 | 4.9 | Comp. | EBL-8 | 5.5 |
| | | | Ex. 8 | | | Ex. 18 | | |
| Ex. 63 | EBL-9 | 10.4 | Comp. | EBL-9 | 5.1 | Comp. | EBL-9 | 5.8 |
| | | | Ex. 9 | | | Ex. 19 | | |
| Ex. 64 | EBL-10 | 10.2 | Comp. | EBL-10 | 5.0 | Comp. | EBL-10 | 5.6 |
| | | | Ex. 10 | | | Ex. 20 | | |
| Ex. 65 | EBL-11 | 9.8 | Comp. | EBL-11 | 4.8 | Comp. | EBL-11 | 5.4 |
| | | | Ex. 31 | | | Ex. 43 | | |
| Ex. 66 | EBL-12 | 10.4 | Comp. | EBL-12 | 5.0 | Comp. | EBL-12 | 5.7 |
| | | | Ex. 32 | | | Ex. 44 | | |
| Ex. 67 | EBL-13 | 9.5 | Comp. | EBL-13 | 4.4 | Comp. | EBL-13 | 5.3 |
| | | | Ex. 33 | | | Ex. 45 | | |
| Ex. 68 | EBL-14 | 9.9 | Comp. | EBL-14 | 4.6 | Comp. | EBL-14 | 5.4 |
| | | | Ex. 34 | | | Ex. 46 | | |
| Ex. 69 | EBL-15 | 9.6 | Comp. | EBL-15 | 5.0 | Comp. | EBL-15 | 5.1 |
| | | | Ex. 35 | | | Ex. 47 | | |
| Ex. 70 | EBL-16 | 9.8 | Comp. | EBL-16 | 5.0 | Comp. | EBL-16 | 5.2 |
| | | | Ex. 36 | | | Ex. 48 | | |
| Ex. 71 | EBL-17 | 9.3 | Comp. | EBL-17 | 4.7 | Comp. | EBL-17 | 5.2 |
| | | | Ex. 37 | | | Ex. 49 | | |
| Ex. 72 | EBL-18 | 9.8 | Comp. | EBL-18 | 5.0 | Comp. | EBL-18 | 5.7 |
| | | | Ex. 38 | | | Ex. 50 | | |
| Ex. 73 | EBL-19 | 9.3 | Comp. | EBL-19 | 4.4 | Comp. | EBL-19 | 4.9 |
| | | | Ex. 39 | | | Ex. 51 | | |
| Ex. 74 | EBL-20 | 10.0 | Comp. | EBL-20 | 4.6 | Comp. | EBL-20 | 5.3 |
| | | | Ex. 40 | | | Ex. 52 | | |
| Ex. 75 | EBL-21 | 9.8 | Comp. | EBL-21 | 4.9 | Comp. | EBL-21 | 5.4 |
| | | | Ex. 41 | | | Ex. 53 | | |
| Ex. 76 | EBL-22 | 10.9 | Comp. | EBL-22 | 5.4 | Comp. | EBL-22 | 6.0 |
| | | | Ex. 42 | | | Ex. 54 | | |
| Comp. | Comp. | 2.4 | | | | | | |
| Ex. 57 | compound- | | | | | | | |
| | 3 | | | | | | | |

### Examples 77 to 98

The organic EL devices were fabricated and evaluated in the same manner as in Example 1 except that the compound BD-4 was used as the dopant material and compounds shown in Table 7 were used as materials of the EBL layer (the first layer). The results are shown in Table 7.

### Comparative Example 58

The organic EL device was fabricated and evaluated in the same manner as in Example 1 except that the compound BD-4 was used as the dopant material and a comparative compound-3 was used as a material of the EBL layer (the first layer). The results are shown in Table 7.

Table 7 also shows the results of the above-mentioned Comparative Examples 1 to 20 and 31 to 54.

TABLE 7

| Dopant: BD-4 | | | Dopant: Comp. compound-1 | | | Dopant: Comp. compound-2 | | |
|---|---|---|---|---|---|---|---|---|
| | First layer | EQE(%) | | First layer | EQE(%) | | First layer | EQE(%) |
| Ex. 77 | EBL-1 | 10.0 | Comp. | EBL-1 | 4.8 | Comp. | EBL-1 | 5.4 |
| | | | Ex. 1 | | | Ex. 11 | | |
| Ex. 78 | EBL-2 | 10.5 | Comp. | EBL-2 | 5.1 | Comp. | EBL-2 | 5.7 |
| | | | Ex. 2 | | | Ex. 12 | | |
| Ex. 79 | EBL-3 | 10.0 | Comp. | EBL-3 | 5.0 | Comp. | EBL-3 | 5.6 |
| | | | Ex. 3 | | | Ex. 13 | | |
| Ex. 80 | EBL-4 | 10.3 | Comp. | EBL-4 | 5.1 | Comp. | EBL-4 | 5.7 |
| | | | Ex. 4 | | | Ex. 14 | | |
| Ex. 81 | EBL-5 | 10.9 | Comp. | EBL-5 | 5.4 | Comp. | EBL-5 | 6.0 |
| | | | Ex. 5 | | | Ex. 15 | | |
| Ex. 82 | EBL-6 | 10.4 | Comp. | EBL-6 | 5.1 | Comp. | EBL-6 | 5.8 |
| | | | Ex. 6 | | | Ex. 16 | | |
| Ex. 83 | EBL-7 | 9.3 | Comp. | EBL-7 | 4.5 | Comp. | EBL-7 | 5.1 |
| | | | Ex. 7 | | | Ex. 17 | | |
| Ex. 84 | EBL-8 | 9.9 | Comp. | EBL-8 | 4.9 | Comp. | EBL-8 | 5.5 |
| | | | Ex. 8 | | | Ex. 18 | | |
| Ex. 85 | EBL-9 | 10.6 | Comp. | EBL-9 | 5.1 | Comp. | EBL-9 | 5.8 |
| | | | Ex. 9 | | | Ex. 19 | | |
| Ex. 86 | EBL-10 | 10.0 | Comp. | EBL-10 | 5.0 | Comp. | EBL-10 | 5.6 |
| | | | Ex. 10 | | | Ex. 20 | | |
| Ex. 87 | EBL-11 | 10.2 | Comp. | EBL-11 | 4.8 | Comp. | EBL-11 | 5.4 |
| | | | Ex. 31 | | | Ex. 43 | | |
| Ex. 88 | EBL-12 | 10.5 | Comp. | EBL-12 | 5.0 | Comp. | EBL-12 | 5.7 |
| | | | Ex. 32 | | | Ex. 44 | | |
| Ex. 89 | EBL-13 | 9.2 | Comp. | EBL-13 | 4.4 | Comp. | EBL-13 | 5.3 |
| | | | Ex. 33 | | | Ex. 45 | | |
| Ex. 90 | EBL-14 | 9.7 | Comp. | EBL-14 | 4.6 | Comp. | EBL-14 | 5.4 |
| | | | Ex. 34 | | | Ex. 46 | | |
| Ex. 91 | EBL-15 | 9.6 | Comp. | EBL-15 | 5.0 | Comp. | EBL-15 | 5.1 |
| | | | Ex. 35 | | | Ex. 47 | | |
| Ex. 92 | EBL-16 | 10.0 | Comp. | EBL-16 | 5.0 | Comp. | EBL-16 | 5.2 |
| | | | Ex. 36 | | | Ex. 48 | | |
| Ex. 93 | EBL-17 | 9.4 | Comp. | EBL-17 | 4.7 | Comp. | EBL-17 | 5.2 |
| | | | Ex. 37 | | | Ex. 49 | | |
| Ex. 94 | EBL-18 | 10.2 | Comp. | EBL-18 | 5.0 | Comp. | EBL-18 | 5.7 |
| | | | Ex. 38 | | | Ex. 50 | | |
| Ex. 95 | EBL-19 | 9.4 | Comp. | EBL-19 | 4.4 | Comp. | EBL-19 | 4.9 |
| | | | Ex. 39 | | | Ex. 51 | | |
| Ex. 96 | EBL-20 | 10.0 | Comp. | EBL-20 | 4.6 | Comp. | EBL-20 | 5.3 |
| | | | Ex. 40 | | | Ex. 52 | | |
| Ex. 97 | EBL-21 | 9.7 | Comp. | EBL-21 | 4.9 | Comp. | EBL-21 | 5.4 |
| | | | Ex. 41 | | | Ex. 53 | | |
| Ex. 98 | EBL-22 | 10.9 | Comp. | EBL-22 | 5.4 | Comp. | EBL-22 | 6.0 |
| | | | Ex. 42 | | | Ex. 54 | | |
| Comp. | Comp. | 2.6 | | | | | | |
| Ex. 58 | compound- | | | | | | | |
| | 3 | | | | | | | |

### Examples 99 to 120

The organic EL devices were fabricated and evaluated in the same manner as in Example 1 except that the compound BD-5 was used as the dopant material and compounds shown in Table 8 were used as materials of the EBL layer (the first layer). The results are shown in Table 8.

### Comparative Example 59

The organic EL device was fabricated and evaluated in the same manner as in Example 1 except that the compound BD-5 was used as the dopant material and a comparative compound-3 was used as a material of the EBL layer (the first layer). The results are shown in Table 8.

Table 8 also shows the results of the above-mentioned Comparative Examples 1 to 20 and 31 to 54.

TABLE 8

| Dopant: BD-5 | | | Dopant: Comp. compound-1 | | | Dopant: Comp. compound-2 | | |
|---|---|---|---|---|---|---|---|---|
| | First layer | EQE(%) | | First layer | EQE(%) | | First layer | EQE(%) |
| Ex. 99 | EBL-1 | 9.8 | Comp. | EBL-1 | 4.8 | Comp. | EBL-1 | 5.4 |
| | | | Ex. 1 | | | Ex. 11 | | |
| Ex. 100 | EBL-2 | 10.5 | Comp. | EBL-2 | 5.1 | Comp. | EBL-2 | 5.7 |
| | | | Ex. 2 | | | Ex. 12 | | |
| Ex. 101 | EBL-3 | 10.4 | Comp. | EBL-3 | 5.0 | Comp. | EBL-3 | 5.6 |
| | | | Ex. 3 | | | Ex. 13 | | |
| Ex. 102 | EBL-4 | 10.5 | Comp. | EBL-4 | 5.1 | Comp. | EBL-4 | 5.7 |
| | | | Ex. 4 | | | Ex. 14 | | |
| Ex. 103 | EBL-5 | 11.0 | Comp. | EBL-5 | 5.4 | Comp. | EBL-5 | 6.0 |
| | | | Ex. 5 | | | Ex. 15 | | |
| Ex. 104 | EBL-6 | 10.8 | Comp. | EBL-6 | 5.1 | Comp. | EBL-6 | 5.8 |
| | | | Ex. 6 | | | Ex. 16 | | |
| Ex. 105 | EBL-7 | 9.4 | Comp. | EBL-7 | 4.5 | Comp. | EBL-7 | 5.1 |
| | | | Ex. 7 | | | Ex. 17 | | |
| Ex. 106 | EBL-8 | 9.9 | Comp. | EBL-8 | 4.9 | Comp. | EBL-8 | 5.5 |
| | | | Ex. 8 | | | Ex. 18 | | |
| Ex. 107 | EBL-9 | 10.8 | Comp. | EBL-9 | 5.1 | Comp. | EBL-9 | 5.8 |
| | | | Ex. 9 | | | Ex. 19 | | |
| Ex. 108 | EBL-10 | 10.4 | Comp. | EBL-10 | 5.0 | Comp. | EBL-10 | 5.6 |
| | | | Ex. 10 | | | Ex. 20 | | |
| Ex. 109 | EBL-11 | 10.3 | Comp. | EBL-11 | 4.8 | Comp. | EBL-11 | 5.4 |
| | | | Ex. 31 | | | Ex. 43 | | |
| Ex. 110 | EBL-12 | 10.5 | Comp. | EBL-12 | 5.0 | Comp. | EBL-12 | 5.7 |
| | | | Ex. 32 | | | Ex. 44 | | |
| Ex. 111 | EBL-13 | 9.5 | Comp. | EBL-13 | 4.4 | Comp. | EBL-13 | 5.3 |
| | | | Ex. 33 | | | Ex. 45 | | |
| Ex. 112 | EBL-14 | 9.9 | Comp. | EBL-14 | 4.6 | Comp. | EBL-14 | 5.4 |
| | | | Ex. 34 | | | Ex. 46 | | |
| Ex. 113 | EBL-15 | 9.9 | Comp. | EBL-15 | 5.0 | Comp. | EBL-15 | 5.1 |
| | | | Ex. 35 | | | Ex. 47 | | |
| Ex. 114 | EBL-16 | 10.0 | Comp. | EBL-16 | 5.0 | Comp. | EBL-16 | 5.2 |
| | | | Ex. 36 | | | Ex. 48 | | |
| Ex. 115 | EBL-17 | 9.4 | Comp. | EBL-17 | 4.7 | Comp. | EBL-17 | 5.2 |
| | | | Ex. 37 | | | Ex. 49 | | |
| Ex. 116 | EBL-18 | 10.0 | Comp. | EBL-18 | 5.0 | Comp. | EBL-18 | 5.7 |
| | | | Ex. 38 | | | Ex. 50 | | |
| Ex. 117 | EBL-19 | 9.6 | Comp. | EBL-19 | 4.4 | Comp. | EBL-19 | 4.9 |
| | | | Ex. 39 | | | Ex. 51 | | |
| Ex. 118 | EBL-20 | 9.8 | Comp. | EBL-20 | 4.6 | Comp. | EBL-20 | 5.3 |
| | | | Ex. 40 | | | Ex. 52 | | |
| Ex. 119 | EBL-21 | 10.1 | Comp. | EBL-21 | 4.9 | Comp. | EBL-21 | 5.4 |
| | | | Ex. 41 | | | Ex. 53 | | |
| Ex. 120 | EBL-22 | 11.0 | Comp. | EBL-22 | 5.4 | Comp. | EBL-22 | 6.0 |
| | | | Ex. 42 | | | Ex. 54 | | |
| Comp. | Comp. | 2.5 | | | | | | |
| Ex. 59 | compound- | | | | | | | |
| | 3 | | | | | | | |

### Examples 121 to 142

The organic EL devices were fabricated and evaluated in the same manner as in Example 1 except that the compound BD-6 was used as the dopant material and compounds shown in Table 9 were used as materials of the EBL layer (the first layer). The results are shown in Table 9.

### Comparative Example 60

The organic EL device was fabricated and evaluated in the same manner as in Example 1 except that the compound BD-6 was used as the dopant material and a comparative compound-3 was used as a material of the EBL layer (the first layer). The results are shown in Table 9.

Table 9 also shows the results of the above-mentioned Comparative Examples 1 to 20 and 31 to 54.

TABLE 9

| Dopant: BD-6 | | | Dopant: Comp. compound-1 | | | Dopant: Comp. compound-2 | | |
|---|---|---|---|---|---|---|---|---|
| | First layer | EQE(%) | | First layer | EQE(%) | | First layer | EQE(%) |
| Ex. 121 | EBL-1 | 10.2 | Comp. | EBL-1 | 4.8 | Comp. | EBL-1 | 5.4 |
| | | | Ex. 1 | | | Ex. 11 | | |
| Ex. 122 | EBL-2 | 10.6 | Comp. | EBL-2 | 5.1 | Comp. | EBL-2 | 5.7 |
| | | | Ex. 2 | | | Ex. 12 | | |
| Ex. 123 | EBL-3 | 10.7 | Comp. | EBL-3 | 5.0 | Comp. | EBL-3 | 5.6 |
| | | | Ex. 3 | | | Ex. 13 | | |
| Ex. 124 | EBL-4 | 11.0 | Comp. | EBL-4 | 5.1 | Comp. | EBL-4 | 5.7 |
| | | | Ex. 4 | | | Ex. 14 | | |
| Ex. 125 | EBL-5 | 11.0 | Comp. | EBL-5 | 5.4 | Comp. | EBL-5 | 6.0 |
| | | | Ex. 5 | | | Ex. 15 | | |
| Ex. 126 | EBL-6 | 10.9 | Comp. | EBL-6 | 5.1 | Comp. | EBL-6 | 5.8 |
| | | | Ex. 6 | | | Ex. 16 | | |
| Ex. 127 | EBL-7 | 9.6 | Comp. | EBL-7 | 4.5 | Comp. | EBL-7 | 5.1 |
| | | | Ex. 7 | | | Ex. 17 | | |
| Ex. 128 | EBL-8 | 10.5 | Comp. | EBL-8 | 4.9 | Comp. | EBL-8 | 5.5 |
| | | | Ex. 8 | | | Ex. 18 | | |
| Ex. 129 | EBL-9 | 10.8 | Comp. | EBL-9 | 5.1 | Comp. | EBL-9 | 5.8 |
| | | | Ex. 9 | | | Ex. 19 | | |
| Ex. 130 | EBL-10 | 10.8 | Comp. | EBL-10 | 5.0 | Comp. | EBL-10 | 5.6 |
| | | | Ex. 10 | | | Ex. 20 | | |
| Ex. 131 | EBL-11 | 10.7 | Comp. | EBL-11 | 4.8 | Comp. | EBL-11 | 5.4 |
| | | | Ex. 31 | | | Ex. 43 | | |
| Ex. 132 | EBL-12 | 10.7 | Comp. | EBL-12 | 5.0 | Comp. | EBL-12 | 5.7 |
| | | | Ex. 32 | | | Ex. 44 | | |
| Ex. 133 | EBL-13 | 9.7 | Comp. | EBL-13 | 4.4 | Comp. | EBL-13 | 5.3 |
| | | | Ex. 33 | | | Ex. 45 | | |
| Ex. 134 | EBL-14 | 10.2 | Comp. | EBL-14 | 4.6 | Comp. | EBL-14 | 5.4 |
| | | | Ex. 34 | | | Ex. 46 | | |
| Ex. 135 | EBL-15 | 10.4 | Comp. | EBL-15 | 5.0 | Comp. | EBL-15 | 5.1 |
| | | | Ex. 35 | | | Ex. 47 | | |
| Ex. 136 | EBL-16 | 10.2 | Comp. | EBL-16 | 5.0 | Comp. | EBL-16 | 5.2 |
| | | | Ex. 36 | | | Ex. 48 | | |
| Ex. 137 | EBL-17 | 10.1 | Comp. | EBL-17 | 4.7 | Comp. | EBL-17 | 5.2 |
| | | | Ex. 37 | | | Ex. 49 | | |
| Ex. 138 | EBL-18 | 10.3 | Comp. | EBL-18 | 5.0 | Comp. | EBL-18 | 5.7 |
| | | | Ex. 38 | | | Ex. 50 | | |
| Ex. 139 | EBL-19 | 9.7 | Comp. | EBL-19 | 4.4 | Comp. | EBL-19 | 4.9 |
| | | | Ex. 39 | | | Ex. 51 | | |
| Ex. 140 | EBL-20 | 10.4 | Comp. | EBL-20 | 4.6 | Comp. | EBL-20 | 5.3 |
| | | | Ex. 40 | | | Ex. 52 | | |
| Ex. 141 | EBL-21 | 10.5 | Comp. | EBL-21 | 4.9 | Comp. | EBL-21 | 5.4 |
| | | | Ex. 41 | | | Ex. 53 | | |
| Ex. 142 | EBL-22 | 11.1 | Comp. | EBL-22 | 5.4 | Comp. | EBL-22 | 6.0 |
| | | | Ex. 42 | | | Ex. 54 | | |
| Comp. | Comp. | 2.6 | | | | | | |
| Ex. 60 | compound- | | | | | | | |
| | 3 | | | | | | | |

### Examples 143 to 164

The organic EL devices were fabricated and evaluated in the same manner as in Example 1 except that the compound BD-7 was used as the dopant material and compounds shown in Table 10 were used as materials of the EBL layer (the first layer). The results are shown in Table 10.

### Comparative Example 61

The organic EL device was fabricated and evaluated in the same manner as in Example 1 except that the compound BD-7 was used as the dopant material and a comparative compound-3 was used as a material of the EBL layer (the first layer). The results are shown in Table 10.

Table 10 also shows the results of the above-mentioned Comparative Examples 1 to 20 and 31 to 54.

TABLE 10

| Dopant: BD-7 | | | Dopant: Comp. compound-1 | | | Dopant: Comp. compound-2 | | |
|---|---|---|---|---|---|---|---|---|
| | First layer | EQE(%) | | First layer | EQE(%) | | First layer | EQE(%) |
| Ex. 143 | EBL-1 | 8.3 | Comp. | EBL-1 | 4.8 | Comp. | EBL-1 | 5.4 |
| | | | Ex. 1 | | | Ex. 11 | | |
| Ex. 144 | EBL-2 | 8.9 | Comp. | EBL-2 | 5.1 | Comp. | EBL-2 | 5.7 |
| | | | Ex. 2 | | | Ex. 12 | | |
| Ex. 145 | EBL-3 | 8.5 | Comp. | EBL-3 | 5.0 | Comp. | EBL-3 | 5.6 |
| | | | Ex. 3 | | | Ex. 13 | | |
| Ex. 146 | EBL-4 | 8.7 | Comp. | EBL-4 | 5.1 | Comp. | EBL-4 | 5.7 |
| | | | Ex. 4 | | | Ex. 14 | | |
| Ex. 147 | EBL-5 | 9.1 | Comp. | EBL-5 | 5.4 | Comp. | EBL-5 | 6.0 |
| | | | Ex. 5 | | | Ex. 15 | | |
| Ex. 148 | EBL-6 | 9.0 | Comp. | EBL-6 | 5.1 | Comp. | EBL-6 | 5.8 |
| | | | Ex. 6 | | | Ex. 16 | | |
| Ex. 149 | EBL-7 | 7.9 | Comp. | EBL-7 | 4.5 | Comp. | EBL-7 | 5.1 |
| | | | Ex. 7 | | | Ex. 17 | | |
| Ex. 150 | EBL-8 | 8.6 | Comp. | EBL-8 | 4.9 | Comp. | EBL-8 | 5.5 |
| | | | Ex. 8 | | | Ex. 18 | | |
| Ex. 151 | EBL-9 | 9.0 | Comp. | EBL-9 | 5.1 | Comp. | EBL-9 | 5.8 |
| | | | Ex. 9 | | | Ex. 19 | | |
| Ex. 152 | EBL-10 | 8.9 | Comp. | EBL-10 | 5.0 | Comp. | EBL-10 | 5.6 |
| | | | Ex. 10 | | | Ex. 20 | | |
| Ex. 153 | EBL-11 | 8.4 | Comp. | EBL-11 | 4.8 | Comp. | EBL-11 | 5.4 |
| | | | Ex. 31 | | | Ex. 43 | | |
| Ex. 154 | EBL-12 | 8.8 | Comp. | EBL-12 | 5.0 | Comp. | EBL-12 | 5.7 |
| | | | Ex. 32 | | | Ex. 44 | | |
| Ex. 155 | EBL-13 | 8.0 | Comp. | EBL-13 | 4.4 | Comp. | EBL-13 | 5.3 |
| | | | Ex. 33 | | | Ex. 45 | | |
| Ex. 156 | EBL-14 | 8.4 | Comp. | EBL-14 | 4.6 | Comp. | EBL-14 | 5.4 |
| | | | Ex. 34 | | | Ex. 46 | | |
| Ex. 157 | EBL-15 | 8.3 | Comp. | EBL-15 | 5.0 | Comp. | EBL-15 | 5.1 |
| | | | Ex. 35 | | | Ex. 47 | | |
| Ex. 158 | EBL-16 | 8.2 | Comp. | EBL-16 | 5.0 | Comp. | EBL-16 | 5.2 |
| | | | Ex. 36 | | | Ex. 48 | | |
| Ex. 159 | EBL-17 | 8.2 | Comp. | EBL-17 | 4.7 | Comp. | EBL-17 | 5.2 |
| | | | Ex. 37 | | | Ex. 49 | | |
| Ex. 160 | EBL-18 | 8.5 | Comp. | EBL-18 | 5.0 | Comp. | EBL-18 | 5.7 |
| | | | Ex. 38 | | | Ex. 50 | | |
| Ex. 161 | EBL-19 | 8.1 | Comp. | EBL-19 | 4.4 | Comp. | EBL-19 | 4.9 |
| | | | Ex. 39 | | | Ex. 51 | | |
| Ex. 162 | EBL-20 | 8.3 | Comp. | EBL-20 | 4.6 | Comp. | EBL-20 | 5.3 |
| | | | Ex. 40 | | | Ex. 52 | | |
| Ex. 163 | EBL-21 | 8.2 | Comp. | EBL-21 | 4.9 | Comp. | EBL-21 | 5.4 |
| | | | Ex. 41 | | | Ex. 53 | | |
| Ex. 164 | EBL-22 | 9.1 | Comp. | EBL-22 | 5.4 | Comp. | EBL-22 | 6.0 |
| | | | Ex. 42 | | | Ex. 54 | | |
| Comp. | Comp. | 2.3 | | | | | | |
| Ex. 61 | compound- | | | | | | | |
| | 3 | | | | | | | |

From the results shown in Tables 4 to 10, it can be seen that the luminous efficiency EQE of the devices of Comparative Examples 1 to 20 and 31 to 54 using the comparative compound-1 or the comparative compound-2 as the dopant material and the compounds EBL-1 to EBL-22 as the material of the first layer ranges from 4.4 to 6.0%, whereas the luminous efficiency EQE of the devices of Examples 1 to 164 using the compounds BD-1 to BD-7 as the dopant material, and the compounds EBL-1 to EBL-22 as the material of the first layer ranges from 9.5 to 11.2%, which is almost double.

In addition, from the results shown in Tables 4 to 10, it can be seen that the luminous efficiency EQE of the devices of Comparative Examples 55 to 61 using the comparative compound-3 as the material of the first layer and the compounds BD-1 to BD-7, respectively, as the dopant material ranges from 2.3 to 2.7%. These Comparative Examples show that even if a compound represented by the formula (A1) is used as the dopant material, the luminous efficiency does not improve unless a compound represented by the formula (B1) or (C1) is used as the material of the first layer.

Taking these results into consideration, it can be seen that the remarkable improvement in the luminous efficiency EQE is an effect obtained by combining the compound represented by the formula (A1), which is the dopant material, with the compound represented by the formula (B1) or (C1), which is the material of the first layer.

### <Synthesis of Compounds>

### Synthesis Example 1: Synthesis of Compound BD-1

BD-1 was synthesized by the synthetic route described below.

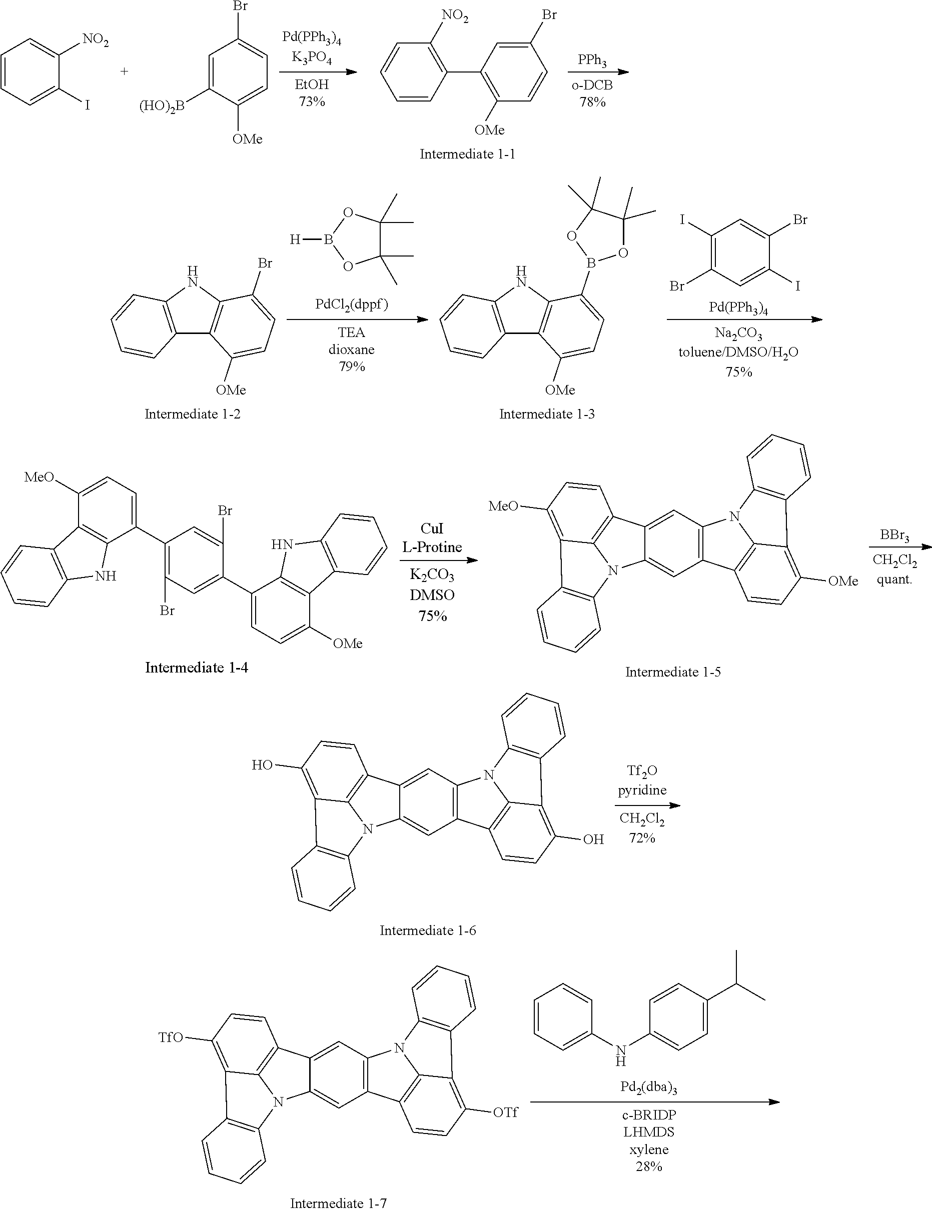

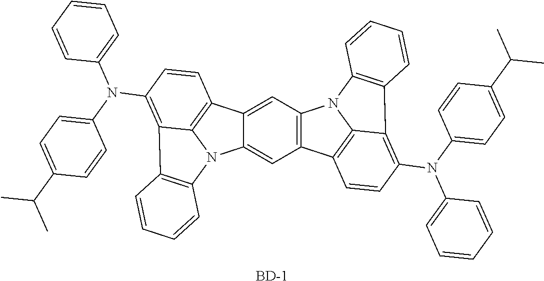 [SMILES: CC(C)c1ccc(N(c2ccccc2)c2ccc3c4cc5c(cc4n4c6ccccc6c2c34)c2ccc(N(c3ccccc3)c3ccc(C(C)C)cc3)c3c4ccccc4n5c23)cc1 ; "BD-1"]

### Synthesis of Intermediate 1-1

Under an argon atmosphere, 2-iodonitrobenzene (9.7 g, 39 mmol), 5-bromo-2-methoxyphenylboronic acid (9.2 g, 40 mmol), tetrakis(triphenylphosphine)palladium(0) (Pd(PPh₃)₄, 1.1 g, 0.975 mmol), and K₃PO₄ (21 g, 97 mmol) were dissolved in ethanol (95 mL) and refluxed for 8 hours. After completion of the reaction, the solvent was concentrated and the residue was purified by column chromatography to obtain a yellow solid (8.8 g, yield: 73%). The obtained solid was intermediate 1-1, which was an intended product, and the result of mass spectrometric analysis was: m/e=308 for a molecular weight of 308.

### Synthesis of Intermediate 1-2

Intermediate 1-1 (7.00 g, 22.7 mmol) was dissolved in o-dichlorobenzene (80 mL), and triphenylphosphine (14.9 g, 56.8 mmol) was added and refluxed for 12 hours. After completion of the reaction, the solvent was concentrated, and the residue was purified by column chromatography to obtain a white solid (5.7 g, yield: 78%). The obtained solid was intermediate 1-2, which was an intended product, and the result of mass spectrometric analysis was: m/e=276 for a molecular weight of 276.

### Synthesis of Intermediate 1-3

Under an argon atmosphere, intermediate 1-2 (5.7 g, 21 mmol), pinacol borane (7.9 g, 62 mmol), dichloro[1,1′-bis(diphenylphosphino)ferrocene]palladium (PdCl₂(dppf), 1.46 g, 2.0 mmol) was dissolved in dioxane (250 mL), and triethylamine (11.5 mL, 83 mmol) was added and refluxed for 5 hours. After completion of the reaction, the solvent was concentrated and the residue was purified by column chromatography to obtain a yellow solid (5.0 g, yield: 75%). The obtained solid was intermediate 1-3, which was an intended product, and the result of mass spectrometric analysis was: m/e=323 for a molecular weight of 323.

### Synthesis of Intermediate 1-4

Under an argon atmosphere, dibromodiiodobenzene (2.5 g, 5.1 mmol), intermediate 1-3 (4.97 g, 15.4 mmol), tetrakis(triphenylphosphine)palladium(0) (Pd(PPh₃)₄, 237 mg, 0.205 mmol) were dissolved in toluene (250 mL) and dimethylsulfoxide (50 mL), and a 2M aqueous solution of Na₂CO₃ (13 mL) was added thereto, and heated and stirred at 90° C. for 24 hours. After completion of the reaction, toluene was removed under reduced pressure, and the precipitated solid was collected by filtration. This solid was washed with methanol and ethyl acetate to obtain a white solid (2.5 g, yield: 75%). The obtained solid was intermediate 1-4, which was an intended product, and the result of mass spectrometric analysis was: m/e=626 for a molecular weight of 626.

### Synthesis of Intermediate 1-5

Under an argon atmosphere, intermediate 1-4 (2.5 g, 3.99 mmol), CuI (76 mg, 0.40 mmol), L-proline (92 mg, 0.80 mmol), and K₂CO₃ (1.38 g, 10 mmol) were suspended in dimethylsulfoxide (80 mL) and heated and at 150° C. stirred for 6 hours. After completion of the reaction, the precipitated solid was collected by filtration. This solid was washed with methanol and ethyl acetate to obtain a brown solid (1.4 g, yield: 75%). The obtained solid was compound 1-5, which was an intended product, and the result of mass spectrometric analysis was: m/e=464 for a molecular weight of 465.

### Synthesis of Intermediate 1-6

Intermediate 1-5 (1.4 g, 3.0 mmol) was dissolved in dichloromethane (150 mL) and a 1M BBr₃ solution in dichloromethane (15 mL, 15 mmol) was added and refluxed for 8 hours. After completion of the reaction, ice water (50 mL) was added, and the precipitate was collected by filtration. This solid was washed with methanol to obtain a white solid (1.4 g). The obtained solid was intermediate 1-6, which was an intended product, and the result of mass spectrometric analysis was: m/e=436 for a molecular weight of 437.

### Synthesis of Intermediate 1-7

Intermediate 1-6 (1.4 g, 3.2 mmol) was suspended in dichloromethane (75 mL) and pyridine (75 mL), and anhydrous triflate (3.8 mL, 22.5 mmol) was added and stirred at room temperature for 8 hours. After completion of the reaction, water (50 mL) was added and the precipitate was collected by filtration. This solid was washed with methanol and ethyl acetate to obtain a white solid (1.8 g, yield: 72%). The obtained solid was intermediate 1-7, which was an intended product, and the result of mass spectrometric analysis was: m/e=700 for a molecular weight of 701.

### Synthesis of BD-1

Under an argon atmosphere, intermediate 1-7 (1.00 g, 1.43 mmol), 4-iPr-N-phenylaniline (754 mg, 3.57 mmol), tris(dibenzylideneacetone)dipaladium(0) (Pd₂(dba)₃, 26 mg, 0.029 mmol), di-tert-butyl(1-methyl-2,2-diphenylcyclopropyl)phosphine (40 mg, 0.11 mmol) were dissolved in xylene (120 mL), and 1M lithium bis(trimethylsilyl)amide solution in tetrahydrofuran (3.6 mL, 3.6 mmol) was added and refluxed for 8 hours. After completion of the reaction, the solid obtained by filtration through celite and distilling off the solvent was purified by column chromatography to obtain a yellow solid (300 mg, yield: 26%). The obtained solid was BD-1, which was an intended product, and the result of mass spectrometric analysis was: m/e=823 for a molecular weight of 823.

### Synthesis Example 2: Synthesis of Compound BD-2

BD-2 was synthesized by the synthetic route described below.

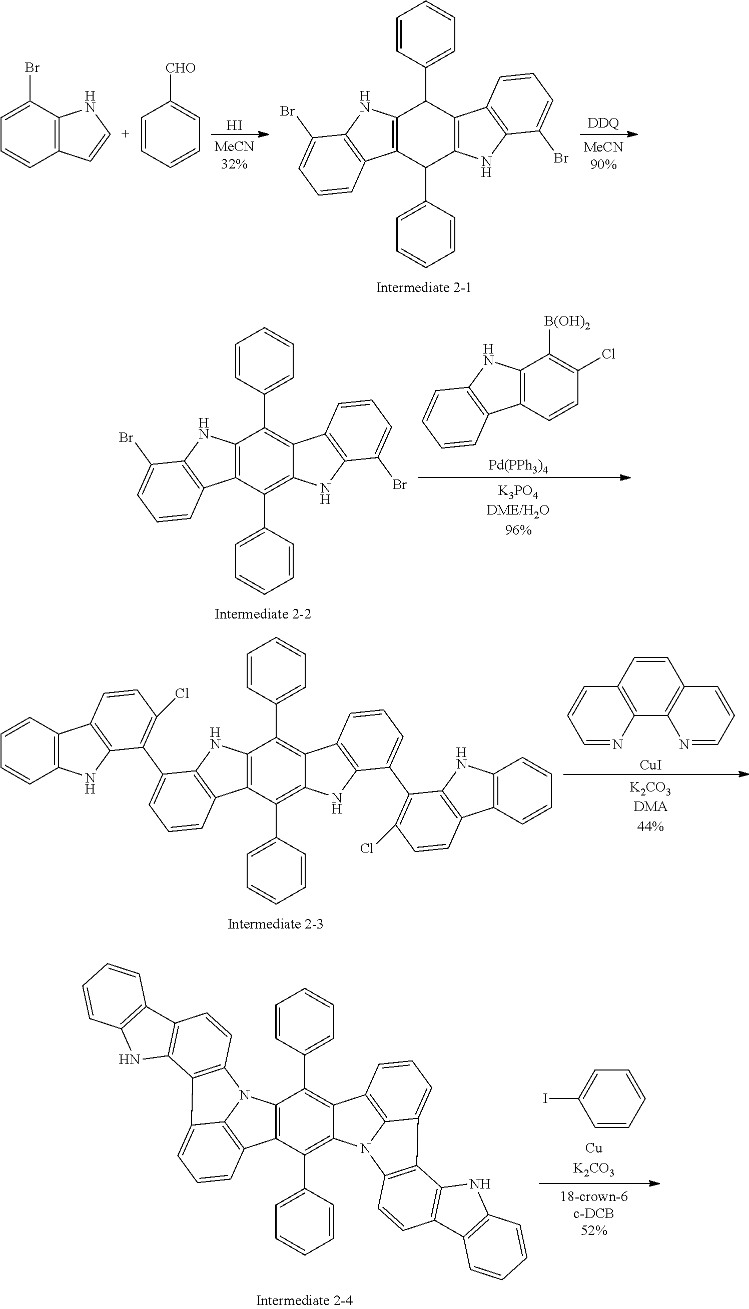

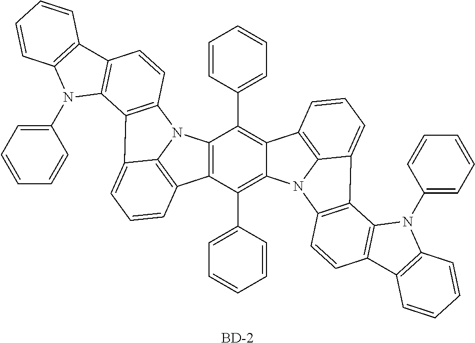 [SMILES: c1ccc(-c2c3c4cccc5c6c7c(ccc6n(c3c(-c3ccccc3)c3c6cccc8c9c%10c(ccc9n(c23)c68)c2ccccc2n%10-c2ccccc2)c45)c2ccccc2n7-c2ccccc2)cc1 ; "BD-2"]

### Synthesis of Intermediate 2-1

7-bromo-1H-indole (10.0 g, 51.0 mmol) was dissolved in acetonitrile (200 mL), and benzaldehyde (5.41 g, 51.0 mmol) and 57% hydroiodic acid (2 mL) were added to the resulting solution, and the resulting mixture was stirred at 80° C. for 8 hours. After completion of the reaction, a precipitated solid was collected by filtration, and the resulting material was washed with acetonitrile to obtain a light yellow solid (4.60 g, yield: 32%). The obtained solid was intermediate 2-1, which was an intended product, and the result of mass spectrometric analysis was: m/e=569 for a molecular weight of 568.

### Synthesis of Intermediate 2-2

Intermediate 2-1 (4.5 g, 7.92 mmol) was suspended in acetonitrile (200 mL), 2,3-dichloro-5,6-dicyano-p-benzoquinone (4.49 g, 19.8 mmol) was added and stirred at 80° C. for 16 hours. After completion of the reaction, a solid was collected by filtration, and the resulting material was washed with acetonitrile to obtain a yellow solid (4.02 g, yield: 90%). The obtained solid was intermediate 2-2, which was an intended product, and the result of mass spectrometric analysis was: m/e=566 for a molecular weight of 566.

### Synthesis of Intermediate 2-3

Under an argon atmosphere, 1,2-dimethoxyethane (80 mL) and water (20 mL) was added to intermediate 2-2 (3.50 g, 6.18 mmol), 2-chloro-9H-carbazolyl-1-boronic acid (4.55 g, 18.5 mmol), tetrakis(triphenylphosphine)palladium(0) (Pd(PPh₃)₄, 970 mg, 0.839 mmol), K₃PO₄ (7.87 g, 37.1 mmol), and stirred at 80° C. for 12 hours. After completion of the reaction, an organic layer was concentrated and a solid was collected by filtration. The resulting material was purified by column chromatography to obtain a yellow solid (4.82 g, yield: 96%). The obtained solid was intermediate 2-3, which was an intended product, and the result of mass spectrometric analysis was: m/e=806 for a molecular weight of 808.

### Synthesis of Intermediate 2-4

Under an argon atmosphere, intermediates 2-3 (4.00 g, 4.95 mmol), iodide copper(I) (566 mg, 2.97 mmol), 1,10-phenanthroline (535 mg, 2.97 mmol), K₂CO₃ (2.74 g, 19.8 mmol) were suspended in N,N-dimethylacetamide (80 mL), and heated and stirred at 160° C. for 8 hours. After completion of the reaction, water was added thereto, and a precipitate was collected by filtration. The resulting material was purified by column chromatography to obtain a yellow solid (1.62 g, yield: 44%). The obtained solid was intermediate 2-4, which was an intended product, and the result of mass spectrometric analysis was: m/e=735 for a molecular weight of 735.

### Synthesis of BD-2

Under an argon atmosphere, intermediate 2-4 (1.00 g, 4.95 mmol), copper powder (346 mg, 5.44 mmol), K₂CO₃ (1.5 g, 10.9 mmol), 18-crown-6-ether (144 mg, 0.544 mmol) were suspended in o-dichlorobenzene (10 mL) and heated and stirred at 170° C. for 12 hours. After completion of the reaction, a precipitate was collected by filtration, and the resulting material was passed through short pass column chromatography. The solvent was distilled off to obtain a yellow solid (630 mg, yield: 52%). The obtained solid was BD-2, which was an intended product, and the result of mass spectrometric analysis was: m/e=888 for a molecular weight of 887.

### Synthesis Example 3: Synthesis of Compound BD-3

BD-3 was synthesized by the synthetic route described below.

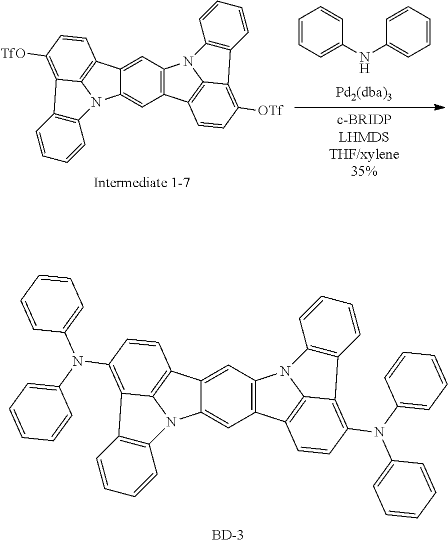 [SMILES: O=S(=O)(Oc1ccc2c3cc4c(cc3n3c5ccccc5c1c23)c1ccc(OS(=O)(=O)C(F)(F)F)c2c3ccccc3n4c12)C(F)(F)F ; c1ccc(Nc2ccccc2)cc1 ; c1ccc(N(c2ccccc2)c2ccc3c4cc5c(cc4n4c6ccccc6c2c34)c2ccc(N(c3ccccc3)c3ccccc3)c3c4ccccc4n5c23)cc1 ; "BD-3"]

### Synthesis of BD-3

Under an argon atmosphere, intermediates 1-7 (0.70 g, 1.00 mmol), diphenylamine (0.420 g, 2.50 mmol), tris(dibenzylideneacetone)diparadium(0) (Pd₂(dba)₃, 21 mg, 0.020 mmol), di-tert-butyl(1-methyl-2,2-diphenylcyclopropyl)phosphine (c-BRIDP, 28 mg, 0.08 mmol) were dissolved in xylene (85 mL), and 1M lithium bis(trimethylsilyl)amide (LHMDS) solution in tetrahydrofuran (2.5 mL, 2.5 mmol was added and refluxed for 8 hours. After completion of the reaction, the solid obtained by filtration through celite and distilling off the solvent was purified by column chromatography to obtain a yellow solid (259 mg, yield: 35%). The obtained solid was BD-3, which was an intended product, and the result of mass spectrometric analysis was: m/e=738 for a molecular weight of 739.

### Synthesis Example 4: Synthesis of Compound BD-4

BD-4 was synthesized by the synthetic route described below.

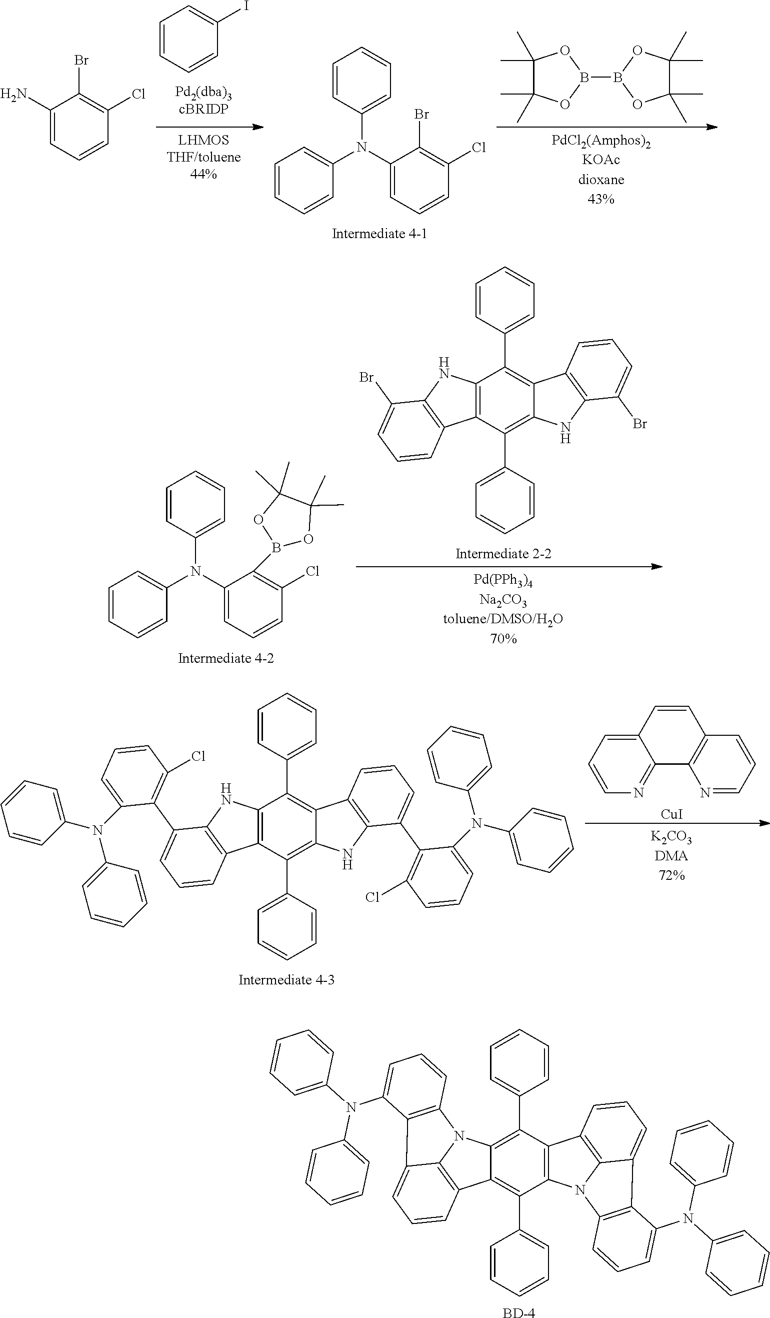

### Synthesis of Intermediate 4-1

Under an argon atmosphere, 2-bromo-3-chloroaniline (6.19 g, 30.0 mmol), iodobenzene (13.5 g, 66.0 mmol), tris(dibenzylideneacetone)dipaladium(0) (Pd₂(dba)₃, 1.37 g, 1.50 mmol), di-tert-butyl(1-methyl-2,2-diphenylcyclopropyl)phosphine (2.12 g, 6.00 mmol) were suspended in toluene (1500 mL) and heated and stirred at 80° C. for 30 minutes. 1M lithium bis(trimethylsilyl)amide solution in tetrahydrofuran (75.0 mL, 75.0 mmol) was added dropwise and heated and stirred at 110° C. for 6 hours. After completion of the reaction, the residue obtained by filtration through celite and concentration was purified by silica gel column chromatography to obtain a white solid (4.73 g, yield: 44%). The obtained solid was intermediate 4-1, which was an intended product, and the result of mass spectrometric analysis was: m/e=359 for a molecular weight of 359.

### Synthesis of Intermediate 4-2

Under an argon atmosphere, intermediate 4-1 (4.66 g, 13.0 mmol), bis(pinacolato)diboron (3.63 g, 14.3 mmol), bis[di-(tert-butyl(4-dimethylaminophenyl)phosphine]dichloropalladium (PdCl₂(Amphos)₂, 276 mg, 0.39 mmol), potassium acetate (3.83 g, 39 mmol) were suspended in dioxane (52 mL) and refluxed for 8 hours. After completion of the reaction, the solvent of the suspension was concentrated by short-pass silica gel column chromatography. The resulting solid was washed with methanol to obtain a white solid (2.27 g, yield: 43%). The obtained solid was intermediate 4-2, which was an intended product, and the result of mass spectrometric analysis was: m/e=405 for a molecular weight of 406.

### Synthesis of Intermediate 4-3

Under an argon atmosphere, intermediate 2-2 (1.02 g, 1.80 mmol), intermediate 4-2 (2.19 g, 5.40 mmol), tetrakis(triphenylphosphine)palladium(0) (Pd(PPh₃)₄, 208 mg, 0.18 mmol) were dissolved in toluene (50 mL) and dimethylsulfoxide (100 mL), and a 2M aqueous solution of Na₂CO₃ (27 mL) was added thereto and heated stirred at 100° C. for 6 hours. After completion of the reaction, the solvent of the suspension was concentrated by short-pass silica gel column chromatography. The resulting residue was washed with ethyl acetate and then washed with toluene to obtain a yellow solid (1.21 g, yield: 70%). The obtained solid was intermediate 4-3, which was an intended product, and the result of mass spectrometric analysis was: m/e=962 for a molecular weight of 964.

### Synthesis of BD-4

Under an argon atmosphere, intermediate 4-3 (1.16 g, 1.20 mmol), iodide copper(I) (0.27 g, 1.44 mmol), 1,10-phenanthroline (0.26 g, 1.44 mmol), K₂CO₃ (1.33 g, 9.6 mmol) were suspended in N,N-dimethylacetamide (220 mL) and heated and stirred at 120° C. for 15 hours. After completion of the reaction, the solvent of the suspension was concentrated by short-pass silica gel column chromatography. The resulting residue was recrystallized with chlorobenzene, washed with toluene and then washed with methanol to obtain a yellow solid (0.77 g, yield: 72%). The obtained solid was BD-4, which was an intended product, and the result of mass spectrometric analysis was: m/e=890 for a molecular weight of 891.

### Synthesis Example 5: Synthesis of Compound BD-5

BD-5 was synthesized by the synthetic route described below.

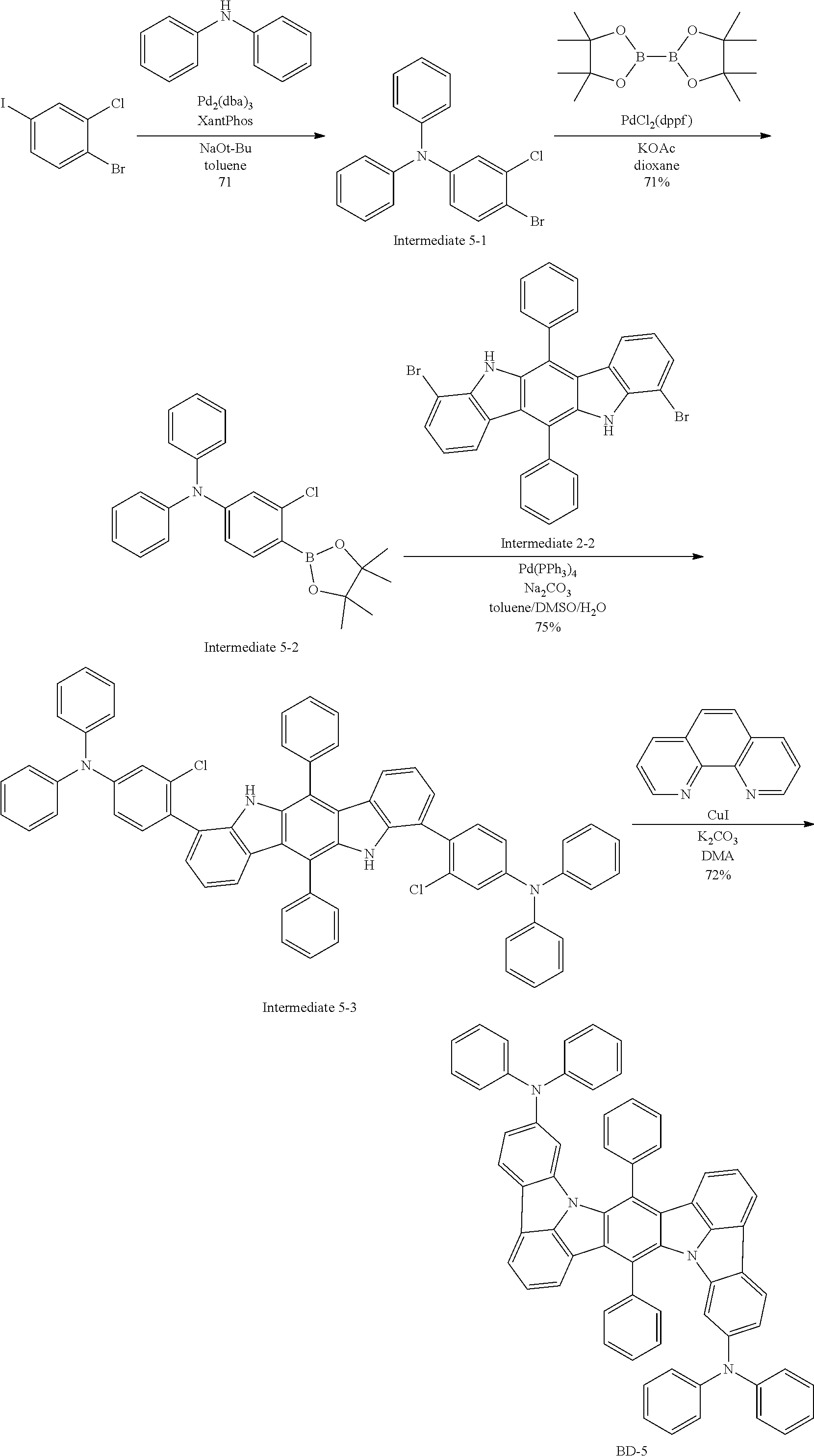

### Synthesis of Intermediate 5-1

Under an argon atmosphere, 1-bromo-2-chloro-4-iodobenzene (17.0 g, 53.6 mmol), diphenylamine (9.07 g, 53.6 mmol), tris(dibenzylideneacetone)dipaladium(0) (Pd₂(dba)₃, 981 mg, 1.07 mmol), 4,5′-bis(diphenylphosphino)-9,9′-dimethylxantene (XantPhos, 1.24 g, 2.14 mmol), NaOt-Bu (5.15 g, 53.6 mmol) were refluxed in toluene (500 mL) for 8 hours. After completion of the reaction, the residue obtained by filtration through celite and concentration was purified by silica gel column chromatography to obtain a white solid (13.6 g, yield: 71%). The obtained solid was intermediate 5-1, which was an intended product, and the result of mass spectrometric analysis was: m/e=359 for a molecular weight of 359.

### Synthesis of Intermediate 5-2

Under an argon atmosphere, intermediate 5-1 (13.6 g, 38.0 mmol), bis(pinacolato)diboron (19.3 g, 76.0 mmol), dichloro[1,1′-bis(diphenylphosphino)ferrocene]palladium (PdCl₂(dppf), 557 mg, 0.761 mmol), potassium acetate (7.46 g, 76 mmol) were suspended in dioxane (400 mL) and refluxed for 7 hours. After completion of the reaction, the solvent of the suspension was concentrated by short-pass silica gel column chromatography. The resulting solid was washed with methanol to obtain a white solid (11.0 g, yield: 71%). The obtained solid was intermediate 5-2, which was an intended product, and the result of mass spectrometric analysis was: m/e=405 for a molecular weight of 406.

### Synthesis of Intermediate 5-3

Under an argon atmosphere, intermediate 2-2 (5.00 g, 8.83 mmol), intermediate 5-2 (10.8 g, 26.5 mmol), tetrakis(triphenylphosphine)palladium(0) (Pd(PPh₃)₄, 1.02 g, 0.883 mmol) were dissolved in toluene (250 mL) and dimethylsulfoxide (500 mL), and a 2M aqueous solution of Na₂CO₃ (130 mL) was added thereto and heated and stirred at 100° C. for 6 hours. After completion of the reaction, the solvent of the suspension was concentrated by short-pass silica gel column chromatography. The resulting residue was washed with ethyl acetate and then washed with toluene to obtain a yellow solid (6.39 g, yield: 75%). The obtained solid was intermediate 5-3, which was an intended product, and the result of mass spectrometric analysis was: m/e=962 for a molecular weight of 964.

### Synthesis of BD-5

Under an argon atmosphere, intermediates 5-3 (6.24 g, 6.47 mmol), iodide copper(I) (1.48 g, 7.77 mmol), 1,10-phenanthroline (1.40 g, 7.77 mmol), K₂CO₃ (7.16 g, 51.8 mmol) were suspended in N,N-dimethylacetamide (1.2 L) and heated and stirred at 120° C. for 15 hours. After completion of the reaction, the solvent of the suspension was concentrated by short-pass silica gel column chromatography. The resulting residue was recrystallized with chlorobenzene, washed with toluene and then washed with methanol to obtain a yellow solid (4.54 g, yield: 79%). The obtained solid was BD-5, which was an intended product, and the result of mass spectrometric analysis was: m/e=890 for a molecular weight of 891.

### Synthesis Example 6: Synthesis of Compound BD-6

BD-6 was synthesized by the synthetic route described below.

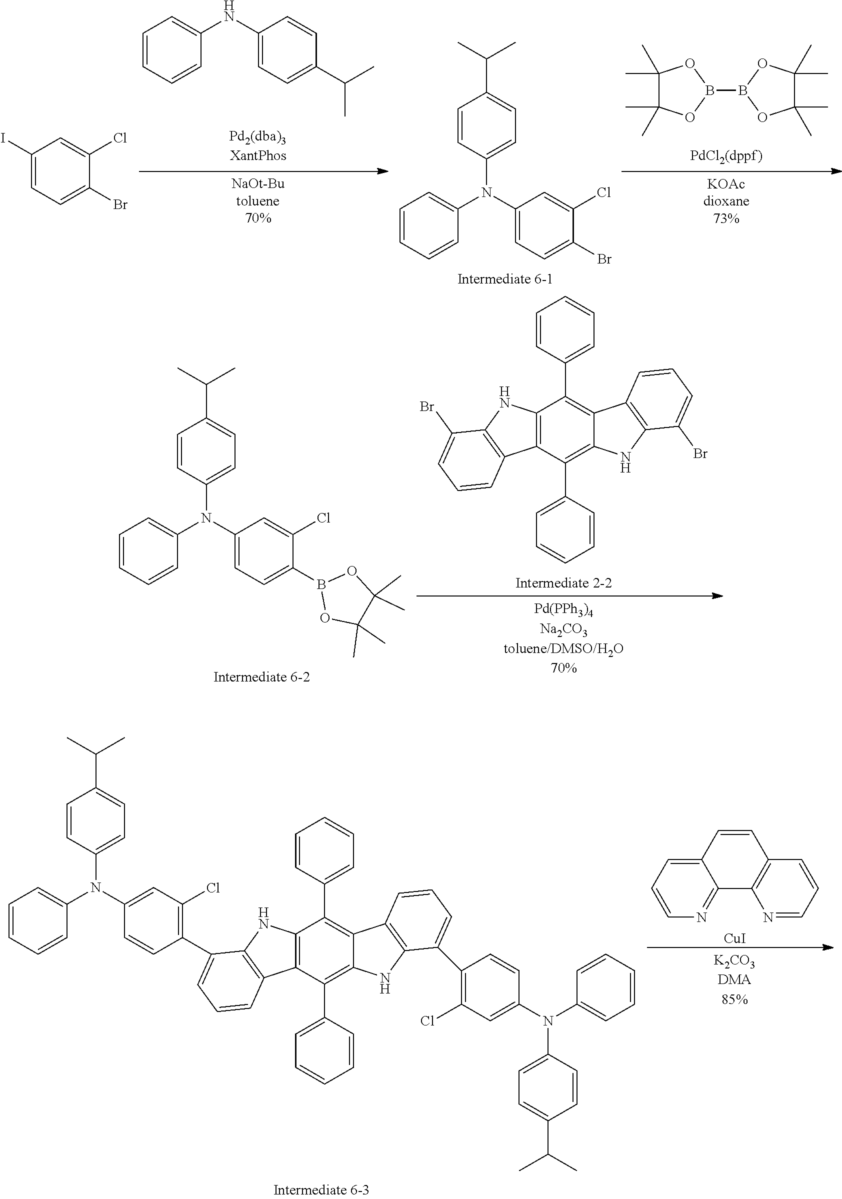

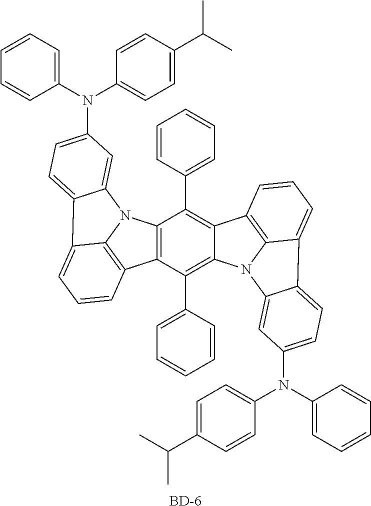

### Synthesis of Intermediate 6-1

Under an argon atmosphere, 1-bromo-2-chloro-4-iodo benzene (17.0 g, 53.6 mmol), 4-isopropyl-N-phenylaniline (11.3 g, 53.6 mmol), tris(dibenzylideneacetone)dipaladium(0) (Pd₂(dba)₃, 981 mg, 1.07 mmol), 4,5′-bis(diphenylphosphino)-9,9′-dimethylxanthene (XantPhos, 1.24 g, 2.14 mmol), NaOt-Bu (5.15 g, 53.6 mmol) were refluxed in toluene (500 mL) for 8 hours. After completion of the reaction, the residue obtained by filtration through celite and concentration was purified by silica gel column chromatography to obtain a white solid (15.0 g, yield: 70%). The obtained solid was intermediate 6-1, which was an intended product, and the result of mass spectrometric analysis was: m/e=401 for a molecular weight of 401.

### Synthesis of Intermediate 6-2

Under an argon atmosphere, intermediate 6-1 (15.0 g, 37.5 mmol), bis(pinacolato)diboron (19.1 g, 75.0 mmol), dichloro[1,1′-bis(diphenylphosphino)ferrocene]palladium (PdCl₂(dppf), 550 mg, 0.75 mmol), potassium acetate (7.36 g, 75 mmol) were suspended in dioxane (400 mL) and refluxed for 7 hours. After completion of the reaction, the solvent of the suspension was concentrated by short-pass silica gel column chromatography. The resulting solid was washed with methanol to obtain a white solid (12.3 g, yield: 73%). The obtained solid was intermediate 6-2, which was an intended product, and the result of mass spectrometric analysis was: m/e=447 for a molecular weight of 448.

### Synthesis of Intermediate 6-3

Under an argon atmosphere, intermediate 2-2 (5.10 g, 9.00 mmol), intermediate 6-2 (12.1 g, 27.0 mmol), tetrakis(triphenylphosphine)palladium(0) (Pd(PPh₃)₄, 1.04 g, 0.90 mmol) were dissolved in toluene (250 mL) and dimethylsulfoxide (500 mL), and a 2M aqueous solution of Na₂CO₃ (135 mL) was added thereto, and heated and stirred at 100° C. for 6 hours. After completion of the reaction, the solvent of the suspension was concentrated by short-pass silica gel column chromatography. The resulting residue was washed with ethyl acetate and then washed with toluene to obtain a yellow solid (6.60 g, yield: 70%). The obtained solid was intermediate 6-3, which was an intended product, and the result of mass spectrometric analysis was: m/e=1046 for a molecular weight of 1048.

### Synthesis of BD-6

Under an argon atmosphere, intermediates 6-3 (6.60 g, 6.30 mmol), iodide copper(I) (1.44 g, 7.56 mmol), 1,10-phenanthroline (1.36 g, 7.56 mmol), K₂CO₃ (6.97 g, 50.4 mmol) were suspended in N,N-dimethylacetamide (1.15 L), and heated and stirred at 120° C. for 15 hours. After completion of the reaction, the solvent of the suspension was concentrated by short-pass silica gel column chromatography. The resulting residue was recrystallized with chlorobenzene, washed with toluene and then washed with methanol to obtain a yellow solid (3.99 g, yield: 65%). The obtained solid was BD-6, which was an intended product, and the result of mass spectrometric analysis was: m/e=974 for a molecular weight of 975.

### Synthesis Example 7: Synthesis of Compound BD-7

BD-7 was synthesized by the synthetic route described below.

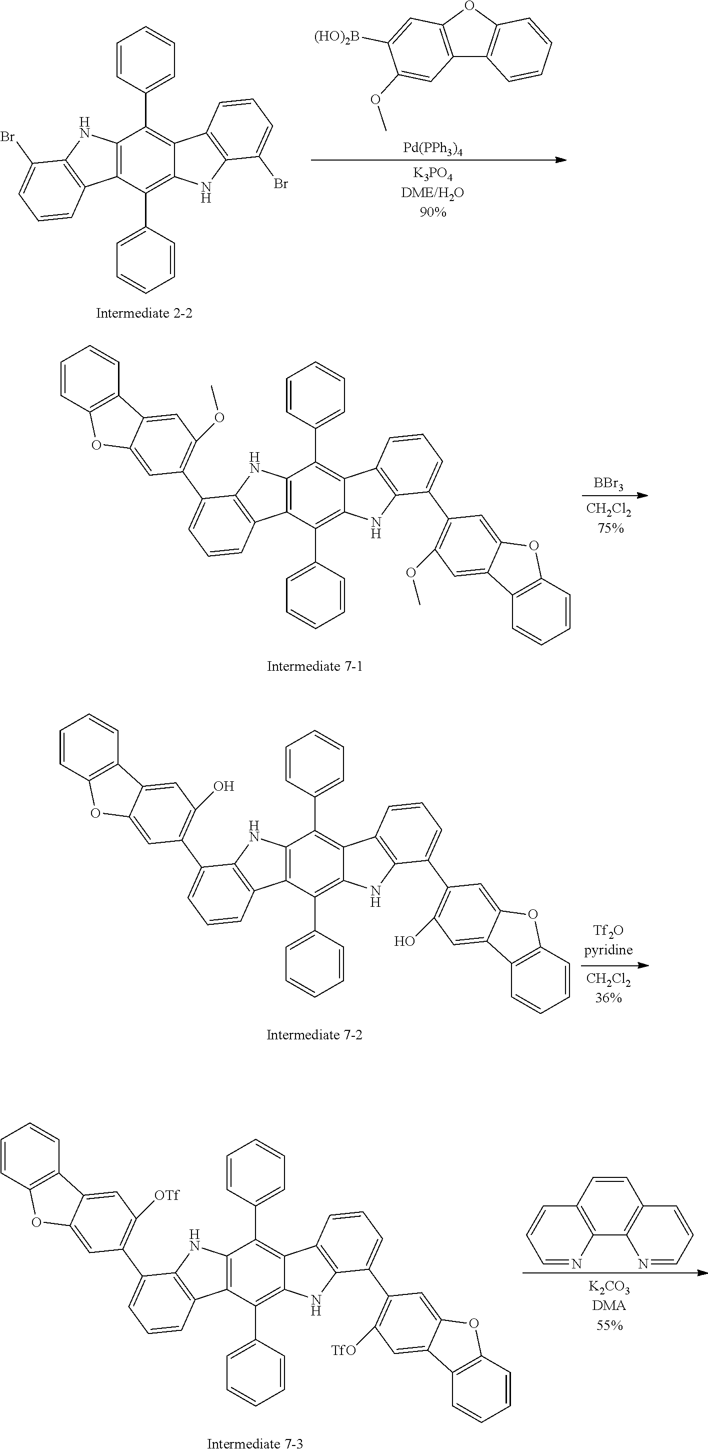

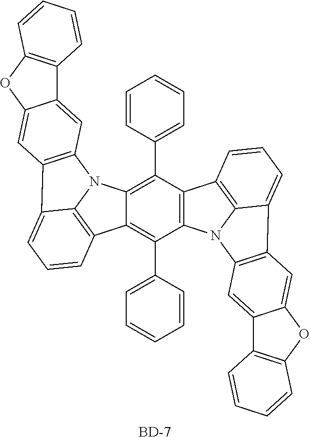 [SMILES: c1ccc(-c2c3c4cccc5c6cc7oc8ccccc8c7cc6n(c3c(-c3ccccc3)c3c6cccc7c8cc9oc%10ccccc%10c9cc8n(c23)c76)c54)cc1 ; "BD-7"]

### Synthesis of Intermediate 7-1

Under an argon atmosphere, 1,2-dimethoxyethane (80 mL) and water (20 mL) were added to intermediate 2-2 (3.00 g, 5.30 mmol), 2-methoxydibenzofuranyl-3-boronic acid (3.85 g, 15.9 mmol), tetrakis(triphenylphosphine)palladium(0) (Pd(PPh₃)₄, 918 mg, 0.795 mmol) and K₃PO₄ (6.74 g, 31.8 mmol), and stirred at 80° C. for 12 hours. After completion of the reaction, an organic layer was concentrated and a solid was collected by filtration. The resulting material was purified by column chromatography to obtain a light yellow solid (3.82 g, yield: 90%). The obtained solid was intermediate 7-1, which was an intended product, and the result of mass spectrometric analysis was: m/e=800 for a molecular weight of 801.

### Synthesis of Intermediate 7-2

Under an argon atmosphere, intermediate 7-1 (3.80 g, 4.74 mmol) was dissolved in dichloromethane (100 mL), and a 1M solution of BBr₃ in dichloromethane (30 mL) was added and stirred for 24 hours. After completion of the reaction, methanol and water were added thereto, and the resulting mixture was subjected to extraction with ethyl acetate. The solvent was distilled off, and the resulting residue was purified by column chromatography to obtain a light yellow solid (2.74 g, yield: 75%). The obtained solid was intermediate 7-2, which was an intended product, and the result of mass spectrometric analysis was: m/e=772 for a molecular weight of 773.

### Synthesis of Intermediate 7-3

Under an argon atmosphere, Intermediate 7-2 (2.50 g, 3.24 mmol) was suspended in dichloromethane (100 mL), and pyridine (2 mL) and trifluoromethanesulfonic anhydride (2.74 g, 9.72 mmol) were added and stirred for 6 hours. After completion of the reaction, water was added thereto, and only an organic layer was concentrated, and a precipitated solid was collected by filtration. The resulting material was purified by column chromatography to obtain a light yellow solid (1.21 g, yield: 36%). The obtained solid was intermediate 7-3, which was an intended product, and the result of mass spectrometric analysis was: m/e=1036 for a molecular weight of 1037.

### Synthesis of BD-7

Under an argon atmosphere, intermediate 7-3 (1.00 g, 0.963 mmol), iodide copper(I) (92 mg, 0.482 mmol), 1,10-phenanthroline (87 mg, 0.482 mmol), K₂CO₃ (532 mg, 3.85 mmol) were suspended in N,N-dimethylacetamide (20 mL) and heated and stirred at 160° C. for 8 hours. After completion of the reaction, water was added thereto, and a precipitate was collected by filtration. The resulting material was purified by column chromatography to obtain a yellow solid (390 mg, yield: 55%). The obtained solid was BD-7, which was an intended product, and the result of mass spectrometric analysis was: m/e=736 for a molecular weight of 737.

Although only some exemplary embodiments and/or examples of this invention have been described in detail above, those skilled in the art will readily appreciate that many modifications are possible in the exemplary embodiments and/or examples without materially departing from the novel teachings and advantages of this invention. Accordingly, many of these modifications are within the scope of the invention.

The documents described in the specification and the specification of Japanese application(s) on the basis of which the present application claims Paris convention priority are incorporated herein by reference in its entirety.

1. An organic electroluminescence device comprising:
a cathode;
an anode; and
an organic layer disposed between the cathode and the anode, wherein
the organic layer comprises an emitting layer and a first layer;
the first layer is disposed between the anode and the emitting layer and is directly adjacent to the emitting layer;
the emitting layer comprises a compound represented by the following formula (A1); and
the first layer comprises a compound represented by the following formula (B1) or the following formula (C1): 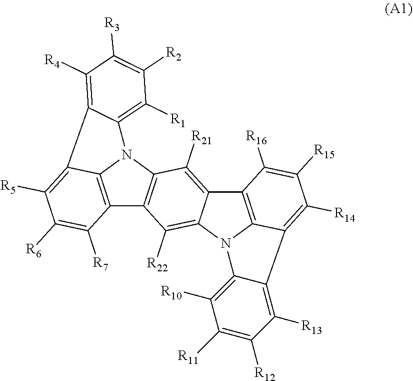
wherein in the formula (A1),
one or more sets of adjacent two or more among R₁ to R₇ and R₁₀ to R₁₆ form a substituted or unsubstituted, saturated or unsaturated ring by bonding with each other, or do not form a substituted or unsubstituted, saturated or unsaturated ring;
R₁ to R₇ and R₁₀ to R₁₆ which do not form the substituted or unsubstituted, saturated or unsaturated ring, and R₂₁ and R₂₂ are independently a hydrogen atom or substituent;
the substituent is
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
Si(R₉₀₁)(R₉₀₂)(R₉₀₃),
O—(R₉₀₄),
S—(R₉₀₅),
N(R₉₀₆)(R₉₀₇),
a halogen atom, a cyano group, a nitro group,
a substituted or unsubstituted aryl group including 6 to 50 ring carbon atoms, or
a substituted or unsubstituted monovalent heterocyclic group including 5 to 50 ring atoms;
R₉₀₁ to R₉₀₇ are independently
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms;
when two or more of each of R₉₀₁ to R₉₀₇ are present, the two or more of each of R₉₀₁ to R₉₀₇ may be the same or different; and
provided that the formula (A1) satisfies one or both of the following requirements (i) and (ii):
(i) one or more sets of adjacent two or more among R₁ to R₇ and R₁₀ to R₁₆ form a substituted or unsubstituted, saturated or unsaturated ring by bonding with each other,
(ii) one or more among R₁ to R₇, R₁₀ to R₁₆, R₂₁ and R₂₂ is the substituent: 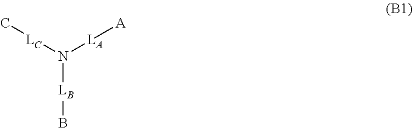
wherein in the formula (B1),
L_{A}, L_{B} and L_{C} are independently a single bond, a substituted or unsubstituted arylene group including 6 to 18 ring carbon atoms, or a substituted or unsubstituted divalent heterocyclic group including 5 to 13 ring atoms;
A, B and C are independently
a substituted or unsubstituted aryl group including 6 to 30 ring carbon atoms,
a substituted or unsubstituted monovalent heterocyclic group including 5 to 30 ring atoms, or
—Si(R′₉₀₁)(R′₉₀₂)(R′₉₀₃);
R′₉₀₁ to R′₉₀₃ are independently a substituted or unsubstituted aryl group including 6 to 30 ring carbon atoms; and
when two or more of each of one or more among R′₉₀₁ to R′₉₀₃ are present, each of the two or more R′901 to R′903 may be the same or different; 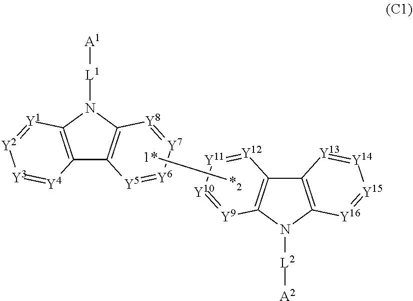
wherein in the formula (C1),
A¹ and A² are independently a substituted or unsubstituted aryl group including 6 to 30 ring carbon atoms, or a substituted or unsubstituted monovalent heterocyclic group including 5 to 30 ring atoms;
one of Y⁵ to Y⁸ is a carbon atom which is bonded to *1;
one of Y⁹ to Y¹² is a carbon atom which is bonded to *2;
Y¹ to Y⁴, Y¹³ to Y¹⁶, Y⁵ to Y⁸ which are not a carbon atom bonded to *1, and Y⁹ to Y¹² which are not a carbon atom bonded to *2 are independently CR;
when a plurality of R's is present, one or more sets of adjacent two or more among the plurality of R's form a substituted or unsubstituted, saturated or unsaturated ring by bonding with each other, or do not form a substituted or unsubstituted, saturated or unsaturated ring;
R which does not form the substituted or unsubstituted, saturated or unsaturated ring is
a hydrogen atom,
a cyano group,
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
a halogen atom, a nitro group,
a substituted or unsubstituted aryl group including 6 to 50 ring carbon atoms, or
a substituted or unsubstituted monovalent heterocyclic group including 5 to 50 ring atoms;
R₉₀₁ to R₉₀₄ are as defined in the formula (A1);
when a plurality of R's is present, the plurality of R's may be the same with or different from each other; and
L¹ and L² are independently a single bond, a substituted or unsubstituted arylene group including 6 to 30 ring carbon atoms, or a substituted or unsubstituted divalent heterocyclic group including 5 to 30 ring atoms.

2. The organic electroluminescence device according to claim 1, wherein the compound represented by the formula (A1) satisfies only the requirement (i).

3. The organic electroluminescence device according to claim 1, wherein the compound represented by the formula (A1) satisfies only the requirement (ii).

4. The organic electroluminescence device according to claim 1, wherein the compound represented by the formula (A1) satisfies the requirements (i) and (ii).

5. The organic electroluminescence device according to claim 1, wherein one or more among R₁ to R₇ and R₁₀ to R₁₆ of the formula (A1) is —N(R₉₀₆)(R₉₀₇).

6. The organic electroluminescence device according to claim 1, wherein two or more of R₁ to R₇ and R₁₀ to R₁₆ of the formula (A1) are —N(R₉₀₆)(R₉₀₇).

7. The organic electroluminescence device according to claim 6, wherein the compound represented by the formula (A1) is a compound represented by the following formula (A10): 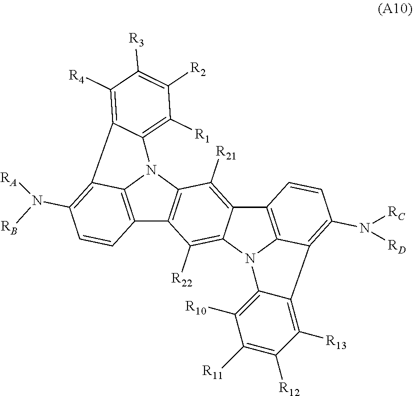
wherein in the formula (A10),
R₁ to R₄, R₁₀ to R₁₃, R₂₁ and R₂₂ are as defined in the formula (A1); and
R_{A}, R_{B}, R_{C} and R_{D} are independently a substituted or unsubstituted aryl group including 6 to 18 ring carbon atoms, or a substituted or unsubstituted monovalent heterocyclic group including 5 to 18 ring atoms.

8. The organic electroluminescence device according to claim 7, wherein the compound represented by the formula (A10) is a compound represented by the following formula (A11): 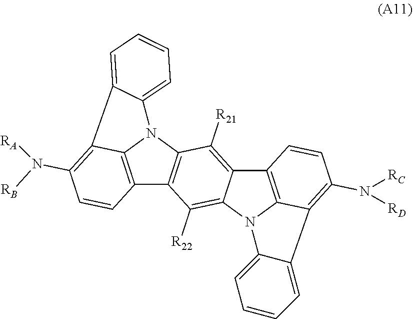
wherein in the formula (A11),
R₂₁, R₂₂, R_{A}, R_{B}, R_{C} and R_{D} are as defined in the formula (A10).

9. The organic electroluminescence device according to claim 7, wherein R_{A}, R_{B}, R_{C} and R_{D} are independently a substituted or unsubstituted aryl group including 6 to 18 ring carbon atoms.

10. The organic electroluminescence device according to claim 7, wherein R_{A}, R_{B}, R_{C} and R_{D} are independently a substituted or unsubstituted phenyl group.

11. The organic electroluminescence device according to claim 1, wherein one or more sets selected from the group consisting of R₁ and R₂, R₂ and R₃, R₃ and R₄, R₁₀ and R₁₁, R₁₁ and R₁₂, and R₁₂ and R₁₃ of the formula (A1) form a ring represented by the following formula (X): 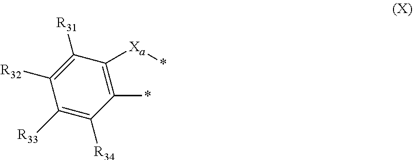
wherein in the formula (X),
two “*”s are respectively bonded to R₁ and R₂, R₂ and R₃, R₃ and R₄, R₁₀ and R₁₁, R₁₁ and R₁₂ or R₁₂ and R₁₃ of the formula (A1);
R₃₁ to R₃₄ are independently a hydrogen atom, a substituted or unsubstituted alkyl group including 1 to 50 carbon atoms, or a substituted or unsubstituted aryl group including 6 to 50 ring carbon atoms;
Xₐ is selected from the group consisting of O, S and N(R₃₅), and two Xₐ's may be the same with or different from each other;
R₃₅ forms a substituted or unsubstituted, saturated or unsaturated ring by bonding with R₃₁, or does not form the ring; and
R₃₅ which does not form the ring is
a hydrogen atom,
a substituted or unsubstituted alkyl group including 1 to 50 carbon atoms,
a substituted or unsubstituted cycloalkyl group including 3 to 50 ring carbon atoms,
a substituted or unsubstituted aryl group including 6 to 50 ring carbon atoms, or
a substituted or unsubstituted monovalent heterocyclic group including 5 to 50 ring atoms.

12. The organic electroluminescence device according to claim 11, wherein the compound represented by the formula (A1) is a compound represented by the following formula (A21): 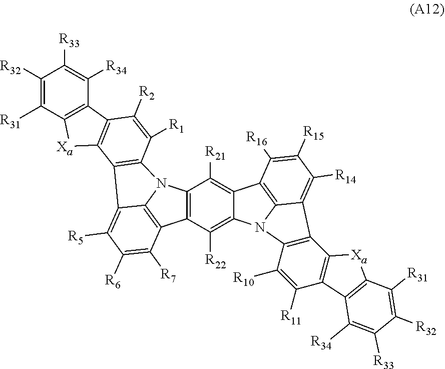
wherein in the formula (A12),
R₁, R₂, R₅ to R₇, R₁₀, R₁₁, R₁₄ to R₁₆, R₂₁, R₂₂, R₃₁ to R₃₄ and Xₐ are as defined in the formula (A1) and the formula (X).

13. The organic electroluminescence device according to claim 1, wherein R₂₁ and R₂₂ are hydrogen atoms.

14. The organic electroluminescence device according to claim 1, wherein the compound represented by the formula (B1) is a compound represented by the following formula (B11): 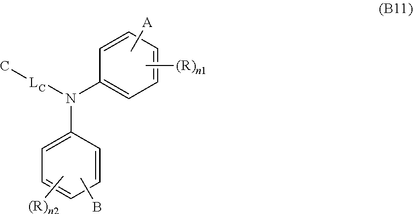
wherein in the formula (B11),
L_{C}, A, B and C are as defined in the formula (B1);
when a plurality of R's is present, one or more sets of adjacent two or more among the plurality of R's form a substituted or unsubstituted, saturated or unsaturated ring by bonding with each other, or do not form a substituted or unsubstituted, saturated or unsaturated ring;
R which does not form the substituted or unsubstituted, saturated or unsaturated ring is
a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
a substituted or unsubstituted aryl group including 6 to 50 ring carbon atoms, or
a substituted or unsubstituted monovalent heterocyclic group including 5 to 50 ring atoms;
R₉₀₁ to R₉₀₄ are as defined in the formula (A1);
n1 and n2 are independently an integer of 0 to 4; and
when a plurality of R's is present, the plurality of R's may be the same with or different from each other.

15. The organic electroluminescence device according to claim 1, wherein two among A to C in the formula (B1) or (B11) are groups represented by the following formula (Y), and the two groups represented by the formula (Y) may be the same or different: 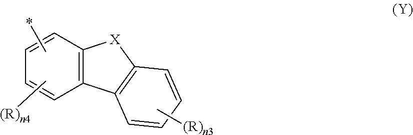
wherein in the formula (Y), X is CR₅₁R₅₂, NR₅₃, an oxygen atom, or a sulfur atom;
when X is CR₅₁R₅₂, R₅₁ and R₅₂ form a substituted or unsubstituted, saturated or unsaturated ring by bonding with each other, or do not form a substituted or unsubstituted, saturated or unsaturated ring;
when a plurality of R's is present, one or more sets of adjacent two or more among the plurality of R's form a substituted or unsubstituted, saturated or unsaturated ring by bonding with each other, or do not form a substituted or unsubstituted, saturated or unsaturated ring;
R which does not form the substituted or unsubstituted, saturated or unsaturated ring is independently
a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
a substituted or unsubstituted aryl group including 6 to 50 ring carbon atoms, or
a substituted or unsubstituted monovalent heterocyclic group including 5 to 50 ring atoms;
R₅₃, and R₅₁ and R₅₂ which do not form the substituted or unsubstituted, saturated or unsaturated ring are independently
a hydrogen atom,
a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
a substituted or unsubstituted aryl group including 6 to 50 ring carbon atoms, or
a substituted or unsubstituted monovalent heterocyclic group including 5 to 50 ring atoms;
R₉₀₁ to R₉₀₄ are as defined in the formula (A1);
n3 is an integer of 0 to 4, and n4 is an integer of 0 to 3;
when a plurality of R's is present, the plurality of R's may be the same as or different from each other; and
“*” is bonded to any of L_{A} to L_{C} in the formula (B1), or L_{C}, a benzene ring which is bonded to A, or a benzene ring which is bonded to B in the formula (B11).

16. The organic electroluminescence device according to claim 1, wherein at least one of A to C in the formula (B1) or (B11) is a group represented by the following formula (Y1) or a group represented by the following formula (Y2): 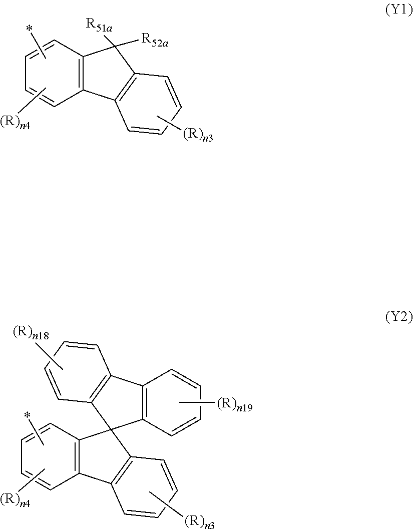
wherein in the formulas (Y1) and (Y2),
“*” is bonded to any of L_{A} to L_{C} in the formula (B1), or L_{C}, a benzene ring which is bonded to A, or a benzene ring which is bonded to B in the formula (B11);
R₅₁ₐ and R₅₂ₐ do not form a substituted or unsubstituted, saturated or unsaturated ring by bonding with each other;
R₅₁ₐ and R₅₂ₐ are independently
a hydrogen atom,
a cyano group,
a substituted or unsubstituted alkyl group including 1 to 50 carbon atoms, a substituted or unsubstituted cycloalkyl group including 3 to 50 ring carbon atoms,
—Si(R₉₀₁)(R₉₀₂)(R₉₀₃),
—O—(R₉₀₄),
a substituted or unsubstituted aryl group including 6 to 50 ring carbon atoms, or
a substituted or unsubstituted monovalent heterocyclic group including 5 to 50 ring atoms;
R₉₀₁ to R₉₀₄ are as defined in the formula (A1);
n4 is an integer of 0 and 3;
n3, n18, and n19 are independently an integer of 0 to 4;
when n3, n4, n18 or n19 is 2 or more, one or more sets of adjacent two or more among a plurality of R's form a substituted or unsubstituted, saturated or unsaturated ring by bonding with each other, or do not form a substituted or unsubstituted, saturated or unsaturated ring;
R which does not form the substituted or unsubstituted, saturated or unsaturated ring is
a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
a substituted or unsubstituted aryl group including 6 to 50 ring carbon atoms, or
a substituted or unsubstituted monovalent heterocyclic group including 5 to 50 ring atoms;
R₉₀₁ to R₉₀₄ are as defined in the formula (A1);
when a plurality of R's is present, the plurality of R's may be the same with or different from each other; and
when two or more groups represented by the formula (Y1) or (Y2) are present, the two or more groups represented by the formula (Y1) or (Y2) may be the same with or different from each other.

17. The organic electroluminescence device according to claim 1, wherein the compound represented by the formula (B1) is a compound represented by the following formula (B12) or (B13): 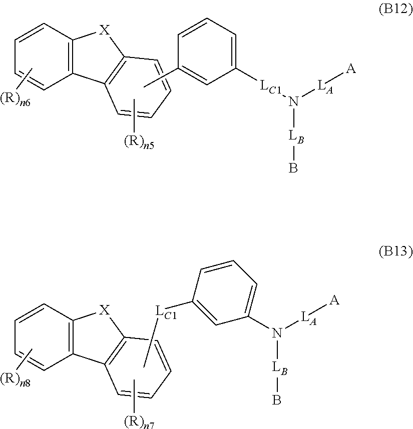
wherein in the formulas (B12) and (B13),
L_{A}, L_{B}, A and B are as defined in the formula (B1);
L_{C1} is an arylene group including 6 to 12 ring carbon atoms;
X is CR₅₁R₅₂, NR₅₃, an oxygen atom, or a sulfur atom;
when X is CR₅₁R₅₂, R₅₁ and R₅₂ form a substituted or unsubstituted, saturated or unsaturated ring by bonding with each other, or do not form a substituted or unsubstituted, saturated or unsaturated ring;
when a plurality of R's is present, one or more sets of adjacent two or more among the plurality of R's form a substituted or unsubstituted, saturated or unsaturated ring by bonding with each other, or do not form a substituted or unsubstituted, saturated or unsaturated ring;
R which does not form the substituted or unsubstituted, saturated or unsaturated ring is independently
a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
a substituted or unsubstituted aryl group including 6 to 50 ring carbon atoms, or
a substituted or unsubstituted monovalent heterocyclic group including 5 to 50 ring atoms;
R₅₃, and R₅₁ and R₅₂ which do not form the substituted or unsubstituted, saturated or unsaturated ring are independently
a hydrogen atom,
a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
a substituted or unsubstituted aryl group including 6 to 50 ring carbon atoms, or
a substituted or unsubstituted monovalent heterocyclic group including 5 to 50 ring atoms;
R₉₀₁ to R₉₀₄ are as defined in the formula (A1);
n5 and n7 are independently an integer of 0 to 3, and n6 and n8 are independently an integer of 0 to 4; and
when a plurality of R's is present, the plurality of R's may be the same with or different from each other.

18. The organic electroluminescence device according to claim 1, wherein the compound represented by the formula (B1) is a compound represented by the following formula (B14) or (B15): 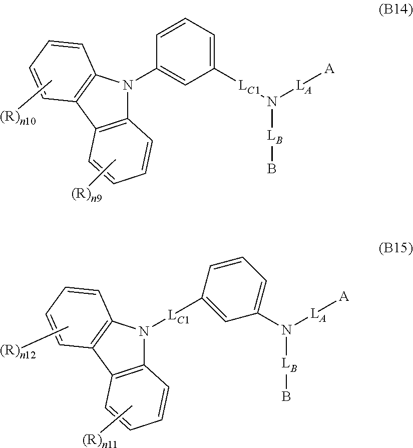
wherein in the formulas (B14) and (B15),
L_{A}, L_{B}, A and B are as defined in the formula (B1);
L_{C1} is an arylene group including 6 to 12 ring carbon atoms;
when a plurality of R's is present, one or more sets of adjacent two or more among the plurality of R's form a substituted or unsubstituted, saturated or unsaturated ring by bonding with each other, or do not form a substituted or unsubstituted, saturated or unsaturated ring;
R which does not form the substituted or unsubstituted, saturated or unsaturated ring is
a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
a substituted or unsubstituted aryl group including 6 to 50 ring carbon atoms, or
a substituted or unsubstituted monovalent heterocyclic group including 5 to 50 ring atoms;
R₉₀₁ to R₉₀₄ are as defined in the formula (A1);
n9 to n12 are independently an integer of 0 to 4; and
when a plurality of R's is present, the plurality of R's may be the same with or different from each other.

19. The organic electroluminescence device according to claim 1, wherein the compound represented by the formula (B1) is a compound represented by the following formula (B16) or (B17): 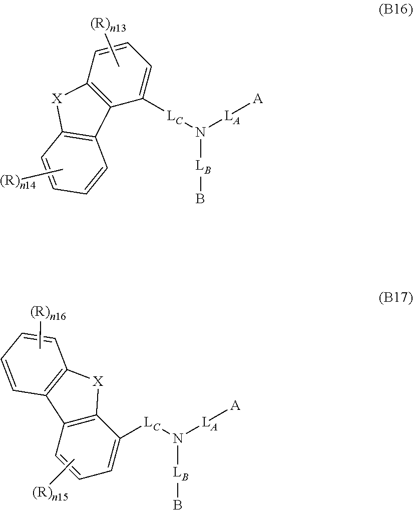
wherein in the formulas (B16) and (B17),
L_{A}, L_{B}, L_{C}, A and B are as defined in the formula (B1);
X is CR₅₁R₅₂, NR₅₃, an oxygen atom, or a sulfur atom;
when X is CR₅₁R₅₂, R₅₁ and R₅₂ form a substituted or unsubstituted, saturated or unsaturated ring by bonding with each other, or do not form a substituted or unsubstituted, saturated or unsaturated ring;
when a plurality of R's is present, one or more sets of adjacent two or more among the plurality of R's form a substituted or unsubstituted, saturated or unsaturated ring by bonding with each other, or do not form a substituted or unsubstituted, saturated or unsaturated ring;
R which does not form the substituted or unsubstituted, saturated or unsaturated ring is independently
a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
a substituted or unsubstituted aryl group including 6 to 50 ring carbon atoms, or
a substituted or unsubstituted monovalent heterocyclic group including 5 to 50 ring atoms;
R₅₃, and R₅₁ and R₅₂ which do not form the substituted or unsubstituted, saturated or unsaturated ring are independently
a hydrogen atom,
a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
a substituted or unsubstituted aryl group including 6 to 50 ring carbon atoms, or
a substituted or unsubstituted monovalent heterocyclic group including 5 to 50 ring atoms;
R₉₀₁ to R₉₀₄ are as defined in the formula (A1);
n13 and n15 are independently an integer of 0 to 3, and n14 and n16 are independently an integer of 0 to 4; and
when a plurality of R's is present, the plurality of R's may be the same with or different from each other.

20. The organic electroluminescence device according to claim 1, wherein the compound represented by the formula (B1) is a compound represented by the following formula (B18): 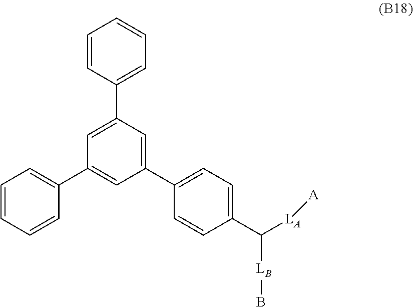
wherein in the formula (B18), L_{A}, L_{B}, A, and B are as defined in the formula (B1).

21. The organic electroluminescence device according to claim 1, wherein the compound represented by the formula (B1) is a compound represented by the following formula (B19): 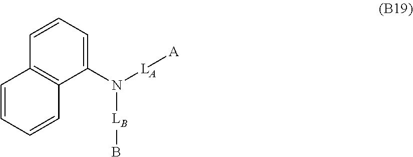
wherein in the formula (B19), L_{A}, L_{B}, A, and B are as defined in the formula (B1).

22. The organic electroluminescence device according to claim 1, wherein the compound represented by the formula (B1) is a compound represented by the following formula (B20): 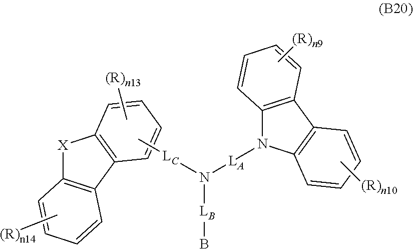
wherein in the formula (B20), L_{A} to L_{C}, and B are as defined in the formula (B1);
X is CR₅₁R₅₂, NR₅₃, an oxygen atom, or a sulfur atom;
when X is CR₅₁R₅₂, R₅₁ and R₅₂ form a substituted or unsubstituted, saturated or unsaturated ring by bonding with each other, or do not form a substituted or unsubstituted, saturated or unsaturated ring;
R₅₃, and R₅₁ and R₅₂ which do not form the substituted or unsubstituted, saturated or unsaturated ring are independently
a hydrogen atom,
a cyano group,
a substituted or unsubstituted alkyl group including 1 to 50 carbon atoms, a substituted or unsubstituted cycloalkyl group including 3 to 50 ring carbon atoms,
—Si(R₉₀₁)(R₉₀₂)(R₉₀₃),
—O—(R₉₀₄),
a substituted or unsubstituted aryl group including 6 to 50 ring carbon atoms, or
a substituted or unsubstituted monovalent heterocyclic group including 5 to 50 ring atoms;
R₉₀₁ to R₉₀₄ are as defined in the formula (A1);
n9, n10, and n14 are independently an integer of 0 to 4;
n13 is an integer of 0 and 3;
when n 9, n10, n13 or n14 is 2 or more, one or more sets of adjacent two or more among a plurality of R's form a substituted or unsubstituted, saturated or unsaturated ring by bonding with each other, or do not form a substituted or unsubstituted, saturated or unsaturated ring;
R which does not form the substituted or unsubstituted, saturated or unsaturated ring is independently
a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
a substituted or unsubstituted aryl group including 6 to 50 ring carbon atoms, or
a substituted or unsubstituted monovalent heterocyclic group including 5 to 50 ring atoms;
R₉₀₁ to R₉₀₄ are as defined in the formula (A1); and
when a plurality of R's is present, the plurality of R's may be the same with or different from each other.

23. The organic electroluminescence device according to claim 1, wherein the compound represented by the formula (B1) is a compound represented by the following formula (B21): 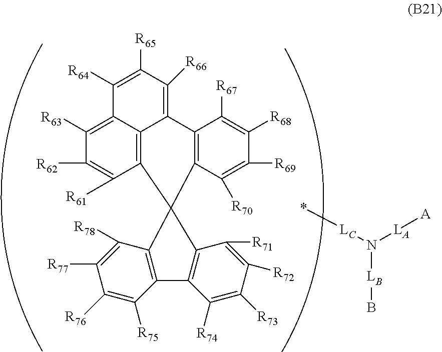
wherein in the formula (B21), L_{A} to L_{C}, A and B are as defined in the formula (B1);
one of R₆₁ to R₇₈ is a single bond which is bonded with “*”;
one or more sets of adjacent two or more among R₆₁ to R₇₈ which are not a single bond bonding with “*” do not form a substituted or unsubstituted, saturated or unsaturated ring by bonding with each other;
R₆₁ to R₇₈ which are not a single bond bonding with “*” are independently a hydrogen atom, or a substituent;
the substituent is
a substituted or unsubstituted alkyl group including 1 to 50 carbon atoms,
a substituted or unsubstituted alkenyl group including 2 to 50 carbon atoms,
a substituted or unsubstituted alkynyl group including 2 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
—S—(R₉₀₅),
—N(R₉₀₆)(R₉₀₇),
a halogen atom, a cyano group, a nitro group,
a substituted or unsubstituted aryl group including 6 to 50 ring carbon atoms, or
a substituted or unsubstituted monovalent heterocyclic group including 5 to 50 ring atoms; and
R₉₀₁ to R₉₀₇ are as defined in the formula (A1).

24. The organic electroluminescence device according to claim 1, wherein the compound represented by the formula (B1) is a compound represented by the following formula (B22): 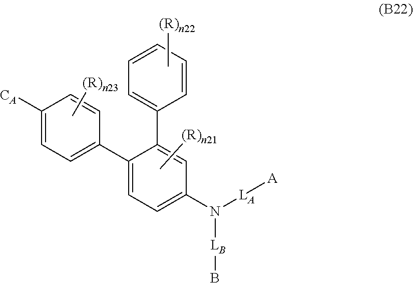
wherein in the formula (B22), L_{A}, L_{B}, A and B are as defined in the formula (B1);
C_{A} is
a substituted or unsubstituted aryl group including 6 to 30 ring carbon atoms, or
a substituted or unsubstituted monovalent heterocyclic group including 5 to 30 ring atoms;
n21 is an integer of 0 to 3;
n22 is an integer of 0 to 5;
n23 is an integer of 0 to 4;
when n21 to n23 are 2 or more, one or more sets of adjacent two or more among two or more R's do not form a substituted or unsubstituted, saturated or unsaturated ring by bonding with each other;
R is
a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
a substituted or unsubstituted aryl group including 6 to 50 ring carbon atoms, or
a substituted or unsubstituted monovalent heterocyclic group including 5 to 50 ring atoms;
R₉₀₁ to R₉₀₄ are as defined in the formula (A1); and
when a plurality of R's is present, the plurality of R's may be the same with or different from each other.

25. The organic electroluminescence device according to claim 1, wherein L_{A}, L_{B} and L_{C} are independently an aromatic hydrocarbon ring group represented by the following formula (L1) or (L2): 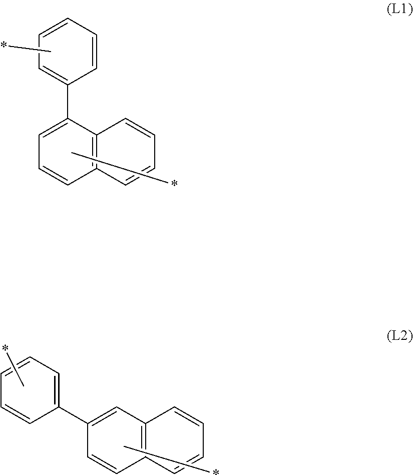
wherein in the formula (L1) or (L2), one of two “*”'s is bonded to nitrogen atom in the formula (B1) and the other is bonded to one of A to C in the formula (B1).

26. The organic electroluminescence device according to claim 1, wherein L_{A}, L_{B} and L_{C} are independently a single bond, or a substituted or unsubstituted arylene group including 6 to 12 ring carbon atoms.

27. The organic electroluminescence device according to claim 17, wherein L_{C1} is a single bond.

28. The organic electroluminescence device according to claim 1, wherein L_{C} is a single bond.

29. The organic electroluminescence device according to claim 1, wherein L_{C} is a phenylene group.

30. The organic electroluminescence device according to claim 1, wherein A is a substituted or unsubstituted aryl group including 6 to 12 ring carbon atoms.

31. The organic electroluminescence device according to claim 1, wherein A is a substituted or unsubstituted phenyl group, a substituted or unsubstituted biphenyl group, or a substituted or unsubstituted naphthyl group.

32. The organic electroluminescence device according to claim 1, wherein A is a phenyl group, a biphenyl group, or a naphthyl group.

33. The organic electroluminescence device according to claim 1, wherein B is a substituted or unsubstituted aryl group including 6 to 12 ring carbon atoms.

34. The organic electroluminescence device according to claim 1, wherein B is a substituted or unsubstituted phenyl group, a substituted or unsubstituted biphenyl group, or a substituted or unsubstituted naphthyl group.

35. The organic electroluminescence device according to claim 1, wherein B is a phenyl group, a biphenyl group, or a naphthyl group.

36. The organic electroluminescence device according to claim 1, wherein the compound represented by the formula (C1) is a compound represented by the following formula (C10), (C11), or (C12): 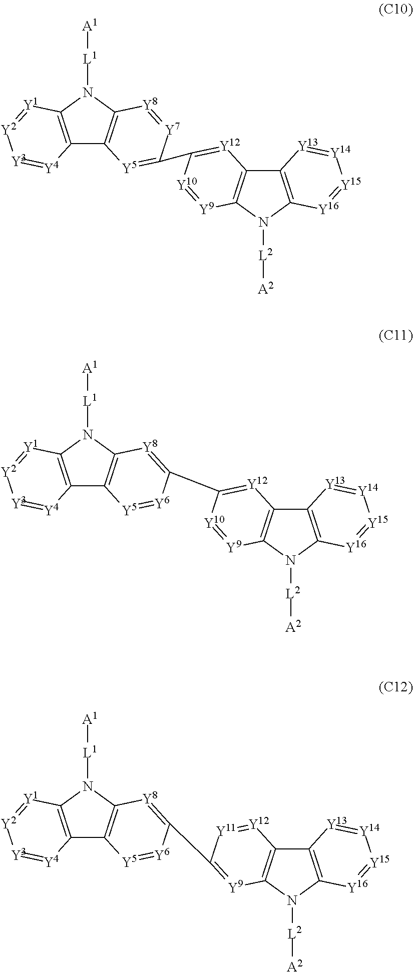
wherein in the formulas (C10), (C11), and (C12), Y¹ to Y¹⁶, A¹, A², L¹ and L² are as defined in the formula (C1).

37. The organic electroluminescence device according to claim 1, wherein in the formula (C1), (C10), (C11) or (C12),
one of A¹ and A² is a substituted or unsubstituted aryl group including 6 to 30 ring carbon atoms, and
the other of A¹ and A² is a substituted or unsubstituted phenyl group, a substituted or unsubstituted biphenyl group, a substituted or unsubstituted terphenyl group, a substituted or unsubstituted naphthyl group, a naphthylphenyl group, a triphenylenyl group, or a 9,9-biphenylfluorenyl group.

38. The organic electroluminescence device according to claim 1, wherein in the above formula (C1), (C10), (C11) or (C12),
one of A¹ and A² is a substituted or unsubstituted aryl group including 6 to 30 ring carbon atoms, and
the other of A¹ and A² is a substituted or unsubstituted phenyl group, a substituted or unsubstituted p-biphenyl group group, a substituted or unsubstituted m-biphenyl group, a substituted or unsubstituted o-biphenyl group, a substituted or unsubstituted 3-naphthylphenyl group, a triphenylenyl group, or a 9,9-biphenylfluorenyl group.

39. The organic electroluminescence device according to claim 1, wherein the organic layer further comprises a second layer,
the second layer is disposed between the cathode and the emitting layer, and
the second layer comprises a compound represented by the following formula (D1): 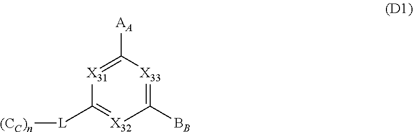
wherein in the formula (D1),
one or more among X₃₁ to X₃₃ are a nitrogen atom, and the others which are not a nitrogen atom are CR;
R is
a hydrogen atom, a cyano group,
a substituted or unsubstituted alkyl group including 1 to 50 carbon atoms,
a substituted or unsubstituted cycloalkyl group including 3 to 50 ring carbon atoms,
—Si(R₉₀₁)(R₉₀₂)(R₉₀₃),
—O—(R₉₀₄),
a substituted or unsubstituted aryl group including 6 to 50 ring carbon atoms, or
a substituted or unsubstituted monovalent heterocyclic group including 5 to 50 ring atoms;
R₉₀₁ to R₉₀₄ are as defined in the formula (A1);
A_{A} is a substituted or unsubstituted aryl group including 6 to 18 ring carbon atoms, or a substituted or unsubstituted monovalent heterocyclic group including 5 to 13 ring atoms;
B_{B} is a substituted or unsubstituted aryl group including 6 to 18 ring carbon atoms, or a substituted or unsubstituted monovalent heterocyclic group including 5 to 13 ring atoms;
L is a single bond, a substituted or unsubstituted (n+1)-valent aromatic hydrocarbon ring group including 6 to 18 ring carbon atoms, or a substituted or unsubstituted (n+1)-valent heterocyclic group including 5 to 13 ring atoms;
C_{C} is independently a substituted or unsubstituted aryl group including 6 to 30 ring carbon atoms, or a substituted or unsubstituted monovalent heterocyclic group including 5 to 60 ring atoms; and
n is an integer of 1 to 3; when n is 2 or more, L is not a single bond.

40. The organic electroluminescence device according to claim 39, wherein the compound represented by the formula (D1) is a compound represented by the following formula (D10): 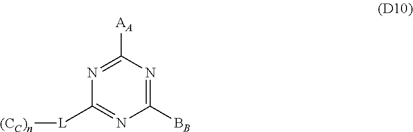
wherein in the formula (D10), A_{A}, B_{B}, C_{C}, L and n are as defined in the formula (D1).

41. An electronic appliance, wherein the organic electroluminescence device according to claim 1 is provided.
